SEQUENCE LISTING (247 sequences)

SEQ ID NO: 1 - protein, 58 aa, synthetic construct
VDNKFNKEVQMAQFEIRKLPNLNHHQSFAFIKSLMDDPSQSANLLAEAKKLNDAQAPK

SEQ ID NO: 2 - protein, 58 aa, synthetic construct
VDNKFNKEAQMAQFEIRKLPNLNHHQSFAFIKSLMDDPSQSANLLAEAKKLNDAQAPK

SEQ ID NO: 3 - protein, 58 aa, synthetic construct
VDNKFNKEVQAAQFEIRKLPNLNHHQSFAFIKSLMDDPSQSANLLAEAKKLNDAQAPK

SEQ ID NO: 4 - protein, 58 aa, synthetic construct
VDNKFNKEVQMAAFEIRKLPNLNHHQSFAFIKSLMDDPSQSANLLAEAKKLNDAQAPK

SEQ ID NO: 5 - protein, 58 aa, synthetic construct
VDNKFNKEVQMAQAEIRKLPNLNHHQSFAFIKSLMDDPSQSANLLAEAKKLNDAQAPK

SEQ ID NO: 6 - protein, 58 aa, synthetic construct
VDNKFNKEVQMAQFEIRALPNLNHHQSFAFIKSLMDDPSQSANLLAEAKKLNDAQAPK

SEQ ID NO: 7 - protein, 58 aa, synthetic construct
VDNKFNKEVQMAQFEIRKLPNLNHAQSFAFIKSLMDDPSQSANLLAEAKKLNDAQAPK

SEQ ID NO: 8 - protein, 58 aa, synthetic construct
VDNKFNKEVQMAQFEIRKLPNLNHHQAFAFIKSLMDDPSQSANLLAEAKKLNDAQAPK

SEQ ID NO: 9 - protein, 58 aa, synthetic construct
VDNKFNKEVQMAQFEIRKLPNLNHHQSFAFIASLMDDPSQSANLLAEAKKLNDAQAPK

SEQ ID NO: 10 - protein, 58 aa, unidentified
VDNKFNKEVQMAQFEIRKLPNLNHHQSFAFIKSLADDPSQSANLLAEAKKLNDAQAPK

SEQ ID NO: 11 - protein, 58 aa, synthetic construct
VDNKFNKELQSAQWEIRALTNLNHHQKFAFIESLRDDPSQSANLLAEAKKLNDAQAPK

SEQ ID NO: 12 - protein, 58 aa, synthetic construct
VDNKFNKEIQHAQWEIRHLPNLNHHQQFAFIKSLIDDPSQSANLLAEAKKLNDAQAPK

SEQ ID NO: 13 - protein, 58 aa, synthetic construct
VDNKFNKELQTAQLEIRTLPNLNHHQIFAFIAKLRDDPSQSANLLAEAKKLNDAQAPK

SEQ ID NO: 14 - protein, 58 aa, synthetic construct
VDNKFNKEFQLAQREIRELPNLNHHQQFAFIHKLLDDPSQSANLLAEAKKLNDAQAPK

SEQ ID NO: 15 - protein, 58 aa, synthetic construct
VDNKFNKETQVAQKEIRTLPNLNHHQIFAFLNKLIDDPSQSANLLAEAKKLNDAQAPK

SEQ ID NO: 16 - protein, 58 aa, synthetic construct
VDNKFNKEVQWAQKEIRSLPNLNHHQVFAFIRSLLDDPSQSANLLAEAKKLNDAQAPK

SEQ ID NO: 17 - protein, 58 aa, synthetic construct
VDNKFNKELQYAQKEIRRLPNLNHHQVFAFINKLIDDPSQSANLLAEAKKLNDAQAPK

SEQ ID NO: 18 - protein, 58 aa, synthetic construct
VDNKFNKEIQVAQSEIRALPNLNHHQIFAFIKKLLDDPSQSANLLAEAKKLNDAQAPK

SEQ ID NO: 19 - protein, 58 aa, synthetic construct
VDNKFNKEVQIAQREIRSLPNLNHHQVFAFIKKLLDDPSQSANLLAEAKKLNDAQAPK

SEQ ID NO: 20 - protein, 58 aa, synthetic construct
VDNKFNKERQYAVNEIRQLPNLNHHQIFAFIKSLLDDPSQSANLLAEAKKLNDAQAPK

SEQ ID NO: 21 - protein, 58 aa, synthetic construct
VDNKFNKEVQYAQREIRNLPNLNHHQAFAFIKSLIDDPSQSANLLAEAKKLNDAQAPK

SEQ ID NO: 22 - protein, 58 aa, synthetic construct
VDNKFNKEIQSAQVEIRALPNLNHHQKFAFINSLLDDPSQSANLLAEAKKLNDAQAPK

SEQ ID NO: 23 - protein, 58 aa, synthetic construct
VDNKFNKELQHAQIEIRKLPNLNHHQKFAFINKLHDDPSQSANLLAEAKKLNDAQAPK

SEQ ID NO: 24 - protein, 58 aa, synthetic construct
VDNKFNKEVQFAQREIRKLPNLNHHQQFAFIHSLLDDPSQSANLLAEAKKLNDAQAPK

SEQ ID NO: 25 - protein, 58 aa, synthetic construct
VDNKFNKELQSAQKEIRALPNLNHHQRFAFINSLLDDPSQSANLLAEAKKLNDAQAPK

SEQ ID NO: 26 - protein, 58 aa, synthetic construct
VDNKFNKEVQIAQKEIRELPNLNHHQKFAFIKSLLDDPSQSANLLAEAKKLNDAQAPK

SEQ ID NO: 27 - protein, 58 aa, synthetic construct
VDNKFNKELQHAQREIRQLPNLNHHQVFAFIASLIDDPSQSANLLAEAKKLNDAQAPK

SEQ ID NO: 28 - protein, 58 aa, synthetic construct
VDNKFNKEIQDAQKEIRTLPNLNHHQKFAFIQSLIDDPSQSANLLAEAKKLNDAQAPK

SEQ ID NO: 29 - protein, 58 aa, synthetic construct
VDNKFNKELQAAQKEIRTLPNLNHHQVFAFINKLIDDPSQSANLLAEAKKLNDAQAPK

SEQ ID NO: 30 - protein, 58 aa, synthetic construct
VDNKFNKEKQRAQWEIRQLPNLNHHQKFAFIQSLIDDPSQSANLLAEAKKLNDAQAPK

SEQ ID NO: 31 - protein, 58 aa, synthetic construct
VDNKFNKERQQAAYEIRHLPNLNHHQVFAFIKKLLDDPSQSANLLAEAKKLNDAQAPK

SEQ ID NO: 32 - protein, 58 aa, synthetic construct
VDNKFNKEKQAAQREIRALPNLNHHQIFAFIHKLLDDPSQSANLLAEAKKLNDAQAPK

SEQ ID NO: 33 - protein, 58 aa, synthetic construct
VDNKFNKELQSAQREIRQLPNLNHHQKFAFINSLLDDPSQSANLLAEAKKLNDAQAPK

SEQ ID NO: 34 - protein, 58 aa, synthetic construct
VDNKFNKEYQIAQWEIRRLPNLNHHQKFAFIASLLDDPSQSANLLAEAKKLNDAQAPK

SEQ ID NO: 35 - protein, 58 aa, synthetic construct
VDNKFNKELQNAQREIRALPNLNHHQQFAFIHKLIDDPSQSANLLAEAKKLNDAQAPK

SEQ ID NO: 36 - protein, 58 aa, synthetic construct
VDNKFNKEDQRAQREIRALPNLNHHQKFAFINKLLDDPSQSANLLAEAKKLNDAQAPK

SEQ ID NO: 37 - protein, 58 aa, synthetic construct
VDNKFNKETQIAAQEIRHLPNLNHHQRFAFIKSLLDDPSQSANLLAEAKKLNDAQAPK

SEQ ID NO: 38 - protein, 58 aa, synthetic construct
VDNKFNKEVQIAQTEIRSLPNLNHHQVFAFIQSLIDDPSQSANLLAEAKKLNDAQAPK

SEQ ID NO: 39 - protein, 58 aa, synthetic construct
VDNKFNKEIQNAQWEIRHLPNLNHHQTFAFIQKLIDDPSQSANLLAEAKKLNDAQAPK

SEQ ID NO: 40 - protein, 58 aa, synthetic construct
VDNKFNKEVQTAQWEIRHLPNLNHHQKFAFIRSLSDDPSQSANLLAEAKKLNDAQAPK

SEQ ID NO: 41 - protein, 58 aa, synthetic construct
VDNKFNKEQQFAQQEIRKLPNLNHHQRFAFIRSLIDDPSQSANLLAEAKKLNDAQAPK

SEQ ID NO: 42 - protein, 58 aa, synthetic construct
VDNKFNKELQAAQVEIRKLPNLNHHQTFAFIKSLIDDPSQSANLLAEAKKLNDAQAPK

SEQ ID NO: 43 - protein, 58 aa, synthetic construct
VDNKFNKEVQIAQLEIRRLPNLNHHQKYAFIKSLADDPSQSANLLAEAKKLNDAQAPK

SEQ ID NO: 44 - protein, 58 aa, synthetic construct
VDNKFNKEFQSAQVEIRHLPNLNHHQKFAFIQSLIDDPSQSANLLAEAKKLNDAQAPK

SEQ ID NO: 45 - protein, 58 aa, synthetic construct
VDNKFNKEKQQAQYEIRHLPNLNHHQAFAFIQKLIDDPSQSANLLAEAKKLNDAQAPK

SEQ ID NO: 46 - protein, 58 aa, synthetic construct
VDNKFNKEYQYAQKEIRTLPNLNHHQRFAFIKSLLDDPSQSANLLAEAKKLNDAQAPK

SEQ ID NO: 47 - protein, 58 aa, synthetic construct
VDNKFNKEYQQAQQEIRVLPNLNHHQKFAFIQKLIDDPSQSANLLAEAKKLNDAQAPK

SEQ ID NO: 48 - protein, 58 aa, synthetic construct
VDNKFNKEQQYAQIEIRQLPNLNHHQKFAFIKSLIDDPSQSANLLAEAKKLNDAQAPK

SEQ ID NO: 49 - protein, 58 aa, synthetic construct
VDNKFNKEQQEAQLEIRRLPNLNHHQVFAFIKSLLDDPSQSANLLAEAKKLNDAQAPK

SEQ ID NO: 50 - protein, 58 aa, synthetic construct
VDNKFNKEFQIAQREIRDLPNLNHHQIFAFIQSLIDDPSQSANLLAEAKKLNDAQAPK

SEQ ID NO: 51 - protein, 58 aa, synthetic construct
VDNKFNKEQQIAQYEIRKLPNLNHHQTFAFIKSLLDDPSQSANLLAEAKKLNDAQAPK

SEQ ID NO: 52 - protein, 58 aa, synthetic construct
VDNKFNKEQQWAQREIRSLPNLNHHQVFAFINSLLDDPSQSANLLAEAKKLNDAQAPK

SEQ ID NO: 53 - protein, 58 aa, synthetic construct
VDNKFNKELQYAQVEIRKLPNLNHHQRFAFIKSLLDDPSQSANLLAEAKKLNDAQAPK

SEQ ID NO: 54 - protein, 58 aa, synthetic construct
VDNKFNKEYQEAQLEIRNLPNLNHHQIFAFINKLIDDPSQSANLLAEAKKLNDAQAPK

SEQ ID NO: 55 - protein, 58 aa, synthetic construct
VDNKFNKELQKAQQEIRHLPNLNHHQTFAFIKKLLDDPSQSANLLAEAKKLNDAQAPK

SEQ ID NO: 56 - protein, 58 aa, synthetic construct
VDNKFNKEHQVAQREIRALPNLNHHQKFAFIQSLIDDPSQSANLLAEAKKLNDAQAPK

SEQ ID NO: 57 - protein, 58 aa, synthetic construct
VDNKFNKEFQQAAREIRVLPNLNHHQIFAFIKSLLDDPSQSANLLAEAKKLNDAQAPK

SEQ ID NO: 58 - protein, 58 aa, synthetic construct
VDNKFNKELQAAQVEIRTLPNLNHHQVFAFIQKLIDDPSQSANLLAEAKKLNDAQAPK

SEQ ID NO: 59 - protein, 58 aa, synthetic construct
VDNKFNKELQFAQREIRALPNLNHHQIFAFIRSLLDDPSQSANLLAEAKKLNDAQAPK

SEQ ID NO: 60 - protein, 58 aa, synthetic construct
VDNKFNKEVQHAQKEIRALPNLNHHQAFAFIAKLLDDPSQSANLLAEAKKLNDAQAPK

SEQ ID NO: 61 - protein, 58 aa, synthetic construct
VDNKFNKELQVAQHEIRFLPNLNHHQKFAFINKLIDDPSQSANLLAEAKKLNDAQAPK

SEQ ID NO: 62 - protein, 58 aa, synthetic construct
VDNKFNKEVQIAQLEIRALPNLNHHQIFAFIKKLHDDPSQSANLLAEAKKLNDAQAPK

SEQ ID NO: 63 - protein, 58 aa, synthetic construct
VDNKFNKEFQAAQQEIRRLPNLNHHQAFAFIRSLLDDPSQSANLLAEAKKLNDAQAPK

SEQ ID NO: 64 - protein, 58 aa, synthetic construct
VDNKFNKETQQAQREIRKLPNLNHHQQFAFIKSLLDDPSQSANLLAEAKKLNDAQAPK

SEQ ID NO: 65 - protein, 58 aa, synthetic construct
VDNKFNKELQSAQTEIQHLPNLNHAQQFAFILSLIDDPSQSANLLAEAKKLNDAQAPK

SEQ ID NO: 66 - protein, 58 aa, synthetic construct
VDNKFNKELQSAQREIRILPNLNHHQRFAFINSLLDDPSQSANLLAEAKKLNDAQAPK

SEQ ID NO: 67 - protein, 58 aa, synthetic construct
VDNKFNKELQSAQWEIRALPNLNHHQKFAFIESLRDDPSQSANLLAEAKKLNDAQAPK

SEQ ID NO: 68 - protein, 55 aa, synthetic construct
VDNKFNKEVQMAQFEIRKLPNLNHHQSFAFIKSLMDDPSQSANLLAEAKKLNDAQ

SEQ ID NO: 69 - skipped

SEQ ID NO: 70 - protein, 53 aa, synthetic construct
NKEVQMAQFEIRKLPNLNHHQSFAFIKSLMDDPSQSANLLAEAKKLNDAQAPK

SEQ ID NO: 71 - protein, 53 aa, synthetic construct
NKELQSAQTEIQHLPNLNHAQQFAFILSLIDDPSQSANLLAEAKKLNDAQAPK

SEQ ID NO: 72 - protein, 53 aa, synthetic construct
NKEIQHAQWEIRHLPNLNHHQQFAFIKSLIDDPSQSANLLAEAKKLNDAQAPK

SEQ ID NO: 73 - protein, 53 aa, synthetic construct
NKEVQIAQTEIRSLPNLNHHQVFAFIQSLIDDPSQSANLLAEAKKLNDAQAPK

SEQ ID NO: 74 - protein, 58 aa, synthetic construct
VDNKFNKEQFYARDEIDLLPNLNEDQKWAFYMSLIDDPSQSANLLAEAKKLNDAQAPK

SEQ ID NO: 75 - protein, 58 aa, synthetic construct
VDNKFNKEFWIAESEIESLPNLNIYQKWAFKYSLADDPSQSANLLAEAKKLNDAQAPK

SEQ ID NO: 76 - protein, 121 aa, synthetic construct
GSSHHHHHHYYLEVDNKFNKEVQMAQFEIRKLPNLNHHQSFAFIKSLMDDPSQSANLLAEAKKLNDAQAPKVDYVLAEAKVLANRELDKYGVSDYYKNLINNAKTVEGVKALIDEILAALP

SEQ ID NO: 77 - protein, 121 aa, synthetic construct
GSSHHHHHHYYLEVDNKFNKEQFYARDEIDLLPNLNEDQKWAFYMSLIDDPSQSANLLAEAKKLNDAQAPKVDYVLAEAKVLANRELDKYGVSDYYKNLINNAKTVEGVKALIDEILAALP

SEQ ID NO: 78 - protein, 121 aa, synthetic construct
GSSHHHHHHYYLEVDNKFNKEFWIAESEIESLPNLNIYQKWAFKYSLADDPSQSANLLAEAKKLNDAQAPKVDYVLAEAKVLANRELDKYGVSDYYKNLINNAKTVEGVKALIDEILAALP

SEQ ID NO: 79 - protein, 72 aa, synthetic construct
GSSHHHHHHYYLEVDNKFNKEVQMAQFEIRKLPNLNHHQSFAFIKSLMDDPSQSANLLAEAKKLNDAQAPKC

SEQ ID NO: 80 - protein, 72 aa, synthetic construct
GSSHHHHHHYYLEVDNKFNKEQFYARDEIDLLPNLNEDQKWAFYMSLIDDPSQSANLLAEAKKLNDAQAPKC

SEQ ID NO: 81 - protein, 145 aa, synthetic construct
GSSHHHHHHYYLEVDNKFNKEVQMAQFEIRKLPNLNHHQSFAFIKSLMDDPSQSANLLAEAKKLNDAQAPKGGGSGGGGSGGGGSGVDNKFNKEQFYARDEIDLLPNLNEDQKWAFYMSLIDDPSQSANLLAEAKKLNDAQAPKC

SEQ ID NO: 82 - protein, 145 aa, synthetic construct
GSSHHHHHHYYLEVDNKFNKEQFYARDEIDLLPNLNEDQKWAFYMSLIDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKC

SEQ ID NO: 83 - protein, 143 aa, synthetic construct
VDNKFNKEVQMAQFEIRKLPNLNHHQSFAFIKSLMDDPSQSANLLAEAKKLNDAQAPKQAPKGGGSGGGGSGGGGSGVDNKFNKEQFYARDEIDLLPNLNEDQKWAFYMSLIDDPSQSANLLAEAKKLNDAQAPKYYHHHHHH

SEQ ID NO: 84 - protein, 143 aa, synthetic construct
VDNKFNKEQFYARDEIDLLPNLNEDQKWAFYMSLIDDPSQSANLLAEAKKLNDAQAPKQAPKGGGSGGGGSGGGGSGVDNKFNKEVQMAQFEIRKLPNLNHHQSFAFIKSLMDDPSQSANLLAEAKKLNDAQAPKYYHHHHHH

SEQ ID NO: 85 - protein, 212 aa, synthetic construct
VDNKFNKENQFADEEIAALPNLNFYQKWAFIRKLMDDPSQSANLLAEAKKLNDAQAPKGGGSGGGGSGGGGSGVDNKFNKEFWIAESEIESLPNLNIYQKWAFKYSLADDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKYYHHHHHH

SEQ ID NO: 86 - protein, 212 aa, synthetic construct
VDNKFNKENQFADEEIAALPNLNFYQKWAFIRKLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKYYHHHHHH

SEQ ID NO: 87 - protein, 212 aa, synthetic construct
VDNKFNKENQFADEEIAALPNLNFYQKWAFIRKLMDDPSQSANLLAEAKKLNDAQAPKGGGSGGGGSGGGGSGVDNKFNKEQFYARDEIDLLPNLNEDQKWAFYMSLIDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKYYHHHHHH

SEQ ID NO: 88 - protein, 139 aa, synthetic construct
VDNKFNKENQFADEEIAALPNLNFYQKWAFIRKLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKYYHHHHHH

SEQ ID NO: 89 - protein, 139 aa, synthetic construct; identical to SEQ ID NO: 88

SEQ ID NO: 90 - protein, 139 aa, synthetic construct
VDNKFNKENQFADEEIAALPNLNFYQKWAFIRKLMDDPSQSANLLAEAKKLNDAQAPKGGGSGGGGSGGGGSGVDNKFNKEVQYAQREIRNLPNLNHHQAFAFIKSLIDDPSQSANLLAEAKKLNDAQAPKYYHHHHHH

SEQ ID NO: 91 - protein, 139 aa, synthetic construct
VDNKFNKENQFADEEIAALPNLNFYQKWAFIRKLMDDPSQSANLLAEAKKLNDAQAPKGGGSGGGGSGGGGSGVDNKFNKELQSAQKEIRALPNLNHHQRFAFINSLLDDPSQSANLLAEAKKLNDAQAPKYYHHHHHH

SEQ ID NO: 92 - protein, 139 aa, synthetic construct
VDNKFNKENQFADEEIAALPNLNFYQKWAFIRKLMDDPSQSANLLAEAKKLNDAQAPKGGGSGGGGSGGGGSGVDNKFNKEVQIAQTEIRSLPNLNHHQVFAFIQSLIDDPSQSANLLAEAKKLNDAQAPKYYHHHHHH

SEQ ID NO: 93 - protein, 139 aa, synthetic construct
VDNKFNKENQFADEEIAALPNLNFYQKWAFIRKLMDDPSQSANLLAEAKKLNDAQAPKGGGSGGGGSGGGGSGVDNKFNKELQSAQTEIQHLPNLNHAQQFAFILSLIDDPSQSANLLAEAKKLNDAQAPKYYHHHHHH

SEQ ID NO: 94 - protein, 139 aa, synthetic construct
VDNKFNKENQFADEEIAALPNLNFYQKWAFIRKLMDDPSQSANLLAEAKKLNDAQAPKGGGSGGGGSGGGGSGVDNKFNKELQYAQKEIRRLPNLNHHQVFAFINKLIDDPSQSANLLAEAKKLNDAQAPKYYHHHHHH

SEQ ID NO: 95 - protein, 139 aa, synthetic construct
VDNKFNKENQFADEEIAALPNLNFYQKWAFIRKLMDDPSQSANLLAEAKKLNDAQAPKGGGSGGGGSGGGGSGVDNKFNKEVQWAQKEIRSLPNLNHHQVFAFIRSLLDDPSQSANLLAEAKKLNDAQAPKYYHHHHHH

SEQ ID NO: 96 - protein, 139 aa, synthetic construct
VDNKFNKENQFADEEIAALPNLNFYQKWAFIRKLMDDPSQSANLLAEAKKLNDAQAPKGGGSGGGGSGGGGSGVDNKFNKELQSAQWEIRALTNLNHHQKFAFIESLRDDPSQSANLLAEAKKLNDAQAPKYYHHHHHH

SEQ ID NO: 97 - protein, 139 aa, synthetic construct
VDNKFNKENQFADEEIAALPNLNFYQKWAFIRKLMDDPSQSANLLAEAKKLNDAQAPKGGGSGGGGSGGGGSGVDNKFNKELQSAQREIRILPNLNHHQRFAFINSLLDDPSQSANLLAEAKKLNDAQAPKYYHHHHHH

SEQ ID NO: 98 - protein, 139 aa, synthetic construct
VDNKFNKENQFADEEIAALPNLNFYQKWAFIRKLMDDPSQSANLLAEAKKLNDAQAPKGGGSGGGGSGGGGSGVDNKFNKETQQAQREIRKLPNLNHHQQFAFIKSLLDDPSQSANLLAEAKKLNDAQAPKYYHHHHHH

SEQ ID NO: 99 - protein, 139 aa, synthetic construct
VDNKFNKENQFADEEIAALPNLNFYQKWAFIRKLMDDPSQSANLLAEAKKLNDAQAPKGGGSGGGGSGGGGSGVDNKFNKEIQHAQWEIRHLPNLNHHQQFAFIKSLIDDPSQSANLLAEAKKLNDAQAPKYYHHHHHH

SEQ ID NO: 100 - protein, 139 aa, synthetic construct
VDNKFNKENQFADEEIAALPNLNFYQKWAFIRKLMDDPSQSANLLAEAKKLNDAQAPKGGGSGGGGSGGGGSGVDNKFNKELQSAQWEIRALPNLNHHQKFAFIESLRDDPSQSANLLAEAKKLNDAQAPKYYHHHHHH

SEQ ID NO: 101 - protein, 270 aa, synthetic construct
MMVDNKFNKENQFADEEIAALPNLNFYQKWAFIRKLMDDPSQSANLLAEAKKLNDAQAPKGGGSGGGGSGGGGSGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSGGGGSGGGGSGVDNKFNKEVQMAQFEIRKLPNLNHHQSFAFIKSLMDDPSQSANLLAEAKKLNDAQAPKYYHHHHHH

SEQ ID NO: 102 - protein, 194 aa, synthetic construct
MMVDNKFNKENQFADEEIAALPNLNFYQKWAFIRKLMDDPSQSANLLAEAKKLNDAQAPKGGGSGVDNKFNKENQFADEEIAALPNLNFYQKWAFIRKLMDDPSQSANLLAEAKKLNDAQAPKGGGSGVDNKFNKEVQMAQFEIRKLPNLNHHQSFAFIKSLMDDPSQSANLLAEAKKLNDAQAPKYYHHHHHH

SEQ ID NO: 103 - protein, 204 aa, synthetic construct
MMVDNKFNKENQFADEEIAALPNLNFYQKWAFIRKLMDDPSQSANLLAEAKKLNDAQAPKGGGSGGGGSGVDNKFNKENQFADEEIAALPNLNFYQKWAFIRKLMDDPSQSANLLAEAKKLNDAQAPKGGGSGGGGSGVDNKFNKEVQMAQFEIRKLPNLNHHQSFAFIKSLMDDPSQSANLLAEAKKLNDAQAPKYYHHHHHH

SEQ ID NO: 104 - protein, 214 aa, synthetic construct
MMVDNKFNKENQFADEEIAALPNLNFYQKWAFIRKLMDDPSQSANLLAEAKKLNDAQAPKGGGSGGGGSGGGGSGVDNKFNKENQFADEEIAALPNLNFYQKWAFIRKLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKYYHHHHHH

SEQ ID NO: 105 - protein, 214 aa, synthetic construct
MMVDNKFNKENQFADEEIAALPNLNFYQKWAFIRKLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKGGGSGGGGSGGGGSGVDNKFNKENQFADEEIAALPNLNFYQKWAFIRKLMDDPSQSANLLAEAKKLNDAQAPKYYHHHHHH

SEQ ID NO: 106 - protein, 214 aa, synthetic construct
MMVDNKFNKEVQMAQFEIRKLPNLNHHQSFAFIKSLMDDPSQSANLLAEAKKLNDAQAPKGGGSGGGGSGGGGSGVDNKFNKENQFADEEIAALPNLNFYQKWAFIRKLMDDPSQSANLLAEAKKLNDAQAPKGGGSGGGGSGGGGSGVDNKFNKENQFADEEIAALPNLNFYQKWAFIRKLMDDPSQSANLLAEAKKLNDAQAPKYYHHHHHH

SEQ ID NO: 107-108 - skipped (2 entries)

SEQ ID NO: 109 - protein, 124 aa, synthetic construct
VDNKFNKENQFADEEIAALPNLNFYQKWAFIRKLMDDPSQSANLLAEAKKLNDAQAPKVDNKFNKEVQMAQFEIRKLPNLNHHQSFAFIKSLMDDPSQSANLLAEAKKLNDAQAPKYYHHHHHH

SEQ ID NO: 110 - protein, 124 aa, synthetic construct
VDNKFNKEVQMAQFEIRKLPNLNHHQSFAFIKSLMDDPSQSANLLAEAKKLNDAQAPKVDNKFNKENQFADEEIAALPNLNFYQKWAFIRKLMDDPSQSANLLAEAKKLNDAQAPKYYHHHHHH

SEQ ID NO: 111 - protein, 125 aa, synthetic construct
VDNKFNKENQFADEEIAALPNLNFYQKWAFIRKLMDDPSQSANLLAEAKKLNDAQAPKGVDNKFNKEVQMAQFEIRKLPNLNHHQSFAFIKSLMDDPSQSANLLAEAKKLNDAQAPKYYHHHHHH

SEQ ID NO: 112 - protein, 125 aa, synthetic construct
VDNKFNKEVQMAQFEIRKLPNLNHHQSFAFIKSLMDDPSQSANLLAEAKKLNDAQAPKGVDNKFNKENQFADEEIAALPNLNFYQKWAFIRKLMDDPSQSANLLAEAKKLNDAQAPKYYHHHHHH

SEQ ID NO: 113 - protein, 125 aa, synthetic construct; identical to SEQ ID NO: 111

SEQ ID NO: 114 - protein, 113 aa, synthetic construct
MNKENQFADEEIAALPNLNFYQKWAFIRKLMDDPSQSANLLAEAKKLNDAQGNKEVQMAQFEIRKLPNLNHHQSFAFIKSLMDDPSQSANLLAEAKKLNDAQAPKYYHHHHHH

SEQ ID NO: 115 - protein, 115 aa, synthetic construct
MNKENQFADEEIAALPNLNFYQKWAFIRKLMDDPSQSANLLAEAKKLNDAQGVDNKFNKEVQMAQFEIRKLPNLNHHQSFAFIKSLMDDPSQSANLLAEAKKLNDAQYYHHHHHH

SEQ ID NO: 116 - protein, 118 aa, synthetic construct
MNKENQFADEEIAALPNLNFYQKWAFIRKLMDDPSQSANLLAEAKKLNDAQGVDNKFNKEVQMAQFEIRKLPNLNHHQSFAFIKSLMDDPSQSANLLAEAKKLNDAQAPKYYHHHHHH

SEQ ID NO: 117 - protein, 120 aa, synthetic construct
GDNKFNKENQFADEEIAALPNLNFYQKWAFIRKLMDDPSQSANLLAEAKKLNDAQAPKGNKEVQMAQFEIRKLPNLNHHQSFAFIKSLMDDPSQSANLLAEAKKLNDAQAPKYYHHHHHH

SEQ ID NO: 118 - protein, 122 aa, synthetic construct
GDNKFNKENQFADEEIAALPNLNFYQKWAFIRKLMDDPSQSANLLAEAKKLNDAQAPKGVDNKFNKEVQMAQFEIRKLPNLNHHQSFAFIKSLMDDPSQSANLLAEAKKLNDAQYYHHHHHH

SEQ ID NO: 119 - protein, 125 aa, synthetic construct
GDNKFNKENQFADEEIAALPNLNFYQKWAFIRKLMDDPSQSANLLAEAKKLNDAQAPKGVDNKFNKEVQMAQFEIRKLPNLNHHQSFAFIKSLMDDPSQSANLLAEAKKLNDAQAPKYYHHHHHH

SEQ ID NO: 120 - protein, 12 aa, synthetic construct
  VARIANT 1: note = X is H
  VARIANT 2: note = X is A or H
  VARIANT 4: note = X is A, I, K, Q, R, S, T, or V
  VARIANT 5: note = X is F or Y
  VARIANT 8: note = J is I or L
  VARIANT 9: note = X is A, E, H, K, L, N, Q, or R
  VARIANT 10: note = X is K or S
  VAR_SEQ 12: note = X is A, H, I, L, M, R, or S
XXQXXAFJXXLX

SEQ ID NO: 121 - protein, 10 aa, synthetic construct
VQMAQFEIRK

SEQ ID NO: 122 - protein, 12 aa, synthetic construct
HHQSFAFIKSLM

SEQ ID NO: 123 - protein, 10 aa, synthetic construct
QFYARDEIDL

SEQ ID NO: 124 - protein, 12 aa, synthetic construct
EDQKWAFYMSLI

SEQ ID NO: 125 - protein, 10 aa, synthetic construct
FWIAESEIES

SEQ ID NO: 126 - protein, 12 aa, synthetic construct
IYQKWAFKYSLA

SEQ ID NO: 127 - protein, 10 aa, synthetic construct
  VAR_SEQ 1: note = X is A, D, F, H, I, K, L, Q, R, T, V, or Y
  VAR_SEQ 3: note = X is A, D, E, F, H, I, K, L, M, N, Q, R, S, T, V, W, or Y
  VAR_SEQ 5: note = X is A, Q or V
  VAR_SEQ 6: note = X is A, F, H, I, K, L, N, Q, R, S, T, V, W, or Y
  VAR_SEQ 9: note = X is Q or R
  VAR_SEQ 10: note = X is A, D, E, F, H, I, K, N, Q, R, S, T, or V
XQXAXXEIXX

SEQ ID NO: 128 - protein, 15 aa, synthetic construct
  VARIANT 1: note = X is A, R, N, D, C, Q, E, G, H, I, L, K, M, F, P, S, T, W, Y, V or deleted
  VARIANT 14: note = X is A, R, N, D, C, Q, E, G, H, I, L, K, M, F, P, S, T, W, Y, or V
  VARIANT 15: note = X is A, R, N, D, C, Q, E, G, H, I, L, K, M, F, P, S, T, W, Y, or V
XXXQXXAFXXXLXXX

SEQ ID NO: 129 - protein, 14 aa, synthetic construct
NKEVQMAQFEIRKL

SEQ ID NO: 130 - protein, 15 aa, synthetic construct
NHHQSFAFIKSLMDD

SEQ ID NO: 131 - protein, 14 aa, synthetic construct
NKEQFYARDEIDLL

SEQ ID NO: 132 - protein, 15 aa, synthetic construct
NEDQKWAFYMSLIDD

SEQ ID NO: 133 - protein, 14 aa, synthetic construct
NKEFWIAESEIESL

SEQ ID NO: 134 - protein, 15 aa, synthetic construct
NIYQKWAFKYSLADD

SEQ ID NO: 135 - protein, 21 aa, synthetic construct
  VARIANT 1: note = X is a sequence of 1 to 14 amino acids where each amino acid can be independently A, R, N, D, C, G, Q, E, H, I, L, K, M, F, P, S, T, W, Y, or V
  VARIANT 2: note = X is A, R, N, D, C, Q, E, G, H, I, L, K, M, F, P, S, T, W, Y, or V
  VARIANT 19: note = X is A, R, N, D, C, Q, E, G, H, I, L, K, M, F, P, S, T, W, Y, V, or deleted
  VARIANT 20: note = X is A, R, N, D, C, Q, E, G, H, I, L, K, M, F, P, S, T, W, Y, V, or deleted
  VARIANT 21: note = X is A, R, N, D, C, Q, E, G, H, I, L, K, M, F, P, S, T, W, Y, V, or deleted
XXQSANLLAEAKKLNDAQXXX

SEQ ID NO: 136 - protein, 16 aa, synthetic construct
QSANLLAEAKKLNDAQ

SEQ ID NO: 137 - protein, 21 aa, synthetic construct
  VARIANT 19: note = X is A or deleted
  VARIANT 20: note = X is P or deleted
  VARIANT 21: note = X is K or deleted
PSQSANLLAEAKKLNDAQXXX

SEQ ID NO: 138 - protein, 5 aa, synthetic construct
VDNKF

SEQ ID NO: 139 - protein, 5 aa, synthetic construct
GGGSG

SEQ ID NO: 140 - protein, 5 aa, synthetic construct
GGGGS

SEQ ID NO: 141 - protein, 5 aa, synthetic construct
GGSGG

SEQ ID NO: 142 - protein, 5 aa, synthetic construct
GSGGG

SEQ ID NO: 143 - protein, 5 aa, synthetic construct
SGGGG

SEQ ID NO: 144 - protein, 10 aa, synthetic construct
GGGSGGGGSG

SEQ ID NO: 145 - protein, 15 aa, synthetic construct
GGGSGGGGSGGGGSG

SEQ ID NO: 146 - protein, 20 aa, synthetic construct
GGGSGGGGSGGGGSGGGGSG

SEQ ID NO: 147 - protein, 10 aa, synthetic construct
GGSGGGGSGG

SEQ ID NO: 148 - protein, 15 aa, synthetic construct
GGSGGGGSGGGGSGG

SEQ ID NO: 149 - protein, 20 aa, synthetic construct
GGSGGGGSGGGGSGGGGSGG

SEQ ID NO: 150 - protein, 27 aa, synthetic construct
VQMAQFEIRKLPNLNHHQSFAFIKSLM

SEQ ID NO: 151 - protein, 27 aa, synthetic construct
AQMAQFEIRKLPNLNHHQSFAFIKSLM

SEQ ID NO: 152 - protein, 27 aa, synthetic construct
VQAAQFEIRKLPNLNHHQSFAFIKSLM

SEQ ID NO: 153 - protein, 27 aa, synthetic construct
VQMAAFEIRKLPNLNHHQSFAFIKSLM

SEQ ID NO: 154 - protein, 27 aa, synthetic construct
VQMAQAEIRKLPNLNHHQSFAFIKSLM

SEQ ID NO: 155 - protein, 27 aa, synthetic construct
VQMAQFEIRALPNLNHHQSFAFIKSLM

SEQ ID NO: 156 - protein, 27 aa, synthetic construct
VQMAQFEIRKLPNLNHAQSFAFIKSLM

SEQ ID NO: 157 - protein, 27 aa, synthetic construct
VQMAQFEIRKLPNLNHHQAFAFIKSLM

SEQ ID NO: 158 - protein, 27 aa, synthetic construct
VQMAQFEIRKLPNLNHHQSFAFIASLM

SEQ ID NO: 159 - protein, 27 aa, synthetic construct
VQMAQFEIRKLPNLNHHQSFAFIKSLA

SEQ ID NO: 160 - protein, 27 aa, synthetic construct
LQSAQWEIRALTNLNHHQKFAFIESLR

SEQ ID NO: 161 - protein, 27 aa, synthetic construct
IQHAQWEIRHLPNLNHHQQFAFIKSLI

SEQ ID NO: 162 - protein, 27 aa, synthetic construct
LQTAQLEIRTLPNLNHHQIFAFIAKLR

SEQ ID NO: 163 - protein, 27 aa, synthetic construct
FQLAQREIRELPNLNHHQQFAFIHKLL

SEQ ID NO: 164 - protein, 27 aa, synthetic construct
TQVAQKEIRTLPNLNHHQIFAFLNKLI

SEQ ID NO: 165 - protein, 27 aa, synthetic construct
VQWAQKEIRSLPNLNHHQVFAFIRSLL

SEQ ID NO: 166 - protein, 27 aa, synthetic construct
LQYAQKEIRRLPNLNHHQVFAFINKLI

SEQ ID NO: 167 - protein, 27 aa, synthetic construct
IQVAQSEIRALPNLNHHQIFAFIKKLL

SEQ ID NO: 168 - protein, 27 aa, synthetic construct
VQIAQREIRSLPNLNHHQVFAFIKKLL

SEQ ID NO: 169 - protein, 27 aa, synthetic construct
RQYAVNEIRQLPNLNHHQIFAFIKSLL

SEQ ID NO: 170 - protein, 27 aa, synthetic construct
VQYAQREIRNLPNLNHHQAFAFIKSLI

SEQ ID NO: 171 - protein, 27 aa, synthetic construct
IQSAQVEIRALPNLNHHQKFAFINSLL

SEQ ID NO: 172 - protein, 27 aa, synthetic construct
LQHAQIEIRKLPNLNHHQKFAFINKLH

SEQ ID NO: 173 - protein, 27 aa, synthetic construct
VQFAQREIRKLPNLNHHQQFAFIHSLL

SEQ ID NO: 174 - protein, 27 aa, synthetic construct
LQSAQKEIRALPNLNHHQRFAFINSLL

SEQ ID NO: 175 - protein, 27 aa, synthetic construct
VQIAQKEIRELPNLNHHQKFAFIKSLL

SEQ ID NO: 176 - protein, 27 aa, synthetic construct
LQHAQREIRQLPNLNHHQVFAFIASLI

SEQ ID NO: 177 - protein, 27 aa, synthetic construct
IQDAQKEIRTLPNLNHHQKFAFIQSLI

SEQ ID NO: 178 - protein, 27 aa, synthetic construct
LQAAQKEIRTLPNLNHHQVFAFINKLI

SEQ ID NO: 179 - protein, 27 aa, synthetic construct
KQRAQWEIRQLPNLNHHQKFAFIQSLI

SEQ ID NO: 180 - protein, 27 aa, synthetic construct
RQQAAYEIRHLPNLNHHQVFAFIKKLL

SEQ ID NO: 181 - protein, 27 aa, synthetic construct
KQAAQREIRALPNLNHHQIFAFIHKLL

SEQ ID NO: 182 - protein, 27 aa, synthetic construct
LQSAQREIRQLPNLNHHQKFAFINSLL

SEQ ID NO: 183 - protein, 27 aa, synthetic construct
YQIAQWEIRRLPNLNHHQKFAFIASLL

SEQ ID NO: 184 - protein, 27 aa, synthetic construct
LQNAQREIRALPNLNHHQQFAFIHKLI

SEQ ID NO: 185 - protein, 27 aa, synthetic construct
DQRAQREIRALPNLNHHQKFAFINKLL

SEQ ID NO: 186 - protein, 27 aa, synthetic construct
TQIAAQEIRHLPNLNHHQRFAFIKSLL

SEQ ID NO: 187 - protein, 27 aa, synthetic construct
VQIAQTEIRSLPNLNHHQVFAFIQSLI

SEQ ID NO: 188 - protein, 27 aa, synthetic construct
IQNAQWEIRHLPNLNHHQTFAFIQKLI

SEQ ID NO: 189 - protein, 27 aa, synthetic construct
VQTAQWEIRHLPNLNHHQKFAFIRSLS

SEQ ID NO: 190 - protein, 27 aa, synthetic construct
QQFAQQEIRKLPNLNHHQRFAFIRSLI

SEQ ID NO: 191 - protein, 27 aa, synthetic construct
LQAAQVEIRKLPNLNHHQTFAFIKSLI

SEQ ID NO: 192 - protein, 27 aa, synthetic construct
VQIAQLEIRRLPNLNHHQKYAFIKSLA

SEQ ID NO: 193 - protein, 27 aa, synthetic construct
FQSAQVEIRHLPNLNHHQKFAFIQSLI

SEQ ID NO: 194 - protein, 27 aa, synthetic construct
KQQAQYEIRHLPNLNHHQAFAFIQKLI

SEQ ID NO: 195 - protein, 27 aa, synthetic construct
YQYAQKEIRTLPNLNHHQRFAFIKSLL

SEQ ID NO: 196 - protein, 27 aa, synthetic construct
YQQAQQEIRVLPNLNHHQKFAFIQKLI

SEQ ID NO: 197 - protein, 27 aa, synthetic construct
QQYAQIEIRQLPNLNHHQKFAFIKSLI

SEQ ID NO: 198 - protein, 27 aa, synthetic construct
QQEAQLEIRRLPNLNHHQVFAFIKSLL

SEQ ID NO: 199 - protein, 27 aa, synthetic construct
FQIAQREIRDLPNLNHHQIFAFIQSLI

SEQ ID NO: 200 - protein, 27 aa, synthetic construct
QQIAQYEIRKLPNLNHHQTFAFIKSLL

SEQ ID NO: 201 - protein, 27 aa, synthetic construct
QQWAQREIRSLPNLNHHQVFAFINSLL

SEQ ID NO: 202 - protein, 27 aa, synthetic construct
LQYAQVEIRKLPNLNHHQRFAFIKSLL

SEQ ID NO: 203 - protein, 27 aa, synthetic construct
YQEAQLEIRNLPNLNHHQIFAFINKLI

SEQ ID NO: 204 - protein, 27 aa, synthetic construct
LQKAQQEIRHLPNLNHHQTFAFIKKLL

SEQ ID NO: 205 - protein, 27 aa, synthetic construct
HQVAQREIRALPNLNHHQKFAFIQSLI

SEQ ID NO: 206 - protein, 27 aa, synthetic construct
FQQAAREIRVLPNLNHHQIFAFIKSLL

SEQ ID NO: 207 - protein, 27 aa, synthetic construct
LQAAQVEIRTLPNLNHHQVFAFIQKLI

SEQ ID NO: 208 - protein, 27 aa, synthetic construct
LQFAQREIRALPNLNHHQIFAFIRSLL

SEQ ID NO: 209 - protein, 27 aa, synthetic construct
VQHAQKEIRALPNLNHHQAFAFIAKLL

SEQ ID NO: 210 - protein, 27 aa, synthetic construct
LQVAQHEIRFLPNLNHHQKFAFINKLI

SEQ ID NO: 211 - protein, 27 aa, synthetic construct
VQIAQLEIRALPNLNHHQIFAFIKKLH

SEQ ID NO: 212 - protein, 27 aa, synthetic construct
FQAAQQEIRRLPNLNHHQAFAFIRSLL

SEQ ID NO: 213 - protein, 27 aa, synthetic construct
TQQAQREIRKLPNLNHHQQFAFIKSLL

SEQ ID NO: 214 - protein, 27 aa, synthetic construct
LQSAQTEIQHLPNLNHAQQFAFILSLI

SEQ ID NO: 215 - protein, 27 aa, synthetic construct
LQSAQREIRILPNLNHHQRFAFINSLL

SEQ ID NO: 216 - protein, 27 aa, synthetic construct
LQSAQWEIRALPNLNHHQKFAFIESLR

SEQ ID NO: 217 - protein, 27 aa, synthetic construct
LQSAQTEIQHLPNLNHAQQFAFILSLI

SEQ ID NO: 218 - protein, 27 aa, synthetic construct
IQHAQWEIRHLPNLNHHQQFAFIKSLI

SEQ ID NO: 219 - protein, 27 aa, synthetic construct
VQIAQTEIRSLPNLNHHQVFAFIQSLI

SEQ ID NO: 220 - protein, 27 aa, synthetic construct
QFYARDEIDLLPNLNEDQKWAFYMSLI

SEQ ID NO: 221 - protein, 27 aa, synthetic construct
FWIAESEIESLPNLNIYQKWAFKYSLA

SEQ ID NO: 222 - DNA, 121 bp, synthetic construct
  source 1..121: mol_type = other DNA
  misc_difference 21..23: note = nnn occupied by a codon for any one of the 20 standard amino acids, except C and P, with each codon selected with equal probability
  misc_difference 33..35: note = nnn occupied by a codon for I with a 60% probability or a codon for any one of H, Y, K, or D with 10% probability each
  misc_difference 42..44: note = nnn occupied by a codon for any one of the 20 standard amino acids, except C and P, with each codon selected with equal probability
  misc_difference 45..47: note = nnn occupied by a codon for any one of the 20 standard amino acids, except C and P, with each codon selected with equal probability
  misc_difference 51..53: note = nnn occupied by a codon for any one of the 20 standard amino acids, except C and P, with each codon selected with equal probability
  misc_difference 54..56: note = nnn occupied by a codon for any one of the 20 standard amino acids, except C and P, with each codon selected with equal probability
  misc_difference 72..74: note = nnn occupied by a codon for any one of the 20 standard amino acids, except C and P, with each codon selected with equal probability
  misc_difference 75..77: note = nnn occupied by a codon for any one of the 20 standard amino acids, except C and P, with each codon selected with equal probability
  misc_difference 84..86: note = nnn occupied by a codon for any one of the 20 standard amino acids, except C and P, with each codon selected with equal probability
  misc_difference 87..89: note = nnn occupied by a codon for any one of the 20 standard amino acids, except C and P, with each codon selected with equal probability
  misc_difference 93..95: note = nnn occupied by a codon for any one of the 20 standard amino acids, except C and P, with each codon selected with equal probability
  misc_difference 96..98: note = nnn occupied by a codon for any one of the 20 standard amino acids, except C and P, with each codon selected with equal probability
  misc_difference 99..101: note = nnn occupied by a codon for any one of the 20 standard amino acids, except C and P, with each codon selected with equal probability
gcgctttggcttgggtcatcnnntaaactnnnnnngaaggcnnnnnnttgnnnnnngttcaggttcggcagnnnnnngatctcnnnnnncgcnnnnnnnnnttctttgttgaatttgttgt

SEQ ID NO: 223 - DNA, 39 bp, synthetic construct
  source 1..39: mol_type = other DNA
  misc_difference 21..23
gatgaagccctcgaggtagacaacaaattcaacaaagaa

SEQ ID NO: 224 - DNA, 41 bp, synthetic construct
  source 1..41: mol_type = other DNA
ttagcttctgctagcaagttagcgctttggcttgggtcatc

SEQ ID NO: 225 - protein, 15 aa, synthetic construct
GGGGSGGGGSGGGGS

SEQ ID NO: 226 - protein, 58 aa, synthetic construct
VDNKFNKENQFADEEIAALPNLNFYQKWAFIRKLMDDPSQSANLLAEAKKLNDAQAPK

SEQ ID NO: 227 - DNA, 65 bp, synthetic construct
  source 1..65: mol_type = other DNA
  misc_difference 21..23: note = nnn occupied by a codon for A with 10 % probability or a codon for any of the other 19 standard amino acids, except M, C, P or G, with 90% probability split equally between each of the 15
  misc_difference 24..26: note = nnn occupied by a codon for Q with 10 % probability or a codon for any of the other 19 standard amino acids, except M, C, P or G, with 90% probability split equally between each of the 15
  misc_difference 27..29: note = nnn occupied by a codon for I with 15 % probability or a codon for any of the other 19 standard amino acids, except M, C, P or G, with 85% probability split equally between each of the 15
  misc_difference 33..35: note = nnn occupied by a codon for A with 10 % probability or a codon for any of the other 19 standard amino acids, except M, C, P or G, with 90% probability split equally between each of the 15
  misc_difference 36..38: note = nnn occupied by a codon for A with 10 % probability or a codon for any of the other 19 standard amino acids, except M, C, P or G, with 90% probability split equally between each of the 15
  misc_difference 45..47: note = nnn occupied by a codon for R with 90 % probability or a codon for any of the other 19 standard amino acids, except M, C, P or G, with 10% probability split equally between each of the 15
  misc_difference 48..50: note = nnn occupied by a codon for A with 10 % probability or a codon for any of the other 19 standard amino acids, except M, C, P or G, with 90% probability split equally between each of the 15
acaacaaattcaacaaagaannnnnnnnngcgnnnnnngagatcnnnnnnctgccgaacctgaac

SEQ ID NO: 228 - DNA, 69 bp, synthetic construct
  source 1..69: mol_type = other DNA
  misc_difference 19..21: note = nnn occupied by a codon for I with 15 % probability or a codon for any of the other 19 standard amino acids, except M, C, P or G, with 85% probability split equally between each of the 15
  misc_difference 25..27: note = nnn occupied by a codon for S with 5 % probability or a codon for any of the other 19 standard amino acids, except M, C, P or G, with 15% probability split equally between each of the 15
  misc_difference 28..30: note = nnn occupied by a codon for A with 10 % probability or a codon for any of the other 19 standard amino acids, except M, C, P or G, with 90% probability split equally between each of the 15
  misc_difference 31..33: note = nnn occupied by a codon for I with 90 % probability or a codon for any of the other 19 standard amino acids, except M, C, P or G, with 10% probability split equally between each of the 15
  misc_difference 40..42: note = nnn occupied by a codon for F with 90 % probability or a codon for any of the other 19 standard amino acids, except M, C, P or G, with 10% probability split equally between each of the 15
  misc_difference 43..45: note = nnn occupied by a codon for A with 90 % probability or a codon for any of the other 19 standard amino acids, except M, C, P or G, with 10% probability split equally between each of the 15
  misc_difference 49..51: note = nnn occupied by a codon for H with 65 % probability or a codon for any of the other 19 standard amino acids, except M, C, P or G, with 35% probability split equally between each of the 15
  misc_difference 52..54: note = nnn occupied by a codon for H with 90 % probability or a codon for any of the other 19 standard amino acids, except M, C, P or G, with 10% probability split equally between each of the 15
actctggctcggatcatcnnncagnnnnnnnnngaatgcnnnnnnctgnnnnnngttcaggttcggcag

SEQ ID NO: 229 - protein, 281 aa, synthetic construct
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFHIPKATLKDSGSYFCRGLFGSKNVSSETVNITITQGLAVSTISSFFPPGYQGGGSGVDNKFNKETQEASWEIFTLPNLNGRQVAAFISSLLDDPSQSANLLAEAKKLNDAQAPKGGGSGGLNDIFEAQKIEWHEHHHHHH

SEQ ID NO: 230 - protein, 10 aa, synthetic construct
VQMAQFEIRK

SEQ ID NO: 231 - protein, 12 aa, synthetic construct
HHQSFAFIKSLM

SEQ ID NO: 232 - protein, 14 aa, synthetic construct
NKEVQMAQFEIRKL

SEQ ID NO: 233 - protein, 15 aa, synthetic construct
NHHQSFAFIKSLMDD

SEQ ID NO: 234 - protein, 10 aa, synthetic construct
QFYARDEIDL

SEQ ID NO: 235 - protein, 12 aa, synthetic construct
EDQKWAFYMSLI

SEQ ID NO: 236 - protein, 14 aa, synthetic construct
NKEQFYARDEIDLL

SEQ ID NO: 237 - protein, 15 aa, synthetic construct
NEDQKWAFYMSLIDD

SEQ ID NO: 238 - protein, 10 aa, synthetic construct
FWIAESEIES

SEQ ID NO: 239 - protein, 12 aa, synthetic construct
IYQKWAFKYSLA

SEQ ID NO: 240 - protein, 14 aa, synthetic construct
NKEFWIAESEIESL

SEQ ID NO: 241 - protein, 15 aa, synthetic construct
NIYQKWAFKYSLADD

SEQ ID NO: 242 - protein, 131 aa, synthetic construct
VDNKFNKEVQMAQFEIRKLPNLNHHQSFAFIKSLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPK

SEQ ID NO: 243 - protein, 131 aa, synthetic construct
VDNKFNKEQFYARDEIDLLPNLNEDQKWAFYMSLIDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPK

SEQ ID NO: 244 - protein, 131 aa, synthetic construct
VDNKFNKEFWIAESEIESLPNLNIYQKWAFKYSLADDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPK

SEQ ID NO: 245 - DNA, 17 bp, synthetic construct
  source 1..17: mol_type = other DNA
gacgagtttaaaaacac

SEQ ID NO: 246 - DNA, 17 bp, synthetic construct
  source 1..17: mol_type = other DNA
gagcttaataatttctt

SEQ ID NO: 247 - DNA, 17 bp, synthetic construct
  source 1..17: mol_type = other DNA
gtgaccaattcagtgaa

(19) United States
(12) Patent Application Publication
(10) Pub. No.: US 2025/0333445 A1
(43) Pub. Date: Oct. 30, 2025

(54) NOVEL POLYPEPTIDES

(71) Applicant: Oncopeptides Innovation 1 AB, Stockholm (SE)

(72) Inventors: Per-Ake NYGREN, Stockholm (SE); Kim Anh GIANG, Stockholm (SE); Johan NILVEBRANT, Stockholm (SE); Fredrik LEHMANN, Stockholm (SE); Stefan Svensson GELIUS, Stockholm (SE)

(73) Assignee: Oncopeptides Innovation 1 AB, Stockholm (SE)

(21) Appl. No.: 18/870,286

(22) PCT Filed: May 31, 2023
(86) PCT No.: PCT/EP2023/064624

§ 371 Date: Nov. 27, 2024

(30) Foreign Application Priority Data
May 31, 2022 (GB) 2208027.9
Oct. 6, 2022 (GB) 2214718.5

(51) Int. Cl. C07K 14/00; A61K 45/06; A61K 47/66; A61P 37/02; C12N 15/10

(52) U.S. Cl. CPC C07K 14/001; A61K 45/06; A61K 47/66; A61P 37/02; C12N 15/1037; C07K 2319/21

(57) ABSTRACT

The invention provides a CD16a-binding polypeptide which comprises at least one motif that binds to CD16a, wherein said polypeptide comprises the following structure: [N-terminal portion]-[Helix 1]-[Separating portion]-[Helix 2]-[C-terminal portion] the CD16a-binding motif being the portion [Helix 1]-[Separating portion]-[Helix 2]. The invention further provides pharmaceutical compositions comprising the CD16a-binding polypeptide, and the use of the CD16a-binding polypeptide or pharmaceutical compositions as a medicament, particularly for use in the treatment or prophylaxis of cancers such as multiple myeloma.

### FIELD OF THE INVENTION

The present invention relates to immune cell-engaging polypeptides comprising at least one CD16a-binding polypeptide. The present invention also relates to pharmaceutical compositions comprising said immune cell-engaging polypeptides, and their use in the treatment and/or prophylaxis of cancer.

### BACKGROUND OF THE INVENTION

Immunotherapy has proven to be an effective treatment of several cancers with approved therapies constituting monoclonal and bispecific antibodies, immunomodulatory drugs and CAR-T treatments. Despite activity of these treatments, however, some patients exhibit very short responses or fail to respond to treatment. Side effects from some immunotherapies can be severe, especially side effects related to an exacerbated cytokine release. Indeed, cytokine release syndrome is one of the most common serious adverse effects of T cell-engaging immunotherapeutic agents (Shimabukuro-Vornhagen A et al. Cytokine release syndrome. J Immunother Cancer. 2018; 6(1):56. doi:10.1186/s40425-018-0343-9). Many patients will also, eventually, become resistant to available treatments. Thus despite recent advances, there is still a need for additional treatment options in cancer immunotherapy.

One apparent obstacle with current treatment modalities is suboptimal distribution to tumorous tissue. Rates of tissue distribution are negatively correlated with molecular size, and so larger molecules such as antibodies have less efficient tumour penetration than smaller ones. Another issue is immune evasion by cancer cells, which often involves inhibitory immune signals in the tumour environment. Examples of such signals include: the production of immunosuppressive cytokines and other molecules, such as TGFβ or VEGF; cell-mediated immunosuppression, e.g. via tumour-derived regulatory T cells; modulation of antigen presentation and MHICI expression; or altered expression of other ligands, e.g. increased expression of inhibitory checkpoint ligands (such as Programmed death-ligand 1 (PD-L1) and HLA-E), which reduces tumour cell killing by CD8+ T cells and natural killer (NK) cells (Vinay D et al, Immune evasion in cancer: Mechanistic basis and therapeutic strategies, Seminars in Cancer Biology. 2015:35 (Supplement):S185, https://doi.org/10.1016/j.semcancer.2015.03.004; Ben-Shmuel A et al, Unleashing Natural Killer Cells in the Tumor Microenvironment—The Next Generation of Immunotherapy?Front Immunol. 2020; 11:275, doi:10.3389/fimmu.2020.00275).

NK cells are a component of the innate immune system whose functions include cytokine secretion and cell killing via secretion of perforin- and granzyme-containing cytolytic granules. They are capable of antibody-dependent cellular cytotoxicity (ADCC) when target cells, such as tumour cells, are bound by IgG antibodies. Binding of the Fc antibody region to the CD16 receptor (FCγRIII) on NK cells overrides inhibitory signals, triggering cytokine secretion and lysis of the target cell (Vivier E et al, Functions of natural killer cells, Nat Immunol. 2008; 9:503, https://doi.org/10.1038/ni1582; Pallmer K and Oxenius A, Recognition and regulation of T cells by NK cells, Front Immunol 2016, 7:251, https://doi.org/10.3389/fimmu.2016.00251).

CD16 is expressed in two forms, CD16a and CD16b, which vary in their expression patterns and affinities for IgG Fc. CD16a is the form predominantly expressed on NK cells, as well as being found on monocytes. CD16b, in contrast, is mostly found on neutrophils, though its expression can also be induced on eosinophils. Despite the high degree of sequence similarity (around 96%) between CD16a and CD16b, CD16a has a much higher binding affinity for IgG. (Roberts J T and Barb A W, A single amino acid distorts the Fc 7 receptor IIIb/CD16b structure upon binding immunoglobulin G1 and reduces affinity relative to CD16a, J Biol Chem. 2018; 293(51):19899-19908, doi:10.1074/jbc.RA118.005273). Genotypic variation of the CD16a (FcγRIIIa) receptor itself can also alter its binding affinity: the FcγRIIIa-176V/F polymorphism (rs396991) (in some publications where the leader sequence is excluded, position 176 is reported as position 158 and this numbering is also used in the Examples herein) results in either a valine (V) or phenylalanine (F) at position 176, giving rise to variable binding phenotypes (F/F: low affinity; V/V or V/F: high affinity, and therefore higher NK cell-mediated ADCC). (Chong K T et al., Distribution of the FcγRIIIa 176 F/V polymorphism amongst healthy Chinese, Malays and Asian Indians in Singapore, Br J Clin Pharmacol 2006; 63(3): 328-332, doi: 10.1111/j.1365-2125.2006.02771.x).

Recently, there has been increasing interest in harnessing the NK cell response for cancer immunotherapy. These include use of checkpoint inhibitor blockers, the ex vivo expansion and administration of NK cells, production of CAR-NK cells (analogous to CAR-T cell therapies), and the use of bi- or multivalent NK cell engagers that cross-link NK cells to cancer cells expressing specific antigens (Hofer E and Koehl U, Natural Killer Cell-Based Cancer Immunotherapies: From Immune Evasion to Promising Targeted Cellular Therapies, Front Immunol. 2017; 8:745, https://doi.org/10.3389/fimmu.2017.00745).

Antibody-based NK engagers under development include Affimed's AFM13 (an anti-CD16a/CD30 tetravalent bispecific antibody) and AFM24 (an anti-CD16a/EGFR IgG₁-scFv fusion antibody). Both AFM13 and AFM24 have additionally been tested for their ability to induce tumour cell killing by macrophages via antibody-dependent cellular phagocytosis (ADCP) (Wingert S et al, Preclinical evaluation of AFM24, a novel CD16A-specific innate immune cell engager targeting EGFR-positive tumors. MAbs. 2021; 13(1):1950264. doi:10.1080/19420862.2021.1950264; Wingert S et al, CD16A-Specific Tetravalent Bispecific Immune Cell Engagers Potently Induce Antibody-Dependent Cellular Phagocytosis (ADCP) on Macrophages, Blood 2018; 132 (Supplement 1):1111, https://doi.org/10.1182/blood-2018-99-118427). Other bi- and multivalent NK engagers include the camelid VHH antibody-derived BiKEs and TriKEs, such as GT Biopharma's GTB-3650 which contains CD33- and CD16a-targeting regions joined to the costimulatory molecule IL15.

A further bispecific cell engager under development is R07297089. R07297089 is a bispecific tetravalent antibody targeting CD16a and BCMA (B-Cell Maturation Antigen). BCMA is highly expressed on MM cells. The properties of the antibody compound were described in Kakiuchi-Kiyota et al. (Leukemia (2022) 36:1006-1014; https://doi.org/10.1038/s41375-021-01478-w), The findings from a Phase I dose-escalation study of R07297089 in patients with Relapsed/Refractory Multiple Myeloma (Study registration NCT04434469) were reported by Plesner et al. (Poster Abstract 2755, Session 653, American Society of Hematology (ASH) Conference, 12 Dec. 2021).

Despite this progress, antibody-derived NK engagers still share the inherent difficulties associated with existing antibody cancer therapeutics, especially with regard to immunogenicity and tissue penetration. There therefore remains a need for improved cancer immunotherapeutics that can be delivered more efficiently to the tumour, while retaining the target specificity of antibodies and antibody-based drugs and avoiding potential side-effects.

The present invention seeks to address the afore-mentioned needs.

### SUMMARY OF THE INVENTION

The present invention provides a CD16a-binding polypeptide which comprises at least one motif that binds to CD16a, wherein said polypeptide comprises the following structure:
[N-terminal portion]-[Helix 1]-[Separating portion]-[Helix 2]-[C-terminal portion]
the CD16a binding motif being the portion [Helix 1]-[Separating portion]-[Helix 2].

In particular, the invention provides a CD16a-binding polypeptide, wherein:
Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein,
a) X₉ is A, D, F, H, I, K, L, Q, R, T, V or Y;
   X₁₀ is Q;
   X₁₁ is A, D, E, F, H, I, K, L, M, N, Q, R, S, T, V, W or Y;
   X₁₃ is A, Q, or V;
   X₁₄ is A, F, H, I, K, L, N, Q, R, S, T, V, W, or Y;
   X₁₇ is Q or R;
   X₁₅ is A, D, E, F, H, I, K, N, Q, R, S, T or V;
   X₂₄ is H;
   X₂₅ is A or H;
   X₂₇ is A, I, K, Q, R, S, T or V;
   X₂₈ is F or Y;
   X₃₁ is I or L;
   X₃₂ is A, E, H, K, L, N, Q or R;
   X₃₃ is K or S; and
   X₃₅ is A, H, I, L, M, R or S; or
b) X₉ is V; X₁₀ is Q; X₁₁ is M; X₁₃ is Q; X₁₄ is F; X₁₇ is R; X₁₈ is K; X₂₄ is H; X₂₅ is H; X₂₇ is S; X₂₈ is F; X₃₁ is I; X₃₂ is K; X₃₃ is S and X₃₅ is M, and optionally wherein within Helix 1 and Helix 2, at least 1 and no more than 5 (for example at least 1 and no more than 3) of the XI residues are replaced by an alternative residue, and/or at least 1 and no more than 5 (for example at least 1 and no more than 3) of the residues not labelled as X₁ are replaced by an alternative residue; or
c) X₉ is Q; X₁₀ is F; X₁₁ is Y; X₁₃ is R; X₁₄ is D; X₁₇ is D; X₁₈ is L; X₂₄ is E; X₂₅ is D; X₂₇ is K; X₂₈ is W; X₃₁ is Y; X₃₂ is M; X₃₃ is S and X₃₅ is I, and optionally wherein within Helix 1 and Helix 2, at least 1 and no more than 5 (for example at least 1 and no more than 3) of the X₁ residues are replaced by an alternative residue, and/or at least 1 and no more than 5 (for example at least 1 and no more than 3) of the residues not labelled as X₁ are replaced by an alternative residue; or
d) X₉ is F; X₁₀ is W; X₁ is I; X₁₃ is E; X₁₄ is S; X₁₇ is E; X₁₈ is S; X₂₄ is I; X₂₅ is Y; X₂₇ is K; X₂₈ is W; X₃₁ is K; X₃₂ is Y; X₃₃ is S and X₃₅ is A, and optionally wherein within Helix 1 and Helix 2, at least 1 and no more than 5 (for example at least 1 and no more than 3) of the X₁ residues are replaced by an alternative residue, and/or at least 1 and no more than 5 (for example at least 1 and no more than 3) of the residues not labelled as X₁ are replaced by an alternative residue.

The invention also provides a CD16a-binding polypeptide wherein:
i) Helix 1 comprises the sequence X₉X₁₀X₁₁AX₁₃X₁₄EIX₁₇X₁₈ and Helix 2 has the sequence X₂₄X₂₅QX₂₇X₂₈AFX₃₁X₃₂SLX₃₅, wherein:
a*) X₉ is V; X₁₀ is Q; X₁₁ is M; X₁₃ is Q; X₁₄ is F; X₁₇ is R; X₁₈ is K; X₂₄ is H; X₂₅ is H; X₂₇ is S; X₂₈ is F; X₃₁ is I; X₃₂ is K; and X₃₅ is M;
b*) X₉ is Q; X₁₀ is F; X₁₁ is Y; X₁₃ is R; X₁₄ is D; X₁₇ is D; X₁₈ is L; X₂₄ is E; X₂₅ is D; X₂₇ is K; X₂₈ is W; X₃₁ is Y; X₃₂ is M; and X₃₅ is I; or
c*) X₉ is F; X₁₀ is W; X₁₁ is I; X₁₃ is E; X₁₄ is S; X₁₇ is E; X₁₈ is S; X₂₄ is I; X₂₅ is Y; X₂₇ is K; X₂₈ is W; X₃₁ is K; X₃₂ is Y; and X₃₅ is A;
or ii) Helix 1 and Helix 2 are defined as in i), wherein within Helix 1 and Helix 2, at least 1 and no more than 3 of the Xₙ residues are replaced by an alternative residue, and/or at least 1 and no more than 3 of residues not labelled as Xₙ are replaced by an alternative residue.

The present inventors have surprisingly found that CD16a-binding polypeptides of the invention based on a non-antibody scaffold are effective in engaging NK cells and triggering ADCC-mediated cancer cell killing.

The invention therefore provides a novel CD16a engager that shows promise as an anti-cancer immunotherapeutic.

The invention further provides a CD16a-binding polypeptide, which consists of a CD16a-binding polypeptide of the invention; and optionally comprising an additional binding moiety (for example 1, 2, 3, or more additional binding moiety(ies)).

The invention further provides a CD16a-binding oligomer, which comprises at least two CD16a-binding polypeptides of the invention.

The present invention further provides a CD16a-binding polypeptide as defined herein, or CD16a-binding oligomer as defined herein, which further comprises an additional functional portion (for example at least one, at least two, or at least three; for example 1, 2, 3, 4 or 5 additional functional portions.

Therefore, present invention further provides, in embodiments where the additional functional portion is an additional binding moiety, a bispecific engager or a multispecific engager comprising an additional binding moiety.

The invention further provides:
a nucleic acid molecule encoding the CD16a-binding polypeptide or CD16a-binding oligomer of the invention;
an expression vector comprising such a nucleic acid molecule;
a host cell comprising such a nucleic acid molecule or vector.

The invention further provides a method of making the CD16a-binding polypeptide or CD16a-binding oligomer of the invention.

The invention also provides a pharmaceutical composition comprising a CD16a-binding polypeptide, CD16a-binding oligomer, CD16a binder-drug conjugate, nucleic acid molecule or expression vector of the invention.

The invention further provides a CD16a-binding polypeptide, CD16a-binding oligomer, CD16a binder-drug conjugate, nucleic acid molecule, expression vector or pharmaceutical composition of the invention for use in medicine, in particular in the treatment of a cancer.

The invention also provides the use of a CD16a-binding polypeptide, CD16a-binding oligomer, CD16a binder-drug conjugate, nucleic acid molecule, expression vector or pharmaceutical composition of the invention for the manufacture of a medicament for the treatment of cancer.

The invention also provides a method of treating cancer in which the method comprises administering to a patient in need thereof a CD16a-binding polypeptide, CD16a-binding oligomer, CD16a binder-drug conjugate, nucleic acid molecule, expression vector or pharmaceutical composition of the invention.

The invention further provides a kit comprising a CD16a-binding polypeptide, CD16a-binding oligomer, CD16a binder-drug conjugate, nucleic acid molecule, expression vector, pharmaceutical composition of the invention and, optionally, one or more further therapeutic agent(s). Such a kit finds particular use in the treatment and/or prophylaxis of cancer.

### DESCRIPTION OF THE DRAWINGS

FIG. 1 shows (a) schematic domain construction of the binding polypeptides described in the present disclosure; (b) examples of results from a phage-ELISA experiment used to screen individual clones present in the phage output after selection cycle four. In (b), assay responses to coating antigens (1) HSA, (2) Streptavidin, (3) SLAMF7 and (4) hCD16a F158 were measured and representative results for clones corresponding to CD16a-binding polypeptides A10, H09 and A11, respectively, are shown.

FIG. 2 shows an SDS-PAGE analysis of the expressed and IMAC-purified His₆-CD16a-binding polypeptide -ABDWT constructs corresponding to SEQ ID 78 (lane 1), SEQ ID 76 (lane 2), and SEQ ID 77 (lane 3). Lane M, marker proteins with molecular masses in kilodaltons.

FIG. 3 shows sensorgrams obtained after injection of the three purified hCD16a binding variants His₆-A10-ABD_{WT} [SEQ ID 76], His₆-H09-ABD_{WT} [SEQ ID 78] and His6-A11-ABD_{WT} [SEQ ID 77] at concentrations ranging from 5 nM to 2.56 M over sensor chip flow cell surfaces immobilised with hCD16a F158 or hCD16a V158.

FIG. 4 shows the resulting sensorgrams from the pairwise epitope binding assay using fusion proteins His₆-A10-ABD_{WT} [SEQ ID 76], His6-H09-ABD_{WT} [SEQ ID 78] and His₆-A11-ABD_{WT} [SEQ ID 77] in competitive binding studies to hCD16a F158 and hCD16a V158.

FIG. 5 shows an SDS-PAGE analysis of different expressed and IMAC-purified hCD16a-binding constructs. (a) His₆-A10-Cys [SEQ ID 79](Lane 1), His₆-All-Cys [SEQ ID 80](Lane 2), A10-A11-His₆ [SEQ ID 83](Lane 3), A11-A10-His₆ [SEQ ID 84](Lane 4), His₆-A10-A11-Cys [SEQ ID 81](Lane 5) and His₆-A11-A10-Cys [SEQ ID 82](Lane 6). Larger molecular weight bands visible in lanes 1, 2, 5 and 6 correspond to an expected presence of also dimeric species, held together via a disulfide bond formed between the C-terminal cysteines present in these constructs. (b) Anti-BCMA-A10-A10-His₆ [SEQ ID 86](Lane 1). Lane M, marker proteins with molecular masses in kilodaltons.

FIG. 6 shows sensorgrams obtained after injection of hCD16a F158 or hCD16a V158 over immobilised hCD16a monomeric binding variants His₆-A10-Cys [SEQ ID 79] and His₆-A11-Cys [SEQ ID 80] and heterodimeric (bi-paratopic) binding variants His₆-A10-A11-Cys [SEQ ID 81] and His₆-A11-A10-Cys [SEQ ID 82], respectively.

FIG. 7 shows sensorgrams obtained after injection of the two purified hCD16a bi-paratopic binding variants A10-A11-His₆ [SEQ ID 83] and A11-A10-His₆ [SEQ ID 84] at concentrations ranging from 0.5 nM to 32 nM over sensor chip flow cell surfaces containing immobilised hCD16a F158 or hCD16a V158, respectively.

FIG. 8 shows sensorgrams obtained from single cycle kinetics experiments performed with a fusion protein of SEQ ID 86 containing the heterodimeric A10-A10 CD16a-binding polypeptide combination attached to the 1-E6 BCMA binding polypeptide [SEQ ID 86] which was injected at five concentrations ranging from 12 to 1000 nM over sensor chip surfaces containing immobilized hCD16a F158 or hCD16a V158 proteins, respectively.

FIG. 9 shows an SDS-PAGE analysis of the purified potential dual engager protein anti-hBCMA-H09-A10-His₆ [SEQ ID 85](Lane 1). Lane M, marker proteins with molecular masses in kilodaltons.

FIG. 10 shows the experimental set-up (a) and resulting sensorgrams (b) obtained after successive injections of the dual engager construct anti-hBCMA-H09-A10-His₆ [SEQ ID 85](injection I) and either hCD16a F158 or V158 (injection III) over a sensor chip surface containing immobilised hBCMA-rabbit Fc (hBCMA-rFc) fusion protein.

FIG. 11 shows relative IFN□ secretion in cell media in cocultures of MM.1 S and PBMC exposed to dual engagers for 4 h. Engagers evaluated were Anti-BCMA-A10-His₆ [SEQ ID 88], Anti-BCMA-H09-A10-His₆ [SEQ ID 85], Anti-BCMA-A10-A10-His₆ [SEQ ID 86], Anti-BCMA-A11-A10-His₆ [SEQ ID 87] and their corresponding non BCMA binding counterparts (null).

FIG. 12 shows CD spectra (from before and after heating) and thermal melting curves for CD16a-binding polypeptides A10, H09 and A11 [SEQ ID 1, 74 and 75], all equipped with a C-terminal His₆ tag.

FIG. 13 shows sensorgrams obtained after injection of 14 alanine-substituted variants (polypeptide-YY-His₆ format), at a common concentration of 200 nM), over a sensor chip surface containing CD16a (F158) protein.

FIG. 14 shows CD spectra (from before and after heating) for 14 alanine-substituted variants (polypeptide-YY-His₆ format) of CD16a-binding polypeptide A10.

FIG. 15a shows thermal melting curves for 14 alanine-substituted variants (polypeptide-YY-His₆ format) of CD16a-binding polypeptide A10.

FIG. 15b shows the results of binding experiments of a fusion proteins containing the polypeptide with SEQ ID 1 or one of 13 alanine-substituted variants (polypeptide-YY-His₆ format) of CD16a-binding polypeptide A10 variants with CD16a F158.

FIG. 15c shows a plot of the respective on-rate (M−1 s−1) and off-rate (s−1) kinetic constants for the compounds analysed in FIG. 15b.

FIG. 16a shows responses in a Jurkat-Lucia™ NFAT-CD16 reporter assay in the presence or absence of the BCMA positive MM.1S cell line. Responses were normalized to the maximal response of elotuzumab.

FIG. 16b shows the enhancement of NK cell mediated cell killing of a BCMA positive MM.1S cell line. Responses were normalized to the maximal response mediated by a belantamab biosimilar.

FIG. 17a shows responses in a Jurkat-Lucia™ NFAT-CD16 reporter assay in the presence or absence of the BCMA positive MM.1S cell line. Responses were normalized to the maximal response of elotuzumab.

FIG. 17b shows the enhancement of NK cell mediated cell killing of a BCMA positive MM.1S cell line. Responses were normalized to the maximal response mediated by a belantamab biosimilar.

FIG. 18 shows responses in a Jurkat-Lucia™ NFAT-CD16 reporter assay in the presence or absence of the BCMA positive MM.1S cell line. Responses were normalized to the maximal response of elotuzumab.

FIG. 19a shows responses in a Jurkat-Lucia™ NFAT-CD16 reporter assay in the presence or absence of the BCMA positive MM.1S cell line. Responses were normalized to the maximal response of elotuzumab.

FIG. 19b shows the enhancement of NK cell mediated cell killing of a BCMA positive MM.1S cell line. Responses were normalized to the maximal response mediated by a belantamab biosimilar.

FIG. 20a shows responses in a Jurkat-Lucia™ NFAT-CD16 reporter assay in the presence or absence of the BCMA positive MM.1S cell line. Responses were normalized to the maximal response of elotuzumab.

FIG. 20b shows the enhancement of NK cell mediated cell killing of a BCMA positive MM.1S cell line. Responses were normalized to the maximal response mediated by a belantamab biosimilar.

FIG. 21a shows results of a cell killing assay with NK cells and MM.1s cells.

FIG. 21b show results from a 8-hour flow cytometry-based killing assay with NK cells and MM.1s (E:T 5:1), treated with increasing concentrations of any of three dual engager constructs of the invention.

FIG. 22 illustrates hBCMA target specificity of the dual engager construct with SEQ ID 85 in an in vitro multiple myeloma cell cytotoxicity assay.

FIG. 23 shows the sequences and binding data for example compounds of the invention.

FIG. 24 shows the sequences of binding motifs for compounds of the invention.

### DETAILED DESCRIPTION

The present inventors have found that polypeptides as disclosed herein are effective binders of CD16a and effectively engage immune cells. The polypeptides have been found to trigger strong ADCC responses against cancer cells in an in vitro model. Notably, the inventors have found that such anti-cancer responses compare favourably in the model with those obtained using the monoclonal antibody elotuzumab, which is approved for treatment of multiple myeloma. The inventors have further found that such anti-cancer responses compare favourably in the model with those obtained using a biosimilar of belantamab mafodotin, which is an antibody-drug conjugate for treatment of multiple myeloma, and obtained using daratumumab, a monoclonal antibody which is approved for treatment of multiple myeloma.

In its broadest aspect, the invention provides a CD16a-binding polypeptide which comprises at least one motif that binds to CD16a, wherein said polypeptide comprises the following structure:
[N-terminal portion]-[Helix 1]-[Separating portion]-[Helix 2]-[C-terminal portion];
the CD16a binding motif being the portion [Helix 1]-[Separating portion]-[Helix 2].

The various elements of the polypeptides of the invention will now be described in further detail:

### Overall Structure

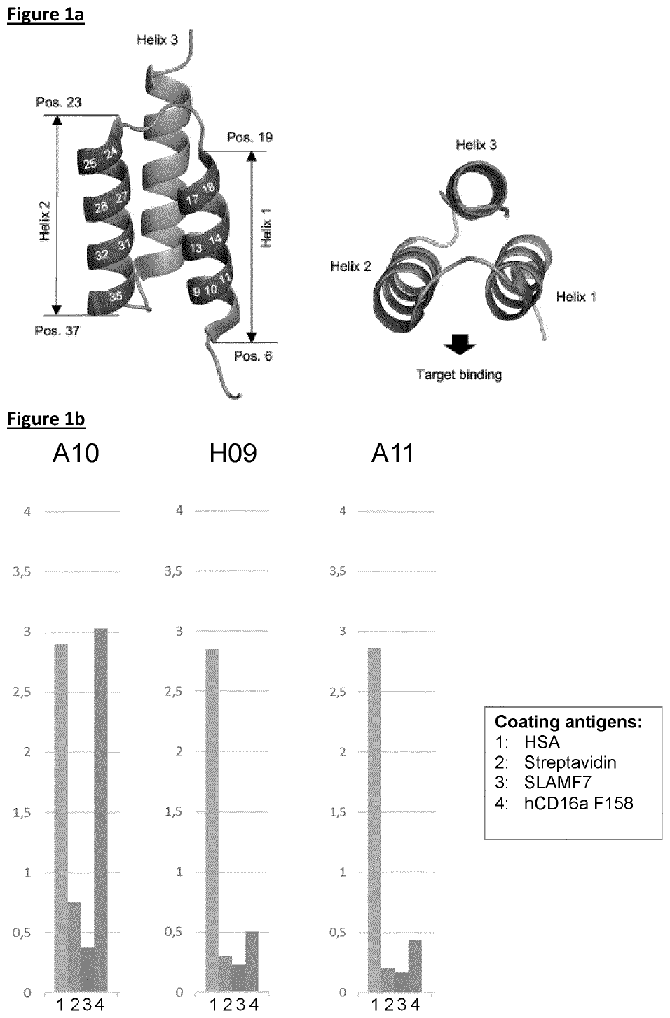

In an embodiment, the polypeptides of the present invention may be based on three-helix scaffolds, sometimes referred to as ‘affibodies’. Affibodies are small (around 6.5 kDa) engineered affinity ligands, based on the Z-domain polypeptide, which is a mutated version of the B-domain in the immunoglobulin-binding region of staphylococcal protein A (Nord K et al., Binding proteins selected from combinatorial libraries of an a-helical bacterial receptor domain, Nature Biotech, 1997:15:772, doi: 10.1038/nbt0897-772). In a full length affibody, the C-terminal portion includes [Second separating portion]-[Helix 3]—[C-terminal sequence]. The general structure of an affibody is shown in FIG. 1a.

The portions of the molecules of the invention referred to as Helix 1 and Helix 2 (and Helix 3, when present) are generally helical in structure. In some rare embodiments, it can be found that the structure established by the sequence with particular residues can be not strictly helical. Such compounds are to be considered within the broadest aspect of the invention. More preferably, the residues in the Helix 1 and Helix 2 portions do result in those structures being helical, in the sense of being alpha-helical.

### Sequences of the Polypeptides of the Invention

The sequence of the CD16a-binding polypeptides as disclosed herein may be expressed in terms of their constituent amino acids or in terms of nucleic acid sequences encoding polypeptides having those amino acid sequences. In the context of the present disclosure, the term “amino acid” encompasses any naturally occurring amino acid or unnatural amino acid. The term “unnatural amino acid” as used herein refers to non-proteinogenic (i.e. non-encoded) amino acids, which may either be found in nature or are chemically synthesised (for example citrulline, hydroxyproline, beta-alanine, ornithine, norleucine, 3-nitrotyrosine, pyroglutamic acid, or nitroarginine). It includes α, β, γ and δ amino acids. It includes an amino acid in any chiral configuration. The amino acid may, especially, be a naturally occurring a amino acid. The amino acid may, especially, be a naturally occurring L amino acid. The amino acid may, especially, be a naturally occurring L-α amino acid.

Within a polypeptide chain (for example a CD16a-binding polypeptide as disclosed herein), the amino acids are linked by peptide bonds between the carboxyl group of one amino acid and the amine group of the next amino acid in the chain. An individual amino acid is called a “residue” or “amino acid residue” once it is linked in a polypeptide chain.

The amino acid sequences herein are shown with the N-terminus to the left, and where sequences are set out across multiple lines, the N-terminus is to the top left. Unless indicated otherwise, the amino acid residues in the sequences are L-amino acids.

The amino acid sequences listed in the application are shown using standard letter abbreviations for amino acids.

The specific sequences given herein relate to specific embodiments of the invention.

The present disclosure also includes derivatives of all the sequences described herein (for example, derivatives of each of the CD16a-binding polypeptide and CD16a-binding oligomer sequences described here). Derivatives of the sequences described herein are preferably derivatives wherein from 1 to 5 (for example 1, 2 or 3 amino acid residues) may be replaced by an alternative residue, for example a different naturally occurring amino acid or a different unnatural amino acid; or a different naturally occurring amino acid excluding methionine or a different unnatural amino acid. Preferably, an unnatural amino acid according to the present invention is one that is isosteric with a naturally occurring amino acid, for example norleucine.

For example, in one embodiment, one or more (for example each) methionine residues of the sequences described herein may be replaced by a different naturally occurring amino acid or unnatural amino acid, such as an amino acid selected from isoleucine, leucine, glutamine, and norleucine; and especially isoleucine and norleucine. For example, from 1 to 5 methionine residues, from 1 to 3 methionine residues (for example 1, 2 or 3 methionine residues), or 1 or 2 methionine residues, or 1 methionine residue, when present, may be replaced by a different naturally occurring amino acid or unnatural amino acid, such as an amino acid selected from isoleucine, leucine, glutamine, and norleucine; and especially isoleucine and norleucine.

For example, in embodiments wherein X₁₁ may be or is methionine, the residue at X₁₁ may be replaced by a different naturally occurring amino acid or unnatural amino acid, such as an amino acid selected from isoleucine, leucine, glutamine, and norleucine; and especially isoleucine and norleucine. For example, in embodiments wherein X₃₅ may be or is methionine, the residue at X₃₅ may be replaced by a different naturally occurring amino acid or unnatural amino acid, such as an amino acid selected from isoleucine, leucine, glutamine, and norleucine; and especially isoleucine and norleucine. For example, in embodiments wherein X₁₁ and X₃₅ may be or are methionine, the residues at X₁₁ and X₃₅ may be replaced by a different naturally occurring amino acid or unnatural amino acid, such as an amino acid selected from isoleucine, leucine, glutamine, and norleucine; and especially isoleucine and norleucine. For example, in embodiments wherein X₃₂ may be or is methionine, the residue at X₃₂ may be replaced by a different naturally occurring amino acid or unnatural amino acid, such as an amino acid selected from isoleucine, leucine, glutamine, and norleucine; and especially isoleucine and norleucine.

Alternatively, or additionally, in certain embodiments, the sequences described herein (for example, the CD16a-binding polypeptide and CD16a-binding oligomer sequences described here) may contain amino acid substitutions wherein one or more residues is replaced by an unnatural amino acid.

For example, in one embodiment, one or more residues of the sequences described herein (for example, the CD16a-binding polypeptide and CD16a-binding oligomer sequences described here) may be replaced by an unnatural amino acid, for example norleucine. For example, from 1 to 15 residues may be replaced by unnatural amino acid(s), for example from 1 to 10 residues (for example 1, 2, 3, 4, 5, 6, 7, 8, 9 or 10 residues), from 1 to 5 residues (for example 1, 2, 3, 4 or 5), from 1 to 3 residues (for example 1, 2, or 3), or 1 residue may be replaced by unnatural amino acid(s) (for example norleucine).

For example, in one embodiment, one or more leucine residues of the sequences described herein (for example, the CD16a-binding polypeptide and CD16a-binding oligomer sequences described here) may be replaced by an unnatural amino acid, and preferably norleucine. For example, from 1 to 5 leucine residues, from 1 to 3 leucine residues (for example 1, 2 or 3 leucine residues), or 1 or 2 leucine residues, or 1 leucine residue, when present, may be replaced by unnatural amino acid (for example norleucine). For example, in certain embodiments, in the sequences described herein (for example, the CD16a-binding polypeptide and CD16a-binding oligomer sequences described here), at a position at which a leucine residue is recited, the polypeptide has the sequence with the leucine residue independently substituted for an unnatural amino acid, and preferably norleucine.

Alternatively, or additionally, in one embodiment, one or more methionine residues of the sequences described herein (for example, the CD16a-binding polypeptide and CD16a-binding oligomer sequences described here) may be replaced by an unnatural amino acid, and preferably norleucine. For example, from 1 to 5 methionine residues, from 1 to 3 methionine residues (for example 1, 2 or 3 methionine residues), or 1 or 2 methionine residues, or 1 methionine residue, when present, may be replaced by unnatural amino acid (for example norleucine). For example, in certain embodiments, in the sequences described herein (for example, the CD16a-binding polypeptide and CD16a-binding oligomer sequences described here), at a position at which a methionine residue is recited, the polypeptide has the sequence with the methionine residue independently substituted for an unnatural amino acid, and preferably norleucine.

For example, in embodiments wherein X₁₁ may be or is methionine, the residue at X₁₁ may be replaced by an unnatural amino acid(s) (for example norleucine). For example, in embodiments wherein X₃₅ may be or is methionine, the residue at X₃₅ may be replaced by an unnatural amino acid(s) (for example norleucine). For example, in embodiments wherein X₁₁ and X₃₅ may be or are methionine, the residues at X₁₁ and X₃₅ may be replaced by unnatural amino acids (for example norleucine). For example, in embodiments wherein X₃₂ may be or is methionine, the residue at X₃₂ may be replaced by an unnatural amino acid(s) (for example norleucine).

In certain embodiments, one or more methionine residues of the sequences described herein (for example, the CD16a-binding polypeptide and CD16a-binding oligomer sequences described here) may be oxidised, for example in the form of methionine sulfoxide (“Met(O)”). For example, from 1 to 5 methionine residues, from 1 to 3 methionine residues (for example 1, 2 or 3 methionine residues), or 1 or 2 methionine residues, or 1 methionine residue, when present, may be oxidised (for example may be Met(O)). For example, in embodiments wherein X₁₁ may be or is methionine, when present the methionine at X₁₁ may be oxidised (for example Met(O)). For example, in embodiments wherein X₃₅ may be or is methionine, when present the methionine may be oxidised (for example Met(O)). For example, in embodiments wherein X₁₁ and X₃₅ may be or are methionine, when present the methionines at X₁₁ and X₃₅ may be oxidised (for example Met(O)). For example, in embodiments wherein X₃₂ may be or is methionine, when present the methionine may be oxidised (for example Met(O))

Alternatively, or additionally, in certain embodiments, the sequences described herein (for example, the CD16a-binding polypeptide and CD16a-binding oligomer sequences described here) comprise a peptide purification tag or moiety (for example a histidine-tag (for example a polyhistidine tag optionally comprising tyrosine) or a methionine-tag (for example a single methionine tag or a polymethionine tag)), a signalling tag or moiety (for example a glycine residue, or a signal peptide, for example selected from signal peptides of OmpA, DsbA, PhoA, and PelB), a fluorophore tag (for example Alexa448), or a tag or moiety to assist conjugation (a cysteine tag (for example a single cysteine at the C or N terminal)). Such tags and/or moieties may preferably be present at the N-terminal and/or the C-terminal of the CD16a-binding polypeptide and CD16a-binding oligomer sequences described herein.

Therefore, the sequences described herein (for example, the CD16a-binding polypeptide and CD16a-binding oligomer sequences described here) may further comprise an additional sequence of at least 1 histidine residue (and optionally at least 1 tyrosine residue) and/or at least 1 methionine residue; for example at least 4, at least 5, or at least 6 histidine residues (and optionally at least 1 tyrosine residue, for example 1, 2 or 3 tyrosine residues) and/or at least 1 methionine residue. In one embodiment, the sequences described herein (for example, the CD16a-binding polypeptide and CD16a-binding oligomer sequences described here) may further comprise an additional sequence of at least 6 histidine residues and optionally at least 1 tyrosine residue (for example 6 histidine residues (e.g. HHHHHH) or 6 histidine residues and two tyrosine residues (e.g. YYHHHHHHH)) and/or at least 1 methionine residue (for example 1 or 2 methionine residues). A peptide purification tag or moiety, for example a histidine-tag or a methionine-tag as described above, may preferably be present at the N-terminal and/or the C-terminal of the CD16a-binding polypeptide and CD16a-binding oligomer sequences described herein. For example, an additional sequence of at least 6 histidine residues (for example 6 histidine residues; or 6 histidine residues and two tyrosine residues) and/or at least 1 methionine residue (for example 1 or 2 methionine residues) may be present at the N-terminal and/or the C-terminal of the CD16a-binding polypeptide and CD16a-binding oligomer sequences described herein.

The sequences described herein (for example, the CD16a-binding polypeptide and CD16a-binding oligomer sequences described here) may further comprise an additional sequence of at least one cysteine (for example one cysteine) at the N-terminal or the C-terminal. The sequences described herein (for example, the CD16a-binding polypeptide and CD16a-binding oligomer sequences described here) may further comprise a fluorophore tag (for example a 448Alexa tag) at the N-terminal or the C-terminal. The sequences described herein (for example, the CD16a-binding polypeptide and CD16a-binding oligomer sequences described here) may further comprise a signal peptide, for example selected from OmpA, DsbA, PhoA, and PelB, athe the N-terminal or the C-terminal, preferably the N-terminal. The sequences described herein (for example, the CD16a-binding polypeptide and CD16a-binding oligomer sequences described here) may further comprise an additional sequence of at least one glycine (for example one glycine) at the N-terminal or the C-terminal.

In certain embodiments, the CD16a-binding polypeptide is one wherein the CD16a binding efficacy is at least 1%, at least 5%, or preferably at least 10% (more preferably at least 15%, 20% 25% or 50%) of SEQ ID NO: 1, 74 or 75. When a CD16a-binding polypeptide is described herein as having CD16a binding efficacy that is at least X % of a specific peptide (e.g. SEQ IS NO: 1, 74 or 75), it is understood that the IC₅₀ concentration of the polypeptide for binding to the CD16a receptor is no more than 100/X times the IC₅₀ concentration for the specific peptide (SEQ ID NO: 1, 74 or 75) to the CD16a receptor, when measured under the same conditions.

For example, if the binding efficacy of a CD16a-binding polypeptide is at least 5%, and more preferably at least 10%, 20%, 25% or 50% of the CD16a binding efficacy of the specific peptide (e.g. SEQ ID NO: 1, 74 or 75), that is to say that the IC₅₀ concentration of the alternative polypeptide for binding to the CD16a receptor is no more than 20 times and more preferably 10 times, 5 times, 4 times or 2 times, respectively, the IC₅₀ concentration for the specific peptide (e.g. SEQ ID NO: 1, 74 or 75) to the CD16a receptor, when measured under the same conditions.

In certain embodiments, alternatively, or additionally, the CD16a-binding polypeptide is one that competes with SEQ ID NO: 1, 74 and/or 75.

The CD16a-binding polypeptides of the present invention have binding affinity for the CD16a receptor; and may optionally have binding affinity for the CD16b receptor. For example, in certain embodiments the CD16a-binding polypeptides of the present invention have similar binding affinity for the CD16a receptor and CD16b receptor; in certain embodiments, the CD16a-binding polypeptides of the present invention have stronger binding affinity for the CD16a receptor than the CD16b receptor and in certain embodiments, the CD16a-binding polypeptides of the present invention have stronger binding affinity for the CD16b receptor than the CD16a receptor.

The CD16a-binding polypeptides of the present invention may have binding affinity to one or both genotypic variation of the CD16a (FcγRIIIa) receptor: the FcγRIIIa-176V/F polymorphism (rs396991) (in some publications where the leader sequence is excluded, position 176 is reported as position 158 and this numbering is also used in the Examples herein). Preferably, the CD16a-binding polypeptides of the present invention have binding affinity for both genotypic variation of the CD16a (FcγRIIIa) receptor: the FcγRIIIa-176V/F polymorphism (rs396991). In certain embodiments, the CD16a-binding polypeptides of the present invention have a stronger binding affinity for the valine (V) position 176 phenotype, or have a stronger binding affinity for the phenylalanine (F) position 176 phenotype, or have similar binding at both the F and the V position 176 phenotype.

The present invention provides a CD16a-binding polypeptide which comprises at least one motif that binds to CD16a, wherein said polypeptide comprises the following structure:
[N-terminal portion]-[Helix 1]-[Separating portion]-[Helix 2]—[C-terminal portion]
the CD16a binding motif being the portion [Helix 1]-[Separating portion]-[Helix 2].

Preferably, Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein,
a) X₉ is A, D, F, H, I, K, L, Q, R, T, V or Y;
   X₁₀ is Q;
   X₁ is A, D, E, F, H, I, K, L, M, N, Q, R, S, T, V, W or Y;
   X₁₃ is A, Q, or V;
   X₁₄ is A, F, H, I, K, L, N, Q, R, S, T, V, W or Y;
   X₁₇ is Q or R;
   X₁₈ is A, D, E, F, H, I, K, N, Q, R, S, T or V;
   X₂₄ is H;
   X₂₅ is A or H;
   X₂₇ is A, I, K, Q, R, S, T or V;
   X₂₈ is F or Y;
   X₃₁ is I or L;
   X₃₂ is A, E, H, K, L, N, Q or R;
   X₃₃ is K or S; and
   X₃₅ is A, H, I, L, M, R or S; or
b) X₉ is V; X₁₀ is Q; X₁₁ is M; X₁₃ is Q; X₁₄ is F; X₁₇ is R; X₁₈ is K; X₂₄ is H; X₂₅ is H; X₂₇ is S; X₂₈ is F; X₃₁ is I; X₃₂ is K; X₃₃ is S and X₃₅ is M, and optionally wherein within Helix 1 and Helix 2, at least 1 and no more than 5 (for example at least 1 and no more than 3) of the Xₙ residues are replaced by an alternative residue, and/or at least 1 and no more than 5 (for example at least 1 and no more than 3) of the residues not labelled as X₁ are replaced by an alternative residue; or
c) X₉ is Q; X₁₀ is F; X₁₁ is Y; X₁₃ is R; X₁₄ is D; X₁₇ is D; X₁₈ is L; X₂₄ is E; X₂₅ is D; X₂₇ is K; X₂₈ is W; X₃₁ is Y; X₃₂ is M; X₃₃ is S and X₃₅ is I, and optionally wherein within Helix 1 and Helix 2, at least 1 and no more than 5 (for example at least 1 and no more than 3) of the X₁ residues are replaced by an alternative residue, and/or at least 1 and no more than 5 (for example at least 1 and no more than 3) of the residues not labelled as XI are replaced by an alternative residue; or
d) X₉ is F; X₁₀ is W; X₁ is I; X₁₃ is E; X₁₄ is S; X₁₇ is E; X₁₈ is S; X₂₄ is I; X₂₅ is Y; X₂₇ is K; X₂₈ is W; X₃₁ is K; X₃₂ is Y; X₃₃ is S and X₃₅ is A, and optionally wherein within Helix 1 and Helix 2, at least 1 and no more than 5 (for example at least 1 and no more than 3) of the XI residues are replaced by an alternative residue, and/or at least 1 and no more than 5 (for example at least 1 and no more than 3) of the residues not labelled as X₁ are replaced by an alternative residue.

In certain embodiments, the CD16a-binding polypeptide is one wherein Helix 1 comprises the sequence X₉X₁₀X₁₁AX₁₃X₁₄EIXX₁₅ and Helix 2 comprises the sequence X₂₄X₂₅QX₂₇X₂₈AFX₃₁X₃₂X₃₃LX₃₅, wherein, X₉ is A, D, F, H, I, K, L, Q, R, T, V or Y; X₁₀ is Q; X₁₁ is A, D, E, F, H, I, K, L, M, N, Q, R, S, T, V, W or Y; X₁₃ is A, Q or V; X₁₄ is A, F, H, I, K, L, N, Q, R, S, T, V, W or Y; X₁₇ is Q or R; X₁₈ is A, D, E, F, H, I, K, N, Q, R, S, T or V; X₂₄ is H; X₂₅ is A or H; X₂₇ is A, I, K, Q, R, S, T or V; X₂₈ is F or Y; X₃₁ is I or L; X₃₂ is A, E, H, K, L, N, Q or R; X₃₃ is K or S; and X₃₅ is A, H, I, L, MR or S.

In a further embodiment, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is D, F, H, I, K, L, Q, R, T, V or Y; X₁₀ is Q; X₁₁ is A, D, E, F, H, I, K, L, M, N, Q, R, S, T, V, W or Y; X₁₃ is A, Q or V; X₁₄ is F, H, I, K, L, N, Q, R, S, T, V, W or Y; X₁₇ is R or Q; X₁₈ is A, D, E, F, H, I, K, N, Q, R, S, T or V; X₂₄ is H; X₂₅ is H or A; X₂₇ is A, I, K, Q, R, T, S or V; X₂₈ is F or Y; X₃₁ is I or L; X₃₂ is A, E, H, K, L, N, Q or R; X₃₃ is K or S; and X₃₅ is A, H, I, L, M, R or S.

In a further embodiment, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is D, F, H, I, K, L, Q, R, T, V or Y; X₁₀ is Q; X₁₁ is A, D, E, F, H, I, K, L, M, N, Q, R, S, T, V, W or Y; X₁₃ is A, Q or V; X₁₄ is F, H, I, K, L, N, Q, R, S, T, V, W or Y; X₁₇ is R; X₁₈ is A, D, E, F, H, K, N, Q, R, S, T or V; X₂₄ is H; X₂₅ is H; X₂₇ is A, I, K, Q, R, T, S or V; X₂₈ is F or Y; X₃₁ is I or L; X₃₂ is A, E, H, K, N, Q or R; X₃₃ is K or S; and X₃₅ is A, H, I, L, M, R or S.

In a further embodiment, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is D, F, H, I, K, L, Q, R, T, V or Y; X₁₀ is Q; Xₙ is A, D, E, F, H, I, K, L, N, Q, R, S, T, V, W or Y; X₁₃ is A, Q or V; X₁₄ is H, I, K, L, N, Q, R, S, T, V, W or Y; X₁₇ is R or Q; X₁₈ is A, D, E, F, H, I, K, N, Q, R, S, T or V; X₂₄ is H; X₂₅ is H or A; X₂₇ is A, I, K, Q, R, T or V; X₂₈ is F or Y; X₃₁ is I or L; X₃₂ is A, E, H, K, L, N, Q or R; X₃₃ is K or S; and X₃₅ is A, H, I, L, R or S.

In a further embodiment, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is D, F, H, I, K, L, Q, R, T, V or Y; X₁₀ is Q; X₁₁ is A, D, E, F, H, I, K, L, N, Q, R, S, T, V, W or Y; X₁₃ is A, Q or V; X₁₄ is H, I, K, L, N, Q, R, S, T, V, W or Y; X₁₇ is R; X₁₈ is A, D, E, F, H, K, N, Q, R, S, T or V; X₂₄ is H; X₂₅ is H; X₂₇ is A, I, K, Q, R, T, or V; X₂₈ is F or Y; X₃₁ is I or L; X₃₂ is A, E, H, K, N, Q or R; X₃₃ is K or S; and X₃₅ is A, H, I, L, R or S.

In certain preferred embodiments, the CD16a-binding polypeptide of the invention has a fast binding or “on” rate for the CD16a receptor. For example, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is D, F, H, I, K, L, Q, R, T, V or Y; X₁₀ is Q; X₁₁ is A, D, E, F, H, I, K, N, Q, R, S, T, V, W or Y; X₁₃ is A, Q or V; X₁₄ is H, I, K, L, N, Q, R, S, V, W or Y; X₁₇ is R; X₁₈ is A, D, E, F, H, K, N, Q, R, S or T; X₂₄ is H; X₂₅ is H; X₂₇ is A, I, K, Q, R, T or V; X₂₈ is F; X₃₁ is I or L; X₃₂ is A, E, H, K, N, Q or R; X₃₃ is K or S; and X₃₅ is H, I, L, R or S.

In certain preferred embodiments, the CD16a-binding polypeptide of the invention has a slow dissociation rate or “off” rate for the CD16a receptor. For example, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is D, F, H, I, K, L, Q, T, V or Y; X₁₀ is Q; Xₙ is A, D, E, F, H, I, K, L, N, Q, R, S, T, V, W or Y; X₁₃ is A or Q; X₁₄ is H, I, K, L, Q, R, S, T, V, W or Y; X₁₇ is R; X₁₈ is A, D, E, F, H, K, N, Q, R, S, T or V; X₂₄ is H; X₂₅ is H; X₂₇ is A, I, K, Q, R, T or V; X₂₈ is F or Y; X₃₁ is I or L; X₃₂ is A, E, H, K, N, Q or R; X₃₃ is K or S; and X₃₅ is A, H, I, L or R.

In certain preferred embodiments, the CD16a-binding polypeptide of the invention has a high binding affinity for the CD16a receptor, for example a K_{D} for the CD16a receptor of less than 250 nM. For example, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is D, F, H, I, K, L, Q, R, T, V or YX₁₀ is Q; X₁₁ is A, D, E, F, H, I, K, N, Q, R, S, T, V, W or Y; X₁₃ is A or Q; X₁₄ is H, I, K, L, Q, R, S, V, W or Y; X₁₇ is R; X₁₈ is A, F, H, K, N, Q, R, S or T; X₂₄ is H; X₂₅ is H; X₂₇ is A, I, K, Q, R, T or V; X₂₈ is F or Y; X₃₁ is I or L; X₃₂ is E, H, K, N, Q or R; X₃₃ is K or S; and X₃₅ is A, H, I, L, R or S.

In certain preferred embodiments, the CD16a-binding polypeptide of the invention have an especially fast binding or “on” rate for the CD16a receptor. For example, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is F, L, Q, T or Y; X₁₀ is Q; X₁₁ is A, F, H, I, L, N, Q, S, or Y; X₁₃ is A or Q; X₁₄ is I, K, Q, R or V; X₁₇ is R; X₁₈ is A, E, H, K, Q, R, T or V; X₂₄ is H; X₂₅ is H; X₂₇ is A, I, K, Q, R or V; X₂₈ is F; X₃₁ is I; X₃₂ is A, H, K, N, Q or R; X₃₃ is K or S; and X₃₅ is H, I or L.

In certain preferred embodiments, the CD16a-binding polypeptide of the invention has an especially slow dissociation rate or “off” rate for the CD16a receptor. For example, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is I, L, Q, T or V; X₁₀ is Q; X₁₁ is A, E, F, H, I, S, V or Y; X₁₃ is Q; X₁₄ is K, L, R, V, W or Y; X₁₇ is R; X₁₈ is A, H, K, Q, R, S or T; X₂₄ is H; X₂₅ is H; X₂₇ is I, K, Q, R, T or V; X₂₈ is F; X₃₁ is I or L; X₃₂ is K, N or R; X₃₃ is K or S; and X₃₅ is I or L.

In certain preferred embodiments, the CD16a-binding polypeptide of the invention has an especially high binding affinity for the CD16a receptor, for example a K_{D} for the CD16a receptor of less than 100 nM. For example, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is I, L, Q or V; X₁₀ is Q; X₁₁ is A, E, H, I, S, W or Y; X₁₃ is Q; X₁₄ is K, L, R, V, W or Y; X₁₇ is R; X₁₈ is A, H, K, Q, R, S, or T; X₂₄ is H; X₂₅ is H; X₂₇ is K, Q, R, T or V; X₂₈ is F; X₃₁ is I; X₃₂ is K, N or R; X₃₃ is K or S; and X₃₅ is I or L.

In certain preferred embodiments, the CD16a-binding polypeptide of the invention has at least two of the following: a fast binding or “on” rate for the CD16a receptor; and/or a slow dissociation rate or “off” rate for the CD16a receptor; and/or a high binding affinity for the CD16a receptor, for example a K_{D} for the CD16a receptor of less than 100 nM. For example, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is I, L, Q or V; X₁₀ is Q; X₁₁ is A, E, H, I, S or Y; X₁₃ is Q; X₁₄ is K, L, R, V, W or Y; X₁₇ is R; X₁₈ is H, K, Q, R, S or T; X₂₄ is H; X₂₅ is H; X₂₇ is K, Q, R, T or V; X₂₈ is F; X₃₁ is I; X₃₂ is N or K; X₃₃ is K or S; and X₃₅ is I or L.

In certain preferred embodiments, the CD16a-binding polypeptide of the invention has an especially slow dissociation rate or “off” rate for the CD16a receptor and has a high binding affinity for the CD16a receptor, for example a K_{D} for the CD16a receptor of less than 100 nM. For example, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is L or V; X₁₀ is Q; X₁₁ is A, I, S or Y; X₁₃ is Q; X₁₄ is K, R or V; X₁₇ is R; X₁₈ is K, Q, R, S or T; X₂₄ is H; X₂₅ is H; X₂₇ is K, R or V; X₂₈ is F; X₃₁ is I; X₃₂ is N or K; X₃₃ is K or S; and X₃₅ is I or L.

In certain preferred embodiments, the CD16a-binding polypeptide of the invention binds the CD16a and CD16b receptor. For example, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is D, H, I, K, L, Q, T or V; X₁₀ is Q; X₁₁ is A, D, E, F, H, I, K, N, R, S, V, W or Y; X₁₃ is Q; X₁₄ is K, L, Q, R, S, V, W or Y; X₁₇ is R; X₁₈ is A, H, K, N, Q, R, S or T; X₂₄ is H; X₂₅ is H; X₂₇ is A, I, K, Q, R, T or V; X₂₈ is F or Y; X₃₁ is I or L; X₃₂ is A, E, H, K, N, Q or R; X₃₃ is K or S; and X₃₅ is A, I, L, or R.

In certain preferred embodiments, the CD16a-binding polypeptide of the invention has a binding preference for the CD16a receptor compared to the CD16b receptor. For example, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is K, Q or Y; X₁₀ is Q; X₁₁ is I or Q; X₁₃ is Q; X₁₄ is W or Y; X₁₇ is R; X₁₈ is H, K or R; X₂₄ is H; X₂₅ is H; X₂₇ is A, K or T; X₂₈ is F; X₃₁ is I; X₃₂ is A, K or Q; X₃₃ is K or S; and X₃₅ is I or L;

In certain preferred embodiments, the CD16a-binding polypeptide of the invention has a binding preference for the CD16b receptor compared to the CD16a receptor. For example, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is L, V or Y; X₁₀ is Q; X₁₁ is I, N or Q; X₁₃ is Q; X₁₄ is K, R or Q; X₁₇ is R; X₁₈ is E, A or V; X₂₄ is H; X₂₅ is H; X₂₇ is K or Q; X₂₈ is F; X₃₁ is I; X₃₂ is H, K or Q; X₃₃ is K or S; and X₃₅ is I or L

In certain very preferred embodiments, the CD16a-binding polypeptide of the invention, is especially active in functional assays, e.g. a CD16 reporter assay and/or a cell killing assay. For example, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is L, Q, T or V; X₁₀ is Q; X₁₁ is I, V or Y; X₁₃ is Q; X₁₄ is K, R or Y; X₁₇ is R; X₁₈ is K, R, S or T; X₂₄ is H; X₂₅ is H; X₂₇ is I, T or V; X₂₈ is F; X₃₁ is I or L; X₃₂ is K or N; X₃₃ is K or S; and X₃₅ is I or L.

In certain especially preferred embodiments, the CD16a-binding polypeptide of the invention, is very especially active in functional assays, e.g. a CD16 reporter assay and/or a cell killing assay. For example. For example, the CD16a-binding polypeptide is one wherein Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is L, T or V; X₁₀ is Q; X₁₁ is I, V or Y; X₁₃ is Q; X₁₄ is K or R; X₁₇ is R; X₁₈ is R, S or T; X₂₄ is H; X₂₅ is H; X₂₇ is I or V; X₂₈ is F; X₃₁ is I or L; X₃₂ is K or N; X₃₃ is K; and X₃₅ is I or L.

In certain embodiments of the present invention, the CD16a-binding polypeptide is one that does not have a methionine residue at position X₁₁ or at X₃₅, for example a CD16a-binding polypeptide wherein:

Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein:

X₉ is A, D, F, H, I, K, L, Q, R, T, V or Y; X₁₀ is Q; X₁₁ is A, D, E, F, H, I, K, L, N, Q, R, S, T, V, W or Y or an unnatural amino acid (for example norleucine); X₁₃ is A, Q, or V; X₁₄ is A, F, H, I, K, L, N, Q, R, S, T, V, W or Y; X₁₇ is Q or R; X₁₈ is A, D, E, F, H, I, K, N, Q, R, S, T or V; X₂₄ is H; X₂₅ is A or H; X₂₇ is A, I, K, Q, R, S, T or V; X₂₈ is F or Y; X₃₁ is I or L; X₃₂ is A, E, H, K, L, N, Q or R; X₃₃ is K or S; and X₃₅ is A, H, I, L, R, S or an unnatural amino acid (for example norleucine); or

X₉ is A, D, F, H, I, K, L, Q, R, T, V or Y; X₁₀ is Q; X₁₁ is A, D, E, F, H, I, K, L, N, Q, R, S, T, V, W, Y, isoleucine, leucine, glutamine, or norleucine (for example A, D, E, F, H, I, K, L, N, Q, R, S, T, V, W, Y, isoleucine or norleucine); X₁₃ is A, Q, or V; X₁₄ is A, F, H, I, K, L, N, Q, R, S, T, V, W, or Y; X₁₇ is Q or R; X₁₈ is A, D, E, F, H, I, K, N, Q, R, S, T or V; X₂₄ is H; X₂₅ is A or H; X₂₇ is A, I, K, Q, R, S, T, or V; X₂₈ is F or Y; X₃₁ is I or L; X₃₂ is A, E, H, K, L, N, Q or R; X₃₃ is K or S; and X₃₅ is A, H, I, L, R, S, isoleucine, leucine, glutamine, or norleucine (for example, A, H, I, L, R, S, isoleucine or norleucine); or

X₉ is A, D, F, H, I, K, L, Q, R, T, V or Y; X₁₀ is Q; X₁₁ is A, D, E, F, H, I, K, L, N, Q, R, S, T, V, W or Y; X₁₃ is A, Q, or V; X₁₄ is A, F, H, I, K, L, N, Q, R, S, T, V, W or Y; X₁₇ is Q or R; X₁₈ is A, D, E, F, H, I, K, N, Q, R, S, T or V; X₂₄ is H; X₂₅ is A or H; X₂₇ is A, I, K, Q, R, S, Tor V; X₂₈ is F or Y; X₃₁ is I or L; X₃₂ is A, E, H, K, L, N, Q or R; X₃₃ is K or S; and X₃₅ is A, H, I, L, R or S.

In a further embodiment, that does not have a methionine residue at position X₁₁ or at X₃₅, a CD16a-binding polypeptide the CD16a-binding polypeptide is one wherein: Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is A, D, F, H, I, K, L, Q, R, T, V or Y; X₁₀ is Q; Xₙ is A, D, E, F, H, I, K, L, N, Q, R, S, T, V, W Y or an unnatural amino acid (for example norleucine); X₁₃ is A, Q or V; X₁₄ is A, F, H, I, K, L, N, Q, R, S, T, V, W or Y; X₁₇ is Q or R; X₁₈ is A, D, E, F, H, I, K, N, Q, R, S, T or V; X₂₄ is H; X₂₅ is A or H; X₂₇ is A, I, K, Q, R, S, T, or V; X₂₈ is F or Y; X₃₁ is I or L; X₃₂ is A, E, H, K, L, N, Q or R; X₃₃ is K or S; and X₃₅ is A, H, I, L, R, S or an unnatural amino acid (for example norleucine); or

X₉ is A, D, F, H, I, K, L, Q, R, T, V or Y; X₁₀ is Q; Xₙ is A, D, E, F, H, I, K, L, N, Q, R, S, T, V, W, Y, isoleucine, leucine, glutamine, or norleucine (for example A, D, E, F, H, I, K, L, N, Q, R, S, T, V, W, Y, isoleucine or norleucine); X₁₃ is A, Q or V; X₁₄ is A, F, H, I, K, L, N, Q, R, S, T, V, W, or Y; X₁₇ is Q or R; X₁₈ is A, D, E, F, H, I, K, N, Q, R, S, T or V; X₂₄ is H; X₂₅ is A or H; X₂₇ is A, I, K, Q, R, S, or V; X₂₈ is F or Y; X₃₁ is I or L; X₃₂ is A, E, H, K, L, N, Q, or R; X₃₃ is K or S; and X₃₅ is A, H, I, L, R, S, isoleucine, leucine, glutamine, or norleucine (for example, A, H, I, L, R, S, isoleucine or norleucine); or

X₉ is A, D, F, H, I, K, L, Q, R, T, V or Y; X₁₀ is Q; X₁₁ is A, D, E, F, H, I, K, L, N, Q, R, S, T, V, W or Y; X₁₃ is A, Q or V; X₁₄ is A, F, H, I, K, L, N, Q, R, S, T, V, W or Y; X₁₇ is Q or R; X₁₈ is A, D, E, F, H, I, K, N, Q, R, S, T or V; X₂₄ is H; X₂₅ is A or H; X₂₇ is A, I, K, Q, R, S, T or V; X₂₈ is F or Y; X₃₁ is I or L; X₃₂ is A, E, H, K, L, N, Q or R; X₃₃ is K or S; and X₃₅ is A, H, I, L, R or S

In a further embodiment, that does not have a methionine residue at position X₁₁ or at X₃₅, a CD16a-binding polypeptide the CD16a-binding polypeptide is one wherein:

Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, wherein, X₉ is D, F, H, I, K, L, Q, R, T, V or Y; X₁₀ is Q; X₁₁ is A, D, E, F, H, I, K, L, N, Q, R, S, T, V, W, Y or an unnatural amino acid (for example norleucine); X₁₃ is A, Q or V; X₁₄ is F, H, I, K, L, N, Q, R, S, T, V, W or Y; X₁₇ is R or Q; X₁₈ is A, D, E, F, H, I, K, N, Q, R, S, T or V; X₂₄ is H; X₂₅ is H or A; X₂₇ is A, I, K, Q, R, T, S or V; X₂₈ is F or Y; X₃₁ is I or L; X₃₂ is A, E, H, K, L, N, Q or R; X₃₃ is K or S; and X₃₅ is A, H, I, L, R, S or an unnatural amino acid (for example norleucine); or

X₉ is D, F, H, I, K, L, Q, R, T, V or Y; X₁₀ is Q; X₁₁ is A, D, E, F, H, I, K, L, N, Q, R, S, T, V, W Y, isoleucine, leucine, glutamine, or norleucine (for example A, D, E, F, H, I, K, L, N, Q, R, S, T, V, W Y, isoleucine or norleucine); X₁₃ is A, Q or V; X₁₄ is F, H, I, K, L, N, Q, R, S, T, V, W or Y; X₁₇ is R or Q; X₁₈ is A, D, E, F, H, I, K, N, Q, R, S, T or V; X₂₄ is H; X₂₅ is H or A; X₂₇ is A, I, K, Q, R, T, S or V; X₂₈ is F or Y; X₃₁ is I or L; X₃₂ is A, E, H, K, L, N, Q or R; X₃₃ is K or S; and X₃₅ is A, H, I, L, R S, isoleucine, leucine, glutamine, or norleucine (for example A, H, I, L, R, S, isoleucine or norleucine); or

X₉ is D, F, H, I, K, L, Q, R, T, V or Y; X₁₀ is Q; X₁₁ is A, D, E, F, H, I, K, L, N, Q, R, S, T, V, W or Y; X₁₃ is A, Q or V; X₁₄ is F, H, I, K, L, N, Q, R, S, T, V, W or Y; X₁₇ is R or Q; X₁₈ is A, D, E, F, H, I, K, N, Q, R, S, T or V; X₂₄ is H; X₂₅ is H or A; X₂₇ is A, I, K, Q, R, T, S or V; X₂₈ is F or Y; X₃₁ is I or L; X₃₂ is A, E, H, K, L, N, Q or R; X₃₃ is K or S; and X₃₅ is A, H, I, L, R or S.

In certain embodiments, the CD16a-binding polypeptide is one wherein the CD16a binding efficacy is at least 1%, at least 5%, or preferably at least 10% (more preferably at least 15%, 20% 25% or 50%) of SEQ ID NO: 1 (i.e. binding efficacy of the peptide of SEQ ID NO: 1 to the CD16a receptor, when measured under the same conditions). Preferably, the CD16a-binding polypeptide is one wherein the CD16a binding efficacy is at least 10% of SEQ ID NO: 1.

In certain embodiments, alternatively, or additionally, the CD16a-binding polypeptide is one that competes with SEQ ID NO: 1.

In advantageous embodiments of CD16a-binding polypeptides of the invention, Proline (P) and Cysteine (C) are not present in Helix 1 or Helix 2 of a CD16a binding polypeptide of the present invention. Advantageously Glycine (G) is also not present in Helix 1 or Helix 2 of a CD16a binding polypeptide of the present invention.

In certain embodiments, a CD16a-binding polypeptide of the invention is one wherein:
Helix 1 comprises the sequence X₆X₇X₈X₉X₁₀X₁₁AX₁₃X₁₄EIX₁₇X₁₈X₁₉ and/or
Helix 2 comprises the sequence X₂₃X₂₄X₂₅QX₂₇X₂₈AFX₃₁X₃₂X₃₃LX₃₅X₃₆X₃₇,
wherein, X₆ is any naturally occurring amino acid (preferably D, E, N or Q; more preferably N) or is absent (for example X₆ is any naturally occurring amino acid (preferably D, E, N or Q; more preferably N)); X₇ is any naturally occurring amino acid (preferably H, K or R; more preferably K) or is absent (for example X₇ is any naturally occurring amino acid (preferably H, K or R; more preferably K)); X₈ is any naturally occurring amino acid (preferably D, E, N or Q; more preferably E) or is absent (for example X₈ is any naturally occurring amino acid (preferably D, E, N or Q; more preferably E)); X₁₉ is any naturally occurring amino acid (preferably G, A, V, L or I; more preferably L) or is absent (for example X₁₉ is any naturally occurring amino acid (preferably G, A, V, L or I; more preferably L)); X₂₃ is any naturally occurring amino acid (preferably D, E, N or Q; more preferably N) or is absent (for example X₂₃ is any naturally occurring amino acid (preferably D, E, N or Q; more preferably N); X₃₆ is any naturally occurring amino acid (preferably D, E, N or Q; more preferably D) or is absent (for example X₃₆ is any naturally occurring amino acid (preferably D, E, N or Q; more preferably D)); and X₃₇ is any naturally occurring amino acid (preferably D, E, N or Q; more preferably D) or is absent (for example X₃₇ is naturally occurring amino acid (preferably D, E, N or Q; more preferably D)).

In certain embodiments, a CD16a-binding polypeptide of the invention is one wherein:
Helix 1 comprises the sequence X₆X₇X₈X₉X₁₀X₁₁AX₁₃X₁₄EIX₁₇X₁₈X₁₉ and/or
Helix 2 comprises the sequence X₂₃X₂₄X₂₅QX₂₇X₂₈AFX₃₁X₃₂X₃₃LX₃₅X₃₆X₃₇,
wherein, X₆ is D, E, N or Q (more preferably N) or is absent (for example X₆ is D, E, N or Q, and more preferably N); X₇ is H, K or R (preferably K) or is absent (for example X₇ is H, K or R; and more preferably K); X₈ is D, E, N or Q (preferably E) or is absent (for example X₈ is D, E, N or Q; and more preferably E); X₁₉ is G, A, V, L or I (preferably L) or is absent (for example X₁₉ is G, A, V, L or I; and more preferably L); X₂₃ is D, E, N or Q (preferably N) or is absent (for example X₂₃ is D, E, N or Q; and more preferably N); X₃₆ is D, E, N or Q more preferably D) or is absent (for example X₃₆ is D, E, N or Q; and more preferably D); and X₃₇ is D, E, N or Q (preferably D) or is absent (for example X₃₇ is D, E, N or Q; and more preferably D).

In certain embodiments of the present invention, such as the embodiments described above:
Helix 1 comprises the sequence X₆X₇X₈X₉X₁₀X₁₁AX₁₃X₁₄EIX₁₇X₁₈X₁₉ and/or
Helix 2 comprises the sequence X₂₃X₂₄X₂₅QX₂₇X₂₈AFX₃₁X₃₂X₃₃LX₃₅X₃₆X₃₇.

In such embodiments, X₆ may be any naturally occurring amino acid or is absent; X₇ may be any naturally occurring amino acid or is absent; X₈ may be any naturally occurring amino acid or is absent; X₁₉ may be any naturally occurring amino acid or is absent; X₂₃ may be any naturally occurring amino acid or is absent; X₃₆ may be any naturally occurring amino acid or is absent; and X₃₇ may be any naturally occurring amino or is absent.

More preferably, X₆ may be any naturally occurring amino acid; X₇ may be any naturally occurring amino acid; X₈ may be any naturally occurring amino acid; X₁₉ may be any naturally occurring amino acid; X₂₃ may be any naturally occurring amino acid; X₃₆ may be any naturally occurring amino acid; and X₃₇ may be any naturally occurring amino.

In another preferred embodiment, X₆ may be D, E, N, Q or is absent; X₇ may be H, K, R or is absent; X₈ may be any D, E, N, Q or is absent; X₁₉ may be G, A, V, L, I or is absent; X₂₃ may G, A, V, L, I or is absent; X₃₆ may be G, A, V, L, I or is absent; and X₃₇ may G, A, V, L, I or is absent. More preferably, X₈ may be E or is absent; X₁₉ may be L or is absent; X₂₃ may be D or is absent; X₃₆ may be D or is absent; and X₃₇ may be D or is absent.

In an even more preferred embodiment, X₆ may be D, E, N or Q; X₇ may be H, K or R; X₈ may be any D, E, N or Q; X₁₉ may be G, A, V, L or I; X₂₃ may G, A, V, L, or I; X₃₆ may be G, A, V, L or I; and X₃₇ may G, A, V, L or I.

In another preferred embodiment, X₆ may be N or is absent; X₇ may be K or is absent; X₈ may be E or is absent; X₁₉ may be L or is absent; X₂₃ may be D or is absent; X₃₆ may be D or is absent; and X₃₇ may be D or is absent.

In an especially preferred embodiment, X₆ is N; X₇ is K; and X₈ is E. In another especially preferred embodiment, X₃₆ is D; and X₃₇ is D.

In a very especially preferred embodiment, X₆ is N; X₇ is K; X₈ is E; X₁₉ is L; X₂₃ is D; X₃₆ is D; and X₃₇ is D. In such embodiments,
Helix 1 comprises the sequence NKEX₉X₁₀X₁₁AX₁₃X₁₄EIX₁₇X₁₈L and/or
Helix 2 comprises the sequence DX₂₄X₂₅QX₂₇X₂₈AFX₃₁X₃₂X₃₃LX₃₅DD.

In one embodiment, the CD16a-binding polypeptide is one wherein:
Helix 1 comprises the sequence NKEVQMAQFEIRKL and Helix 2 comprises the sequence NHHQSFAFIKSLMDD;
and optionally wherein, at least 1 and no more than 5 (for example 1, 2, 3, 4 or 5; or for example, at least 1 and no more than 3 (for example 1, 2, or 3)) residues in the sequence of Helix 1 and/or Helix 2 are replaced by an alternative residue (for example replaced by an alternative residue that is a conservative replacement).

In certain embodiments, such a CD16a-binding polypeptide has a CD16a binding efficacy of at least 1%, at least 5%, or at least 10% (for example at least 15%, 20% 25% or 50%) of SEQ ID NO: 1. Preferably, such a CD16a-binding polypeptide has a CD16a binding efficacy that is at least 10% of SEQ ID NO: 1. In certain embodiments, alternatively, or additionally, such a CD16a-binding polypeptide is one that competes with SEQ ID NO: 1.

In one embodiment, the CD16a-binding polypeptide is one wherein:
Helix 1 comprises the sequence NKEQFYARDEIDLL and Helix 2 comprises the sequence NEDQKWAFYMSLIDD;
and optionally wherein, at least 1 and no more than 5 (for example 1, 2, 3, 4 or 5; or for example, at least 1 and no more than 3 (for example 1, 2, or 3)) residues in the sequence of Helix 1 and/or Helix 2 are replaced by an alternative residue (for example replaced by an alternative residue that is a conservative replacement).

In certain embodiments, such a CD16a-binding polypeptide has a CD16a binding efficacy of at least 1%, at least 5%, or at least 10% (for example at least 15%, 20% 25% or 50%) of SEQ ID NO: 74. Preferably, such a CD16a-binding polypeptide has a CD16a binding efficacy that is at least 10% of SEQ ID NO: 74. In certain embodiments, alternatively, or additionally, such a CD16a-binding polypeptide is one that competes with SEQ ID NO: 74.

In one embodiment, the CD16a-binding polypeptide is one wherein:
Helix 1 comprises the sequence NKEFWIAESEIESL and Helix 2 comprises the sequence NIYQKWAFKYSLADD;
and optionally wherein, at least 1 and no more than 5 (for example 1, 2, 3, 4 or 5; or for example, at least 1 and no more than 3 (for example 1, 2, or 3)) residues in the sequence of Helix 1 and/or Helix 2 are replaced by an alternative residue (for example replaced by an alternative residue that is a conservative replacement).

In certain embodiments, such a CD16a-binding polypeptide has a CD16a binding efficacy of at least 1%, at least 5%, or at least 10% (for example at least 15%, 20% 25% or 50%) of SEQ ID NO: 75. Preferably, such a CD16a-binding polypeptide has a CD16a binding efficacy that is at least 10% of SEQ ID NO: 75. In certain embodiments, alternatively, or additionally, such a CD16a-binding polypeptide is one that competes with SEQ ID NO: 75.

In one embodiment the CD16a binding motif, being the portion [Helix 1]-[Separating portion]-[Helix 2], is (i.e. has a sequence):

X₆X₇X₈X₉X₁₀X₁₁AX₁₃X₁₄EIX₁₇X₁₈X₁₉X₂₀X₂₁X₂₂X₂₃X₂₄X₂₅QX₂₇X₂₈AFX₃₁
X₃₂X₃₃LX₃₅X₃₆X₃₇

wherein
X₂₀ is any naturally occurring amino acid, X₂₁ is any naturally occurring amino acid; and X₂₂ is any naturally occurring amino acid, and wherein optionally one or two (for example, optionally 1) of X₂₀, X₂₁ or X₂₂ are absent; and the other residues are as defined above.

More preferably, X₆ may be D, E, N, Q or is absent; X₇ may be H, K, R or is absent; X₈ may be any D, E, N, Q or is absent; X₉, X₁₀, X₁₁, X₁₃, X₁₄, X₁₇ and X₁₈ are as defined above; X₁₉ may be G, A, V, L, I or is absent; X₂₀ may be S, T, M, P, F, Y or W (for example P or T); X₂₁ may be D, E, N or Q; X₂₂ may be G, A, V, L, I or is absent; X₂₃ may G, A, V, L, I or is absent; X₃₆ may be G, A, V, L, I or is absent; and X₃₇ may G, A, V, L, I or is absent; and wherein optionally one or two (for example 30 optionally 1) of X₂₀, X₂₁, X₂₂ are absent.

Preferably, X₆ may be D, E, N or Q; X₇ may be H, K or R; X₈ may be any D, E, N or Q; X₉, X₁₀, X₁₁, X₁₃, X₁₄, X₁₇ and X₁₈ are as defined above; X₁₉ may be G, A, V, L or I; X₂₀ may S, T, M, P, F, Y or W (for example P or T); X₂₁ may be D, E, N or Q; and X₂₂ may be G, A, V, L, or I; X₂₃ may G, A, V, L or I; X₃₆ may be G, A, V, L or I; and X₃₇ may G, A, V, L or I; and wherein optionally one or two (for example optionally 1) of X₂₀, X₂₁, X₂₂ are absent.

For example, X₆ may be D, E, N or Q; X₇ may be H, K or R; X₈ may be any D, E, N or Q; X₉, X₁₀, X₁₁, X₁₃, X₁₄, X₁₇ and X₁₈ are as defined above; X₁₉ may be G, A, V, L or I; X₂₀ may be S, T, M, P, F, Y or W (for example P or T); X₂₁ may be D, E, N or Q; and X₂₂ may be G, A, V, L, or I; X₂₃ may G, A, V, L or I; X₃₆ may be G, A, V, L or I; and X₃₇ may G, A, V, L or I.

More preferably, X₈ may be E or is absent; X₉, X₁₀, X₁₁, X₁₃, X₁₄, X₁₇ and X₁₈ are as defined above; X₁₉ may be L or is absent; X₂₃ may be D or is absent; X₂₄, X₂₅, X₂₇, X₂₈, X₃₁, X₃₂, X₃₃ and X₃₅ are as defined above, and X₃₆ may be D or is absent; and X₃₇ may be D or is absent.

In an especially preferred embodiment, X₆ is N; X₇ is K; X₈ is E; X₉, X₁₀, X₁₁, X₁₃, X₁₄, X₁₇ and X₁₈ are as defined above; X₁₉ is L; X₂₀ is S, T, M, P, F, Y or W (for example P or T), X₂₁ is D, E, N or Q (for example N); and X₂₂ is G, A, V, L or, I (for example L); X₂₃ is D; X₃₆ is D; and X₃₇ is D. Even more preferably, X₆ is N; X₇ is K; X₈ is E; X₉, X₁₀, X₁₁, X₁₃, X₁₄, X₁₇ and X₁₈ are as defined above; X₁₉ is L; X₂₀ is P or T; X₂₁ is N; and X₂₂ is L; X₂₃ is D; X₃₆ is D; and X₃₇ is D.

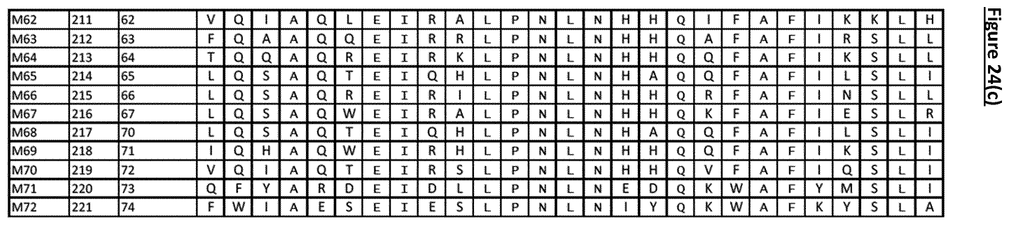

For example, the CD16a binding motif is selected from the group consisting of SEQ ID Nos. 150 to 221, as shown in FIG. 24.

For example, the CD16a binding motif is selected from the group consisting of

| | | Motif is |
|---|---|---|
| | SEQ | found in |
| Sequence | ID | Example Comp |
| IQHAQWEIRHLPNLNHHQQFAFIKSLI | 161 | 12 |
| LQYAQKEIRRLPNLNHHQVFAFINKLI | 166 | 17 |
| VQIAQREIRSLPNLNHHQVFAFIKKLL | 168 | 19 |
| LQAAQKEIRTLPNLNHHQVFAFINKLI | 178 | 29 |
| LQSAQREIRQLPNLNHHQKFAFINSLL | 182 | 33 |
| QQEAQLEIRRLPNLNHHQVFAFIKSLL | 198 | 49 |
| QQIAQYEIRKLPNLNHHQTFAFIKSLL | 200 | 51 |
| LQYAQVEIRKLPNLNHHQRFAFIKSLL | 202 | 53 |
| TQVAQKEIRTLPNLNHHQIFAFLNKLI | 164 | 15 |

In a polypeptide of FIG. 24 and the table above, the N-terminus of Helix 1 may further comprise the sequence X₆X₇X₈; wherein X₆, X₇ and X₈ are as defined above, and preferably are NKE (i.e. X₆ is N; X₇ is K; X₈ is E). In a polypeptide of FIG. 24 and the table above, the C-terminus of Helix 2 may further comprise the sequence X₃₆X₃₇; wherein X₃₆ and X₃₇ are as defined above, and preferably are DD i.e. X₃₆ is D; and X₃₇ is D).

In a polypeptide of FIG. 24 and the table above, optionally from 1 to 5 residues (for example 1, 2, 3, 4 or 5), preferably 1, 2 or 3 residues, in the sequence are replaced by an alternative residue. Preferably, in embodiments wherein optionally from 1 to 5 residues (for example 1, 2, 3, 4 or 5), preferably 1, 2 or 3 residues, in the sequence are replaced by an alternative residue, the replacement residue is a conservative replacement.

In a preferred embodiment, the sequence is the one of SEQ ID 166, 168, 178, 182, or 202 (i.e. as found in one of Example Compounds 17, 19, 29, 33, and 53) (and optionally from 1 to 5 residues (for example 1, 2, 3, 4 or 5), preferably 1, 2 or 3 residues, in the sequence are replaced by an alternative residue, the replacement residue is a conservative replacement).

In another preferred embodiment, the sequence is the one of SEQ ID No. 164, 166, 168, or 200, or SEQ ID No. 164, 166 or 168 (i.e. as found in one of Example Compounds 15, 17, 19, 51, or as found in one of Example Compounds 15, 17, 19) (and optionally from 1 to 5 residues (for example 1, 2, 3, 4 or 5), preferably 1, 2 or 3 residues, in the sequence are replaced by an alternative residue, the replacement residue is a conservative replacement).

In preferred embodiments, the motif sequence additionally has the residues NKE at positions X₆X₇X₈ (i.e. X₆ is N; X₇ is K; X₈ is E). In preferred embodiments, the motif sequence additionally has the residues DD at its positions X₃₆X₃₇ (i.e. X₃₆ is D; and X₃₇ is D). For example, the motif sequences have NKE at positions X₆X₇X₈ and DD at positions X₃₆X₃₇ (i.e. X₆ is N; X₇ is K; X₈ is E; X₃₆ is D; and X₃₇ is D). Therefore, in preferred embodiments, the motif sequence may be selected from:

NKEX₉X₁₀X₁₁AX₁₃X₁₄EIX₁₇X₁₈X₁₉X₂₀X₂₁X₂₂X₂₃X₂₄X₂₅QX₂₇X₂₈AFX₃₁
X₃₂X₃₃LX₃₅X₃₆X₃₇;
X₆X₇X₈X₉X₁₀X₁₁AX₁₃X₁₄EIX₁₇X₁₈X₁₉X₂₀X₂₁X₂₂X₂₃X₂₄X₂₅QX₂₇X₂₈AF
X₃₁X₃₂X₃₃LX₃₅DD;
and
NKEX₉X₁₀X₁₁AX₁₃X₁₄EIX₁₇X₁₈X₁₉X₂₀X₂₁X₂₂X₂₃X₂₄X₂₅QX₂₇X₂₈AFX₃₁
X₃₂X₃₃LX₃₅DD.

As mentioned above, in embodiments of the invention a number of residues may each be substituted by an alternative residue, as described herein. For example, in the embodiments described above, even when not explicitly mentioned, optionally 1 to 5 (for example 1, 2, 3, 4 or 5), and preferably optionally 1 to 3 (for example 1, 2, or 3), residues in the sequence of Helix 1 and/or Helix 2 defined above are replaced by an alternative residue (for example replaced by an alternative residue that is a conservative replacement). In any such alternative polypeptides of the invention with alternative residues in place, binding to the CD16a receptor is maintained. For example, the CD16a binding efficacy is at least 1% of the binding efficacy of the peptide of SEQ ID NO: 1 (i.e. binding efficacy of the peptide of SEQ ID NO: 1 to the CD16a receptor, when measured under the same conditions (as described above)). Alternatively, or additionally, for example, in any such alternative polypeptides of the invention with alternative residues in place, the CD16a-binding polypeptide is one that competes with SEQ ID NO: 1.

In certain preferred embodiments, the binding efficacy is at least 5%, and more preferably at least 10%, 20%, 25% or 50% of the binding efficacy of the peptide of SEQ ID NO: 1 (i.e. binding efficacy of the peptide of SEQ ID NO: 1 to the CD16a receptor, when measured under the same conditions).

In advantageous embodiments of CD16a-binding polypeptides of the invention, Proline (P) and Cysteine (C) are not present in CD16a binding motif of a CD16a binding polypeptide of the present invention. Advantageously Glycine (G) is also not present in the CD16a binding motif of a CD16a binding polypeptide of the present invention.

In certain embodiments of the invention, the CD16a-binding polypeptide is one wherein:
i) Helix 1 comprises the sequence X₉X₁₀X₁₁AX₁₃X₁₄EIX₁₇X₁₈ and Helix 2 has the sequence X₂₄X₂₅QX₂₇X₂₈AFX₃₁X₃₂SLX₃₅, wherein,
   a*) X₉ is V; X₁₀ is Q; X₁₁ is M; X₁₃ is Q; X₁₄ is F; X₁₇ is R; X₁₈ is K; X₂₄ is H; X₂₅ is H; X₂₇ is S; X₂₈ is F; X₃₁ is I; X₃₂ is K; and X₃₅ is M;
   b*) X₉ is Q; X₁₀ is F; X₁₁ is Y; X₁₃ is R; X₁₄ is D; X₁₇ is D; X₁₈ is L; X₂₄ is E; X₂₅ is D; X₂₇ is K; X₂₈ is W; X₃₁ is Y; X₃₂ is M; and X₃₅ is I; or
   c*) X₉ is F; X₁₀ is W; X₁₁ is I; X₁₃ is E; X₁₄ is S; X₁₇ is E; X₁₈ is S; X₂₄ is I; X₂₅ is Y; X₂₇ is K; X₂₈ is W; X₃₁ is K; X₃₂ is Y; and X₃₅ is A;
or ii) Helix 1 and Helix 2 are defined as in i), wherein within Helix 1 and Helix 2, at least 1 and no more than 5 (for example 1, 2, 3, 4 or 5; or for example, at least 1 and no more than 3) of the Xₙ residues are replaced by an alternative residue, and/or at least 1 and no more than 5 (for example 1, 2, 3, 4 or 5; or for example, at least 1 and no more than 3) of the residues not labelled as Xₙ are replaced by an alternative residue.

In another embodiment, a*) X₉ is V; X₁₀ is Q; X₁₁ is I or norleucine; X₁₃ is Q; X₁₄ is F; X₁₇ is R; X₁₈ is K; X₂₄ is H; X₂₅ is H; X₂₇ is S; X₂₈ is F; X₃₁ is I; X₃₂ is K; and X₃₅ is I or norleucine; In an embodiment, Helix 1 comprises the sequence KEX₉X₁₀X₁₁AX₁₃X₁₄EIX₁₇X₁₈ and Helix 2 comprises the sequence X₂₄X₂₅QX₂₇X₂₈AFX₃₁X₃₂SLX₃₅D, for example Helix 1 comprises the sequence NKEX₉X₁₀X₁₁AX₁₃X₁₄EIX₁₇X₁₈L and Helix 2 comprises the sequence NX₂₄X₂₅QX₂₇X₂₈AFX₃₁X₃₂SLX₃₅DD.

As mentioned above, full identity with the sequences set out for (i) is not required (though in one preferred embodiment of the invention, the peptide does have the exact recited sequence). Of the residues denoted with an Xₙ label, in certain embodiments, at least 1 and no more than 8 of the residues may be replaced with an alternative residue. The replaced residues may be in Helix 1 or in Helix 2, or there may be 1 or more replaced residues in each of Helix 1 and Helix 2 (for example, there may be replaced residue in one of them and 1 or 2 replaced residues in the other). For example, there may be 1, 2, 3, 4, 5, 6, 7 or 8 (for example 1, 2, 3, 4 or 5, or 1, 2 or 3) replaced residues in the residues denoted with an Xₙ label, for example 1, 2, 3, 4 or 5, for example 1 or 2, or 1. As there are 15 residues with an X₁ label, a peptide with 5 residues replaced has 67% sequence identity with the recited sequence. For replacement of 3 residues, it is 80% and for replacement of 1 residue it is 93% sequence identity. For replacement of 2 residues, it is 87% and for replacement of 1 residue it is 93% sequence identity. In an embodiment in which there are 14 residues with an Xₙ label, a peptide with 5 residues replaced has 64% sequence identity with the recited sequence. For replacement of 3 residues, it is 79% and for replacement of 1 residue it is 93% sequence identity. For replacement of 2 residues, it is 86% and for replacement of 1 residue it is 93% sequence identity.

Of the residues not labelled as an Xₙ residue, at least 1 and no more than 5 of the residues may be replaced with an alternative residue. Preferably, at least 1 and no more than 3 of the residues may be replaced with an alternative residue. The replaced residues may be in either Helix 1 or in Helix 2, or there may be 1 or more replaced residues in each of Helix 1 and Helix 2 (for example there may be replaced residue in one of them and 1 or 2 replaced residues in the other). For example, there may be 1, 2 or 3 replaced residues in the residues not denoted with an X₁ label, particularly 1 or 2, for example 1. As there are 8 residues without an X₁ label, a peptide with 3 residues replaced has 63% sequence identity with the recited sequence. For replacement of 2 residues, it is 75% and for replacement of 1 residue it is 88% sequence identity.

In an embodiment, the total number of residues in the Helix 1 and Helix 2 portions that are replaced is at least 1 and no more than 11, (for example no more than 10, for example no more than 9, for example no more than 8, for example no more than 7), for example at least 1 and no more than 6, for example at least 1 and no more than 5, at least 1 and no more than 4, for example at least 1 and no more than 3, for example 1 at least 1 and no more than 2. Particularly, there may be 1, 2, 3, 4, 5 or 6 replacement residues in total in those portions.

For example, an amino acid residue replacement for a residue denoted as Xₙ or a residue not denoted as Xₙ may be a conservative replacement. That is to say that a residue is replaced with another residue in the same class, for example: An aliphatic residue (Glycine (G), Alanine (A), Valine (V), Leucine (L) or Isoleucine (I)) may be replaced with another aliphatic residue. A hydroxyl-, sulphur- or selenium-containing residue (Serine (S), Cysteine (C), Selenocysteine (U), Threonine (T) or Methionine (M)) may be replaced with another hydroxyl-, sulphur- or selenium-containing residue. An aromatic residue (Phenylalanine (F), Tyrosine (Y) or Tryptophan (W)) may be replaced with another aromatic residue. A basic residue (Histidine (H), Lysine (K), Arginine (R)) may be replaced with another basic residue. An acidic residue or amide (Aspartate (D), Glutamate (E), Asparagine (N), Glutamine (Q)) may be replaced with another acidic residue or amide.

Alternatively, an amino acid residue replacement for a residue denoted as Xₙ or a residue not denoted as Xₙ may be a non-conservative replacement, i.e. a residue is replaced with another residue in a different class, for example an aliphatic residue (Glycine (G), Alanine (A), Valine (V), Leucine (L) or Isoleucine (I)) may be replaced with an aromatic residue (Phenylalanine (F), Tyrosine (Y) or Tryptophan (W)), or for example replaced by an amino acid with opposite characteristics, for example replacement of a Lysine (K) residue with an Aspartic acid (D) residue.

In one embodiment, the CD16a-binding polypeptide is one wherein:
a*) Helix 1 comprises the sequence VQMAQFEIRK (SEQ ID No 230) and Helix 2 comprises the sequence HHQSFAFIKSLM (SEQ ID No 231). For example, Helix 1 comprises the sequence NKEVQMAQFEIRKL (SEQ ID No 232) and Helix 2 comprises the sequence NHHQSFAFIKSLMDD (SEQ ID No 233).

In such embodiments, optionally at least 1 and no more than 5 (for example 1, 2, 3, 4 or 5; or for example at least 1 and no more than 3 (for example 1, 2, or 3)) residues in the sequence of Helix 1 and/or Helix 2 are replaced by an alternative residue (for example replaced by an alternative residue that is a conservative replacement);

In a further embodiment, the CD16a-binding polypeptide is one wherein:
b*) Helix 1 comprises the sequence QFYARDEIDL (SEQ ID No 234) and Helix 2 comprises the sequence EDQKWAFYMSLI (SEQ ID No 235). For example, Helix 1 comprises the sequence NKEQFYARDEIDLL (SEQ ID No 236) and Helix 2 comprises the sequence NEDQKWAFYMSLIDD (SEQ ID No 237).

In such embodiments, optionally at least 1 and no more than 5 (for example 1, 2, 3, 4 or 5; or for example at least 1 and no more than 3 (for example 1, 2, or 3)) residues in the sequence of Helix 1 and/or Helix 2 are replaced by an alternative residue (for example replaced by an alternative residue that is a conservative replacement);

In a further embodiment, the CD16a-binding polypeptide is one wherein:
c*) Helix 1 comprises the sequence FWIAESEIES (SEQ ID No 238) and Helix 2 comprises the sequence IYQKWAFKYSLA (SEQ ID No 239). For example, Helix 1 comprises the sequence NKEFWIAESEIESL(SEQ ID No 240) and Helix 2 comprises the sequence NIYQKWAFKYSLADD (SEQ ID No 241).

In such embodiments, optionally at least 1 and no more than 5 (for example 1, 2, 3, 4 or 5; or for example at least 1 and no more than 3 (for example 1, 2, or 3)) residues in the sequence of Helix 1 and/or Helix 2 are replaced by an alternative residue (for example replaced by an alternative residue that is a conservative replacement); As discussed above, a number of residues may each be substituted by an alternative residue.

The CD16a-binding polypeptide of the invention has the overall structure [N-terminal portion]-[Helix 1]-[Separating portion]-[Helix 2]—[C-terminal portion].

The separating portion may be a sequence of from 1 to 5 (for example 1, 2, 3, 4 or 5) naturally occurring amino acids. Preferably, the separating portion is a sequence of from 2 to 5 (for example 2, 3, 4 or 5) naturally occurring amino acids. For example, the separating portion is a sequence of from 3 to 5 (for example 3, 4 or 5) naturally occurring amino acids. Preferably the separating portion is a sequence of 3 amino acids.

In certain embodiments, the separating portion has the sequence X₂₀X₂₁X₂₂, wherein X₂₀ is any naturally occurring amino acid, X₂₁ is any naturally occurring amino acid; and X₂₂ is any naturally occurring amino acid; and wherein optionally one or two of X₂₀, X₂₁ or X₂₂ are absent. For example, none of X₂₀, X₂₁ or X₂₂ is absent, one of X₂₀, X₂₁ or X₂₂ is absent, or two of X₂₀, X₂₁ or X₂₂ are absent. More preferably, none of X₂₀, X₂₁ or X₂₂ is absent or one of X₂₀, X₂₁ or X₂₂ is absent. Most preferably none of X₂₀, X₂₁ or X₂₂ is absent, i.e. the separating portion has the sequence X₂₀X₂₁X₂₂, wherein X₂₀ is any naturally occurring amino acid, X₂₁ is any naturally occurring amino acid; and X₂₂ is any naturally occurring amino acid.

Preferably, X₂₀ is S, T, M, P, F, Y or W (for example P or T); X₂₁ is D, E, N or Q; and X₂₂ is G, A, V, L or I; wherein optionally one or two (for example one) of X₂₀, X₂₁ or X₂₂ are absent.

In preferred embodiments, X₂₀ is S, T, M, P, F, Y or W (for example P or T), X₂₁ is D, E, N or Q; and X₂₂ is G, A, V, L, I. More preferably, X₂₀ is P or T; X₂₁ is N; and X₂₂ is L. For example, the separating portion has the sequence PNL or TNL.

The N-terminal portion may be absent or may be a sequence of 1 to 15 naturally occurring amino acids. For example, the N-terminal portion may be absent or may be a sequence of from 1 to 10, from 1 to 8, from 1 to 6, or from 1 to 5 naturally occurring amino acids, for example 1, 2, 3, 4 or 5 naturally occurring amino acids.

In certain embodiments, the N-terminal portion has the sequence X₁X₂X₃X₄X₅, wherein X₁ is any naturally occurring amino acid or is absent; X₂ is any naturally occurring amino acid or is absent; X₃ is any naturally occurring amino acid or is absent; X₄ is any naturally occurring amino acid or is absent; and X₈ is any naturally occurring amino acid or is absent.

Preferably, the N-terminal portion has the sequence X₁X₂X₃X₄X₅, wherein X₁ is G, A, V, L, I or is absent, X₂ is D, E, N, Q or is absent, X₃ is D, E, N, Q or is absent, X₄ is H, K, R or is absent, and X₈ is F, Y, W or is absent. More preferably, X₁ is V, G or absent (for example V or absent); X₂ is D or absent; X₃ is N or absent; X₄ is K or absent; and X₅ is F or absent.

In certain embodiments, preferably none of X₁, X₂, X₃, X₄ and X₈ is absent; X₁ is absent and the other residues are not absent; X₁ and X₂ are absent and the other residues are not absent; X₁, X₂ and X₃ are absent and the other residues are not absent; X₁, X₂, X₃ and X₄ are absent and the other residue is not absent; or all of X₁, X₂, X₃, X₄ and X₈ are absent.

In certain especially preferred embodiments, the N-terminal portion has the sequence X₁X₂X₃X₄X₅ wherein X₁ is V or G (preferably V), X₂ is D, X₃ is N, X₄ is K, and X₈ is F; X₁ is absent, X₂ is D, X₃ is N, X₄ is K, and X₈ is F; X₁ is absent, X₂ is absent, X₃ is N, X₄ is K, and X₈ is F; X₁ is absent, X₂ is absent, X₃ is absent, X₄ is K, and X₈ is F; X₁ is absent, X₂ is absent, X₃ is absent, X₄ is absent, and X₈ is F; or X₁ is absent, X₂ is absent, X₃ is absent, X₄ is absent, and X₈ is absent. For example, X₁ is V or G (preferably V), X₂ is D, X₃ is N, X₄ is K, and X₈ is F; or X₁ is absent, X₂ is absent, X₃ is absent, X₄ is absent, and X₈ is absent, i.e. the N-terminal portion has the sequence VDNKF, GDNKF or is absent; and more preferably has the sequence VDNKF or is absent.

In one preferred embodiment, X₁ is V or G (preferably V), X₂ is D, X₃ is N, X₄ is K, and X₈ is F, i.e. the N-terminal portion has the sequence VDNKF or GDNKF; and more preferably the N-terminal portion has the sequence VDNKF.

In one preferred embodiment, X₁ is absent, X₂ is absent, X₃ is absent, X₄ is absent, and X₈ is absent, i.e. the N-terminal portion is absent.

The C-terminal portion may be absent or may be a sequence of 1 to 50 naturally occurring amino acids. For example, the C-terminal portion may be absent or may be a sequence of from 1 to 40, 1 to 35, 1 to 30, from 1 to 25, from 1 to 22, or from 1 to 21 naturally occurring amino acids. Preferably, the C-terminal portion may be absent or may be a sequence of from 10 to 35, 10 to 30, from 15 to 25, from 18 to 22, from 1 to 21 naturally occurring amino acids, for example 18, 19, 20, 21 or 22 naturally occurring amino acids. Preferably, the C-terminal portion has a sequence such that it enhances target binding by the CD16a-binding motif of Helix 1 and Helix 2.

In certain embodiments, the C-terminal portion is absent or has the sequence X₃₈X₃₉QSANLLAEAKKLNDAQX₅₆X₅₇X₅₈, wherein X₃₈ is a sequence of 1 to 14 naturally occurring amino acids, X₃₉ is any naturally occurring amino acid, X₅₆ is any naturally occurring amino acid or is absent, X₅₇ is any naturally occurring amino acid or is absent, and X₅₈ is any naturally occurring amino acid or is absent, and optionally wherein from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement.

In certain embodiments, X₃₈ is a sequence of 1 to 9, 1 to 7 or 1 to 5 naturally occurring amino acids. More preferably X₃₈ is a sequence of 1 to 4, for example 1, 2, 3 or 4 amino acids. In one especially preferred embodiment, X₃₈ is any naturally occurring amino acid (i.e. any single (one) naturally occurring amino acid residue). In one preferred embodiment, the C-terminal portion has the sequence X₃₈X₃₉QSANLLAEAKKLNDAQX₅₆X₅₇X₅₈, wherein X₃₈ is P, X₃₉ is S, T, M, P, F, Y or W, X₅₆ is G, A, V, L, I or is absent, X₅₇ is P or is absent, and X₅₈ is H, K, R or is absent, and optionally wherein from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement. In another preferred embodiment, the C-terminal portion has the sequence PSQSANLLAEAKKLNDAQX₅₆X₅₇X₅₈, wherein X₅₆ is G, A, V, L, I or is absent, X₅₇ is P or is absent, and X₅₈ is H, K, R or is absent, and optionally wherein from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement.

In an especially preferred embodiment, the C-terminal portion has the X₃₈X₃₉QSANLLAEAKKLNDAQX₅₆X₅₇X₅₈, wherein X₃₈ is P, X₃₉ is S, X₅₆ is A or is absent, X₅₇ is P or is absent, and X₅₈ is K or is absent, and optionally wherein from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement. In another especially preferred embodiment, the C-terminal portion has the PSQSANLLAEAKKLNDAQX₅₆X₅₇X₅₈, wherein X₅₆ is A or is absent, X₅₇ is P or is absent, and X₅₈ is K or is absent, and optionally wherein from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement.

For example, in certain embodiments the C-terminal portion has the sequence X₃₈X₃₉QSANLLAEAKKLNDAQX₅₆X₅₇X₅₈, wherein X₃₈ is P, and X₃₉ is S; and: X₅₆ is A, X₅₇ is P, and X₅₈ is K; or X₅₆ is A, or X₅₇ is P, and X₅₈ is absent; orX56 is A, X₅₇ is absent, and X₅₈ is absent; or X₅₆ is absent, X₅₅ is absent, and X₅₈ is absent. In such embodiments optionally from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement. Or, for example, the C-terminal portion has the sequence PSQSANLLAEAKKLNDAQX₅₆X₅₇X₅₈, wherein X₅₆ is A, X₅₇ is P, and X₅₈ is K; or X₅₆ is A, or X₅₇ is P, and X₅₈ is absent; or X₅₆ is A, X₅₇ is absent, and X₅₈ is absent; or X₅₆ is absent, X₅₇ is absent, and X₅₈ is absent. In such embodiments optionally from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement.

In one preferred the C-terminal portion has the sequence X₃₈X₃₉QSANLLAEAKKLNDAQX₅₆X₅₇X₅₈, wherein X₃₈ is P, and X₃₉ is S; and X₅₆ is A, X₅₇ is P, and X₅₈ is K. In such embodiments optionally from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement. Or, for example, the C-terminal portion has the sequence PSQSANLLAEAKKLNDAQX₅₆X₅₇X₅S, wherein X₅₆ is A, X₅₇ is P, and X₅₈ is K. In such embodiments optionally from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement.

In an alternative preferred embodiment the C-terminal portion has the sequence X₃₈X₃₉QSANLLAEAKKLNDAQX₅₆X₅₇X₅₈, wherein X₃₈ is P, and X₃₉ is S; and: X₅₆ is absent, X₅₇ is absent, and Xss is absent. In such embodiments optionally from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement. Or, for example, For example, the C-terminal portion has the sequence PSQSANLLAEAKKLNDAQX₅₆X₅₇X₅₈, X₅₆ is absent, X₅₇ is absent, and X₅₈ is absent. In such embodiments optionally from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement.

In one embodiment, the N-terminal portion has the sequence X₁X₂X₃X₄X₅ wherein X₁ is V or G (preferably V), X₂ is D, X₃ is N, X₄ is K, and X₈ is F; X₁ is absent, X₂ is D, X₃ is N, X₄ is K, and X₈ is F; X₁ is absent, X₂ is absent, X₃ is N, X₄ is K, and X₈ is F; X₁ is absent, X₂ is absent, X₃ is absent, X₄ is K, and X₈ is F; X₁ is absent, X₂ is absent, X₃ is absent, X₄ is absent, and X₈ is F; or X₁ is absent, X₂ is absent, X₃ is absent, X₄ is absent, and X₈ is absent (for example, X₁ is V or G (preferably V), X₂ is D, X₃ is N, X₄ is K, and X₈ is F, or X₁ is absent, X₂ is absent, X₃ is absent, X₄ is absent, and X₈ is absent); and the C-terminal portion has the sequence X₃₈X₃₉QSANLLAEAKKLNDAQX₅₆X₅₇X₅₈, wherein X₃₈ is P, X₃₉ is S, T, M, P, F, Y or W, X₅₆ is G, A, V, L, I or is absent, X₅₇ is P, and X₅₈ is H, K, or R, and optionally wherein from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement; and more preferably X₃₈ is P, X₃₉ is S, X₅₆ is A, X₅₇ is P, and X₅₈ is K, and optionally from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement.

In another embodiment, the N-terminal portion has the sequence X₁X₂X₃X₄X₈ wherein X₁ is V or G (preferably V), X₂ is D, X₃ is N, X₄ is K, and X₈ is F; X₁ is absent, X₂ is D, X₃ is N, X₄ is K, and X₈ is F; X₁ is absent, X₂ is absent, X₃ is N, X₄ is K, and X₈ is F; X₁ is absent, X₂ is absent, X₃ is absent, X₄ is K, and X₈ is F; X₁ is absent, X₂ is absent, X₃ is absent, X₄ is absent, and X₈ is F; or X₁ is absent, X₂ is absent, X₃ is absent, X₄ is absent, and X₈ is absent (for example, X₁ is V or G (preferably V), X₂ is D, X₃ is N, X₄ is K, and X₈ is F, or X₁ is absent, X₂ is absent, X₃ is absent, X₄ is absent, and X₈ is absent); and the C-terminal portion has the sequence PSQSANLLAEAKKLNDAQX₅₆X₅₇X₅₈, wherein X₅₆ is G, A, V, L, I or is absent, X₅₇ is P, and X₅₈ is H, K, or R, and optionally wherein from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement; and more preferably X₅₆ is A, X₅₇ is P, and X₅₈ is K, and optionally from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement.

In one very preferred embodiment, X₁ is V or G (preferably V), X₂ is D, X₃ is N, X₄ is K, and X₈ is F; and X₅₆ is G, A, V, L or I (preferably A), X₅₇ is P, and X₅₈ is H, K, or R (preferably K).

In an alternative very preferred embodiment X₁ is absent, X₂ is absent, X₃ is absent, X₄ is absent, and X₈ is absent; and X₅₆ is G, A, V, L or I (preferably A), X₅₇ is P, and X₅₈ is H, K, or R (preferably K).

In another preferred embodiment, the N-terminal portion has the sequence X₁X₂X₃X₄X₅, wherein X₁ is G, A, V, L, or I, X₂ is D, E, N, or Q, X₃ is D, E, N, or Q, X₄ is H, K, or R, and X₈ is F, Y, or W (for example, X₁ is V or G (preferably V), X₂ is D, X₃ is N, X₄ is K, and X₈ is F); and the C-terminal portion has sequence X₃₈X₃₉QSANLLAEAKKLNDAQX₅₆X₅₇X₅₈, wherein X₃₈ is P, X₃₉ is S, T, M, P, F, Y or W (preferably S); and X₅₆ is G, A, V, L or I (preferably A), X₅₇ is P, and X₅₈ is H, K, or R (preferably K); X₅₆ is G, A, V, L or I (preferably A), X₅₇ is P, and X₅₈ is absent; X₅₆ is G, A, V, L or I (preferably A), X₅₇ is absent, and X₅₈ is absent; or X₅₆ is absent, X₅₇ is absent, and X₅₈ is absent. More preferably, X₅₆ is G, A, V, L or I (preferably A), X₅₇ is P, and X₅₈ is H, K, or R (preferably K); or X₅₆ is absent, X₅₇ is absent, and X₅₈ is absent. In such embodiments optionally from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement.

In one preferred embodiment, the N-terminal portion has the sequence X₁X₂X₃X₄X₅, wherein X₁ is G, A, V, L, or I, X₂ is D, E, N, or Q, X₃ is D, E, N, or Q, X₄ is H, K, or R, and X₈ is F, Y, or W (for example, X₁ is V or G (preferably V), X₂ is D, X₃ is N, X₄ is K, and X₈ is F); and the C-terminal portion has sequence PSQSANLLAEAKKLNDAQX₅₆X₅₇X₅₈, wherein X₅₆ is G, A, V, L or I (preferably A), X₅₇ is P, and X₅₈ is H, K, or R (preferably K); X₅₆ is G, A, V, L or I (preferably A), X₅₇ is P, and X₅₈ is absent; X₅₆ is G, A, V, L or I (preferably A), X₅₇ is absent, and X₅₈ is absent; or X₅₆ is absent, X₅₇ is absent, and X₅₈ is absent. More preferably, X₅₆ is G, A, V, L or I (preferably A), X₅₇ is P, and X₅₈ is H, K, or R (preferably K); or X₅₆ is absent, X₅₇ is absent, and X₅₈ is absent. In such embodiments optionally from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement.

In one very preferred embodiment, X₁ is V or G (preferably V), X₂ is D, X₃ is N, X₄ is K, and X₈ is F; and X₅₆ is absent, X₅₇ is absent, and X₅₈ is absent.

In an alternative very preferred embodiment, X₁ is V or G (preferably V), X₂ is D, X₃ is N, X₄ is K, and X₈ is F; X₅₆ is G, A, V, L or I (preferably A), X₅₇ is P, and X₅₈ is H, K, or R (preferably K).

In certain embodiments, (i) the separating portion has the sequence X₂₀X₂₁X₂₂; and/or the N-terminal portion has the sequence X₁X₂X₃X₄X₅; and/or the C-terminal portion has the sequence PSQSANLLAEAKKLNDAQX₅₆X₅₇X₅₈;
wherein, in said separating portion, X₂₀ is P or T; X₂₁ is N; X₂₂ is L;
wherein in said N-terminal portion, X₁ is V or G (preferably V), or absent; X₂ is D or absent; X₃ is N or absent; X₄ is K or absent; X₈ is F or absent;
and wherein in said C-terminal portion, X₅₆ is A or absent; X₅₇ is P or absent; X₅₈ is K or absent.

Alternatively, (ii) the separating portion, N-terminal portion, and C-terminal portion are as defined in (i) above, wherein optionally
(a) within each portion 1, 2 or 3 residues are replaced by an alternative residue; or
(b) within those portions taken together at least 1 and no more than 5 (for example, 1, 2, 3, 4, or 5) residues are replaced by an alternative residue.

In such embodiments, preferably the separating portion has the sequence PNL or TNL. Alternatively, or additionally, in such embodiments, the N-terminal portion has the sequence VDNKF.

In one embodiment, the N-terminal portion may comprise the sequence X₁X₂X₃X₄X₅, wherein:
X₁ is V, or absent; X₂ is D or absent; X₃ is N or absent; X₄ is K or absent; X₅ is F or absent.

For example, the N-terminal portion may comprise the sequence VDNKF.

The separating portion may comprise the sequence X₂₀X₂₁X₂₂, wherein:
X₂₀ is P or T; X₂₁ is N; X₂₂ is L.

For example, the separating portion may comprise the sequence PNL.

The C-terminal portion may comprise the sequence PSQSANLLAEAKKLNDAQX₅₆X₅₇X₅₈, wherein:
X₅₆ is A or absent; X₅₇ is P or absent; X₅₈ is K or absent.

For example, the C-terminal portion may comprise the sequence PSQSANLLAEAKKLNDAQAPK or PSQSANLLAEAKKLNDAQ. The C-terminal portion may have the structure [Second separating portion]-[Helix 3]—[C-terminal sequence]. For example:
the Second separating portion comprises the sequence PS;
the Helix 3 portion comprises the sequence QSANLLAEAKKLNDAQ;
and the C-terminal sequence comprises the sequence APK or is absent.

In such embodiments, within those five portions (N-terminal portion, separating portion, second separating portion, Helix 3, and C-terminal sequence) taken together, at least 1 and no more than 8 (for example 5) of the residues may be replaced by an alternative residue. For example, the number of replaced residues is at least 1 and no more than 7, at least 1 and no more than 6, at least 1 and no more than 5, at least 1 and no more than 4, for example at least 1 and no more than 3, for example at least 1 and no more than 2. Particularly, there may be 1, 2, 3, 4 or 5 replacement residues in total in those portions.

Thus, in such embodiments, across the entire CD16a-binding polypeptide of the invention (i.e. across the entire overall structure [N-terminal portion]-[Helix 1]-[Separating portion]-[Helix 2]—[C-terminal portion]), at least 1 and no more than 11 of the residues may be replaced by an alternative residue. For example, the number of replaced residues is at least 1 and no more than 10, for example at least 1 and no more than 9, for example at least 1 and no more than 8, for example at least 1 and no more than 7, for example at least 1 and no more than 6. Particularly, there may be 1, 2, 3, 4, 5, 6, 7, 8, 9, 10 or 11 replacement residues in total across the entire overall structure [N-terminal portion]-[Helix 1]-[Separating portion]-[Helix 2]—[C-terminal portion].

In advantageous embodiments of CD16a-binding polypeptides of the invention, where the C-terminal portion comprises a helical or substantially helical region (for example Helix 3 as described immediately above; for example the sequence QSANLLAEAKKLNDAQ), Proline (P) and Cysteine (C) are not present in that helical or substantially helical region. In another embodiment, additionally Glycine (G) is also not present in that helical or substantially helical region for example Helix 3 as described immediately above; for example the sequence QSANLLAEAKKLNDAQ).

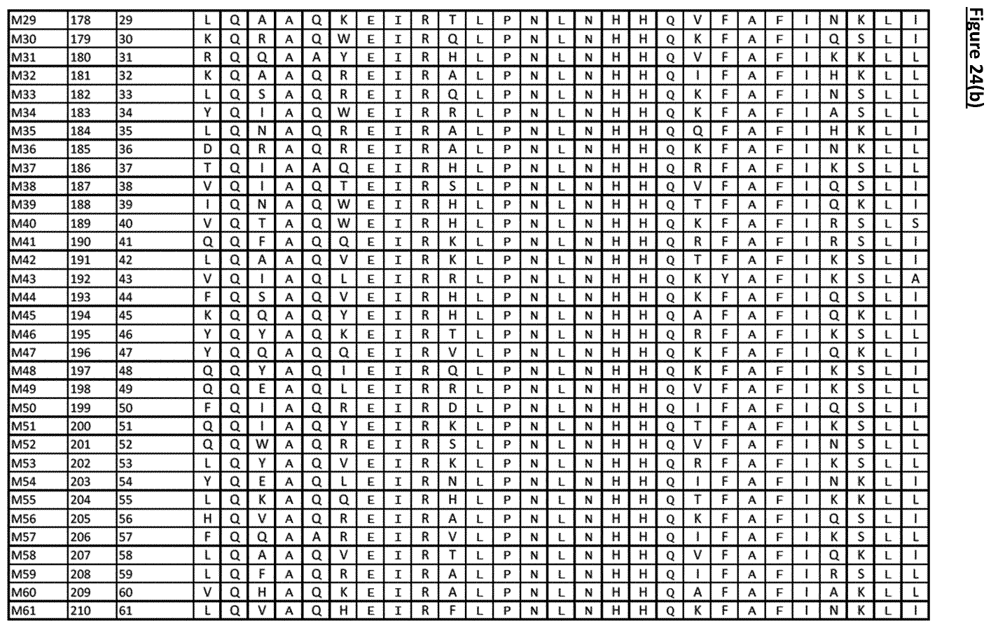

In one aspect of the invention, the CD16a binding polypeptide comprises a sequence selected from SEQ ID NO. 1 to 75 as shown in the table FIG. 23. In such sequences, optionally from 1 to 5 (preferably optionally 1, 2 or 3) residues in the sequence are replaced by an alternative residue, and preferably a residue that is a conservative replacement. In one embodiment, the CD16a binding polypeptide comprises a sequence selected from SEQ ID NO. 1 to 75. In one embodiment, the CD16a binding polypeptide has a sequence selected from the group consisting of SEQ ID NO. 1 to 75.

In one aspect of the invention, the CD16a binding polypeptide comprises a sequence selected from SEQ ID NO. 1 to 73 as shown in the table FIG. 23. In such sequences, optionally from 1 to 5 (preferably optionally 1, 2 or 3) residues in the sequence are replaced by an alternative residue, and preferably a residue that is a conservative replacement. In one embodiment, the CD16a binding polypeptide comprises a sequence selected from SEQ ID NO. 1 to 73. In one embodiment, the CD16a binding polypeptide has a sequence selected from the group consisting of SEQ ID NO. 1 to 73.

In one aspect of the invention, the CD16a binding polypeptide comprises a sequence selected from SEQ ID NO. 11 to 73 as shown in the table FIG. 23. In such sequences, optionally from 1 to 5 (preferably optionally 1, 2 or 3) residues in the sequence are replaced by an alternative residue, and preferably a residue that is a conservative replacement. In one embodiment, the CD16a binding polypeptide comprises a sequence selected from SEQ ID NO. 11 to 73. In one embodiment, the CD16a binding polypeptide has a sequence selected from the group consisting of SEQ ID NO. 11 to 73.

In one aspect of the invention, the CD16a binding polypeptide comprises a sequence selected from SEQ ID NO. 1 to 67 as shown in the table FIG. 23. In such sequences, optionally from 1 to 5 (preferably optionally 1, 2 or 3) residues in the sequence are replaced by an alternative residue, and preferably a residue that is a conservative replacement. In one embodiment, the CD16a binding polypeptide comprises a sequence selected from SEQ ID NO. 11 to 67. In one embodiment, the CD16a binding polypeptide has a sequence selected from the group consisting of SEQ ID NO. 1 to 67.

In one aspect of the invention, the CD16a binding polypeptide comprises a sequence selected from SEQ ID NO. 19, 33, 17, 29, 53, 16, 25, 15, 51, 36, 49, 55, 43, 24, 56, 12, 28, 21, 59, 52, 32, 18, 27, 35 and 11 as shown in the table FIG. 23. In such sequences, optionally from 1 to 5 (preferably optionally 1, 2 or 3) residues in the sequence are replaced by an alternative residue, and preferably a residue that is a conservative replacement. In one embodiment, the CD16a binding polypeptide comprises a sequence selected from of SEQ ID NO. 19, 33, 17, 29, 53, 16, 25, 15, 51, 36, 49, 55, 43, 24, 56, 12, 28, 21, 59, 52, 32, 18, 27, 35 and 11. In one embodiment, the CD16a binding polypeptide has a sequence selected from the group consisting of SEQ ID NO. 19, 33, 17, 29, 53, 16, 25, 15, 51, 36, 49, 55, 43, 24, 56, 12, 28, 21, 59, 52, 32, 18, 27, 35 and 11.

In one aspect of the invention, the CD16a binding polypeptide comprises a sequence selected from SEQ ID NO. 34, 45 and 51 as shown in the table FIG. 23. In such sequences, optionally from 1 to 5 (preferably optionally 1, 2 or 3) residues in the sequence are replaced by an alternative residue, and preferably a residue that is a conservative replacement. In one embodiment, the CD16a binding polypeptide comprises a sequence selected from of SEQ ID NO. 34, 45 and 51. In one embodiment, the CD16a binding polypeptide has a sequence selected from the group consisting of SEQ ID NO. 34, 45 and 51.

In one aspect of the invention, the CD16a binding polypeptide comprises a sequence selected from SEQ ID NO. 26, 35 and 47 as shown in the table FIG. 23. In such sequences, optionally from 1 to 5 (preferably optionally 1, 2 or 3) residues in the sequence are replaced by an alternative residue, and preferably a residue that is a conservative replacement. In one embodiment, the CD16a binding polypeptide comprises a sequence selected from of SEQ ID NO. 26, 35 and 47. In one embodiment, the CD16a binding polypeptide has a sequence selected from the group consisting of SEQ ID NO. 26, 35 and 47.

In one aspect of the invention, the CD16a binding polypeptide comprises a sequence selected from SEQ ID NO. 12, 17, 19, 29, 33, 49, 51, and 53 as shown in the table FIG. 23. In such sequences, optionally from 1 to 5 (preferably optionally 1, 2 or 3) residues in the sequence are replaced by an alternative residue, and preferably a residue that is a conservative replacement. In one embodiment, the CD16a binding polypeptide comprises a sequence selected from SEQ ID NO. 12, 17, 19, 29, 33, 49, 51, and 53. In one embodiment, the CD16a binding polypeptide has a sequence selected from the group consisting of SEQ ID NO. 12, 17, 19, 29, 33, 49, 51, and 53.

In one aspect of the invention, the CD16a binding polypeptide comprises a sequence selected from SEQ ID NO. 17, 19, 29, 33, and 53 as shown in the table FIG. 23. In such sequences, optionally from 1 to 5 (preferably optionally 1, 2 or 3) residues in the sequence are replaced by an alternative residue, and preferably a residue that is a conservative replacement. In one embodiment, the CD16a binding polypeptide comprises a sequence selected from of SEQ ID NO. 17, 19, 29, 33, and 53. In one embodiment, the CD16a binding polypeptide has a sequence selected from the group consisting of SEQ ID NO. 17, 19, 29, 33, and 53.

In one aspect of the invention, the CD16a binding polypeptide comprises a sequence selected from SEQ ID NO. 15, 17, 19, and 51 as shown in the table FIG. 23. In such sequences, optionally from 1 to 5 (preferably optionally 1, 2 or 3) residues in the sequence are replaced by an alternative residue, and preferably a residue that is a conservative replacement. In one embodiment, the CD16a binding polypeptide comprises a sequence selected from of SEQ ID NO. 15, 17, 19, and 51. In one embodiment, the CD16a binding polypeptide has a sequence selected from the group consisting of SEQ ID NO. 15, 17, 19, and 51.

In one aspect of the invention, the CD16a binding polypeptide comprises a sequence selected from SEQ ID NO. 15, 17 and 19 as shown in the table FIG. 23. In such sequences, optionally from 1 to 5 (preferably optionally 1, 2 or 3) residues in the sequence are replaced by an alternative residue, and preferably a residue that is a conservative replacement. In one embodiment, the CD16a binding polypeptide comprises a sequence selected from of SEQ ID NO. 15, 17 and 19. In one embodiment, the CD16a binding polypeptide has a sequence selected from the group consisting of SEQ ID NO. 15, 17 and 19.

In one aspect of the invention, the CD16a binding polypeptide comprises the sequence:

a*)
[SEQ ID NO: 1]
VDNKFNKEVQMAQFEIRKLPNLNHHQSFAFIKSLMDDPSQSANLLA
EAKKLNDAQAPK;

In a further aspect of the invention the CD16a binding polypeptide comprises the sequence:

b*)
[SEQ ID NO: 2]
VDNKFNKEQFYARDEIDLLPNLNEDQKWAFYMSLIDDPSQSANLL
AEAKKLNDAQAPK;

In a further aspect of the invention the CD16a binding polypeptide comprises the sequence:

c*)
[SEQ ID NO: 3]
VDNKFNKEFWIAESEIESLPNLNIYQKWAFKYSLADDPSQSANLLA
EAKKLNDAQAPK.

As discussed above, a number of residues in the polypeptides of the invention may be substituted by an alternative residue. For example, in a polypeptide as set out in a*), b*) or c*) immediately above, at least 1 and no more than 5 of the residues may be replaced by an alternative residue. For example, the number of replaced residues is at least 1 and no more than 4, for example at least 1 and no more than 3, for example 1 at least 1 and no more than 2. Particularly, there may be 1, 2, 3, 4 or 5 replacement residues in total in those portions. As there are 58 residues in polypeptides of this aspect of the invention, a peptide with 5 residues replaced has 91% sequence identity (91.4%) with the recited sequence. For replacement of 4 residues, it is 93% (93.1%), for replacement of 3 residues, it is 95% (94.8%), for replacement of 2 residues it is 97% (96.6%) and for replacement of 1 residue it is 98% sequence identity (98.3%).

As mentioned above, in an embodiment, a*) Helix 1 comprises the sequence VQMAQFEIRK and Helix 2 comprises the sequence HHQSFAFIKSLM. In that embodiment, as in others, a number of residues may each be substituted by an alternative residue, as described herein. In any such alternative polypeptides of the invention with alternative residues in place, binding to the CD16a receptor is maintained. For example, the CD16a binding efficacy is at least 1% of the binding efficacy of the peptide of SEQ ID NO: 1 to the CD16a receptor, when measured under the same conditions.

With 1% binding efficacy, it is understood that the IC₅₀ concentration of the alternative polypeptide for binding to the CD16a receptor is no more than 100 times the IC₅₀ concentration for the peptide of SEQ ID NO: 1 to the CD16a receptor, when measured under the same conditions.

More preferably, the binding efficacy is at least 5%, 10%, 20%, 25% or 50% of the binding efficacy of the peptide of SEQ ID NO: 1 to the CD16a receptor, when measured under the same conditions. That is to say that the IC₅₀ concentration of the alternative polypeptide for binding to the CD16a receptor is no more than 20 times, 10 times, 5 times, 4 times or 2 times the IC₅₀ concentration for the peptide of SEQ ID NO: 1 to the CD16a receptor, when measured under the same conditions.

As also mentioned above, in an embodiment, b*) Helix 1 comprises the sequence QFYARDEIDL and Helix 2 comprises the sequence EDQKWAFYMSLI. In that embodiment, as in others, a number of residues may each be substituted by an alternative residue, as described herein. In any such alternative polypeptides of the invention with alternative residues in place, binding to the CD16a receptor is maintained. For example, the CD16a binding efficacy is at least 1% of the binding efficacy of the peptide of SEQ ID NO: 74 to the CD16a receptor, when measured under the same conditions.

With 1% binding efficacy, it is understood that the IC₅₀ concentration of the alternative polypeptide for binding to the CD16a receptor is no more than 100 times the IC₅₀ concentration for the peptide of SEQ ID NO: 74 to the CD16a receptor, when measured under the same conditions.

More preferably, the binding efficacy is at least 5%, 10%, 20%, 25% or 50% of the binding efficacy of the peptide of SEQ ID NO: 74 to the CD16a receptor, when measured under the same conditions. That is to say that the IC₅₀ concentration of the alternative polypeptide for binding to the CD16a receptor is no more than 20 times, 10 times, 5 times, 4 times or 2 times the IC₅₀ concentration for the peptide of SEQ ID NO: 74 to the CD16a receptor, when measured under the same conditions.

As also mentioned above, in an embodiment, c*) Helix 1 comprises the sequence FWIAESEIES and Helix 2 comprises the sequence IYQKWAFKYSLA. In that embodiment, as in others, a number of residues may each be substituted by an alternative residue, as described herein. In any such alternative polypeptides of the invention with alternative residues in place, binding to the CD16a receptor is maintained. For example, the CD16a binding efficacy is at least 1% of the binding efficacy of the peptide of SEQ ID NO: 75 to the CD16a receptor, when measured under the same conditions.

With 1% binding efficacy, it is understood that the IC₅₀ concentration of the alternative polypeptide for binding to the CD16a receptor is no more than 100 times the IC₅₀ concentration for the peptide of SEQ ID NO: 75 to the CD16a receptor, when measured under the same conditions.

More preferably, the binding efficacy is at least 5%, 10%, 20%, 25% or 50% of the binding efficacy of the peptide of SEQ ID NO: 75 to the CD16a receptor, when measured under the same conditions. That is to say that the IC₅₀ concentration of the alternative polypeptide for binding to the CD16a receptor is no more than 20 times, 10 times, 5 times, 4 times or 2 times the IC₅₀ concentration for the peptide of SEQ ID NO: 75 to the CD16a receptor, when measured under the same conditions.

The invention further provides a CD16a-binding polypeptide, wherein the CD16a-binding polypeptide consists of one motif that binds to CD16a, wherein said polypeptide consists of the following structure:
[N-terminal portion]-[Helix 1]-[Separating portion]-[Helix 2]—[C-terminal portion]
the CD16a binding motif being the portion [Helix 1]-[Separating portion]-[Helix 2](and optionally the peptide further comprises one or more additional functional portions as described below (for example 1, 2, 3, 4, 5, 6 ore more); for example one, two or three additional functional portions).

The invention further provides a CD16a-binding polypeptide of the invention, wherein the CD16a-binding polypeptide consists of the CD16a-binding polypeptide (and optionally further comprises one or more additional functional portions as described below (for example 1, 2, 3, 4, 5, 6 ore more); for example one, two or three additional functional portions).

In one very preferred embodiment the CD16a-binding polypeptide (for example a CD16a-binding polypeptide of the invention described above or below) consists of one motif that binds to CD16a, wherein said polypeptide consists of the following structure:
[N-terminal portion]-[Helix 1]-[Separating portion]-[Helix 2]—[C-terminal portion]
the CD16a binding motif being the portion [Helix 1]-[Separating portion]-[Helix 2](and optionally further comprises one or more additional functional portions as described below (for example 1, 2, 3, 4, 5, 6 ore more); for example one, two or three additional functional portions).

In one very preferred embodiment the CD16a-binding polypeptide consists of a CD16a-binding polypeptide of the invention (and optionally further comprises one or more additional functional portions as described below (for example 1, 2, 3, 4, 5, 6 ore more); for example one, two or three additional functional portions).

For the avoidance of doubt, in embodiments wherein a CD16a-binding polypeptide consists of a CD16a-binding motif and/or a CD16a-binding polypeptide, the CD16a-binding polypeptide is not connected to a further CD16a-binding polypeptide, i.e. the CD16a-binding polypeptide is not a portion of a CD16a-binding oligomer of the invention.

In another very preferred embodiment the CD16a-binding polypeptide (for example a CD16a-binding polypeptide of the invention described above or below) consists of one motif that binds to CD16a, wherein said polypeptide consists of the following structure:
[N-terminal portion]-[Helix 1]-[Separating portion]-[Helix 2]—[C-terminal portion]
the CD16a binding motif being the portion [Helix 1]-[Separating portion]-[Helix 2], and wherein the polypeptide further comprises one or more additional functional portions as described below (for example 1, 2, 3, 4, 5, 6 ore more); for example one, two or three additional functional portions). In another very preferred embodiment the CD16a-binding consists of a CD16a-binding polypeptide of the invention, and further comprises one or more additional functional portions as described below (for example 1, 2, 3, 4, 5, 6 or more); for example 1, 2 or 3 additional functional portions (preferably 1 or 2 additional functional portions).

### Multimeric CD16a-Binding Polypeptides: CD16a-Binding Oligomers

The present invention further provides CD16a-binding oligomers which comprises at least two (i.e. two, or more than two, for example 2, 3, 4, 5, 6 or more; preferably 2, 3 or 4) CD16a-binding polypeptides of the present invention. In one preferred embodiment, CD16a-binding oligomer of the present invention comprises two CD16a-binding polypeptides of the present invention. In another embodiment, a CD16a-binding oligomer of the present invention comprises at least three, at least four, at least 5 or at least 6 CD16a-binding polypeptides of the present invention, for example 3, 4, 5 or 6 or more CD16a-binding polypeptides of the present invention.

The CD16a-binding polypeptides of a CD16a-binding oligomers of the invention may optionally be connected via one or more linkers. A CD16a-binding oligomer of the present invention is a multimeric binder, as it has multiple CD16a binding motifs, i.e. it has a CD16a binding motif ([Helix 1]-[Separating portion]-[Helix 2]) in each CD16a-binding polypeptide it comprises.

This aspect of the invention may also be defined such that the CD16a binding polypeptide constitutes two or more CD16a-binding moieties or CD16a-binding peptides, optionally connected via one or more linkers, for example two, three, four, five, six or more CD16a-binding moieties or CD16a-binding peptides, optionally connected via one or more linkers, for example two, three, or four CD16a-binding moieties optionally connected via one or more linkers. This definition is used in the numbered embodiments of the invention below to refer to the CD16a-binding oligomers aspects of the invention.

A CD16a-binding oligomer of the present invention comprises, at least, a first CD16a-binding polypeptide which is a CD16a-binding polypeptide of the present invention, and a second CD16a-binding polypeptide which is a CD16a-binding polypeptide of the present invention. The first and second CD16a-binding polypeptides may have the same sequence. Alternatively, the first and second CD16a-binding polypeptides may have different sequences. A CD16a-binding oligomer of the present invention may optionally further comprise a third CD16a-binding polypeptide which is a CD16a-binding polypeptide of the present invention. The third CD16a-binding polypeptides may have the same sequence as the first and/or second CD16a-binding polypeptides sequences. Alternatively, the third CD16a-binding polypeptides may have a different sequence to the first and second CD16a-binding polypeptide. A CD16a-binding oligomer of the present invention may optionally further comprise a fourth CD16a-binding polypeptide which is a CD16a-binding polypeptide of the present invention. The fourth CD16a-binding polypeptides may have the same sequence as the first and/or second and/or third CD16a-binding polypeptides sequences. Alternatively, the fourth CD16a-binding polypeptide may have a different sequence to the first, second and third CD16a-binding polypeptides.

In one preferred embodiment, a CD16a-binding oligomer of the present invention comprises a first CD16a-binding polypeptide that comprises a first binding motif selected from SEQ ID NOs. 150 to 221 (and wherein optionally from 1 to 5 (preferably 1, 2 or 3) residues in the sequence are replaced by an alternative residue, and preferably a residue that is a conservative replacement); and a second CD16a-binding polypeptide that comprises a second binding motif selected from SEQ ID NOs. 150 to 221 (and wherein optionally from 1 to 5 (preferably 1, 2 or 3) residues in the sequence are replaced by an alternative residue, and preferably a residue that is a conservative replacement). The first and second CD16a-binding motifs may have the same sequence or a different sequence.

In one preferred embodiment, a CD16a-binding oligomer of the present invention comprises a first CD16a-binding polypeptide that has a sequence selected from SEQ ID NOs. 1-75 (and wherein optionally from 1 to 5 (preferably 1, 2 or 3) residues in the sequence are replaced by an alternative residue, and preferably a residue that is a conservative replacement); and a second CD16a-binding polypeptide that has sequence selected from SEQ ID NOs. 1-75 (and wherein optionally from 1 to 5 (preferably 1, 2 or 3) residues in the sequence are replaced by an alternative residue, and preferably a residue that is a conservative replacement). The first and second CD16a-binding polypeptide may have the same sequence or a different sequence.

In another embodiment, a CD16a-binding oligomer of the present invention comprises a first CD16a-binding polypeptide that has a sequence selected from SEQ ID NOs. 1-75 (and wherein optionally from 1 to 5 (preferably 1, 2 or 3) residues in the sequence are replaced by an alternative residue, and preferably a residue that is a conservative replacement); a second CD16a-binding polypeptide that has sequence selected from SEQ ID NOs. 1-75 (and wherein optionally from 1 to 5 (preferably 1, 2 or 3) residues in the sequence are replaced by an alternative residue, and preferably a residue that is a conservative replacement); and a third CD16a-binding polypeptide that has sequence selected from SEQ ID NOs. 1-75 (and wherein optionally from 1 to 5 (preferably 1, 2 or 3) residues in the sequence are replaced by an alternative residue, and preferably a residue that is a conservative replacement). The first, second and third CD16a-binding polypeptide may have the same sequence, or each have a different sequence, or two sequences may be the same and one may be different). Optionally, a fourth CD16a-binding polypeptide that has sequence selected from SEQ ID NOs. 1-75 (and wherein optionally from 1 to 5 (preferably 1, 2 or 3) residues in the sequence are replaced by an alternative residue, and preferably a residue that is a conservative replacement) may be present; which may be the same as any one of the first, second, or third sequences, or may be different.

In one preferred embodiment, a CD16a-binding oligomer of the present invention comprises a first CD16a-binding polypeptide that has a sequence selected from SEQ ID NOs. 1, 74 or 75 (and wherein optionally from 1 to 5 (preferably 1, 2 or 3) residues in the sequence are replaced by an alternative residue, and preferably a residue that is a conservative replacement); and a second CD16a-binding polypeptide that has sequence selected from SEQ ID NOs. 1, 74 or 75 (and wherein optionally from 1 to 5 (preferably 1, 2 or 3) residues in the sequence are replaced by an alternative residue, and preferably a residue that is a conservative replacement). The first and second CD16a-binding polypeptide may have the same sequence or a different sequence (for example, the first CD16a-binding polypeptide may have SEQ ID NO. 1; and the second CD16a-binding polypeptide may have SEQ ID NO. 1; or the first CD16a-binding polypeptide may have SEQ ID NO. 74; and the second CD16a-binding polypeptide may have SEQ ID NO. 1; the first CD16a-binding polypeptide may have SEQ ID NO. 75; and the second CD16a-binding polypeptide may have SEQ ID NO. 1).

The CD16a-binding polypeptides in a CD16a-binding oligomer of the present invention may be separated by a linker. For example, each CD16a-binding polypeptide in a CD16a-binding oligomer of the present invention may be separated by a linker. Preferably, the linker is a linker as defined herein, for example a flexible amino acid linker, a rigid amino acid linker or cleavable amino acid linker or non-amino acid linker. Where a CD16a-binding oligomer of the present invention comprises more than one linker, the linkers may be the same, or may be different.

Preferably, a linker for a CD16a-binding oligomer of the present invention comprises or has a sequence of 1 to 50 (for example 1 to 25, for example 1, 2, 3, 4, 5, 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T (preferably G and S).

In one preferred embodiment, a linker for a CD16a-binding oligomer of the present invention is G or comprises or has sequence GGGSG, GGGGS, GGSGG, GSGGG and/or SGGGG; for example the linker is G or comprises or has the sequence

GGGSG,
GGGSGGGGSG,
GGGSGGGGSGGGGSG,
GGGSGGGGSGGGGSGGGGSG,
GGGGS,
GGGGSGGGGS,
GGGGSGGGGSGGGGS,
GGGGSGGGGSGGGGSGGGGS,
GGSGG,
GGSGGGGSGG,
GGSGGGGSGGGGSGG,
GGSGGGGSGGGGSGGGGSGGGGSGG,
GSGGG,
GSGGGGSGGG,
GSGGGGSGGGGSGGG,
GSGGGGSGGGGSGGGGSGGG,
SGGGG,
SGGGGSGGGG,
SGGGGSGGGGSGGGG,
or
SGGGGSGGGGSGGGGSGGGG.

In one preferred embodiment, a linker for a CD16a-binding oligomer of the present invention is G or comprises or has sequence GGGSG; for example the linker comprises or has the sequence GGGSG, GGGSGGGGSG, GGGSGGGGSGGGGSG or GGGSGGGGSGGGGSGGGGSG. In another embodiment, a linker for a CD16a-binding oligomer of the present invention comprises or has sequence GGGGS; for example the linker comprises or has the sequence GGGGS, GGGGSGGGGS, GGGGSGGGGSGGGGS or GGGGSGGGGSGGGGSGGGGS.

Alternatively, CD16a-binding polypeptides in a CD16a-binding oligomer of the present invention may not be separated by a linker (i.e. they may be directly attached to each other).

In one preferred embodiment, a CD16a-binding oligomer does not comprise a linker.

In one embodiment, the CD16a-binding oligomer of the present invention comprises at least 2 (for example 2) CD16a-binding polypeptides, and the CD16a-binding oligomer comprises the following structure:
[N-terminal portion]-[Helix 1]-[Separating portion]-[Helix 2]—[C-terminal portion]-[linker]-[N-terminal portion]-[Helix 1]-[Separating portion]-[Helix 2]—[C-terminal portion];
wherein each N-terminal portion in the oligomer may have the same sequence or have different sequences; each C-terminal portion in the oligomer may have the same sequence or have different sequences; each separating portion in the oligomer may have the same sequence or have different sequences; each Helix 1 portion in the oligomer may have the same sequence or have different sequences; and each Helix 2 portion in the oligomer may have the same sequence or have different sequences.

In such embodiments, the linker preferably comprises or has a sequence of 1 to 25 (for example 1, 2, 3, 4, 5, 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T (preferably G and S). For example, the linker is G or comprises or has sequence GGGSG, GGGGS, GGSGG, GSGGG and/or SGGGG; for example wherein the linker is G or comprises or has the sequence GGGSG, GGGSGGGGSG, GGGSGGGGSGGGGSG, GGGSGGGGSGGGGSGGGGSG, GGGGS, GGGGSGGGGS, GGGGSGGGGSGGGGS, GGGGSGGGGSGGGGSGGGGS, GGSGG, GGSGGGGSGG, GGSGGGGSGGGGSGG, GGSGGGGSGGGGSGGGGSGGGGSGG, GSGGG, GSGGGGSGGG, GSGGGGSGGGGSGGG, GSGGGGSGGGGSGGGGSGGG, SGGGG, SGGGGSGGGG, SGGGGSGGGGSGGGG, or SGGGGSGGGGSGGGGSGGGG.

Alternatively, the linker may be absent, i.e. the CD16a-binding oligomer comprises the following structure:
[N-terminal portion]-[Helix 1]-[Separating portion]-[Helix 2]—[C-terminal portion]-[N-terminal portion]-[Helix 1]-[Separating portion]-[Helix 2]—[C-terminal portion].

In one embodiment, the CD16a-binding oligomer of the present invention comprises at least 3 (for example 3) CD16a-binding polypeptides, and the CD16a-binding oligomer comprises the following structure
[N-terminal portion]-[Helix 1]-[Separating portion]-[Helix 2]—[C-terminal portion]-[Linker]-[N-terminal portion]-[Helix 1]-[Separating portion]-[Helix 2]—[C-terminal portion]-[Linker]-[N-terminal portion]-[Helix 1]-[Separating portion]-[Helix 2]—[C-terminal portion];
and wherein each linker portion in the oligomer may have the same sequence or have different sequences; each N-terminal portion in the oligomer may have the same sequence, have different sequences, or two may have the same sequence and one may have a different sequence; each C-terminal portion may have the same sequence, have different sequences, or two may have the same sequence and one may have a different sequence; each separating portion in the oligomer may have the same sequence, have different sequences, or two may have the same sequence and one may have a different sequence; each Helix 1 portion in the oligomer may have the same sequence, have different sequences, or two may have the same sequence and one may have a different sequence; and each Helix 2 portion in the oligomer may have the same sequence, have different sequences, or two may have the same sequence and one may have a different sequence.

In such embodiments, the linker preferably comprises or has a sequence of 1 to 25 (for example 1, 2, 3, 4, 5, 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T (preferably G and S). For example, the linker is G or comprises or has sequence GGGSG, GGGGS, GGSGG, GSGGG and/or SGGGG. Also, for example, the linker is G or comprises or has the sequence GGGSG, GGGSGGGGSG, GGGSGGGGSGGGGSG, GGGSGGGGSGGGGSGGGGSG, GGGGS, GGGGSGGGGS, GGGGSGGGGSGGGGS, GGGGSGGGGSGGGGSGGGGS, GGSGG, GGSGGGGSGG, GGSGGGGSGGGGSGG, GGSGGGGSGGGGSGGGGSGGGGSGG, GSGGG, GSGGGGSGGG, GSGGGGSGGGGSGGG, GSGGGGSGGGGSGGGGSGGG, SGGGG, SGGGGSGGGG, SGGGGSGGGGSGGGG, or SGGGGSGGGGSGGGGSGGGG.

Alternatively, one or both linkers may be absent, i.e. the CD16a-binding oligomer comprises the following structure:
[N-terminal portion]-[Helix 1]-[Separating portion]-[Helix 2]—[C-terminal portion]-[N-terminal portion]-[Helix 1]-[Separating portion]-[Helix 2]—[C-terminal portion]-[Linker]-[N-terminal portion]-[Helix 1]-[Separating portion]-[Helix 2]—[C-terminal portion];
[N-terminal portion]-[Helix 1]-[Separating portion]-[Helix 2]—[C-terminal portion]-[Linker]-[N-terminal portion]-[Helix 1]-[Separating portion]-[Helix 2]—[C-terminal portion]-[N-terminal portion]-[Helix 1]-[Separating portion]-[Helix 2]—[C-terminal portion]; or
[N-terminal portion]-[Helix 1]-[Separating portion]-[Helix 2]—[C-terminal portion]-[N-terminal portion]-[Helix 1]-[Separating portion]-[Helix 2]—[C-terminal portion]-[N-terminal portion]-[Helix 1]-[Separating portion]-[Helix 2]—[C-terminal portion].

In one embodiment, the CD16a-binding oligomer comprises the sequence VDNKFNKEVQMAQFEIRKLPNLNHHQSFAFIKSLMDDPSQSANLLAEAKKLN DAQAPKGGGSGGGGSGGGGSGVDNKFNKEVQMAQFEIRKLPNLNHHQSFAF IKSLMDDPSQSANLLAEAKKLNDAQAPK [SEQ ID NO. 242], and wherein optionally 1, 2 or 3 residues in the sequence are replaced by an alternative residue, and preferably a residue that is a conservative replacement.

In another embodiment, the CD16a-binding oligomer comprises the sequence VDNKFNKEQFYARDEIDLLPNLNEDQKWAFYMSLIDDPSQSANLLAEAKKLN DAQAPKGGGSGGGGSGGGGSGVDNKFNKEVQMAQFEIRKLPNLNHHQSFAF IKSLMDDPSQSANLLAEAKKLNDAQAPK SEQ ID NO. 243], and wherein optionally 1, 2 or 3 residues in the sequence are replaced by an alternative residue, and preferably a residue that is a conservative replacement.

In another embodiment, the CD16a-binding oligomer comprises the sequence VDNKFNKEFWIAESEIESLPNLNIYQKWAFKYSLADDPSQSANLLAEAKKLN DAQAPKGGGSGGGGSGGGGSGVDNKFNKEVQMAQFEIRKLPNLNHHQSFAF IKSLMDDPSQSANLLAEAKKLNDAQAPK [SEQ ID NO. 244], and wherein optionally 1, 2 or 3 residues in the sequence are replaced by an alternative residue, and preferably a residue that is a conservative replacement.

In a further embodiment, a CD16a-binding oligomer of the present invention may comprise two CD16a-binding polypeptides, each of which has a sequence in which Helix 1 comprises the sequence VQMAQFEIRK and Helix 2 comprises the sequence HHQSFAFIKSLM. As set out elsewhere, such sequences may have a number of residues substituted by an alternative residue. For example, each of the two CD16-a binding polypeptides may have the sequence:

[SEQ ID NO: 1]
VDNKFNKEVQMAQFEIRKLPNLNHHQSFAFIKSLMDDPSQSANLLAEA
KKLNDAQAPK.

In an alternative example, a CD16a-binding oligomer of the present invention may comprise two CD16a-binding polypeptides, one of which has a sequence in which Helix 1 comprises the sequence QFYARDEIDL and Helix 2 comprises the sequence EDQKWAFYMSLI, and the other of which has a sequence in which Helix 1 comprises the sequence VQMAQFEIRK and Helix 2 comprises the sequence HHQSFAFIKSLM. As set out elsewhere, such sequences may have a number of residues substituted by an alternative residue. For example, one of the two CD16a binding polypeptides may have the sequence:

[SEQ ID NO: 2]
VDNKFNKEQFYARDEIDLLPNLNEDQKWAFYMSLIDDPSQSANLLAEA
KKLNDAQAPK
and

The other may have the sequence:

[SEQ ID NO: 1]
VDNKFNKEVQMAQFEIRKLPNLNHHQSFAFIKSLMDDPSQSANLLAEA
KKLNDAQAPK.

In an alternative example, a CD16a-binding oligomer of the present invention may comprise two CD16a-binding polypeptides, one of which has a sequence in which Helix 1 comprises the sequence VQMAQFEIRK and Helix 2 comprises the sequence HHQSFAFIKSLM, and the other of which has a sequence in which Helix 1 comprises the sequence FWIAESEIES and Helix 2 comprises the sequence IYQKWAFKYSLA. As set out elsewhere, such sequences may have a number of residues substituted by an alternative residue. For example, one of the two polypeptides may have the sequence:

[SEQ ID NO: 1]
VDNKFNKEVQMAQFEIRKLPNLNHHQSFAFIKSLMDDPSQSANLLAEA
KKLNDAQAPK
and

The other may have the sequence:

[SEQ ID NO: 75]
VDNKFNKEFWIAESEIESLPNLNIYQKWAFKYSLADDPSQSANLLAEA
KKLNDAQAPK.

For example, such a CD16a-binding oligomer may comprise three CD16a-binding polypeptides, each of which has a sequence in which Helix 1 comprises the sequence VQMAQFEIRK and Helix 2 comprises the sequence HHQSFAFIKSLM. As set out elsewhere, such sequences may have a number of residues substituted by an alternative residue. For example, each of the three polypeptides may have the sequence:

[SEQ ID NO: 1]
VDNKFNKEVQMAQFEIRKLPNLNHHQSFAFIKSLMDDPSQSANLLAEA
KKLNDAQAPK.

In an alternative example, a CD16a-binding oligomer of the present invention may comprise three CD16a-binding polypeptides, two of which have a sequence in which Helix 1 comprises the sequence QFYARDEIDL and Helix 2 comprises the sequence EDQKWAFYMSLI, and the other one of which has a sequence in which Helix 1 comprises the sequence VQMAQFEIRK and Helix 2 comprises the sequence HHQSFAFIKSLM. As set out elsewhere, such sequences may have a number of residues substituted by an alternative residue. For example, two of the three polypeptides may have the sequence:

[SEQ ID NO: 74]
VDNKFNKEQFYARDEIDLLPNLNEDQKWAFYMSLIDDPSQSANLLAEA
KKLNDAQAPK
and

The other one may have the sequence:

[SEQ ID NO: 1]
VDNKFNKEVQMAQFEIRKLPNLNHHQSFAFIKSLMDDPSQSANLLAEA
KKLNDAQAPK.

In an alternative example, CD16a-binding oligomer of the present invention may comprise three CD16a-binding polypeptides, one of which has a sequence in which Helix 1 comprises the sequence QFYARDEIDL and Helix 2 comprises the sequence EDQKWAFYMSLI, and the other two of which have a sequence in which Helix 1 comprises the sequence VQMAQFEIRK and Helix 2 comprises the sequence HHQSFAFIKSLM. As set out elsewhere, such sequences may have a number of residues substituted by an alternative residue. For example, one of the three polypeptides may have the sequence:

[SEQ ID NO: 74]
VDNKFNKEQFYARDEIDLLPNLNEDQKWAFYMSLIDDPSQSANLLAEA
KKLNDAQAPK
and

The other two may have the sequence:

[SEQ ID NO: 1]
VDNKFNKEVQMAQFEIRKLPNLNHHQSFAFIKSLMDDPSQSANLLAEA
KKLNDAQAPK.

In an alternative example, CD16a-binding oligomer of the present invention may comprise three CD16a-binding polypeptides, one of which has a sequence in which Helix 1 comprises the sequence VQMAQFEIRK and Helix 2 comprises the sequence HHQSFAFIKSLM, and the other two of which have a sequence in which Helix 1 comprises the sequence FWIAESEIES and Helix 2 comprises the sequence IYQKWAFKYSLA. As set out elsewhere, such sequences may have a number of residues substituted by an alternative residue. For example, one of the three polypeptides may have the sequence:

[SEQ ID NO: 1]
VDNKFNKEVQMAQFEIRKLPNLNHHQSFAFIKSLMDDPSQSANLLAEA
KKLNDAQAPK
and

The other two may have the sequence:

[SEQ ID NO: 75]
VDNKFNKEFWIAESEIESLPNLNIYQKWAFKYSLADDPSQSANLLAEA
KKLNDAQAPK.

In an alternative example, CD16a-binding oligomer of the present invention may comprise three CD16a-binding polypeptides, two of which have a sequence in which Helix 1 comprises the sequence VQMAQFEIRK and Helix 2 comprises the sequence HHQSFAFIKSLM, and the other one of which has a sequence in which Helix 1 comprises the sequence FWIAESEIES and Helix 2 comprises the sequence IYQKWAFKYSLA. As set out elsewhere, such sequences may have a number of residues substituted by an alternative residue. For example, two of the three polypeptides may have the sequence:

[SEQ ID NO: 1]
VDNKFNKEVQMAQFEIRKLPNLNHHQSFAFIKSLMDDPSQSANLLAEA
KKLNDAQAPK
and

The other one may have the sequence:

[SEQ ID NO: 75]
VDNKFNKEFWIAESEIESLPNLNIYQKWAFKYSLADDPSQSANLLAEA
KKLNDAQAPK.

For example, such CD16a-binding oligomer of the present invention may comprise four CD16a-binding polypeptides, each of which has a sequence in which Helix 1 comprises the sequence VQMAQFEIRK and Helix 2 comprises the sequence HHQSFAFIKSLM. As set out elsewhere, such sequences may have a number of residues substituted by an alternative residue. For example, each of the four polypeptides may have the sequence:

[SEQ ID NO: 1]
VDNKFNKEVQMAQFEIRKLPNLNHHQSFAFIKSLMDDPSQSANLLAE
AKKLNDAQAPK.

In an alternative example, CD16a-binding oligomer of the present invention may comprise four CD16a-binding polypeptides, two of which has a sequence in which Helix 1 comprises the sequence QFYARDEIDL and Helix 2 comprises the sequence EDQKWAFYMSLI, and the other two of which has a sequence in which Helix 1 comprises the sequence VQMAQFEIRK and Helix 2 comprises the sequence HHQSFAFIKSLM. As set out elsewhere, such sequences may have a number of residues substituted by an alternative residue. For example, two of the four polypeptides may have the sequence:

[SEQ ID NO: 74]
VDNKFNKEQFYARDEIDLLPNLNEDQKWAFYMSLIDDPSQSANLLAE
AKKLNDAQAPK
and

The other two may have the sequence:

[SEQ ID NO: 1]
VDNKFNKEVQMAQFEIRKLPNLNHHQSFAFIKSLMDDPSQSANLLAE
AKKLNDAQAPK

In an alternative example, CD16a-binding oligomer of the present invention may comprise four CD16a-binding polypeptides, two of which have a sequence in which Helix 1 comprises the sequence VQMAQFEIRK and Helix 2 comprises the sequence HHQSFAFIKSLM, and the other two of which have a sequence in which Helix 1 comprises the sequence FWIAESEIES and Helix 2 comprises the sequence IYQKWAFKYSLA. As set out elsewhere, such sequences may have a number of residues substituted by an alternative residue. For example, two of the four polypeptides may have the sequence:

[SEQ ID NO: 1]
VDNKFNKEVQMAQFEIRKLPNLNHHQSFAFIKSLMDDPSQSANLLAE
AKKLNDAQAPK
and

The other two may have the sequence:

[SEQ ID NO: 75]
VDNKFNKEFWIAESEIESLPNLNIYQKWAFKYSLADDPSQSANLLAE
AKKLNDAQAPK.

In the CD16a-binding oligomer of the present invention described above, which may also be referred to as a multimeric binders, the two or more CD16a-binding moieties are optionally connected via a linker as defined herein. When two or more linkers are present in a CD16a-binding oligomer of the present invention, the linkers may be the same or may be different, for example the linkers may be amino acid sequences and they may have the same amino acid sequence or a different amino acid sequence.

### Linkers

Where present, a linker connects together two or more functional portions (defined further herein below) of the polypeptides of the invention. For example, a linker may connect together two CD16a binding polypeptides of the present invention (for example in a CD16a-binding oligomer of the invention), or a linker may connect together a CD16a binding polypeptides of the present invention and an additional functional portion of the present invention. A linker may also connect together an additional functional of the present invention and an additional functional portion of the present invention in embodiments where more than one additional functional portion is present. The skilled person is aware that different kinds of linkers with varying properties are known in the art, and is able to select the appropriate linker(s) depending on the properties desired. Types of linkers include, for example, flexible amino acid linkers, rigid amino acid linkers and cleavable amino acid linkers; non-amino acid linkers may also be used. For example, linkers may be selected to increase stability or improve folding, to increase expression, improve biological activity, enable targeting, or alter pharmacokinetics. A non-amino acid linker may be referred to as a synthetic linker.

For the avoidance of doubt, in certain embodiments a linker may not connect together two CD16a binding polypeptides of the present invention (for example in a CD16a-binding oligomer of the invention), and/or a linker may not connect together a CD16a binding polypeptides of the present invention and an additional functional portion of the present invention; and/or a linker may not connect together an additional functional of the present invention and an additional functional portion of the present invention in embodiments where more than one additional functional portion is present. For example, in certain embodiments two CD16a binding polypeptides of the present invention (for example in a CD16a-binding oligomer of the invention) may be directly connected; and/or a CD16a binding polypeptide or oligomer of the present invention may be directly connected to additional functional portion of the present invention; and/or an additional functional of the present invention may be directly connected to an additional functional portion of the present invention.

In one embodiment, the CD16a binding polypeptide according to any aspect disclosed herein, or a CD16a-binding oligomer according to any aspect disclosed herein, further comprises at least one linker, such as at least one linker selected from flexible amino acid linkers, rigid amino acid linkers and cleavable amino acid linkers. In one embodiment, said linker is between two or more CD16a-binding polypeptides, for example in a CD16a-binding oligomer of the present invention, or between a CD16a-binding polypeptide or a CD16a-binding oligomer and an additional functional portion, for example an immune signalling molecule or an additional binding moiety (for example as described in further detail below).

In one embodiment, the CD16a binding oligomer according to any aspect disclosed herein comprises at least one linker, such as at least one linker selected from flexible amino acid linkers, rigid amino acid linkers and cleavable amino acid linkers. The linker is between two or more CD16a-binding polypeptides. A further linker between a CD16a-binding polypeptide or a CD16a-binding oligomer and an additional functional portion, for example an immune signalling molecule or an additional binding moiety (for example as described in further detail below), may also be present in a CD16a binding oligomer according to any aspect disclosed herein. A further linker between an additional functional portion (for example an immune signalling molecule or an additional binding moiety (for example as described in further detail below) and an additional functional portion (for example an immune signalling molecule or an additional binding moiety (for example as described in further detail below), may also be present in a CD16a binding oligomer according to any aspect disclosed herein.

In embodiments and aspects of the present invention that comprise more than one linker, each linker may be the same, or may be different, or some linkers may be the same, and some may be different (for example in embodiments having 3 or more linkers (for example 3, 4, 5, 6, 7, 8 or more linkers).

Flexible linkers may be used when the linked domains require some distance and conformational freedom, and may be advantageous in some embodiments of the invention. Such linkers are generally composed of small, non-polar (for example G) or polar (for example S or T) amino acids. Some flexible linkers primarily consist of stretches of G and S residues, for example (GGGGS)p or (GGGSG)p. Other examples include (GGSGG)p, (GSGGG)p or (SGGGG)p. Adjusting the copy number “p” allows optimization of the linker in order to achieve appropriate separation between the functional moieties, or to maintain necessary inter-moiety interaction. In a preferred embodiment, the linker is (GGGSG)p, for example (GGGSG)₁, (GGGSG)₂ or (GGGSG)₃, for example (GGGSG)₃. In an embodiment, the linker is (GGGGS)p, for example (GGGGS)₁, (GGGGS)₂ or (GGGGS)₃, for example (GGGGS)₃. In an embodiment, the linker is (GGSGG)p, for example (GGSGG)₁, (GGSGG)₂ or (GGSGG)₃, for example (GGSGG)₃. In an embodiment, the linker is (GSGGG)p, for example (GSGGG)₁, (GSGGG)₂ or (GSGGG)₃, for example (GSGGG)₃. In an embodiment, the linker is (SGGGG)p, for example (SGGGG)₁, (SGGGG)₂ or (SGGGG)₃, for example (SGGGG)₃. In another, embodiment, the linker is G.

In one embodiment, a linker of the present invention comprises or has a sequence of 1 to 50 (for example 1 to 40, 1 to 30, or 1 to 25, for example 1, 2, 3, 4, 5 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T. In one embodiment, a linker of the present invention comprises or has a sequence of 1 to 50 (for example 1 to 25, for example 15, 16, 17, 18, 19, 20, 21, 22, 23, 24 or 25; or 1 to 20, for example 1, 5, 10, 15 or 20; or 1 to 15, for example 1, 5, 10 or 15) naturally occurring amino acids, for example selected from the group consisting of G and S. For example, a linker of the present invention is G or comprises or has sequence GGGSG; for example the linker comprises or has the sequence GGGSG, GGGSGGGGSG, GGGSGGGGSGGGGSG or GGGSGGGGSGGGGSGGGGSG.

Also, for example, a linker of the present invention is G or comprises or has sequence GGGSG, GGGGS, GGSGG, GSGGG and/or SGGGG. For example, the linker is G or comprises or has the sequence GGGSG, GGGSGGGGSG, GGGSGGGGSGGGGSG, GGGSGGGGSGGGGSGGGGSG, GGGGS, GGGGSGGGGS, GGGGSGGGGSGGGGS, GGGGSGGGGSGGGGSGGGGS, GGSGG, GGSGGGGSGG, GGSGGGGSGGGGSGG, GGSGGGGSGGGGSGGGGSGGGGSGG, GSGGG, GSGGGGSGGG, GSGGGGSGGGGSGGG, GSGGGGSGGGGSGGGGSGGG, SGGGG, SGGGGSGGGG, SGGGGSGGGGSGGGG, or SGGGGSGGGGSGGGGSGGGG.

In one preferred embodiment, a linker of the present invention is G or comprises or has sequence GGGSG; for example the linker comprises or has the sequence GGGSG, GGGSGGGGSG, GGGSGGGGSGGGGSG or GGGSGGGGSGGGGSGGGGSG. In another embodiment, a linker for a CD16a-binding oligomer of the present invention comprises or has sequence GGGGS; for example the linker comprises or has the sequence GGGGS, GGGGSGGGGS, GGGGSGGGGSGGGGS or GGGGSGGGGSGGGGSGGGGS.

Apart from G and S linkers, other flexible linkers are known in the art, such as G and S linkers containing additional amino acid residues, such as T and A, to maintain flexibility, as well as polar amino acid residues to improve solubility. In general, it is known in the art that linker sequence and length may affect the characteristics of the linked moieties, and so the skilled person will be able to select an appropriate linker for use in the binding polypeptides as described herein.

Other types of linkers, such as rigid and/or cleavable linkers, can also be used to connect domains in multidomain constructs to improve or control their biological activity. Such linkers are known in the art (Chen X et al, Fusion protein linkers: Property, design and functionality, Adv Drug Deliv Rev 2013:65:10:1357, doi: 10.1016/j.addr.2012.09.039).

Alternatively, the different binding moieties of the binding polypeptide as described herein (for example two or more CD16a-binding polypeptides, or a CD16a-binding polypeptide and an additional functional portion, for example an immune signalling molecule or an additional binding moiety) may be covalently linked by a chemical linker. Such a chemical linker may be produced by, for example, maleimide or ‘click’ chemistry. The skilled person will be aware of other linkers suitable for use in the binding polypeptides as described herein.

With regard to the description above of binding polypeptides comprising a CD16a-binding polypeptide according to the disclosure, it is to be noted that the designation of first, second and further moieties is made for clarity reasons to distinguish between CD16a-binding polypeptide or polypeptides according to the invention on the one hand, and binding moieties exhibiting other functions on the other hand. These designations are not intended to refer to the actual order of the different regions of the binding polypeptide. Similarly, the designations first and second moiety (or monomer unit) are made for clarity reasons to distinguish between said units. Thus, for example, said first moiety (or monomer unit) may without restriction appear at the N-terminal end, in the middle, or at the C-terminal end of the binding polypeptide.

The CD16a-binding polypeptides in a CD16a-binding oligomer of the present invention may be separated by a linker. For example, each CD16a-binding polypeptides in a CD16a-binding oligomer of the present invention may be separated by a linker. Preferably, the linker is a linker as defined herein. Where a CD16a-binding oligomer of the present invention comprises more than one linker, the linkers may be the same, or may be different.

Preferably, a linker for a CD16a-binding oligomer of the present invention comprises or has a sequence of 1 to 50 (for example 1 to 25, for example 1, 2, 3, 4, 5, 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T (preferably G and S).

In one preferred embodiment, a linker for a CD16a-binding oligomer of the present invention is G or comprises or has sequence GGGSG, GGGGS, GGSGG, GSGGG and/or SGGGG; for example the linker comprises or has the sequence GGGSG, GGGSGGGGSG, GGGSGGGGSGGGGSG or GGGSGGGGSGGGGSGGGGSG. In another embodiment, a linker for a CD16a-binding oligomer of the present invention comprises or has sequence GGGGS; for example the linker comprises or has the sequence GGGGS, GGGGSGGGGS, GGGGSGGGGSGGGGS or GGGGSGGGGSGGGGSGGGGS.

In embodiments of the present invention comprising an additional functional portion, the CD16a-binding polypeptide(s) and additional functional portion(s) in a CD16a-binding polypeptide or a CD16a-binding oligomer of the present invention may be separated by a linker. For example, each additional functional portion and CD16a-binding polypeptide in a CD16a-binding polypeptide or CD16a-binding oligomer of the present invention may be separated by a linker. Preferably, the linker is a linker as defined herein. In embodiments of the present invention comprising two or more additional functional portions, the additional functional portions may be separated by a linker. Preferably, the linker is a linker as defined herein.

Where a CD16a-binding polypeptide or a CD16a-binding oligomer of the present invention comprises more than one linker (i.e. wherein there are at least two CD16a-binding polypeptides and at least one additional functional portion; or wherein there is at least one CD16a-binding polypeptides and at least two additional functional portion), the linkers may be the same, or may be different. Preferably, a linker for a CD16a-binding polypeptide or a CD16a-binding oligomer comprising an additional functional portion comprises or has a sequence of 1 to 50 (for example 1 to 25, for example 1, 2, 3, 4, 5, 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T (preferably G and S).

In one preferred embodiment, a linker for a CD16a-binding polypeptide or a CD16a-binding oligomer comprising an additional functional portion is G or comprises or has sequence GGGSG, GGGGS, GGSGG, GSGGG and/or SGGGG; for example the linker comprises or has the sequence GGGSG, GGGSGGGGSG, GGGSGGGGSGGGGSG or GGGSGGGGSGGGGSGGGGSG. In another embodiment, a linker for a CD16a-binding oligomer of the present invention comprises or has sequence GGGGS; for example the linker comprises or has the sequence GGGGS, GGGGSGGGGS, GGGGSGGGGSGGGGS or GGGGSGGGGSGGGGSGGGGS.

### Additional Functional Portions

CD16a binding polypeptides as disclosed herein may be attached, for example via a linker as described hereinabove, to one or more additional functional portions. CD16a binding oligomers as disclosed herein may be attached, for example via a linker as described hereinabove, to one or more additional functional portions. Therefore, in an embodiment, the at least one CD16a binding polypeptide (or CD16a binding oligomer) is attached to one or more additional functional portions, optionally via a linker as described herein.

For the avoidance of doubt, a CD16a binding polypeptide or CD16a binding oligomer of the present invention may be directly attached to one or more additional functional portions (i.e. not attached via a linker).

A ‘functional portion’, as used herein, refers to a component or ‘moiety’ with a specific desired biological activity.

The one or more additional component(s) (i.e. the one or more additional functional portion) may for example be a signalling molecule. Examples of suitable signalling molecules include immune signalling molecules such as cytokines, for example IL-15 and derivatives thereof.

The one or more additional component(s) (i.e. the one or more additional functional portion) may be one or more additional binding moiety(ies), for example one or more binding partner(s) recognising a cell surface protein or antigen, for example an immune cell surface protein or a cell surface tumour antigen (also referred to as a cancer cell surface antigen). Cell surface tumour antigens may for example be tumour-associated antigens or tumour-specific antigens.

Examples of additional components (i.e. additional functional portions) include additional binding moieties that are binding partners recognising B-cell maturation antigen (BCMA), cytotoxic T-lymphocyte-associated protein 4 (CTLA-4), Programmed cell death protein 1 (PD-1), disintegrin and metalloprotease 17 (ADAM17), Programmed death-ligand 1 (PD-L1), SLAM family member 7 (SLAMF7), Epithelial Cell Adhesion Molecule (EPCAM), Epidermal growth factor receptor (EGFR/ErbB-1), Epidermal growth factor receptor variant 3 (EGFRvIII), erb-b2 tyrosine kinase 2 (ERBB2/HER2/CD340), prostate-specific membrane antigen (PSMA), Claudin8.2 (CLDN18.2), delta like protein 3 (DLL3), mucin 16 (MUC16), mucin 17 (MUC17), mucin 1 (MUC1), Trophoblast glycoprotein (TPBG/5T4/WAIF1), V-set domain-containing T-cell activation inhibitor 1 (B7-H4/VTCN1/B7x/B7S1), cluster of differentiate 20 (CD20), B-Lymphocyte Surface Antigen B4 (CD19), Sialic Acid-Binding Ig-Like Lectin 2 (CD22), TNF receptor superfamily member 8 (CD30), Natural cytotoxicity triggering receptor 1 (NKp46), or NKG2D. Examples of additional components (i.e. additional functional portions) include additional binding moieties that are binding partners recognising a cell surface tumour antigen or cancer cell surface target selected from the group consisting BCMA, ADAM17, SLAMF7, PD-L1, EPCAM, EGFR/ErbB-1, EGFRvIII, ERBB2/HiER2/CD340, PSMA, CLDN18.2, DLL3, MUC16, MUC17, MUC1, TPBG/5T4/WAIF1 and B7-H4/VTCN1/B7x/B7S1. Examples of additional components (i.e. additional functional portions) include additional binding moieties that are binding partners recognising an immune cell surface protein or immune cell surface target selected from the group consisting of CTLA-4, PD-1, NKp46, NKG2D CD20, CD19, CD22 and CD30. Examples of additional components (i.e. additional functional portions) include additional binding moieties that are binding partners recognising a cell surface tumour antigen or cancer cell surface target expressed in haematological malignancies, for example BCMA, CD20, CD19, CD22 or CD30. (For the avoidance of doubt, such examples of additional binding moieties, for example binding partners recognising cell surface proteins or antigens, are non-limiting and are specified here by way of illustration.) The additional binding moiety as referred to in this context is not a CD16a binding polypeptide of the present invention.

In one embodiment, a CD16a binding polypeptide or CD16a-binding oligomer of the present invention is attached to one or more additional functional portion (for example one or more additional binding moiety(ies) and/or signalling molecule), optionally via a linker as described above. For example, the at least one CD16a binding polypeptide or CD16a-binding oligomer is attached to one, two, three, four or more additional functional portions (for example one, two, three, four or more additional binding moieties and/or signalling molecules). In certain embodiments, at least one CD16a binding polypeptide or CD16a-binding oligomer is attached to one or two additional functional portions (for example one or two additional binding moieties or signalling molecules). In certain embodiments, at least one CD16a binding polypeptide or CD16a-binding oligomer is attached to one additional functional portion (for example one additional binding moiety or signalling molecule).

In certain preferred embodiments, an additional function portion is a signalling molecule. A signalling molecule, for example an immune signalling molecule such as a cytokine, for example IL-15 or derivatives thereof, may be attached at the N-terminal end or the C-terminal end of a CD16a-binding polypeptide or a CD16a-binding oligomer, optionally via a linker as described above. One or more signalling molecule(s) may, alternatively, be attached between two CD16a-binding polypeptides in a CD16a-binding oligomer, optionally separated by one or more linking sequences as described above. Preferably, the signalling molecule(s) may be attached at the N-terminal end or the C-terminal end of a CD16a-binding polypeptide or a CD16a-binding oligomer, optionally via a linker as described above. Preferably, the signalling molecule(s) may be attached at the N-terminal end of a CD16a-binding polypeptide or a CD16a-binding oligomer, optionally via a linker as described above.

In certain preferred embodiments, an additional function portion is an additional binding moiety. For example, an additional function portion is an additional binding moiety that is a binding partner recognising one of the following: CTLA-4, PD-1, BCMA, ADAM17, PD-L1, SLAMF7, EPCAM, EGFR/ErbB-1, EGFRvIII, ERBB2/HER2/CD340, PSMA, CLDN18.2, DLL3, MUC16, MUC17, MUC1, TPBG/5T4/WAIF1, B7-H4/VTCN1/B7x/B7S1, CD20, CD19, CD22 or CD30. For example, an additional function portion is an additional binding moiety that is specific for one of the following: CTLA-4, PD-1, BCMA, ADAM17, PD-L1, SLAMF7, EPCAM, EGFR/ErbB-1, EGFRvIII, ERBB2/HER2/CD340, PSMA, CLDN18.2, DLL3, MUC16, MUC17, MUC1, TPBG/5T4/WAIF1, B7-H4/VTCN1/B7x/B7S1, CD20, CD19, CD22 or CD30.

In certain preferred embodiments, an additional binding moiety is specific for a cancer cell surface target (for example a myeloma cell surface antigen, for example BCMA). In certain preferred embodiments, an additional binding moiety is specific for an immune cell surface target (for example a NK cell target, for example NKp46, NKG2D, PD-1, etc).

An additional binding moiety, for example a binding partner recognising the cell surface tumour antigen BCMA, may be attached at the N-terminal end or the C-terminal end of a CD16a-binding polypeptide or a CD16a-binding oligomer, optionally via a linker as described above. The one or more additional binding moiety(ies) may, alternatively, be attached between two CD16a-binding polypeptides in a CD16a-binding oligomer, optionally separated by one or more linking sequences as described above. Preferably, the additional binding moiety(ies), for example a binding partner recognising the cell surface tumour antigen BCMA, may be attached at the N-terminal end or the C-terminal end of a CD16a-binding polypeptide or a CD16a-binding oligomer, optionally via a linker as described above. More preferably, the additional binding moiety(ies), for example a binding partner recognising the cell surface tumour antigen BCMA, may be attached at the N-terminal end of a CD16a-binding polypeptide or a CD16a-binding oligomer, optionally via a linker as described above.

The present inventors have surprisingly found that a ‘dual engager’ polypeptide comprising a CD16a binding polypeptide or a CD16a binding oligomer as disclosed herein can retain its CD16a binding ability when fused to an additional binding moiety targeting the myeloma antigen BCMA. This ‘dual engager’ polypeptide comprising a CD16a binding polypeptide as disclosed herein fused to a BCMA binding moiety is also surprisingly capable of activating NK cells in the presence of BCMA-expressing tumour cells.

In one preferred embodiment, the CD16a-binding polypeptide or CD16a-binding oligomer of the present invention further comprises one or more additional functional portions, for example at least one, at least two, or at least three or at least four additional functional portions. For example, in particular embodiments, CD16a-binding polypeptide or CD16a-binding oligomer of the present invention further comprises 1, 2, 3, 4 or 5 additional functional portions. In especially preferred embodiments, the CD16a-binding polypeptide or CD16a-binding oligomer of the present invention further comprises one, two or three additional functional portions, and most preferably two.

In embodiments wherein the CD16a-binding polypeptide or CD16a-binding oligomer comprises at least two, at least three or at least four additional functional portions (for example 2, 3, 4 or 5 additional functional portions), each additional functional portion may be the same, or may be different, or some additional functional portions may be the same, and some additional functional portions be different (for example in embodiments having at least 3 or at least 4 additional functional portions (for example 3, 4, 5, 6 or more additional functional portions)).

In embodiments wherein the CD16a-binding polypeptide or CD16a-binding oligomer comprises at least two, at least three or at least four additional functional portions (for example 2, 3, 4 or 5 additional functional portions), each additional functional portion may have the same function, or may have different functions, or some additional functional portions may have the same function, and some may have different functions (for example in embodiments having at least 3 or at least 4 additional functional portions (for example 3, 4, 5, 6 or more additional functional portions)).

In embodiments wherein the CD16a-binding polypeptide or CD16a-binding oligomer comprises at least two, at least three or at least four additional functional portions (for example 2, 3, 4 or 5) additional functional portions, a first additional functional portion may comprise an additional binding moiety (for example an additional binding specific for a cancer cell surface target, for example a myeloma cell surface antigen, for example BCMA, or is specific for an immune cell target, for example a NK cell target); and a second additional functional portion may comprise an immune signalling molecule, for example a cytokine, for example IL-15 or derivatives thereof. For example, in one preferred embodiment, a first additional functional portion may comprise an additional binding moiety specific for a cancer cell surface target (for example a myeloma cell surface antigen, for example BCMA) and a second additional functional portion may comprise a cytokine, for example IL-15 or derivatives thereof. In particular, a first additional functional portion may comprise an additional binding moiety specific for BCMA; and second additional functional portion may comprise a cytokine, for example IL-15 or derivatives thereof. Alternatively, for example, in one preferred embodiment, a first additional functional portion may comprise an additional binding moiety specific for a immune cell target (for example a NK cell target) and a second additional functional portion may comprise a cytokine, for example IL-15 or derivatives thereof. In particular, a first additional functional portion may comprise an additional binding moiety specific for NK cell target; and second additional functional portion may comprise a cytokine, for example IL-15 or derivatives thereof.

In a very preferred embodiment, a first additional functional portion may comprise an additional binding moiety (for example an additional binding moiety specific for a cancer cell surface target, for example a myeloma cell surface antigen, for example BCMA, or is specific for an immune cell target, for example a NK cell target); and a second additional functional portion may comprise an additional binding moiety (for example an additional binding moiety specific for a cancer cell surface target, for example a myeloma cell surface antigen, for example BCMA, or is specific for an immune cell target, for example a NK cell target). For example, a first additional functional portion may comprise an additional binding moiety specific for a cancer cell surface target (for example a myeloma cell surface antigen, for example BCMA); and a second additional functional portion may comprise an additional binding moiety specific for a cancer cell surface target (for example a myeloma cell surface antigen, for example BCMA). In particular, a first additional functional portion may comprise an additional binding moiety specific for BCMA; and a second additional functional portion may comprise an additional binding moiety specific BCMA.

Alternatively, for example, a first additional functional portion may comprise an additional binding moiety specific for a cancer cell surface target (for example a myeloma cell surface antigen, for example BCMA); and a second additional functional portion may comprise an additional binding moiety specific for an immune cell surface target (for example a NK cell target). In particular, a first additional functional portion may comprise an additional binding moiety specific for BCMA; and a second additional functional portion may comprise an additional binding moiety specific for a NK cell target.

In another embodiment, a first additional functional portion may comprise an immune signalling molecule, for example a cytokine, for example IL-15 or derivatives thereof; and a second additional functional portion may comprise an immune signalling molecule, for example a cytokine, for example IL-15 or derivatives thereof.

When present, (for example in embodiments wherein the CD16a-binding polypeptide or CD16a-binding oligomer comprises at least three or at least four additional functional portions (for example 3, 4 or 5)) a third additional functional portion may comprises an additional binding moiety (for example an additional binding moiety specific for a cancer cell surface target, for example a myeloma cell surface antigen, for example BCMA, or is specific for an immune cell target, for example a NK cell target), or comprises an immune signalling molecule, for example a cytokine, for example IL-15 or derivatives thereof. Preferably, when present, a third additional functional portion may comprises an additional binding moiety (for example an additional binding moiety specific for a cancer cell surface target, for example a myeloma cell surface antigen, for example BCMA, or is specific for an immune cell target, for example a NK cell target; and especially additional binding moiety specific for a cancer cell surface target, for example a myeloma cell surface antigen, for example BCMA).

When present, (for example in embodiments wherein the CD16a-binding polypeptide or CD16a-binding oligomer comprises at least four additional functional portions (for example 4 or 5)) a fourth additional functional portion may comprises an additional binding moiety (for example an additional binding moiety specific for a cancer cell surface target, for example a myeloma cell surface antigen, for example BCMA, or is specific for an immune cell target, for example a NK cell target), or comprises an immune signalling molecule, for example a cytokine, for example IL-15 or derivatives thereof. Preferably, when present, a fourth additional functional portion may comprises an additional binding moiety (for example an additional binding moiety specific for a cancer cell surface target, for example a myeloma cell surface antigen, for example BCMA, or is specific for an immune cell target, for example a NK cell target; and especially additional binding moiety specific for a cancer cell surface target, for example a myeloma cell surface antigen, for example BCMA).

When present, (for example in embodiments wherein the CD16a-binding polypeptide or CD16a-binding oligomer comprises at least five additional functional portions (for example 5 or 6)) a fifth additional functional portion may comprises an additional binding moiety (for example an additional binding moiety specific for a cancer cell surface target, for example a myeloma cell surface antigen, for example BCMA, or is specific for an immune cell target, for example a NK cell target), or comprises an immune signalling molecule, for example a cytokine, for example IL-15 or derivatives thereof.

In one preferred embodiment, the CD16a-binding polypeptide or CD16a-binding oligomer comprises at least three (for example 3, 4 or 5) additional functional portions, a first additional functional portion may comprise an additional binding moiety (for example an additional binding moiety specific for a cancer cell surface target, for example a myeloma cell surface antigen, for example BCMA, or is specific for an immune cell target, for example a NK cell target); and a second additional functional portion may comprise an immune signalling molecule, for example a cytokine, for example IL-15 or derivatives thereof; and a third additional functional portion may comprises an additional binding moiety (for example an additional binding moiety specific for a cancer cell surface target, for example a myeloma cell surface antigen, for example BCMA, or is specific for an immune cell target, for example a NK cell target).

In one embodiment, a first additional functional portion may comprise an additional binding moiety specific for a cancer cell surface target (for example a myeloma cell surface antigen, for example BCMA); and a second additional functional portion may comprise a cytokine, for example IL-15 or derivatives thereof; and a third additional functional portion may comprises an additional binding moiety specific for a cancer cell surface target (for example a myeloma cell surface antigen, for example BCMA) or a immune cell target (for example a NK cell target). In particular, a first additional functional portion may comprise an additional binding moiety specific for BCMA; and a second additional functional portion may comprise a cytokine, for example IL-15 or derivatives thereof; and a third additional functional portion may comprise an additional binding moiety specific for a cancer cell surface target (for example a myeloma cell surface antigen, for example BCMA). In another embodiment, a first additional functional portion may comprise an additional binding moiety specific for BCMA; and a second additional functional portion may comprise a cytokine, for example IL-15 or derivatives thereof; and a third additional functional portion may comprise an additional binding moiety specific for an immune cell target (for example a NK cell target).

For example, a first additional functional portion may comprise an additional binding moiety specific for a cancer cell surface target (for example a myeloma cell surface antigen, for example BCMA); and a second additional functional portion may an additional binding moiety specific for a cancer cell surface target (for example a myeloma cell surface antigen, for example BCMA); and a third additional functional portion may comprises an additional binding moiety specific for a cancer cell surface target (for example a myeloma cell surface antigen, for example BCMA) or a immune cell target (for example a NK cell target). In particular, a first additional functional portion may comprise an additional binding moiety specific for BCMA; and a second additional functional portion may comprise an additional binding moiety specific for BCMA; and a third additional functional portion may comprise an additional binding moiety specific for a cancer cell surface target (for example a myeloma cell surface antigen, for example BCMA). In another embodiment, a first additional functional portion may comprise an additional binding moiety specific for BCMA; and a second additional functional portion may comprise an additional binding moiety specific for BCMA; and a third additional functional portion may comprise an additional binding moiety specific for an immune cell target (for example a NK cell target).

In one embodiment, a CD16a-binding polypeptide or CD16a-binding oligomer comprising an additional binding moiety of the present invention has an additional binding moiety separated from the CD16a-binding polypeptide or the CD16a-binding oligomer by a linker. A linker may be any linker defined hereinabove. For example, a linker selected from flexible amino acid linkers, rigid amino acid linkers, cleavable amino acid linkers and non-amino acid linkers.

Preferably, a linker for a CD16a-binding polypeptide or CD16a-binding oligomer comprising an additional binding moiety comprises or has a sequence of 1 to 50 (for example 1 to 25, for example 1, 2, 3, 4, 5, 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T (preferably G and S).

In one preferred embodiment, a linker for CD16a-binding polypeptide or CD16a-binding oligomer comprising an additional binding moiety of the present invention is G or comprises or has sequence GGGSG, GGGGS, GGSGG, GSGGG and/or SGGGG; for example the linker is G or comprises or has the sequence GGGSG, GGGSGGGGSG, GGGSGGGGSGGGGSG or GGGSGGGGSGGGGSGGGGSGGGGGS, GGGGSGGGGS, GGGGSGGGGSGGGGS, GGGGSGGGGSGGGGSGGGGS, GGSGG, GGSGGGGSGG, GGSGGGGSGGGGSGG, GGSGGGGSGGGGSGGGGSGGGGSGG, GSGGG, GSGGGGSGGG, GSGGGGSGGGGSGGG, GSGGGGSGGGGSGGGGSGGG, SGGGG, SGGGGSGGGG, SGGGGSGGGGSGGGG, or SGGGGSGGGGSGGGGSGGGG.

In one preferred embodiment, a linker for a CD16a-binding oligomer of the present invention is G or comprises or has sequence GGGSG; for example the linker comprises or has the sequence GGGSG, GGGSGGGGSG, GGGSGGGGSGGGGSG or GGGSGGGGSGGGGSGGGGSG. In another embodiment, a linker for a CD16a-binding oligomer of the present invention comprises or has sequence GGGGS; for example the linker comprises or has the sequence GGGGS, GGGGSGGGGS, GGGGSGGGGSGGGGS GGGGSGGGGSGGGGSGGGGS.

Alternatively, in one embodiment, a CD16a-binding polypeptide or CD16a-binding oligomer comprising an additional binding moiety of the present invention has an additional binding moiety that is not separated from the CD16a-binding polypeptide or the CD16a-binding oligomer by a linker (i.e. the CD16a-binding polypeptide or CD16a-binding oligomer is directly attached to an additional binding moiety).

In one preferred embodiment, a CD16a-binding polypeptide or CD16a-binding oligomer comprising an additional binding moiety of the present invention does not comprise a linker. In one preferred embodiment, the CD16a-binding polypeptide or CD16a-binding oligomer is directly attached to an additional binding moiety.

In certain embodiments, a CD16a-binding polypeptide of the present invention comprising an additional functional portion comprises the following structure:
[N-terminal portion]-[Helix 1]-[Separating portion]-[Helix 2]—[C-terminal portion]-[linker]-[additional functional portion];
[additional functional portion]-[linker]-[N-terminal portion]-[Helix 1]-[Separating portion]-[Helix 2]—[C-terminal portion]
[N-terminal portion]-[Helix 1]-[Separating portion]-[Helix 2]—[C-terminal portion]-[linker]-[additional functional portion]-[additional functional portion];
[additional functional portion]-[additional functional portion]-[linker]-[N-terminal portion]-[Helix 1]-[Separating portion]-[Helix 2]—[C-terminal portion]; or
[additional functional portion]-[linker]-[N-terminal portion]-[Helix 1]-[Separating portion]-[Helix 2]—[C-terminal portion]-[linker]-[additional functional portion];
wherein, when more than one additional functional portion is present, each additional functional may be the same, or may be different.

In such embodiments, the linker preferably comprises or has a sequence of 1 to 25 (for example 1, 2, 3, 4, 5, 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T (preferably G and S). For example, the linker is G or comprises or has sequence GGGSG, GGGGS, GGSGG, GSGGG and/or SGGGG; for example the linker is G or comprises or has the sequence GGGSG, GGGSGGGGSG, GGGSGGGGSGGGGSG, GGGSGGGGSGGGGSGGGGSG, GGGGS, GGGGSGGGGS, GGGGSGGGGSGGGGS, GGGGSGGGGSGGGGSGGGGS, GGSGG, GGSGGGGSGG, GGSGGGGSGGGGSGG, GGSGGGGSGGGGSGGGGSGGGGSGG, GSGGG, GSGGGGSGGG, GSGGGGSGGGGSGGG, GSGGGGSGGGGSGGGGSGGG, SGGGG, SGGGGSGGGG, SGGGGSGGGGSGGGG, or SGGGGSGGGGSGGGGSGGGG.

Alternatively, the linker may be absent, i.e. the CD16a-binding polypeptide or CD16a-binding oligomer comprises the following structure:
[N-terminal portion]-[Helix 1]-[Separating portion]-[Helix 2]—[C-terminal portion]-[additional functional portion];
[additional functional portion]-[N-terminal portion]-[Helix 1]-[Separating portion]-[Helix 2]—[C-terminal portion]
[N-terminal portion]-[Helix 1]-[Separating portion]-[Helix 2]—[C-terminal portion]-[additional functional portion]-[additional functional portion];
[additional functional portion]-[additional functional portion]-[N-terminal portion]-[Helix 1]-[Separating portion]-[Helix 2]—[C-terminal portion]; or
[additional functional portion]-[N-terminal portion]-[Helix 1]-[Separating portion]-[Helix 2]—[C-terminal portion]-[additional functional portion].

In such embodiments, more preferably the CD16a-binding polypeptide comprises the following structure:
[additional functional portion]-[linker]-[N-terminal portion]-[Helix 1]-[Separating portion]-[Helix 2]—[C-terminal portion]
[additional functional portion]-[additional functional portion]-[linker]-[N-terminal portion]-[Helix 1]-[Separating portion]-[Helix 2]—[C-terminal portion]; or
[additional functional portion]-[linker]-[N-terminal portion]-[Helix 1]-[Separating portion]-[Helix 2]—[C-terminal portion]-[linker]-[additional functional portion]
(or comprises the following structure
[additional functional portion]-[N-terminal portion]-[Helix 1]-[Separating portion]-[Helix 2]—[C-terminal portion]
[additional functional portion]-[additional functional portion]-[N-terminal portion]-[Helix 1]-[Separating portion]-[Helix 2]—[C-terminal portion]; or
[additional functional portion]-[N-terminal portion]-[Helix 1]-[Separating portion]-[Helix 2]—[C-terminal portion]-[additional functional portion]).

In such embodiments, preferably each additional functional portion is an additional binding moiety specific for a cancer cell surface target (for example a myeloma cell surface antigen, for example BCMA). Most preferably each additional functional portion is an additional binding moiety specific for BCMA,

Even more preferably, the CD16a-binding polypeptide comprises the following structure:
[additional functional portion]-[linker]-[N-terminal portion]-[Helix 1]-[Separating portion]-[Helix 2]—[C-terminal portion]; or
[additional functional portion]-[additional functional portion]-[linker]-[N-terminal portion]-[Helix 1]-[Separating portion]-[Helix 2]—[C-terminal portion].

In such embodiments, preferably each additional functional portion is an additional binding moiety specific for a cancer cell surface target (for example a myeloma cell surface antigen, for example BCMA). Most preferably each additional functional portion is an additional binding moiety specific for BCMA,

In one very especially preferred embodiment of the invention the CD16a-binding polypeptide comprises the following structure:
[additional functional portion]-[additional functional portion]-[linker]-[N-terminal portion]-[Helix 1]-[Separating portion]-[Helix 2]—[C-terminal portion].

In such embodiments, preferably each additional functional portion is an additional binding moiety specific for a cancer cell surface target (for example a myeloma cell surface antigen, for example BCMA). Most preferably each additional functional portion is an additional binding moiety specific for BCMA. Optionally, in such embodiments, the linker may be absent.

In another very especially preferred embodiment of the invention the CD16a-binding polypeptide consists of the following structure:
[additional functional portion]-[additional functional portion]-[linker]-[N-terminal portion]-[Helix 1]-[Separating portion]-[Helix 2]—[C-terminal portion].

In such embodiments, preferably each additional functional portion is an additional binding moiety specific for a cancer cell surface target (for example a myeloma cell surface antigen, for example BCMA). Most preferably each additional functional portion is an additional binding moiety specific for BCMA. Optionally, in such embodiments, the linker may be absent.

In certain embodiments, a CD16a-binding oligomer of the present invention comprising an additional functional portion comprises the following structure:
[N-terminal portion]-[Helix 1]-[Separating portion]-[Helix 2]—[C-terminal portion]-[linker]-[N-terminal portion]-[Helix 1]-[Separating portion]-[Helix 2]—[C-terminal portion]-[linker]-[additional functional portion];
[additional functional portion]-[linker]-[N-terminal portion]-[Helix 1]-[Separating portion]-[Helix 2]—[C-terminal portion]-[linker]-[N-terminal portion]-[Helix 1]-[Separating portion]-[Helix 2]—[C-terminal portion];
[N-terminal portion]-[Helix 1]-[Separating portion]-[Helix 2]—[C-terminal portion]-[linker]-[additional functional portion]-[linker]-[N-terminal portion]-[Helix 1]-[Separating portion]-[Helix 2]—[C-terminal portion];
[N-terminal portion]-[Helix 1]-[Separating portion]-[Helix 2]—[C-terminal portion]-[linker]-[N-terminal portion]-[Helix 1]-[Separating portion]-[Helix 2]—[C-terminal portion]-[linker]-[additional functional portion]-[additional functional portion];
[additional functional portion]-[additional functional portion]-[linker]-[N-terminal portion]-[Helix 1]-[Separating portion]-[Helix 2]—[C-terminal portion]-[linker]-[N-terminal portion]-[Helix 1]-[Separating portion]-[Helix 2]—[C-terminal portion];
[additional functional portion]-[linker]-[N-terminal portion]-[Helix 1]-[Separating portion]-[Helix 2]—[C-terminal portion]-[linker]-[N-terminal portion]-[Helix 1]-[Separating portion]-[Helix 2]—[C-terminal portion]-[-linker]-[additional functional portion];
[additional functional portion]-[linker]-[N-terminal portion]-[Helix 1]-[Separating portion]-[Helix 2]—[C-terminal portion]-[linker]-[additional functional portion]-[linker]-[N-terminal portion]-[Helix 1]-[Separating portion]-[Helix 2]—[C-terminal portion];
[N-terminal portion]-[Helix 1]-[Separating portion]-[Helix 2]—[C-terminal portion]-[linker]-[additional functional portion]-[linker]-[N-terminal portion]-[Helix 1]-[Separating portion]-[Helix 2]—[C-terminal portion]-[linker]-[additional functional portion]; or
[N-terminal portion]-[Helix 1]-[Separating portion]-[Helix 2]—[C-terminal portion]-[linker]-[additional functional portion]-[additional functional portion]-[linker]-[N-terminal portion]-[Helix 1]-[Separating portion]-[Helix 2]—[C-terminal portion]
wherein each linker portion in the oligomer may have the same sequence or have different sequences; each N-terminal portion in the oligomer may have the same sequence or have different sequences; each C-terminal portion in the oligomer may have the same sequence or have different sequences; each separating portion in the oligomer may have the same sequence or have different sequences; each Helix 1 portion in the oligomer may have the same sequence or have different sequences; and each Helix 2 portion in the oligomer may have the same sequence or have different sequences.

In such embodiments, the linker preferably comprises or has a sequence of 1 to 25 (for example 1, 2, 3, 4, 5, 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T (preferably G and S). For example, the linker is G or comprises or has sequence GGGSG, GGGGS, GGSGG, GSGGG and/or SGGGG. Also, for example, the linker is G or comprises or has the sequence GGGSG, GGGSGGGGSG, GGGSGGGGSGGGGSG, GGGSGGGGSGGGGSGGGGSG, GGGGS, GGGGSGGGGS, GGGGSGGGGSGGGGS, GGGGSGGGGSGGGGSGGGGS, GGSGG, GGSGGGGSGG, GGSGGGGSGGGGSGG, GGSGGGGSGGGGSGGGGSGGGGSGG, GSGGG, GSGGGGSGGG, GSGGGGSGGGGSGGG, GSGGGGSGGGGSGGGGSGGG, SGGGG, SGGGGSGGGG, SGGGGSGGGGSGGGG, or SGGGGSGGGGSGGGGSGGGG.

In such embodiments, one or more linker may be absent.

In such embodiments, more preferably the CD16a-binding oligomer comprises the following structure:
[additional functional portion]-[linker]-[N-terminal portion]-[Helix 1]-[Separating portion]-[Helix 2]—[C-terminal portion]-[linker]-[N-terminal portion]-[Helix 1]-[Separating portion]-[Helix 2]—[C-terminal portion];
[additional functional portion]-[additional functional portion]-[linker]-[N-terminal portion]-[Helix 1]-[Separating portion]-[Helix 2]—[C-terminal portion]-[linker]-[N-terminal portion]-[Helix 1]-[Separating portion]-[Helix 2]—[C-terminal portion];
[additional functional portion]-[linker]-[N-terminal portion]-[Helix 1]-[Separating portion]-[Helix 2]—[C-terminal portion]-[linker]-[N-terminal portion]-[Helix 1]-[Separating portion]-[Helix 2]—[C-terminal portion]-[-linker]-[additional functional portion]; or
[additional functional portion]-[linker]-[N-terminal portion]-[Helix 1]-[Separating portion]-[Helix 2]—[C-terminal portion]-[linker]-[additional functional portion]-[linker]-[N-terminal portion]-[Helix 1]-[Separating portion]-[Helix 2]—[C-terminal portion].

Even more preferably the CD16a-binding oligomer comprises the following structure:
[additional functional portion]-[linker]-[N-terminal portion]-[Helix 1]-[Separating portion]-[Helix 2]—[C-terminal portion]-[linker]-[N-terminal portion]-[Helix 1]-[Separating portion]-[Helix 2]—[C-terminal portion]; or
[additional functional portion]-[additional functional portion]-[linker]-[N-terminal portion]-[Helix 1]-[Separating portion]-[Helix 2]—[C-terminal portion]-[linker]-[N-terminal portion]-[Helix 1]-[Separating portion]-[Helix 2]—[C-terminal portion].

In certain embodiments, a CD16a-binding oligomer of the present invention comprising an additional functional portion comprises at least three CD16a-binding polypeptides, and comprises the following structure:
[N-terminal portion]-[Helix 1]-[Separating portion]-[Helix 2]—[C-terminal portion]-[Linker]-[N-terminal portion]-[Helix 1]-[Separating portion]-[Helix 2]—[C-terminal portion]-[Linker]-[N-terminal portion]-[Helix 1]-[Separating portion]-[Helix 2]—[C-terminal portion]-[linker]-[additional functional portion];
[additional functional portion]-[linker]-[N-terminal portion]-[Helix 1]-[Separating portion]-[Helix 2]—[C-terminal portion]-[Linker]-[N-terminal portion]-[Helix 2]-[Separating portion]-[Helix 2]—[C-terminal portion]-[Linker]-[N-terminal portion]-[Helix 1]-[Separating portion]-[Helix 2]—[C-terminal portion];
[N-terminal portion]-[Helix 1]-[Separating portion]-[Helix 2]—[C-terminal portion]-[Linker]-[N-terminal portion]-[Helix 1]-[Separating portion]-[Helix 2]—[C-terminal portion]-[Linker]-[N-terminal portion]-[Helix 1]-[Separating portion]-[Helix 2]—[C-terminal portion]-[linker]-[additional functional portion]-[additional functional portion];
[additional functional portion]-[additional functional portion]-[linker]-[N-terminal portion]-[Helix 1]-[Separating portion]-[Helix 2]—[C-terminal portion]-[Linker]-[N-terminal portion]-[Helix 2]-[Separating portion]-[Helix 2]—[C-terminal portion]-[Linker]-[N-terminal portion]-[Helix 1]-[Separating portion]-[Helix 2]—[C-terminal portion];
wherein each linker portion in the oligomer may have the same sequence, have different sequences, or two may have the same sequence and one may have a different sequence; each N-terminal portion in the oligomer may have the same sequence, have different sequences, or two may have the same sequence and one may have a different sequence; each C-terminal portion may have the same sequence, have different sequences, or two may have the same sequence and one may have a different sequence; each separating portion in the oligomer may have the same sequence, have different sequences, or two may have the same sequence and one may have a different sequence; each Helix 1 portion in the oligomer may have the same sequence, have different sequences, or two may have the same sequence and one may have a different sequence; and each Helix 2 portion in the oligomer may have the same sequence, have different sequences, or two may have the same sequence and one may have a different sequence.

In such embodiments, the linker is preferably one as defined above. In such embodiments, one or more linker may be absent.

In such embodiments, more preferably the CD16a-binding oligomer comprises the following structure:
[additional functional portion]-[linker]-[N-terminal portion]-[Helix 1]-[Separating portion]-[Helix 2]—[C-terminal portion]-[Linker]-[N-terminal portion]-[Helix 2]-[Separating portion]-[Helix 2]—[C-terminal portion]-[Linker]-[N-terminal portion]-[Helix 1]-[Separating portion]-[Helix 2]—[C-terminal portion]; or
[additional functional portion]-[additional functional portion]-[linker]-[N-terminal portion]-[Helix 1]-[Separating portion]-[Helix 2]—[C-terminal portion]-[Linker]-[N-terminal portion]-[Helix 2]-[Separating portion]-[Helix 2]—[C-terminal portion]-[Linker]-[N-terminal portion]-[Helix 1]-[Separating portion]-[Helix 2]—[C-terminal portion].

In the above embodiments defining the structure of the CD16a-binding polypeptide or CD16a-binding oligomer comprising an additional functional portion, each additional function portion may be any one described herein. For example, each additional functional portion may be independently selected from
an additional binding moiety (for example an additional binding moiety specific for a cancer cell surface target, for example a myeloma cell surface antigen, for example BCMA, or an immune cell target, for example a NK cell target); and
an immune signalling molecule, for example a cytokine, for example IL-15 or derivatives thereof.

For example, each additional functional portion may be independently selected from
an additional binding moiety specific for a cancer cell surface target (for example a myeloma cell surface antigen, for example BCMA);
an additional binding moiety specific for an immune cell target (for example a NK cell target); and
an immune signalling molecule, for example a cytokine, for example IL-15 or derivatives thereof.

For example, each additional functional portion may be independently selected from
an additional binding moiety specific for BCMA;
an additional binding moiety specific for a NK cell target; and
an immune signalling molecule, for example a cytokine, for example IL-15 or derivatives thereof.

For example, each additional functional portion may be independently selected from
an additional binding moiety specific for BCMA; and
an additional binding moiety specific for a NK cell target; and
an a cytokine, for example IL-15 or derivatives thereof.

For example, each additional functional portion may be independently selected from
an additional binding moiety specific for BCMA; and
an additional binding moiety specific for a NK cell target; and IL-15 or derivatives thereof.

In one preferred embodiment, each additional functional portion may be independently selected from
an additional binding moiety specific for BCMA; and
an additional binding moiety specific for a NK cell target; and
an a cytokine, for example IL-15 or derivatives thereof.

In one preferred embodiment, each additional functional portion may be independently selected from
an additional binding moiety specific for BCMA; and
an additional binding moiety specific for a NK cell target.

In one preferred embodiment, each additional functional portion is an additional binding moiety specific for BCMA.

In the above embodiments defining the structure of the CD16a-binding polypeptide or CD16a-binding oligomer comprising an additional functional portion, each additional function portion may be an additional binding moiety (for example an additional binding moiety specific for a cancer cell surface target, for example a myeloma cell surface antigen, for example BCMA). For example, each additional function portion may be an additional binding moiety for BCMA.

In a preferred embodiment, an additional binding moiety for BCMA is a hBCMA-binding polypeptide which comprises at least one motif that binds to hBCMA, wherein said polypeptide comprises the following structure:
[N-terminal portion]-[Helix 1]-[Separating portion]-[Helix 2]—[C-terminal portion] the hBCMA binding motif being the portion [Helix 1]-[Separating portion]-[Helix 2].

For example, it is a hBCMA-binding polypeptide as defined in the PCT Application filed in the name of Oncopeptides Innovation 1 AB on 31 May 2023 claiming priority to GB Application No. 2208027.9 and to GB Application No. 2214718.5. The contents of that PCT application are herein incorporated by reference.

In particular, the hBCMA-binding polypeptide is one wherein:
i) Helix 1 comprises the sequence X₉X₁₀X₁₁ADX₁₄EIX₁₇X₁₈ and Helix 2 comprises the sequence FX₂₅QKWAFX₃₁RX₃₃LX₃₅, wherein, independently from each other,
a) X₉ and X₁₀ are any naturally occurring amino acid; X₁₁ is E, F, H, Q, T or Y; X₁₄ is any naturally occurring amino acid; X₁₇ is A, E, Q, S, T or V; X₁₈ is any naturally occurring amino acid; X₂₅ is F or Y; X₃₁ is I, M, or V; X₃₃ is K or S; X₃₅ is I, L, M, or V;
or ii) Helix 1 and Helix 2 are defined as in i), wherein within Helix 1 and Helix 2, at least 1 and no more than 5 (for example at least 1 and no more than 3) of the Xₙ residues are replaced by an alternative residue, and/or at least 1 and no more than 5 (for example at least 1 and no more than 3) of the residues not labelled as Xₙ are replaced by an alternative residue.

For example, the hBCMA-binding polypeptide is one with hBCMA binding efficacy is at least 1% of the sequence

[SEQ ID 226]
VDNKFNKENQFADEEIAALPNLNFYQKWAFIRKLMDDPSQSANLLAE
AKKLNDAQAPK.

In a very preferred embodiment, the hBCMA-binding polypeptide is the polypeptide of SEQ ID 226.

### CD16a Binder-Drug Conjugates

Alternatively or additionally, a CD16a binding polypeptide or CD16a-binding oligomer as disclosed herein may be attached to a therapeutic agent to form a CD16a binder-drug conjugate. Therefore, in an embodiment, the at least one CD16a binding polypeptide or CD16a-binding oligomer is typically covalently attached to one or more therapeutic agent(s), optionally via a linker or linkers as described above. Non-limiting examples of such therapeutic agents include cytotoxic drugs, for example mitomycin C, desmethyltopotecan, SN-38, MMAE, MMAF, doxorubicin, pyrrolobenzodiazepine, amanitin, maytansinoids (for example maytansinoid DM1 or maytansinoid DM4), or duostatins (for example duostatin 5.2).

For the avoidance of doubt, during the generation of a CD16a binder-drug conjugate according to the invention, the one or more therapeutic agent(s) (for example MMAF) is necessarily modified by the reaction between the functional group at the point of attachment (for example —NH, —OH or —SH (for example in a Cys)) and any attachment groups or linkers used. The skilled person will therefore understand that the therapeutic agent in a CD16a binder-drug conjugate (for example a CD16a binder-MMAF conjugate) comprises such a modification.

### Polypeptide Production

The polypeptides of the invention can be manufactured using methods known in the art. For example, they can be prepared by chemical synthesis methods or by recombinant protein production techniques in, for example, bacterial, yeast, insect, fungal, plant or mammalian cells.

Polypeptides of the invention may also be fused, via recombinant or chemical synthesis techniques as described above, to a different molecule with therapeutic potential, for example an immunoglobulin with therapeutic potential.

### Modulation of Polypeptide Properties

The pharmacokinetic properties of the polypeptides of the invention can be modulated by methods known in the art. For example, they can be linked to a moiety extending the plasma half-life, such as a polyethylene glycol polymer, an unstructured polypeptide (such as XTEN or PAS), or an FcRn binding ligand such as serum albumin or the Fc domain of an immunoglobulin.

### Formulations

Polypeptides according to the invention (for example, a CD16a-binding polypeptide or CD16a-binding oligomer of the present invention, or a CD16a binder-drug conjugate) may be present in a formulation and particularly in a pharmaceutical formulation.

In certain embodiments, the invention provides a nucleic acid molecule encoding the CD16a-binding polypeptide or CD16a-binding oligomer of the invention. A nucleic acid molecule encoding the CD16a-binding polypeptide or CD16a-binding oligomer of the invention may be used as a medicament, for example may be used for the treatment of cancer. The nucleic acid molecule may, for example, be a DNA or an RNA molecule, for example a mRNA molecule. Nucleic acid molecules according to the invention may be present in a formulation and particularly in a pharmaceutical formulation. As such, the present invention further provides a formulation and particularly in a pharmaceutical formulation of a nucleic acid molecule (for example, a DNA or RNA, and in particular a mRNA molecule) encoding the CD16a-binding polypeptide or CD16a-binding oligomer of the invention.

Pharmaceutical formulations include for example those suitable for oral, parenteral (including subcutaneous, intradermal, intraosseous infusion, intramuscular, intravascular (bolus or infusion), and intramedullary), or intraperitoneal administration, although the most suitable route may depend upon, for example, the condition and disorder of the subject under treatment.

In one embodiment of the invention, a CD16a binding polypeptide or CD16a binding oligomer (in particular a CD16a binder-drug conjugate) or nucleic acids (for example DNA or RNA molecules of the present invention, for example an mRNA molecule) according to the invention is administered as a pharmaceutical formulation suitable for oral or parenteral (including subcutaneous, intradermal, intraosseous infusion, intramuscular, intravascular (bolus or infusion), and intramedullary) administration.

Pharmaceutical formulations suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. Peptides of the invention may also be presented as a bolus, electuary or paste. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2S, 1988.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Preferably, the formulations may be presented in unit dosage or divided dosage containers, for example sealed ampoules and vials. The formulation may be stored in a freeze-dried (lyophilised) condition requiring only the addition of the sterile liquid carrier, for example saline, a physiologically acceptable solution or water-for-injection, immediately prior to use.

Extemporaneous injection and infusion solutions and suspensions may be prepared from sterile powders, granules or other dry composition. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

### Dosage Regimens

A CD16a-binding polypeptide or CD16a binding oligomer (in particular a CD16a binder-drug conjugate) of the invention, and a pharmaceutical formulation comprising such a polypeptide, oligomer or binder-drug conjugate, or nucleic acid molecules of the invention (for example, DNA or RNA molecules of the present invention, for example a mRNA molecule of the present invention), and pharmaceutical formulations comprising those nucleic acid molecules, find use in the treatment and/or prophylaxis of cancer, for example multiple myeloma.

The amount of a CD16a-binding polypeptide or CD16a binding oligomer (in particular a CD16a binder-drug conjugate) or nucleic acid molecule, required to achieve a therapeutic effect will vary with the particular route of administration and the characteristics of the subject under treatment, for example the species, age, weight, sex, medical conditions, the particular disease and its severity, and other relevant medical and physical factors. An ordinarily skilled physician can readily determine and administer the effective amount of the CD16a-binding polypeptide, CD16a oligomer, CD16a binder-drug conjugate and/or composition comprising the same, or nucleic acid molecule and/or composition comprising the same, required for treatment and/or prophylaxis of cancer.

A CD16a-binding polypeptide, CD16a binding oligomer (in particular a CD16a binder-drug conjugate) or nucleic acid molecule of the invention, or a pharmaceutical formulation thereof, may for example be administered daily, weekly, every second, third or fourth week or even as a high single dose depending on the subject and severity of the cancer to be treated.

A CD16a-binding polypeptide, CD16a binding oligomer (in particular a CD16a binder-drug conjugate) or nucleic acid molecule of the invention, or a pharmaceutical formulation thereof, may for example be administered as a parenteral or oral dosage. Parenteral administration includes intravenous (into a vein, for example a central or a peripheral vein, bolus or infusion), intra-arterial (into an artery, for example a central or a peripheral artery), intraosseous infusion (into the bone marrow), intra-muscular (into muscle), intradermal (into the dermis), and subcutaneous (under the skin) administration. In one preferred embodiment, the dosage of the present invention is administered intravenously or intra-arterially, and more preferably by intravenous infusion (for example central intravenous infusion or peripheral intravenous infusion). In another preferred embodiment, the dosage of the present invention is administered by subcutaneous injection. As such, pharmaceutical formulations especially useful for the present invention are those suitable for intravenous administration, more especially intravenous infusion, or subcutaneous administration.

A CD16a-binding polypeptide, CD16a binding oligomer (in particular a CD16a binder-drug conjugate) or nucleic acid molecule of the invention, and pharmaceutical formulations thereof, may be administered as part of a treatment cycle. In a treatment cycle, said polypeptides may be administered on day 1 of the cycle, wherein the cycle lasts X days, with no further administration of the CD16a-binding polypeptides or CD16a-binding oligomers of the invention for the next X-1 days. X may be, for example, from 1 to 42, for example from 2 to 14 days. Alternatively, the CD16a-binding polypeptides or CD16a-binding oligomers may be administered as a split dose, for example on for example on days 1 and 2 of the cycle.

The cycle may be repeated one or several times depending on the category, class or stage of the cancer to be treated. For example, the cycle may be repeated from 1 to 100 times, for example from 2 to 50 times, for example 8 to 40 times, for example 8 or 16 times. For example, the CD16a-binding polypeptides or CD16a-binding oligomers or nucleic acid molecules of the invention may be administered for 8 repeats of a 7 day cycle, followed by 16 repeats of a 14 day cycle, optionally followed by further repeats of a 28 day cycle. An ordinarily skilled physician or clinician can readily determine the number of cycles of CD16a-binding polypeptide (for example CD16a binder-drug conjugate) required to prevent, counter or arrest the progress of the cancer.

### Combination Treatments

Whilst a CD16a-binding polypeptide or CD16a binding oligomer (in particular a CD16a binder-drug conjugate) or nucleic acid molecules disclosed herein may be used as the sole active ingredient in the present invention, it is also possible for it to be used in combination with one or more further therapeutic agent(s), and the use of such combinations provides one embodiment of the invention. Such further therapeutic agents may be agents useful in the treatment and/or prophylaxis of cancer, or other pharmaceutically active materials. Such agents are known in the art

Non-limiting examples of further therapeutic agents for use in the present invention may include proteasome inhibitors (PIs) (for example carfilzomib, bortezomib or ixazomib), immunomodulatory agents (IMiDs) (for example lenalidomide, thalidomide or pomalidomide), alkylators (for example cyclophosphamide, melphalan, bendamustine or melflufen), anthracyclines (for example doxorubicin), steroids (for example dexamethasone, prednisone or prednisolone), BCL-2 inhibitors (for example venetoclax), histone deacetylase (HDAC) inhibitors (for example panobinostat), anti-CD38 agents (for example daratumumab or isatuximab), immune checkpoint inhibitors (for example a CTLA-4 inhibitor, a PD-1 inhibitor, or a PD-L1 inhibitor), or ADAM17 inhibitors. For example, further therapeutic agents may be selected from proteasome inhibitors (for example carfilzomib or bortezomib), immunomodulatory agents (for example lenalidomide or thalidomide), alkylators (for example melphalan or melfufen), steroids (for example dexamethasone or prednisone), anti-CD38 agents (for example daratumumab), an immune checkpoint inhibitor (for example a CTLA-4 inhibitor, a PD-1 inhibitor, or a PD-L1 inhibitor), and an ADAM17 inhibitor.

The one or more further therapeutic agent(s) may be used simultaneously, sequentially or separately with/from the administration of the dosage of CD16a binding polypeptides, CD16a binding oligomers, CD16a binder-drug conjugates, or nucleic acid molecules of the invention. The individual components of such combinations can be administered separately at different times during the course of therapy or concurrently in divided or single combination forms.

In an embodiment of the invention, the one or more further therapeutic agent(s) are selected from a PI, an IMiD, and a steroid. For example, the one or more further therapeutic agents are a PI (for example bortezomib or carfilzomib), an IMiD (for example lenalidomide, thalidomide and pomalidomide), and a steroid (for example, prednisone, prednisolone and dexamethasone). Preferably, the one or more therapeutic agents are bortezomib, thalidomide, and dexamethasone.

In an embodiment of the invention, the one or more further therapeutic agent(s) are selected from a PI, an alkylator, and a steroid. For example, the one or more further therapeutic agents are a PI (for example bortezomib or carfilzomib), an alkylator (for example cyclophosphamide, melphalan, melflufen or bendamustine), and a steroid (for example, prednisone, prednisolone and dexamethasone). Preferably, the one or more therapeutic agents are bortezomib, melphalan, melflufen, and prednisone.

In an embodiment of the invention, the one or more further therapeutic agent(s) are selected from a PI and a steroid. For example, the one or more therapeutic agents are a PI (for example bortezomib or carfilzomib), and a steroid (for example, prednisone, prednisolone and dexamethasone). Preferably, the PI is bortezomib and the steroid is dexamethasone.

In an embodiment of the invention, the one or more further therapeutic agent(s) are selected from an IMiD and a steroid. For example, the one or more therapeutic agents are an IMiD (for example lenalidomide, thalidomide and pomalidomide), and a steroid (for example, prednisone, prednisolone and dexamethasone). Preferably, the IMiD is lenalidomide and the steroid is dexamethasone.

In another embodiment of the invention, the one or more therapeutic agent(s) may be selected from an NK cell-based or T cell-based therapy.

Alternatively, a CD16a-binding polypeptide or CD16a binding oligomer (in particular a CD16a binder-drug conjugate) or nucleic acid molecule according to the invention may be combined with a therapeutic procedure such as a stem cell transplantation procedure, for example an autologous stem cell transplant or an allogenic stem cell transplant. Therefore, in an embodiment of the invention, a CD16a-binding polypeptide disclosed herein may be combined with an autologous stem cell transplantation procedure. In another embodiment of the invention, a CD16a-binding polypeptide disclosed herein may be combined with an allogenic stem cell transplantation procedure.

The simultaneous, sequential or separate administration of one or more further therapeutic agent (s) or therapeutic procedure with the CD16a binding polypeptides, CD16a binding oligomers, CD16a binder-drug conjugates or nucleic acid molecules of the invention further enhances their effectiveness in the treatment and/or prophylaxis of cancer.

### Kits

The present invention provides a kit comprising a CD16a-binding polypeptide, CD16a binding oligomer (in particular a CD16a binder-drug conjugate) or nucleic acid molecule as disclosed herein, and one or more further therapeutic agents that are useful in the treatment and/or prophylaxis of cancer.

Non-limiting examples of further therapeutic agents for use in a kit of the present invention may include proteasome inhibitors (PIs) (for example carfilzomib, bortezomib or ixazomib), immunomodulatory agents (IMiDs) (for example lenalidomide, thalidomide or pomalidomide), alkylators (for example cyclophosphamide, melphalan, melflufen or bendamustine), anthracyclines (for example doxorubicin), steroids (for example dexamethasone, prednisone or prednisolone), BCL-2 inhibitors (for example venetoclax), histone deacetylase (HDAC) inhibitors (for example panobinostat), anti-CD38 agents (for example daratumumab or isatuximab), immune checkpoint inhibitors (for example a CTLA-4 inhibitor, a PD-1 inhibitor, or a PD-L1 inhibitor), or ADAM17 inhibitors. The one or more further therapeutic agents may for example be selected from proteasome inhibitors (for example carfilzomib or bortezomib), immunomodulatory agents (for example lenalidomide or thalidomide), alkylators (for example melphalan or melflufen), steroids (for example dexamethasone or prednisone), anti-CD38 agents (for example daratumumab), an immune checkpoint inhibitor (for example a CTLA-4 inhibitor, a PD-1 inhibitor, or a PD-L1 inhibitor), and an ADAM17 inhibitor.

In one embodiment of the invention, the kit of the present invention finds use in the treatment and/or prophylaxis of cancer.

For the avoidance of doubt, a CD16a-binding polypeptide, CD16a binding oligomer (in particular a CD16a binder-drug conjugate) or nucleic acid molecule as disclosed herein is present in a kit according to the present invention in a form and quantity suitable for use according to the present invention. Suitable pharmaceutical formulations are described herein. The skilled person can readily determine a quantity of the CD16a binding polypeptide or oligomer (for example in the form of a CD16a binder-drug conjugate) or nucleic acid molecule as disclosed herein suitable for the use according to the present invention.

### Cancers

CD16a-binding polypeptides or CD16a-binding oligomers of the present invention, (in particular CD16a binder-drug conjugates) or nucleic acid molecules of the present invention, as well as pharmaceutical formulations or kits of the present invention comprising said CD16a-binding polypeptides, CD16a binding oligomers, CD16a binder-drug conjugates or nucleic acid molecules, find use in medicine, for example in the treatment and/or prophylaxis of cancer in a subject. Non-limiting examples of cancers include: solid cancers, such as bladder cancer, breast cancer, colorectal cancer, CNS cancers, endometrial cancer, kidney cancer, liver cancer, lung cancer, skin cancer, ovarian pancreatic cancer, prostate cancer, or thyroid cancer; and blood cancers, such as leukaemias (for example acute myeloid leukaemia, acute lymphoblastic leukaemia, chronic myeloid leukaemia, or chronic lymphocytic leukaemia), lymphomas (for example Hodgkin lymphoma, non-Hodgkin lymphoma, cutaneous T-cell lymphoma, small lymphocytic lymphoma, and other high-grade B-cell lymphomas), or plasma cell neoplasms and myelomas (for example, MGUS, plasmacytoma, smouldering myeloma, multiple myeloma, light chain myeloma, or non-secretory myeloma).

Preferably, CD16a-binding polypeptides or CD16a binding oligomers of the present invention (in particular CD16a binder-drug conjugates) or nucleic acid molecules of the present invention, as well as pharmaceutical formulations or kits of the present invention comprising said CD16a-binding polypeptides, oligomers, binder-drug conjugates or nucleic acid molecules, find use in the treatment and/or prophylaxis of blood cancers, such as leukaemias (for example acute myeloid leukaemia, acute lymphoblastic leukaemia, chronic myeloid leukaemia, or chronic lymphocytic leukaemia), lymphomas (for example Hodgkin lymphoma, non-Hodgkin lymphoma, cutaneous T-cell lymphoma, small lymphocytic lymphoma, and other high-grade B-cell lymphomas), or plasma cell neoplasms and myelomas (for example, MGUS, plasmacytoma, smouldering myeloma, multiple myeloma, light chain myeloma, or non-secretory myeloma). Preferably, CD16a-binding polypeptides or CD16a-binding oligomers of the present invention, as well as pharmaceutical formulations or kits of the present invention comprising said CD16a-binding polypeptides or CD16a-binding oligomers, find use in the treatment and/or prophylaxis of myelomas (for example, MGUS, plasmacytoma, smouldering myeloma, multiple myeloma, light chain myeloma, or non-secretory myeloma). Most preferably, CD16a-binding polypeptides, CD16a-binding oligomers, CD16a binder-drug conjugates or nucleic acid molecules of the present invention, as well as pharmaceutical formulations or kits of the present invention comprising said CD16a-binding polypeptides, CD16a-binding oligomers, CD16a binder-drug conjugates or nucleic acid molecules, find use in the treatment and/or prophylaxis of multiple myeloma.

In certain embodiments the CD16a-binding polypeptides or CD16a binding oligomers of the present invention (in particular CD16a binder-drug conjugates) or nucleic acid molecules of the present invention, as well as pharmaceutical formulations or kits of the present invention comprising said CD16a-binding polypeptides, oligomers, binder-drug conjugates or nucleic acid molecules, find use as anti-cancer immunotherapeutics; for example anti-cancer immunotherapeutics for use in the treatment of cancers, and in particular blood cancers, for example a blood cancer as described herein, and in particular multiple myeloma.

Whilst the present invention has been described and illustrated with reference to particular embodiments, it will be appreciated by those of ordinary skill in the art that the invention lends itself to many different variations not specifically illustrated herein.

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

### EXAMPLES

The following Examples illustrate the invention.

### Preparative Example 1: Selection of Binders to hCD16a by Phage Display Using a Phage Library

In this Example, human hCD16a was used as the target in phage display selections using a phage-based affibody library. Individual clones obtained after four selection cycles were assayed for binding to hCD16a in a monoclonal phage-ELISA (enzyme-linked immunosorbent assay) and ELISA-positive clones were DNA sequenced.

### Materials and Methods

### Affibody Library.

An M13 phage display library of affibody molecules was prepared based on the phagemid vector pAffi-1 (Gronwall et al. (2007) J. Biotechnol. 128:162-183), This phagemid, containing a lac promoter and an OmpA signal peptide, is designed for phage display of encoded affibody library members as in-frame fusions to an albumin binding domain (ABD_{WT}), an amber stop codon and a truncated form (residues 249-406) of the M13 phage coat protein 3. A synthetic 121 bases long oligonucleotide (5′-GCGCTTTGGCTTGGGTCATCXXXTAAACTXXXYYYGAAGGCXXXXXXTTG XXXXXXGTTCAGGTTCGGCAGXXXXXXGATCTCXXXXXXCGCXXXXXXX XXTTCTTTGTTGAATTTGTTGT-3)′ [SEQ ID 222] encoding amino acids 3-41 (reverse complementary strand) of the Z domain (Nilsson et al. (1987) Protein Eng. 1:107-113), in which wildtype codons for 14 positions in the domain had been randomized using mixtures of trinucleotide codons building blocks (XXX=equal mixture of codons for all 20 amino acids except Cys and Pro, YYY=60% Ile and 10% each of His, Tyr, Lys and Asp) during the synthesis was used as template for PCR amplification using primers Forward (5′-GATGAAGCCCTCGAGGTAGACAACAAATTCAACAAAGAA-3′) [SEQ ID 223] and Reverse (5′-TTAGCTTCTGCTAGCAAGTTAGCGCTTTGGCTTGGGTCATC-3′) [SEQ ID 224]. Approximately 6.4 μg of XhoI and NheI double-cleaved and gel-purified (Qiagen, Germany) PCR product was ligated to 35 μg of XhoI and NheI double-cleaved and gel-purfied pAffi-1 phagemid vector using T4 DNA ligase. The resulting ligation mixture was desalted using column-purification (Qiagen, Germany), divided into 24 portions and used to electroporate (0.1 cm BioRad cuvettes) 25 μl electrocompetent ER2738 E. coli cells (F′, glnV amber suppressor) (Lucigen, USA). 970 μl of Recovery medium (2% tryptone, 0.5% yeast extract, 10 mM NaCl, 2.5 mM KCl, 10 mM MgCl₂, 10 mM MgSO₄, and 20 mM glucose) was added to electroporated cells which were subsequently pooled (six electroporations per pool) and incubated at 37° C. for 1 h under shaking, after which pools of cells were titrated via spreading of dilution series on Amp (ampicillin) plates and transferred to four 5 litre shakeflasks, each containing one litre of Tryptic Soy Broth+Yeast extract medium (30 g/l tryptic soy broth, 5 g/l yeast extract; TSB+Y) supplemented with 2% glucose and 100 μg/ml Amp. Overnight cultures of cells were pelleted by centrifugation and resuspended in 50 ml of cold 40% glycerol, followed by distribution into 29 tubes of ca. 3.5 ml cell/glycerol solution per tube. From the post-electroporation titrations and the OD600 measurements after the overnight cultivation it was calculated that the library size (diversity) was ca. 3×10¹⁰ and that each 3.5 ml aliquot of cells contained a number of cells corresponding to ca. 10.9× the library size. The tubes with cells were stored at −80° C. until used for phage stock preparation using M13KO7 helper phage.

### Naïve Phage Library Stock Production.

For the production of an affibody-displaying phage stock, 1.7 ml library glycerol stock was distributed and inoculated into four E-flasks containing 750 ml TSB+Y, 1% (w/v) glucose, 10 μg/ml Tet (tetracycline) and 100 μg/ml Carb (carbenicillin) and grown at 37° C. with shaking at 150 rpm until the cultures reached an OD600=1. Cells were infected with 120 μl (multiplicity of infection (MOI) of 5) of M13KO7 helper phage (New England Biolabs), gently swirled and incubated without shaking for 15 min at 37° C., followed by 70 rpm at 37° C. for 15 min. The cells of each culture were centrifuged and resuspended in 750 ml TSB+Y, 100 μg/ml Carb and 1 mM IPTG (Isopropyl β-D-1-thiogalactopyranoside). 25 μg/ml Kan (kanamycin) was added 2 h after inoculation. Cultures were incubated at 37° C. with shaking at 200 rpm for 16-18 h. The phage library stock was harvested by precipitation with 20% (w/v) PEG6000/2.5 M NaCl twice. The titer of the stock was measured by spot titration and polymerase chain reaction-screening was used to analyze the percentage of phage particles carrying the phagemid with affibody insert.

Phage Display Selection of hCD16a Binding Candidates.

Four cycles of panning were performed using biotinylated hCD16a (biotinylated human hCD16a (F158), Avitag, His tag, Acro Biosystems, cat. no. CDA-H82E8 corresponding to residues 17-208 of Uniprot entry P08637) at concentrations 80 nM, 40 nM, 20 nM and 10 nM, respectively, in two separate selection tracks. SA (streptavidin)-coated paramagnetic beads (Dynabeads M-280 Streptavidin, cat. no. 11205D, Invitrogen, Waltham, Massachusetts, USA) were washed twice with PBS (150 mM NaCl, 8 mM Na₂HPO₄, 2 mM NaH₂PO₄·H₂O, pH 7.4). To avoid unspecific binders, all tubes used in the biopanning were pre-treated with 1% (w/v) bovine serum albumin (BSA) in PBS-T (PBS supplemented with 0.05% (v/v) Tween-20, pH 7.4). Furthermore, phage stock in PBS-T was pre-incubated for 30 min at room temperature (RT) under constant end-over-end (eoe) rotation with 0.1% (w/v) BSA and beads, to remove phages carrying binders against SA. Amount of phage stock used was 10¹¹ colony forming units (cfu) in cycles 1 and 4, and 10¹² cfu in cycles 2-3. In rounds 1-2, biotinylated target protein was immobilised on 1 mg or 0.5 mg beads, respectively, for 1 h at RT and end-over-end (eoe). Target-bound beads were incubated with 1% (w/v) BSA in PBS-T for 30 min at RT and eoe and then washed with PBS-T, before the addition of pre-incubated phage stock to perform the selection. In rounds 3-4, pre-incubated phage stock was added to biotinylated target protein to perform the selection in solution, after which phage antigen complexes were captured by incubation with 0.5 mg or 0.4 mg SA-beads, respectively, for 30 min at RT and eoe. The selection step was performed at RT and the incubation time for selection was 3 h (round 1) or 2 h (rounds 2-4) followed by wash with PBS-T eoe at RT for a total of 5 min, 10 min, 15 min or 20 min, for each subsequent cycle. The final wash volume was transferred to new 1% (w/v) BSA pre-treated tubes to remove sticky binders attached to the tube walls. For one of the selection tracks, antigen-binding phages were eluted by incubation with 0.5 M acetic acid, pH 2.8 for 15 min eoe at RT, followed by transfer of the eluate to new pre-treated tubes and neutralization with equal volume 1 M Tris-HCl, pH 8. For the second selection track, antigen-binding phages were eluted by incubating with 0.25 mg/ml trypsin (Gibco Life Technologies) in TBS-T (TRIS buffered saline, 0.1% (v/v) Tween-20) supplemented with 1 mM CaCl₂ for 30 min eoe at RT before transferring the eluate to new pre-treated tubes.

### Phage Stock Amplification.

New phage stocks were generated by growing Escherichia coli XL-1 Blue cells (Agilent) in TSB+Y with 10 μg/ml Tet at 37° C. with shaking at 150 rpm until OD600=0.5-0.8 and then infecting them (100 ml after cycle 1-2, 50 ml after cycle 3) with phage eluate (total volume after cycle 1, half volume after cycle 2-3). Infected cultures were gently swirled, incubated without shaking for 25 min at 37° C., followed by shaking at 70 rpm at 37° C. for 15 min. The cultures were then centrifuged and resuspended in TSB+Y, before plating on blood agar plates (40 g/l blood agar) with 100 μg/ml Carb and 1% (w/v) glucose, and incubation for 16-18 h at 37° C. Bacterial colonies were collected to TSB+Y by scraping and inoculated to 200 ml TSB+Y with 100 μg/ml Carb and grown at 37° C. with shaking at 150 rpm until OD600=0.5-0.8. 30 ml (after cycles 1-2) or 20 ml (after cycle 3) were superinfected with M13KO7 helper phage (MOI of 10), gently swirled, incubated without shaking for 25 min at 37° C., followed by shaking at 70 rpm at 37° C. for 15 min. The cultures were centrifuged and resuspended in 150 ml TSB+Y with 100 μg/ml Carb and 1 mM IPTG. 25 μg/ml Kan was added 2 h after inoculation. Cultures were incubated at 37° C. with shaking at 150 rpm for 16-18 h. The phage library stock was harvested by precipitation with 20% (w/v) PEG6000/2.5 M NaCl twice. The titer of the stock was measured by spot titration on Carb plates and polymerase chain reaction-screening was used to analyze the percentage of phage particles carrying phagemids with the affibody insert.

### Monoclonal Phage Supernatant Preparation.

Following cycle 4, bacterial colonies generating correctly sized PCR products and equally representing the two selection tracks were individually grown in 500 μl TSB+Y/Carb in 96-well deep-well plates at 30° C. with shaking at 250 rpm for 16-18 h. 30 μl of overnight culture was inoculated to 720 μl TSB+Y/Carb in a new 96-well deep-well plate and incubated for 2 h at 37° C., 250 rpm, before superinfection by addition of M13KO7 helper phage (MOI 7) in 100 μl TSB+Y/Carb per well. Plates were incubated for 30 min at 37° C. without shaking. Finally, 150 μl TSB+Y/Carb/IPTG/Kan was added per well. Final concentrations were 100 μg/ml Carb, 1 mM IPTG and 25 μg/ml Kan. The deep-well plates were incubated at 37° C., 250 rpm, for 16-18 h. Phage supernatants were collected by centrifugation the next day.

### Monoclonal Phage-ELISA Screening of Candidates.

A MaxiSorp ELISA plate (Clear Flat-Bottom Immuno Nonsterile 384-Well Plates, cat. no. 464718, Thermo Fisher Scientific) was coated at 4° C. with slow shaking for 16-18 h with 30 μl 10 μg/ml biotinylated hCD16a, 20 μg/ml human serum albumin (HSA; Sigma product no. SRP6182) (for assessment of proper display of the expression cassette containing a tripartite fusion protein including an affibody, an affibody albumin binding domain and the truncated protein 3), 10 μg/ml SA or 10 μg/ml unrelated control protein (human SLAMF7/CRACC/CD319, Fc Tag, Acro Biosystems, cat. no. SL7-H5256 corresponding to residues 23-226 of Uniprot entry Q9NQ25) in 100 mM sodium carbonate buffer pH 9.6 per well (¼ of the wells with each coated protein). The coating solution was removed and the plate was washed two times with PBS-T, and blocked with PBS supplemented with 1% (w/v) BSA for 1 hr at RT with slow shaking. The blocking solution was discarded, and the wells were incubated with 10 μl phage supernatant diluted 1:3 with 20 μl PBS-T for 1 h at RT with slow shaking. The supernatant was removed and the plate washed three times with PBS-T. Following addition of 30 μl 1:5000 α-M13-HRP (Sigma-Aldrich, Stockholm, Sweden) in PBS-T and incubated for 30 min at RT with slow shaking, the plate was washed twice with PBS-T and once with PBS. 30 μl TMB substrate (TMB Substrate Kit, Thermo Fisher Scientific) was added to each well. The reactions were stopped by adding 30 μl 2 M H₂SO₄ after 15-25 min. Absorbance at 450 nm was measured using a CLARIOstar microplate reader (BMG Labtech, Ortenberg, Germany). Candidates that were considered as ELISA-positive clones had high HSA signal and relatively high signal to hCD16a compared to the SA and unrelated target controls.

DNA sequencing. Following ELISA screening, 26 ELISA-positive clones were sent for DNA sequencing by Sanger sequencing (PlateSeq, Eurofins Genomics, Ebersberg, Germany).

### Results

To identify polypeptides binding to hCD16a, an affibody phage library was constructed and used as input in the first cycle in a four-cycle phage display selection campaign as described in Materials and Methods (above). After the fourth cycle of selections, the phage eluates from the acid and trypsin selection tracks were used to infect E. coli cells to obtain individual colonies on Carbenicillin plates. Forty-eight randomly picked colonies (clones) from each track were subjected to binding analyses using a phage-ELISA experiment using microtiter plates in which four test antigens (HSA, streptavidin, SLAMF7 and hCD16a F158) had been coated. Several of the analyzed clones showed strong ELISA signals to HSA and hCD16a, and low signals for the streptavidin and SLAMF7 controls used, as shown for representative clones in FIG. 1b. Such clones were subjected to DNA sequencing which showed that three unique clones had been enriched during the selections. The amino acid sequences of these three CD16a-binding polypeptides, A10, All and H09 are listed in Table 1 below and in the sequence listing as SEQ IDs 1, 74 and 75. Table 1 also lists the sequences of certain other peptides closely based on those that are also disclosed herein, with the corresponding SEQ ID references. Throughout this Examples section, an Example Compound with number X is a polypeptide with the amino sequence of the same number, ie SEQ ID X.

TABLE 1

| | | SEQ |
|---|---|---|
| Name | Sequence | ID |
| A10 | VDNKFNKEVQMAQFEIRKLPNLNHHQSFAFIKSLMDDPSQSAN | 1 |
| | LLAEAKKLNDAQAPK | |
| A11 | VDNKFNKEQFYARDEIDLLPNLNEDQKWAFYMSLIDDPSQSAN | 74 |
| | LLAEAKKLNDAQAPK | |
| H09 | VDNKFNKEFWIAESEIESLPNLNIYQKWAFKYSLADDPSQSANL | 75 |
| | LAEAKKLNDAQAPK | |
| His₆- | GSSHHHHHHYYLEVDNKFNKEVQMAQFEIRKLPNLNHHQSFA | 76 |
| A10- | FIKSLMDDPSQSANLLAEAKKLNDAQAPKVDYVLAEAKVLAN | |
| ABD_{WT} | RELDKYGVSDYYKNLINNAKTVEGVKALIDEILAALP | |
| His₆- | GSSHHHHHHYYLEVDNKFNKEQFYARDEIDLLPNLNEDQKWA | 77 |
| A11- | FYMSLIDDPSQSANLLAEAKKLNDAQAPKVDYVLAEAKVLAN | |
| ABD_{WT} | RELDKYGVSDYYKNLINNAKTVEGVKALIDEILAALP | |
| His₆- | GSSHHHHHHYYLEVDNKFNKEFWIAESEIESLPNLNIYQKWAF | 78 |
| H09- | KYSLADDPSQSANLLAEAKKLNDAQAPKVDYVLAEAKVLANR | |
| ABD_{WT} | ELDKYGVSDYYKNLINNAKTVEGVKALIDEILAALP | |
| His₆- | GSSHHHHHHYYLEVDNKFNKEVQMAQFEIRKLPNLNHHQSFA | 79 |
| A10-Cys | FIKSLMDDPSQSANLLAEAKKLNDAQAPKC | |
| His₆- | GSSHHHHHHYYLEVDNKFNKEQFYARDEIDLLPNLNEDQKWA | 80 |
| A11-Cys | FYMSLIDDPSQSANLLAEAKKLNDAQAPKC | |
| His₆- | GSSHHHHHHYYLEVDNKFNKEVQMAQFEIRKLPNLNHHQSFA | 81 |
| A10- | FIKSLMDDPSQSANLLAEAKKLNDAQAPKGGGSGGGGSGGGG | |
| A11-Cys | SGVDNKFNKEQFYARDEIDLLPNLNEDQKWAFYMSLIDDPSQS | |
| | ANLLAEAKKLNDAQAPKC | |
| His₆- | GSSHHHHHHYYLEVDNKFNKEQFYARDEIDLLPNLNEDQKWA | 82 |
| A11- | FYMSLIDDPSQSANLLAEAKKLNDAQAPKGGGSGGGGSGGGG | |
| A10-Cys | SGVDNKFNKEVQMAQFEIRKLPNLNHHQSFAFIKSLMDDPSQS | |
| | ANLLAEAKKLNDAQAPKC | |
| A10- | VDNKFNKEVQMAQFEIRKLPNLNHHQSFAFIKSLMDDPSQSAN | 83 |
| A11-His₆ | LLAEAKKLNDAQAPKQAPKGGGSGGGGSGGGGSGVDNKFNK | |
| | EQFYARDEIDLLPNLNEDQKWAFYMSLIDDPSQSANLLAEAKK | |
| | LNDAQAPKYYHHHHHH | |
| A11- | VDNKFNKEQFYARDEIDLLPNLNEDQKWAFYMSLIDDPSQSAN | 84 |
| A10-His₆ | LLAEAKKLNDAQAPKQAPKGGGSGGGGSGGGGSGVDNKFNK | |
| | EVQMAQFEIRKLPNLNHHQSFAFIKSLMDDPSQSANLLAEAKK | |
| | LNDAQAPKYYHHHHHH | |
| Anti- | VDNKFNKENQFADEEIAALPNLNFYQKWAFIRKLMDDPSQSAN | 85 |
| BCMA | LLAEAKKLNDAQAPKGGGSGGGGSGGGGSGVDNKFNKEFWIA | |
| (1-E6)- | ESEIESLPNLNIYQKWAFKYSLADDPSQSANLLAEAKKLNDAQ | |
| H09- | APKGGGSGGGGSGGGGSGVDNKFNKEVQMAQFEIRKLPNLNH | |
| A10-His₆ | HQSFAFIKSLMDDPSQSANLLAEAKKLNDAQAPKYYHHHHHH | |
| Anti- | VDNKFNKENQFADEEIAALPNLNFYQKWAFIRKLMDDPSQSAN | 86 |
| BCMA | LLAEAKKLNDAQAPKGGGSGGGGSGGGGSGVDNKFNKEVQM | |
| (1-E6)- | AQFEIRKLPNLNHHQSFAFIKSLMDDPSQSANLLAEAKKLNDA | |
| A10- | QAPKGGGSGGGGSGGGGSGVDNKFNKEVQMAQFEIRKLPNLN | |
| A10-His₆ | HHQSFAFIKSLMDDPSQSANLLAEAKKLNDAQAPKYYHHHHH | |
| | H | |
| Anti- | VDNKFNKENQFADEEIAALPNLNFYQKWAFIRKLMDDPSQSAN | 87 |
| BCMA | LLAEAKKLNDAQAPKGGGSGGGGSGGGGSGVDNKFNKEQFY | |
| (1-E6)- | ARDEIDLLPNLNEDQKWAFYMSLIDDPSQSANLLAEAKKLNDA | |
| A11- | QAPKGGGSGGGGSGGGGSGVDNKFNKEVQMAQFEIRKLPNLN | |
| A10-His₆ | HHQSFAFIKSLMDDPSQSANLLAEAKKLNDAQAPKYYHHHHH | |
| | H | |
| Anti- | VDNKFNKENQFADEEIAALPNLNFYQKWAFIRKLMDDPSQSAN | 88 |
| BCMA | LLAEAKKLNDAQAPKGGGSGGGGSGGGGSGVDNKFNKEVQM | |
| (1-E6)- | AQFEIRKLPNLNHHQSFAFIKSLMDDPSQSANLLAEAKKLNDA | |
| A10-His₆ | QAPKYYHHHHHH | |

Table 1 shows the sequences of certain peptides of the present disclosure with their allocated SEQ ID NOs.

### Biological Example 2

### Initial Binding Studies Using Surface Plasmon Resonance

In this Example, CD16a-binding polypeptides corresponding to SEQ ID 1, 74 and 75 were subcloned, expressed, purified as His₆-affibody-ABD_{WT} fusion proteins [SEQ ID NOs. 76-78] and initially analyzed by surface plasmon resonance for binding to CD16a.

### Materials and Methods

### Subcloning of CD16a-Binding Polypeptides

DNA fragments encoding the three CD16a-binding polypeptides [with SEQ ID 1, 74 and 75] were amplified from the library vector pAffi-1 with specific primers introducing overhangs complementary to ends of linearised expression vectors. In-Fusion HD Cloning Kit (Takara Bio, Gothenburg, Sweden) was used to clone monomeric CD16a-binding polypeptide constructs with N-terminal His₆ tag and C-terminal ABD_{WT}. DNA constructs were sequence verified using Sanger sequencing (Eurofins Genomics). The amino acid sequences of these three CD16a-binding polypeptides are listed in Table 1 in the sequence listing as SEQ IDs 76-78.

### Expression and Purification

E. coli BL21(DE3) cells were transformed with plasmids containing the DNA constructs and cultivated in 10 ml TSB+Y with 25 μg/ml Kan at 37° C. with shaking at 150 rpm for 16-18 h. The cultures were inoculated 1:100 in 200 ml TSB+Y with 25 g/ml Kan and grown at 37° C. with shaking at 150 rpm until OD600=0.6-1. IPTG was added to final concentration 1 mM to induce protein expression. The cultivations were incubated at 25° C. with shaking at 150 rpm for 16-18 h and the cells were harvested by centrifugation. The cell pellets were resuspended in denaturing lysis buffer (7 M guanidinium chloride, 47 mM Na₂HPO₄, 2.65 mM NaH₂PO₄, 10 mM TRIS-HCl, 100 mM NaCl, pH 8). After incubation at 37° C. for 2 h with shaking at 150 rpm, the cells were centrifuged and the denatured protein from the supernatant of the cell lysates was added to tubes containing 3 ml HisPur Cobalt IMAC Resin (cat. no. 89966, Thermo Scientific). Supernatant was incubated with resin for 30 min at RT with end-over-end rotation. Contaminants were removed by washing three times with wash buffer (7 M guanidium chloride, 46.6 mM Na₂HPO₄, 3.4 mM NaH₂PO₄, 300 mM NaCl, 10 mM imidazole, pH 8) and hCD16a binding polypeptides were subsequently eluted with elution buffer (6 M urea, 50 mM NaH₂PO₄, 100 mM NaCl, 30 mM glacial acetic acid, 70 mM sodium acetate, pH 5) by incubation at RT for 10 min with end-over-end rotation. Following IMAC purification, the protein buffer was exchanged to PBS using PD-10 desalting columns (cat. no. 17085101, Cytiva, Uppsala, Sweden). SDS-PAGE analysis was performed to confirm the purity and concentrations, which were measured by absorbance at 280 nm using extinction coefficients calculated from amino acid sequences, of the purified proteins (NuPAGE, 4 to 12%, Bis Tris, Invitrogen, Waltham, Massachusetts, USA). The low molecular weight marker was from Cytiva (Art. no. 17044601). The hCD16a binding variants with N-terminal His₆ and C-terminal ABDWT were expressed as soluble gene products in E. coli BL21(DE3).

Biosensor Analyses of hCD16a Binding Variants with N-Terminal His6 and C-Terminal ABD_{WT}

A Biacore 8K instrument (Cytiva) was used to analyse real-time interaction of hCD16a binding variants (SEQ ID NOs 76-78) with human hCD16a. The protein ligands hCD16a F158 (Cat. no. CDA-H5220, Acro Biosystems), hCD16a V158 (Cat. no. CD8-H52H4, Acro Biosystems) and human serum albumin (HSA) as a positive control (Sigma Art. no. SRP6182) were diluted in 10 mM NaOAc, pH 4.5 and immobilised on a Series S CM5 sensor chip (Cytiva). The sensor chip consists of flow channels made up of two flow cells arranged in series. For each protein ligand and corresponding flow channel, the first flow cell was activated and deactivated to be used as a reference surface and the second flow cell was used to immobilize the protein ligands to the carboxymethylated dextran surface by amine coupling, using the manufacturer's instructions. The hCD16a binding variants were diluted in the running buffer PBS-T (composition, pH) before binding analysis was performed at 25° C. and a flow rate of 30 l/min. After each injection the flow cells were regenerated by an injection of 10 mM HCl. Each hCD16a binding variant was injected over the hCD16a surfaces at concentrations ranging from 5 nM to 2.56 M, in duplicates. The dissociation equilibrium constants (KD values were calculated using a 1:1 Langmuir binding model in BIAevaluation software (Cytiva).

### Results

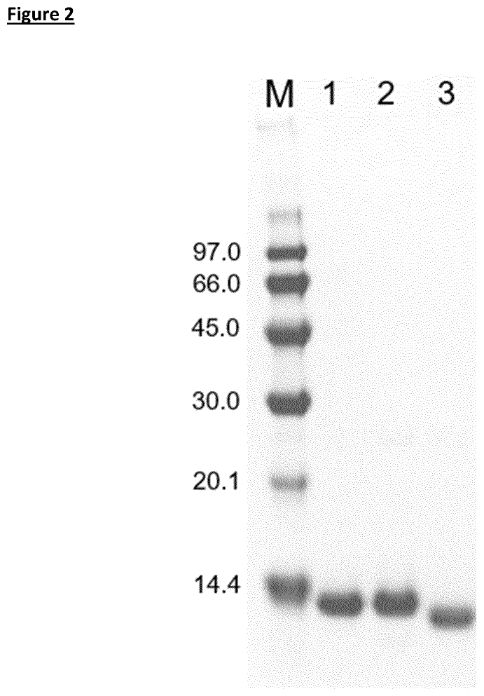

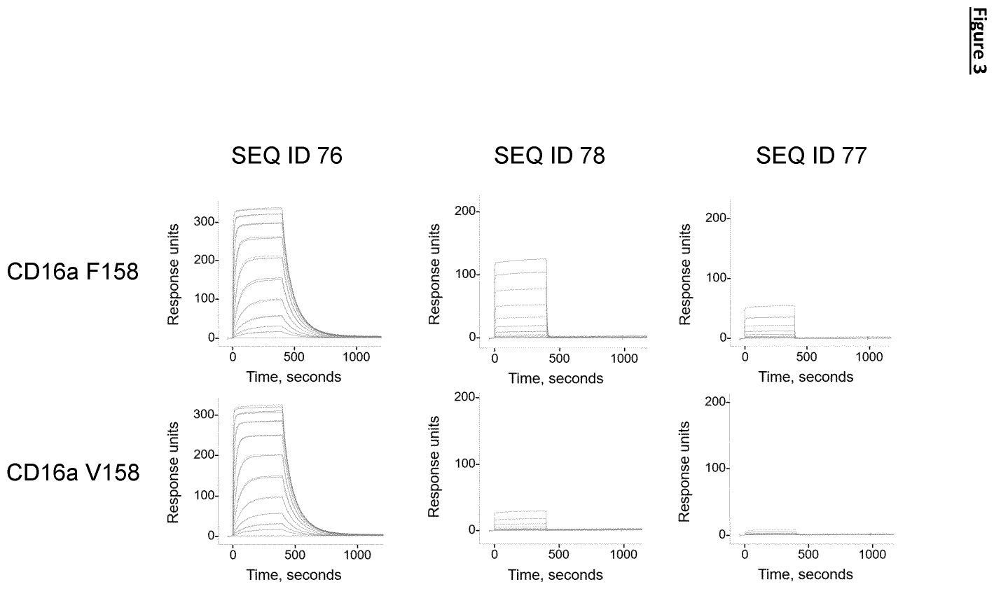

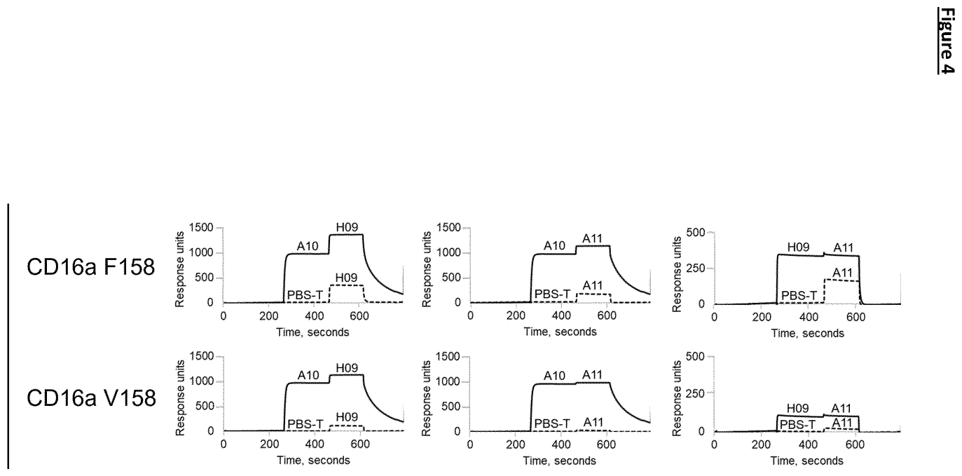

To investigate if the three CD16a-binding polypeptide candidate clones identified via the phage-ELISA and DNA sequencing [SEQ IDs 1, 74 and 75] showed target binding when expressed as soluble proteins, these were subcloned, cytoplasmically expressed in E. coli, and purified as His₆-CD16a-binding polypeptide-ABD_{WT} fusion proteins [SEQ IDs 76-78]. The ABD_{WT} moiety is a small serum albumin binding protein that can be used for affinity chromatography purification and/or immobilization using human serum albumin as ligand, if wanted. Immobilized metal ion affinity chromatography (IMAC)-purified fusion proteins, employing the hexahistidyl (His₆) gene fusion partner in the proteins, were analyzed by SDS-PAGE which showed that all three proteins were of high purity and had approximate molecular weights in accordance with their amino acid sequences (FIG. 2). Injection of the three fusion proteins at different concentrations (from 5 nM to 2.56 μM) over separate sensor chip surfaces containing immobilized hCD16a F158 and hCD16a V158 allotypes, respectively, showed that all three fusion proteins showed binding to both the hCD16a F158 and hCD16a V158 allotypes (FIG. 3).

Analyses of the equilibrium responses for the interactions allowed for a determination of the dissociation equilibrium constant (KD) for the interactions. The fusion protein containing the A10 CD16a-binding polypeptide [SEQ ID NO. 76] showed to bind both the F158 and V158 allotypes with similar affinity, with KD's of approximately 100 and 99 nM, respectively. In contrast, fusion proteins containing either the H09 or All CD16a-binding polypeptides [SEQ IDs 77 and 78], both showed to bind F158 allotype with higher affinity than the V158 allotype. Dissociation equilibrium constants for the interactions were calculated to be 2.8 μM (A11/V158), 8.3 μM (A11/F158), 621 nM (H09/F158) and 4.9 μM (H09/V158).

### Biological Example 3

Binning Studies of the hCD16a Binding Variants Using Surface Plasmon Resonance

In this Example, SPR experiments were performed to investigate if the three identified anti-CD16 affibodies bound to distinct or shared/overlapping epitopes on hCD16a.

### Material and Methods

Binning of hCD16a Binding Variants with N-Terminal His6 and C-Terminal ABD_{WT}.

A Biacore 3000 instrument (Cytiva) was used for competitive binding analyses of the hCD16a binding variants with each other. hCD16a F158 and hCD16a V158 were immobilised to separate flow cell carboxymethylated dextran surfaces on a CM5 sensor chip (Cytiva) by amine coupling, using the manufacturer's instructions. One flow cell was activated and deactivated to be used as a reference cell. The hCD16a binding variants were diluted in the running buffer PBS-T before binding analysis was performed at 25° C. and a flow rate of 30 l/min. After each injection series the flow cells were regenerated by the injection of 10 mM HCl.

### Results

In order to investigate if the identified CD16a-binding polypeptides recognize distinct or overlapping/shared epitopes on hCD16a, an SPR-based binning experiment was performed using sensor chip surfaces containing immobilized hCD16a F158 and hCD16a V158 proteins, respectively. The hCD16a binding variant, here used in the format as the fusion protein His6-A10-ABD_{WT} [SEQ ID 76], was injected at 2 M over the hCD16a surfaces, followed by different dual injections of mixed samples containing His₆-A10-ABD_{WT} [SEQ ID 76] at a retained 2 μM concentration together with either His₆-A11-ABD_{WT} [SEQ ID 77] or His₆-H09-ABD_{WT} [SEQ ID 78], both at 2 M. A similar experiment was performed with His₆-H09-ABD_{WT} [SEQ ID 78] injected at 2 μM in the first pulse, followed by dual injection of a mixed sample containing His₆-H09-ABD_{WT} [SEQ ID 78] at a retained concentration of 2 M together with His₆-A11-ABD_{WT} [SEQ. ID 77] also at 2 μM. As controls, running buffer PBS-T was injected instead of the variants used to retain surface at 2 M concentrations in the second pulse.

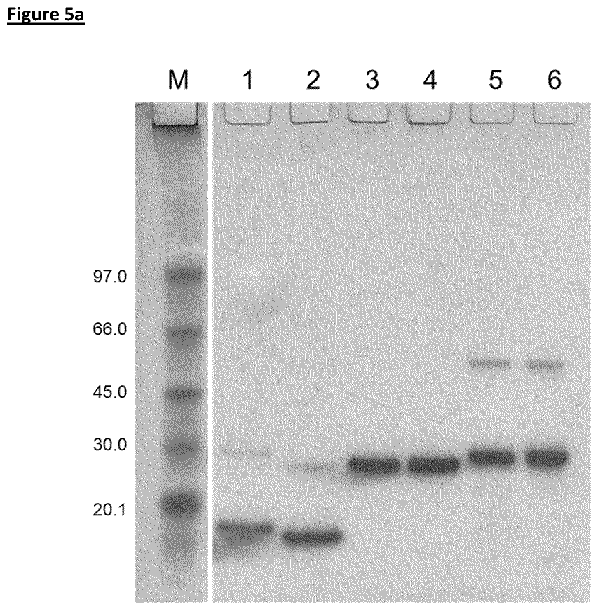

The results of the epitope binning assay showed that a first binding of the fusion protein His₆-A10-ABD_{WT} to hCD16a did not block the binding to hCD16a of subsequently injected fusion proteins His₆-H09-ABD_{WT} or His₆-All-ABD_{WT}, containing the H09 and A11 CD16a-binding polypeptides, respectively, indicating non-overlapping epitopes on hCD16a for variant A10 and either of the variants H09 and A11 (FIG. 4). From the results obtained in a similar experimental set-up using His₆-H09-ABD_{WT} and His₆-All-ABD_{WT} fusion proteins, it could be concluded that the variants H09 and A11 bind to overlapping epitopes (FIG. 4). The mutual epitope binning results were not dependent on the V158 or F158 allotypes of hCD16a.

### Biological Example 4

### Binding Studies of Monomeric, Heterodimeric and Homodimeric CD16a-Binding Polypeptide Constructs Using Surface Plasmon Resonance

In this Example, SPR experiments were performed to compare hCD16a binding properties of a set of monomeric and heterodimeric CD16a-binding polypeptide constructs.

### Material and Methods

### Subcloning of Monomeric, Heterodimeric and Homodimeric CD16a-Binding Polypeptide Constructs.

DNA fragments encoding CD16a-binding polypeptides were amplified using their respective library pAffi-1 phagemid vectors as templates. For construction of heterodimeric CD16a-binding polypeptide constructs, specific primers introducing codons encoding a flexible (GGGGS)₃ inter-CD16a-binding polypeptide linker [SEQ ID 225] as well as nucleotide overhangs complementary to cloning ends of linearised E. coli expression vectors were used. In-Fusion HD Cloning Kit (Takara Bio, Gothenburg, Sweden) was used to clone monomeric and heterodimeric constructs equipped with both an N-terminal His₆ tag and a C-terminal cysteine (Cys) [SEQ IDs 79-82] as well as heterodimeric constructs with only a C-terminal His₆ tag [SEQ IDs 83-84]. In addition, a potential dual engager construct containing a hBCMA-binding polypeptide fused to a homodimeric hCD16a binding arm (two tandemly linked copies of the A10 CD16a-binding polypeptide) was cloned with a C-terminal His₆ tag [SEQ ID 86]. DNA constructs were sequence verified using Sanger sequencing (Eurofins Genomics).

### Expression and Protein Purification of Monomeric, Heterodimeric and Homodimeric CD16a-Binding Polypeptide Constructs

E. coli BL21 (DE3) cells were transformed with plasmids containing the different DNA constructs and cultivated in 10 ml TSB+Y with 25 μg/ml Kan at 37° C. with shaking at 150 rpm for 16-18 h. The cultures were inoculated 1:100 in 200 ml TSB+Y in 25 μg/ml Kan and grown at 37° C. with shaking at 150 rpm until OD600=0.6-1. IPTG was added to final concentration 1 mM to induce protein expression. The cultivations were incubated at 25° C. with shaking at 150 rpm for 16-18 h and the cells were harvested by centrifugation. The cell pellets were resuspended in binding/washing buffer (PBS with 15 mM imidazole, pH 7.4). After cell disruption by sonication, cell debris was removed by centrifugation and each supernatant was applied on 3-4 ml HisPur packed gravity flow columns. Contaminants were removed by washing with six column volumes binding/washing buffer. hCD16a binding polypeptides were subsequently eluted with elution buffer (PBS with 300 mM imidazole, pH 7.4). Following IMAC purification, the protein buffer was exchanged to PBS (pH 7.4) using PD-10 desalting columns (Cytiva). SDS-PAGE analysis was performed to confirm the purity and concentrations, which were measured by absorbance at 280 nm using extinction coefficients calculated from amino acid sequences, of the purified proteins (NuPAGE, 4 to 12%, Bis Tris, Invitrogen).

### Biosensor Analyses of Monomeric, Heterodimeric and Homodimeric CD16a-Binding Polypeptide Constructs.

Using a Biacore 8K instrument (Cytiva), the second flow cell surfaces of four flow channels on a Series S CM5 sensor chip (Cytiva) was separately immobilised with produced N-terminal His6 and C-terminal Cys CD16a-binding polypeptide constructs [SEQ IDs 79-82], by ligand thiol coupling, using the manufacturer's protocol. The first flow cell of each flow channel was activated and deactivated to be used as reference surfaces. Analytes were diluted in the running buffer PBS-T before binding analysis was performed at 4° C. and a flow rate of 30 μl/min. hCD16a F158 and hCD16a V158 proteins were injected over the CD16a-binding polypeptide surfaces at 200 nM. A reverse set-up was used in a second experiment, where the second flow cell surface of each flow channel on a Series S CM5 sensor chip (Cytiva) was separately immobilised with hCD16a F158 and hCD16a V158 by amine coupling, using the manufacturer's protocol. The first flow cell of each flow channel was activated and deactivated to be used as reference surfaces. Analytes were diluted in the running buffer PBS-T before binding analysis was performed at 4° C. and a flow rate of 30 l/min. After each injection the flow cells were regenerated by the injection of 10 mM HCl. Heterodimeric hCD16a binding constructs [SEQ IDs 83-84] were injected over the hCD16a surfaces at concentrations ranging from 0.5 nM to 32 nM. In a third experiment, using a Biacore T200 instrument (Cytiva), hCD16a F158 and hCD16a V158 were immobilised on a carboxymethylated dextran surface on a CM5 sensor chip (Cytiva) by amine coupling, using the manufacturer's instructions. The potential dual engager construct containing an anti-hBCMA polypeptide fused to a homodimeric A10-A10 CD16a-binding polypeptide arm [SEQ ID 86] was diluted in the running buffer PBS-T. Binding analysis was performed at 25° C. and a flow rate of 30 μl/min in a single cycle kinetics set-up at five concentrations ranging 2.5-40 nM. After the final injection, the flow cells were regenerated by the injection of 10 mM HCl.

### Results

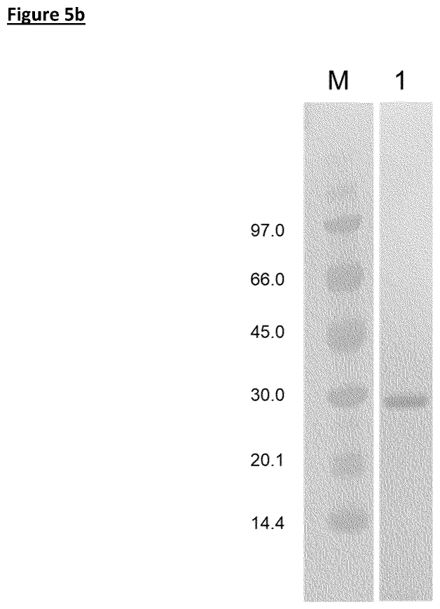

Expression vectors for a set of monomeric and heterodimeric CD16a-binding polypeptideswere constructed. Four constructs, His₆-A10-Cys, His₆-A11-Cys, His₆-A10-A11-Cys and His₆-A11-A10-Cys [SEQ IDs 79-82], corresponded to either monomeric or heterodimeric CD16a-binding polypeptide constructs equipped with both an N-terminal His6 tag and a C-terminal cysteine residue, the latter to be employed for a directed coupling to SPR sensor chips using thiol chemistry. Two heterodimeric constructs, A10-A11-His₆ and A11-A10-His₆ [SEQ IDs 83-84] and one dual engager construct composed of a BCMA-binding polypeptide fused to a homodimeric A10-A10 CD16a-binding polypeptide arm, anti-BCMA-A10-A10-His₆ [SEQ ID 86], were designed to contain only a C-terminal His6 tag. All seven constructs were expressed intracellularly in E. coli BL21(DE3) cells and soluble proteins were purified by IMAC. FIG. 5 shows the SDS-PAGE analysis of each final protein preparation, which demonstrated that these predominantly contained bands corresponding to the expected molecular weights. Larger molecular weight bands visible in lanes 1, 2, 5 and 6 in FIG. 5a correspond to an expected presence of also dimeric species, held together via a disulfide bond formed between the C-terminal cysteines present in these constructs.

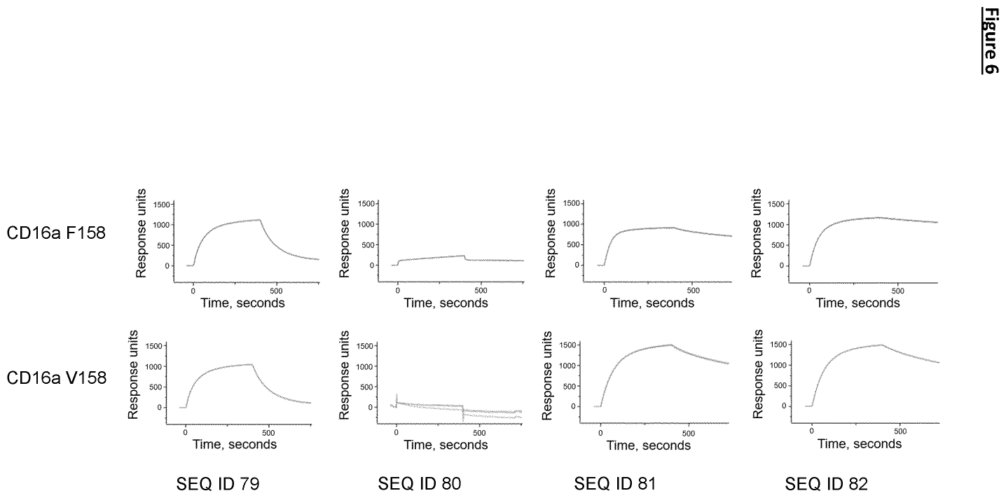

A first series of binding experiments were performed using the His₆-A10-Cys, His₆-All-Cys, His₆-A10-A11-Cys and His₆-A11-A10-Cys proteins, separately immobilized to thiol-activated sensor chip surfaces via their C-terminal cysteine residues. Injection of hCD16a F158 or hCD16a V158 proteins at 200 nM concentrations over sensor chip surfaces containing the monomeric CD16a-binding polypeptide constructs His6-A10-Cys and His6-A11-Cys, respectively, resulted in binding response profiles resembling those obtained in Example 2 using a reversed set-up, with a significantly stronger binding displayed by the A10 CD16a-binding polypeptide (FIG. 6). Injection of hCD16a F158 or hCD16a V158 proteins at 200 nM concentrations over sensor chip surfaces containing the heterodimeric His₆-A10-All-Cys and His₆-A11-A10-Cys proteins resulted in binding profiles characterized by considerably slower off-rate kinetics to both analytes, indicating that the injected monomeric hCD16a proteins were here more firmly bound via co-operative binding with contributions from both CD16a-binding polypeptides in the heterodimeric His₆-A10-A11-Cys and His₆-All-A10-Cys constructs. Interestingly, the sensorgrams indicate that the relative orientation (N-term to C-term) A11-A10 was more productive (slower off-rate kinetics) than the A10-A11 orientation using this particular linker length between the moieties ((GGGGS)₃) [SEQ ID 225]. When also taking the epitope binning results from Example 3 in consideration, showing that the A10 and A1 l CD16a-binding polypeptides bind to non-overlapping epitopes on hCD16a, the results strongly suggest that both the His₆-A10-A11-Cys and His₆-All-A10-Cys proteins are capable of binding to hCD16a via a bi-paratopic effect.

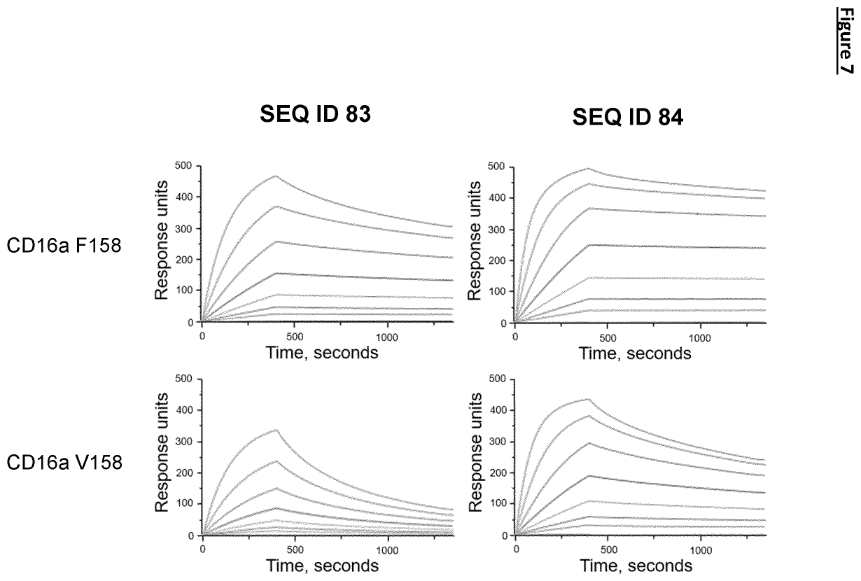

Using a reversed assay format, the two heterodimeric constructs, A10-A11-His₆ [SEQ ID 83] and A11-A10-His₆ [SEQ ID 84] were separately injected at concentrations ranging from 0.5 nM to 32 nM over sensor chip surfaces containing immobilized hCD16a F158 or hCD16a V158 proteins. The results shown in FIG. 7 shows that also in this assay format the binding responses to both hCD16a ligands obtained reflect a stronger binding (slower off-rate kinetics) than observed for any of the individual and monomeric A10 or A11 counterparts as analysed in the same assay format in Example 2 (FIG. 3, data for His₆-A10-ABDWT and His₆-All-ABDWT). This further indicates that a bi-paratopic binding effect to hCD16a can be obtained from combining the A10 and A11 variants into heterodimeric constructs. Further, as also the H09 CD16a-binding polypeptide showed to bind to an epitope not overlapping with that of the A10 CD16a-binding polypeptide (Example 3), heterodimeric variants based on combinations of the A10/H09 variants could from the results in this Example also be expected to show bi-paratopic binding to hCD16a, which is also demonstrated in Example 5.

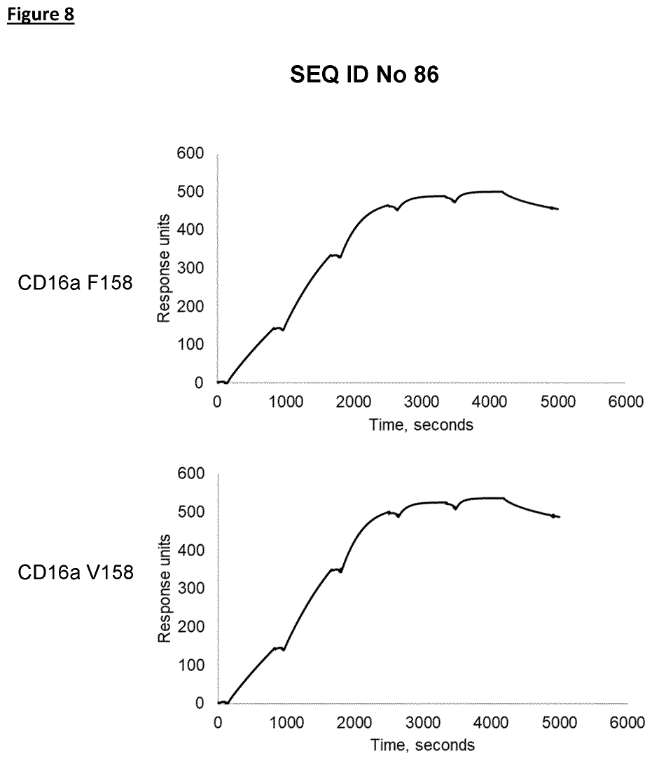

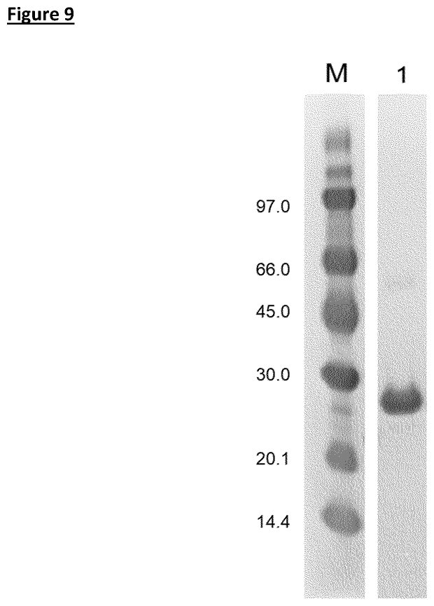

Further, the hCD16a binding properties of a fusion protein containing the homodimeric A10-A10 CD16a-binding polypeptide combination [SEQ ID 86] was also analysed. Here, a single cycle kinetics analysis was performed involving successive injections of the analyte at concentrations ranging from 2.5-40 nM over sensor chip surfaces containing immobilized hCD16a F158 or hCD16a V158 proteins, respectively. The results show that compared with proteins containing a single momomeric A10 CD16a-binding polypeptide moiety (FIG. 3, data for His₆-A10-ABD_{WT}), the protein analysed here containing two tandemly linked A10 CD16a-binding polypeptide moieties display a significantly stronger apparent affinity for both the hCD16a variants, with calculated apparent dissociation constants under these conditions (e.g. the surface density of hCD16a variants on the chip) for hCD16a F158 and hCD16a V158 of 0.4 and 0.3 nM, respectively, indicating that avidity effects from simultaneous and co-operative binding to two sensor chip immobilized CD16 protein molecules by a single injected homodimeric analyte protein are in effect (FIG. 8).

Taken together, the hCD16a binding analyses of this set of monomeric, heterodimeric and homodimeric constructs based on the CD16a-binding polypeptides described herein show that these can be freely combined in new constructs to result in a variety of novel hCD16a-binding proteins with different binding characteristics, including bi-paratopic and bivalent binding. The choice of CD16a-binding polypeptides and their relative order (N-term-to-C term) enables the overall binding properties to be varied. In the set of constructs analyzed in this example a common 15 amino acid linker composed of three tandemly arranged GGGSG motifs (SEQ ID 145) was used to link homodimeric and heteodimeric combinations. Additional hCD16a binding constructs based on alternative polypeptide or chemical linkers (including a direct linkage; i.e. no linker), and inclusion of more than two identical or different CD16a-binding polypeptide moieties are also possible to create using methods known in the art.

### Biological Example 5

### Assessment of a BCMA×hCD16a Dual Engager Construct Using Surface Plasmon Resonance

In this Example, SPR experiments were performed to analyze the bi-specific binding characteristics of a tri-partite fusion protein containing an hBCMA binding polypeptide (herein referred to as 1-E6, with sequence VDNKFNKENQFADEEIAALPNLNFYQKWAFIRKLMDDPSQSANLLAEAKKL NDAQAPK, SEQ ID 226) fused to a hCD16a binding arm consisting of a heterodimeric H09-A10 CD16a-binding polypeptide combination.

### Material and Methods

Subcloning of hBCMA×hCD16a-Binding Polypeptide Dual Engager Construct.

DNA fragments encoding an hBCMA binding polypeptide and two of the hCD16a binding polypeptides [SEQ IDs 1 and 75], were separately amplified from ordered gene fragments (gBlocks Gene Fragments, Integrated DNA Technologies, Leuven, Belgium), using primers introducing overhangs complementary to ends of linearised expression vectors and flexible (GGGGS)₃ linkers [SEQ ID 225] between each affinity protein unit. In-Fusion HD Cloning Kit (Takara Bio, Gothenburg, Sweden) was used to assemble the three gene fragments to construct a tri-partite BCMA×hCD16a dual engager with a C-terminal His6 tag [SEQ. ID 85]. The DNA constructs were sequence verified using Sanger DNA sequencing (Eurofins Genomics).

Expression and Protein Purification of hBCMA×hCD16a CD16a-Binding Polypeptide Dual Engager Construct.

E. coli BL21(DE3) cells were transformed with plasmids containing the DNA constructs and cultivated in 10 ml TSB+Y with 25 μg/ml Kan at 37° C. with shaking at 150 rpm for 16-18 h. The cultures were inoculated 1:100 in 200 ml TSB+Y in 25 g/ml Kan and grown at 37° C. with shaking at 150 rpm until OD600=0.6-1. IPTG was added to final concentration 1 mM to induce protein expression. The cultivations were incubated at 25° C. with shaking at 150 rpm for 16-18 h and the cells were harvested by centrifugation. The cell pellets were resuspended in binding/washing buffer (PBS with 15 mM imidazole, pH 7.4). After cell disruption by sonication, cell debris was removed by centrifugation and each supernatant was applied on 3-4 ml HisPur packed gravity flow columns. Contaminants were removed by washing with 6 column volumes binding/washing buffer. hCD16a binding polypeptides were subsequently eluted with elution buffer (PBS with 300 mM imidazole, pH 7.4). Following IMAC purification, the protein buffer was exchanged to PBS using PD-10 desalting columns (Cytiva). SDS-PAGE analysis was performed to confirm the purity and concentrations, which were measured by absorbance at 280 nm using extinction coefficients calculated from amino acid sequences, of the purified proteins (NuPAGE, 4 to 12%, Bis Tris, Invitrogen).

Evaluation of Dual Target Binding of hBCMA×hCD16a CD16a-Binding Polypeptide Dual Engager Construct.

A Biacore T200 instrument (Cytiva) was used to assess the dual engager construct's simultaneous binding to hBCMA and hCD16a. Human BCMA-rFc (BCMA Protein aa 1-54, Human, Recombinant (ECD, rabbit Fc Tag), cat. no. 10620-H15H, Sino Biological, Eschborn, Germany) was immobilised to a carboxymethylated dextran surface on a CM5 sensor chip (Cytiva) by amine coupling, using the manufacturer's instructions. One flow cell was activated and deactivated to be used as a reference cell. The dual engager construct and hCD16a variants were diluted in the running buffer PBS-T before binding analysis was performed at 25° C. and a flow rate of 30 l/min. After each injection the flow cells were regenerated by the injection of 10 mM HCl.

### Results

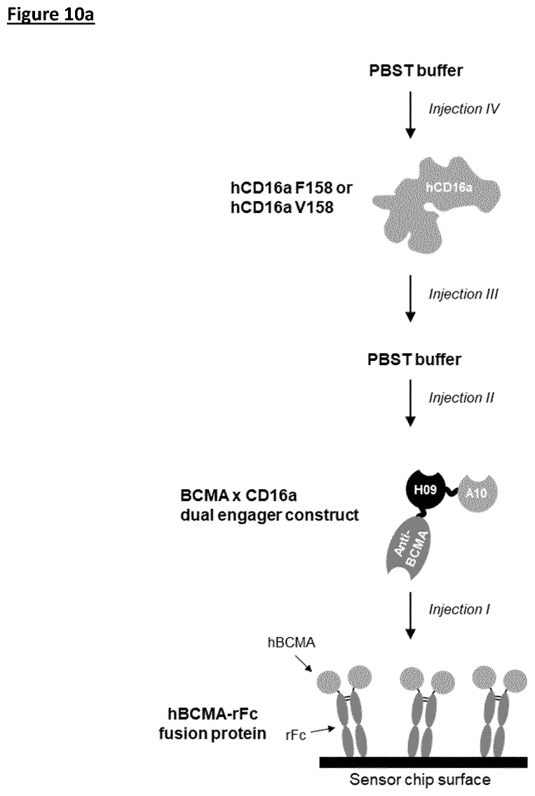

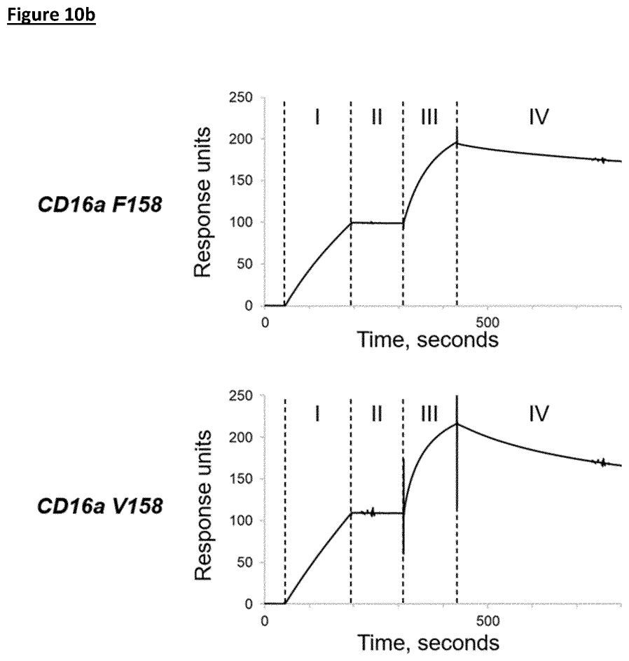

A BCMA×hCD16a dual engager construct containing a BCMA-binding polypeptide genetically fused to a hCD16a binding arm composed of a heterodimeric H09-A10 affibody combination and a C-terminal His₆ tag was constructed and expressed as a soluble gene product in E. coli (DE3). FIG. 9 shows the SDS-PAGE analysis of the final protein preparation, which demonstrated that it predominantly contained the dual engager construct with a band corresponding to the expected molecular weight. The hBCMA×hCD16a dual engager construct was injected at 200 nM over a sensor chip surface containing immobilized hBCMA-rFc protein, followed by successive injections of PBS-T buffer or hCD16a F158/hCD16a V158 proteins at 200 nM as schematically shown in FIG. 10a. Since the injected hCD16a variants could be expected to bind weakly to the rabbit Fc-tag on of the hBCMA-rFc protein, control serial injections with PBS-T buffer only (no hBCMA×hCD16a dual engager during injection I) followed by injections of hCD16a F158 or V158 proteins at 200 nM were performed to allow for a subtraction of any responses corresponding to this background binding. FIG. 10b shows the resulting sensorgrams obtained after subtractions of signals from the reference flow cell (flow cell 1, activated/deactivated) as well as the low response signal (<10 RU) obtained from direct hCD16a binding to the rabbit Fc-tag of the immobilised hBCMA-rFc ligand. These resulting sensorgrams show that the hBCMA×hCD16a dual engager construct is capable of simultaneous binding to both the immobilized hBCMA-rFc ligand and the subsequently injected hCD16a variants. The slow off-rate kinetics seen after completed injections (phase IV in FIG. 10b) indicates that also the H09-A10 affibody combination is capable of bi-paratopic binding to hCD16a, as previously demonstrated for the A10-A11 and A11-A10 combinations (Example 4). Also, as expected from the results obtained in Example 2, a slower hCD16a dissociation rate (FIG. 10b, phase IV) was seen for the interaction with the hCD16a F158 variant than for the hCD16a V158 variant as the H09 affibody variant had been demonstrated to bind stronger to the hCD16a F158 allotype (Example 2).

Taken together, the results show that an affibody-based hCD16a binding arm, here composed of a heterodimeric H09-A10 affibody combination, can retain its hCD16a binding ability also when fused to another polypeptide unit that simultaneously is engaged in binding to a therapeutically relevant target, here exemplified by a genetically fused polypeptide binding to hBCMA which is a target of relevance in multiple myeloma cancer.

### Biological Example 6

### Anti-BCMA Engagers Induce Immune Cell Activation in the Presence of Tumour Cells

Experiments were performed to evaluate the propensity of different anti-BCMA engagers to evoke an IFNγ response in cocultures of human PBMCs and the BCMA positive multiple myeloma cell line MM.1 S. Thawed and overnight rested PBMCs (4W-270, Lonza) were co-cultured with MM.1S cells (CRL-2974, ATCC) at effector to target ratio of 25:1 in V shaped 96-well plate (249935, Thermo Scientific). Total 4×10⁵ cells in 200 μl/well were treated with 200 nM anti-BCMA engagers or 200 nM Elotuzumab (300 mg Empliciti, Bristol-Myers Squibb) for 4 hours at 37° C. in a humidified 5% CO₂ incubator. Supernatant was harvested followed by centrifugation 600 g for 5 min. IFN-7 level was determined by enzyme-linked immunoassay (ELISA) using Human IFN-7 ELISA MAX™ Deluxe Set (430104, BioLegend). Data was processed using Excel and plotted using GraphPad Prism 9.

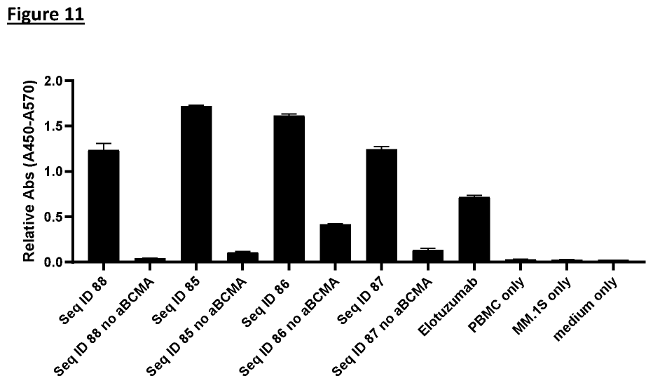

Anti-BCMA engager constructs containing a BCMA-binding polypeptide (herein termed 1-E6) genetically fused to a hCD16a binding arm composed of either monomeric A10 (Seq ID 88), homodimeric A10 (Seq ID 86), heterodimeric H09-A10 (Seq ID 85) or heterodimeric A11-A10 (Seq ID 87) affibodies were evaluated. Corresponding constructs harbouring a null non-BCMA-binding polypeptide with high sequence identity with the BCMA-binding polypeptide were also evaluated. The SLAMF7 monoclonal antibody Elotuzumab was used as a positive control and single cultures of PBMC and MM.1S served as negative controls. The dual engagers containing a BCMA-binding polypeptide all evoked an IFNγ response in cocultures of PBMC and MM.1S cells, which for all engagers was larger than the response of the positive control Elotuzumab (FIG. 11). Dual engager constructs devoid of BCMA binding showed no or limited IFNγ response in cocultures of PBMC and MM.1S cells. These experiments illustrate the BCMA dependent activation of PBMC against the BCMA positive MM.1S myeloma cell line induced by the anti-BCMA engagers described herein.

### Biological Example 7

### Assessment of Secondary Structure Contents and Thermal Denaturation Profiles of CD16a-Binding Polypeptides Using Circular Dichroism

In this Example, the three CD16a-binding polypeptides A10, H09 and A11 [SEQ ID 1, 74 and 75] were subcloned, expressed, purified as polypeptide-His₆ fusion proteins to assess the secondary structure contents and their thermal denaturation profiles by circular dichroism (CD) spectroscopy.

### Materials and Methods

### Cloning of the CD16a-Binding Polypeptides

The DNA fragments encoding the three CD16a-binding polypeptides [SEQ ID 1, 74 and 75] were amplified from their respective pAffi-1 library vectors with specific primers, designed to introduce overhangs complementary to ends of linearised expression vectors. In-Fusion HD Cloning Kit (Takara Bio, Gothenburg, Sweden) was used to clone monomeric polypeptide constructs with a C-terminal His₆ tag. The DNA constructs were sequence verified using Sanger sequencing (Eurofins Genomics).

### Expression and Purification

E. coli BL21 (DE3) cells were transformed with plasmids containing the DNA constructs and cultivated in 10 ml TSB+Y with 25 μg/ml Kan at 37° C. with shaking at 150 rpm for 16-18 h. The cultures were inoculated 1:100 in 200 ml TSB+Y with 25 g/ml Kan and grown at 37° C. with shaking at 150 rpm until OD₆₀₀=0.6-1. IPTG was added to final concentration 1 mM to induce protein expression. The cultivations were incubated at 25° C. with shaking at 150 rpm for 16-18 h and the cells were harvested by centrifugation. The cell pellets were resuspended in denaturing lysis buffer (7 M guanidinium chloride, 47 mM Na₂HPO₄, 2.65 mM NaH₂PO₄, 10 mM TRIS-HCl, 100 mM NaCl, pH 8). After incubation at 37° C. for 2 h with shaking at 150 rpm, the cells were centrifuged and the denatured protein from the supernatant of the cell lysates was added to tubes containing 3 ml HisPur Cobalt IMAC Resin (cat. no. 89966, Thermo Scientific). Supernatants were incubated with resin for 30 min at RT with eoe rotation. Contaminants were removed by washing three times with wash buffer (7 M guanidium chloride, 46.6 mM Na₂HPO₄, 3.4 mM NaH₂PO₄, 300 mM NaCl, 10 mM imidazole, pH 8) and CD16a binding polypeptide variants were subsequently eluted with elution buffer (6 M urea, 50 mM NaH₂PO₄, 100 mM NaCl, 30 mM glacial acetic acid, 70 mM sodium acetate, pH 5) by incubation at RT for 10 min with eoe rotation. Following IMAC purification, the protein buffer was exchanged to PBS using PD-10 desalting columns (cat. no. 17085101, Cytiva, Uppsala, Sweden). SDS-PAGE analysis was performed to confirm the purity and concentrations, which were measured by absorbance at 280 nm using extinction coefficients calculated from amino acid sequences, of the purified proteins (NuPAGE, 4 to 12%, Bis Tris, Invitrogen, Waltham, Massachusetts, USA).

### CD Analyses

A Chirascan CD Spectrometer (Applied Photophysics, Leatherhead, United Kingdom) was used to determine the secondary structure content of the three CD16a-binding polypeptides, by recording the CD spectra at a wavelength range of 195-260 nm at 20° C., as well as their thermal denaturation profiles, by recording the CD ellipticity at 221 nm during heating from 20 to 92° C.

### Results

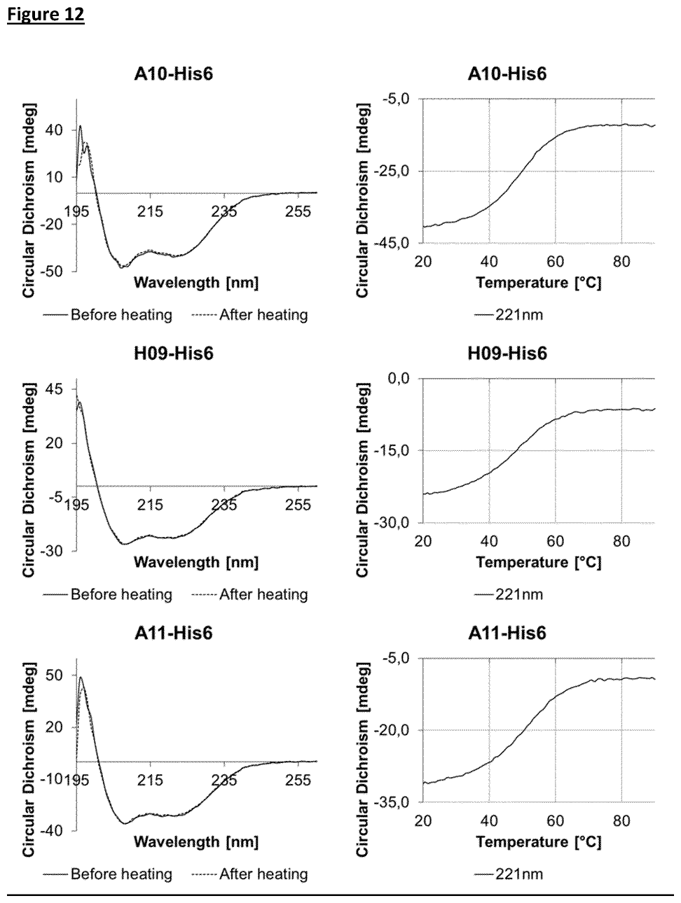

The CD spectra obtained showed that the analysed polypeptides all had a high α-helix secondary structure content at 20° C. Thermal denaturation profiles (FIG. 12) showed melting temperatures (Tm) of 49° C. for clone A10-His₆, 48° C. for clone H09-His₆, and 50° C. for clone A11-His₆, determined by variable temperature measurement. Further, the CD profiles of heated (92° C.) and subsequently cooled (20° C.) samples showed to overlap almost perfectly. Taken together, the results support that the three CD16a-binding polypeptides generated contain predominantly a-helical secondary structures and are capable of refolding following heat denaturation.

### Biological Example 8

### Alanine Scan of the CD16a-Binding Clone A10

In this Example, an alanine scan of the polypeptide variant A10 [SEQ ID 1] was performed to investigate the relative importance of the amino acid occupancies at the positions randomized during library construction for the binding interaction of A10 with CD16a.

### Materials and Methods

### Site-Directed Mutagenesis of the CD16a-Binding Clone A10.

The DNA fragment encoding the CD16a-binding polypeptide clone A10 [SEQ ID 1] was subjected to site directed mutagenesis by using the expression plasmid containing the A10-His₆ construct as the template in individual mutagenesis PCR reactions, where each of the original codons in the 14 variable positions were substituted to alanine codons using primers designed for each position. The resulting 14 alanine substitution variants were sequence verified using Sanger sequencing (Eurofins Genomics).

### Expression and Purification

E. coli BL21 (DE3) cells were transformed with plasmids encoding the 14 alanine variants, as well as the wild-type A10, each equipped with a C-terminal His₆ tag, and cultivated in 10 ml TSB+Y with 25 μg/ml Kan at 37° C. with shaking at 150 rpm for 16-18 h. The cultures were inoculated 1:100 in 200 ml TSB+Y with 25 μg/ml Kan and grown at 37° C. with shaking at 150 rpm until OD₆₀₀=0.6-1. IPTG was added to final concentration 1 mM to induce protein expression. The cultivations were incubated at 25° C. with shaking at 150 rpm for 16-18 h and the cells were harvested by centrifugation. The cell pellets were resuspended in binding/washing buffer (PBS with 15 mM imidazole, pH 7.4). After cell disruption by sonication, cell debris was removed by centrifugation and each supernatant of the cell lysates was added to tubes containing 3 ml HisPur Cobalt IMAC Resin (cat. no. 89966, Thermo Scientific). Supernatants were incubated with resin for 30 min at RT with eoe rotation. Contaminants were removed by washing three times with wash buffer and A10 variants were subsequently eluted with elution buffer (PBS with 300 mM imidazole, pH 7.4) by incubation at RT for 10 min with eoe rotation. Following IMAC purification, the polypeptides were buffer exchanged to PBS using PD-10 desalting columns (cat. no. 17085101, Cytiva, Uppsala, Sweden). SDS-PAGE analysis was performed to confirm the purity and concentrations, which were measured by absorbance at 280 nm using extinction coefficients calculated from amino acid sequences, of the purified proteins (NuPAGE, 4 to 12%, Bis Tris, Invitrogen, Waltham, Massachusetts, USA).

### Biosensor Analyses of the Alanine Variants

A Biacore T200 instrument (Cytiva) was used to analyse the interactions between CD16a and the His₆ tagged wild-type A10 polypeptide and the 14 alanine variants. The protein ligand hCD16a F158 (Cat. no. CDA-H5220, Acro Biosystems) was immobilised (1430 RU) on a Series S CM5 sensor chip (Cytiva) by amine coupling, using the manufacturer's instructions. One flow cell was activated and deactivated to be used as a reference cell. Analytes were diluted in the running buffer PBS-T before binding analysis was performed at 25° C. and a flow rate of 30 l/min. After each injection series the flow cells were regenerated by an injection of 10 mM HCl. The wildtype A10-His₆ construct and the 14 alanine variants were injected over the CD16a (F158) surface at a common concentration of 200 nM.

### Results:

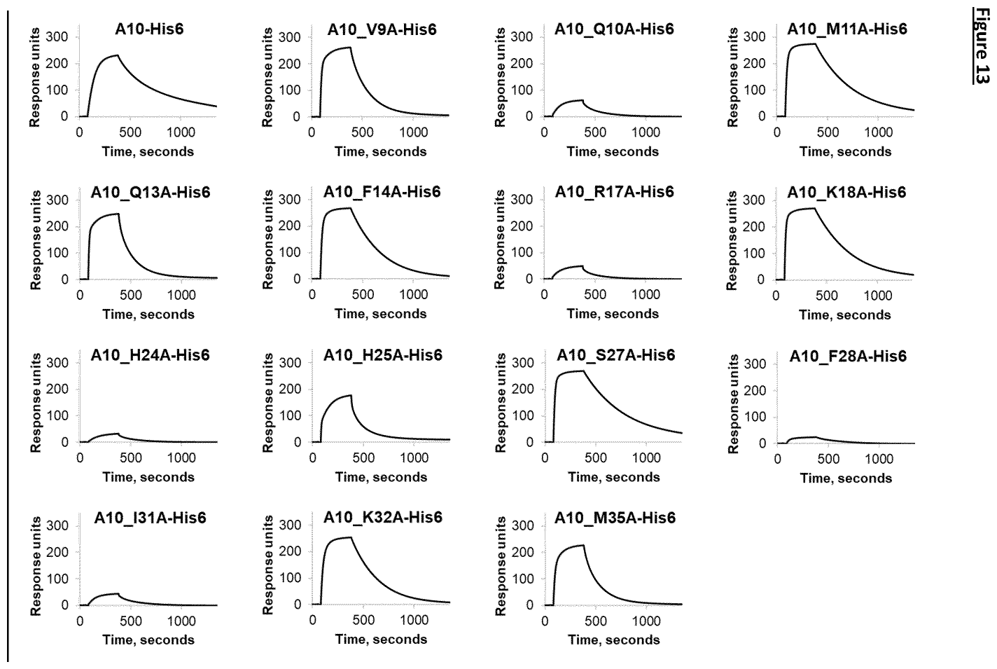

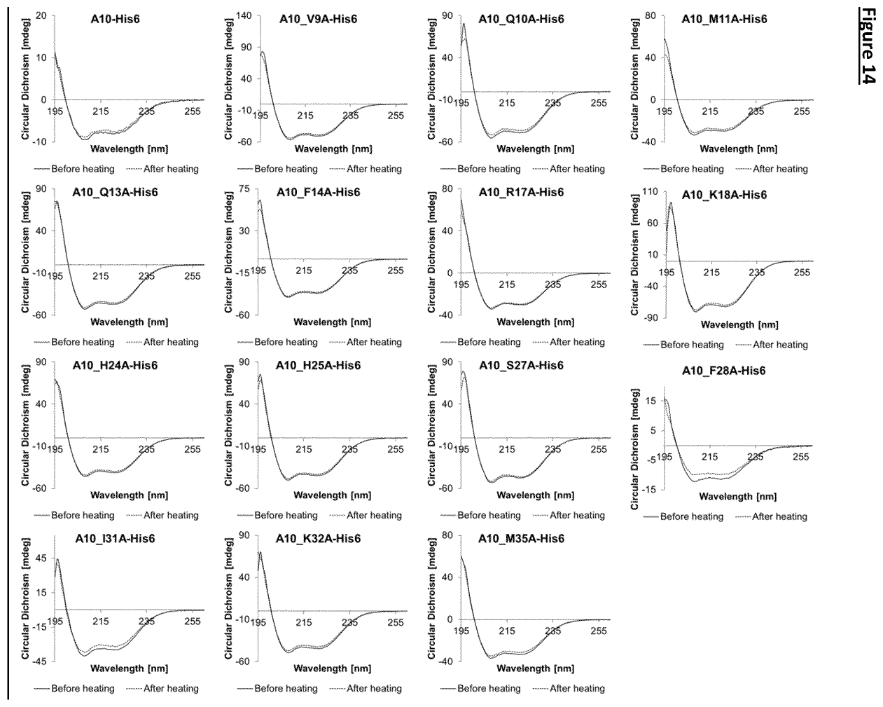

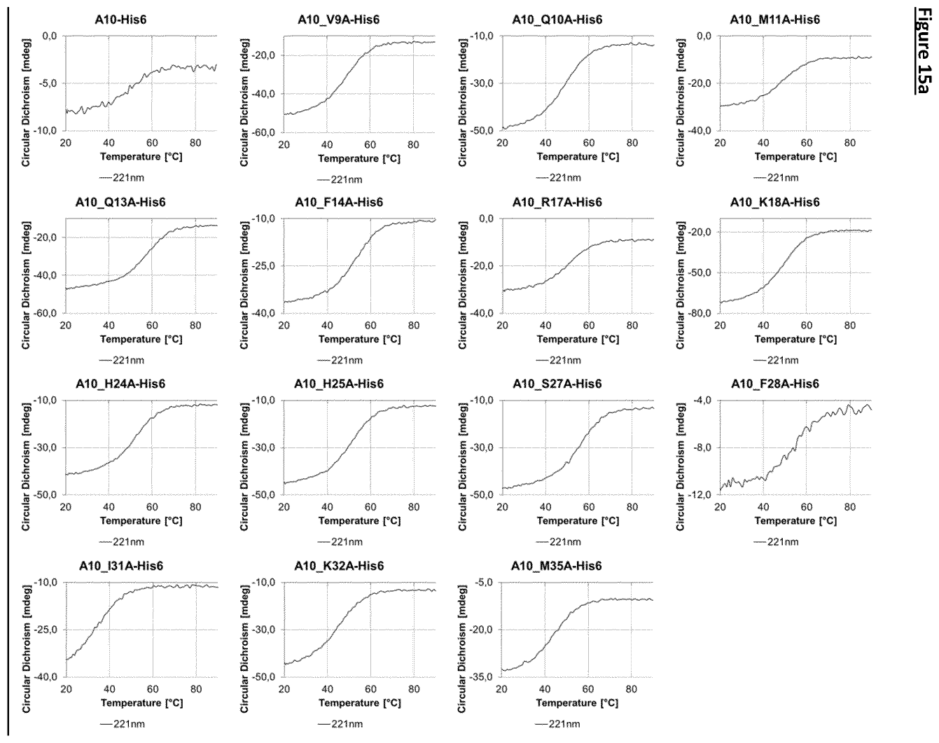

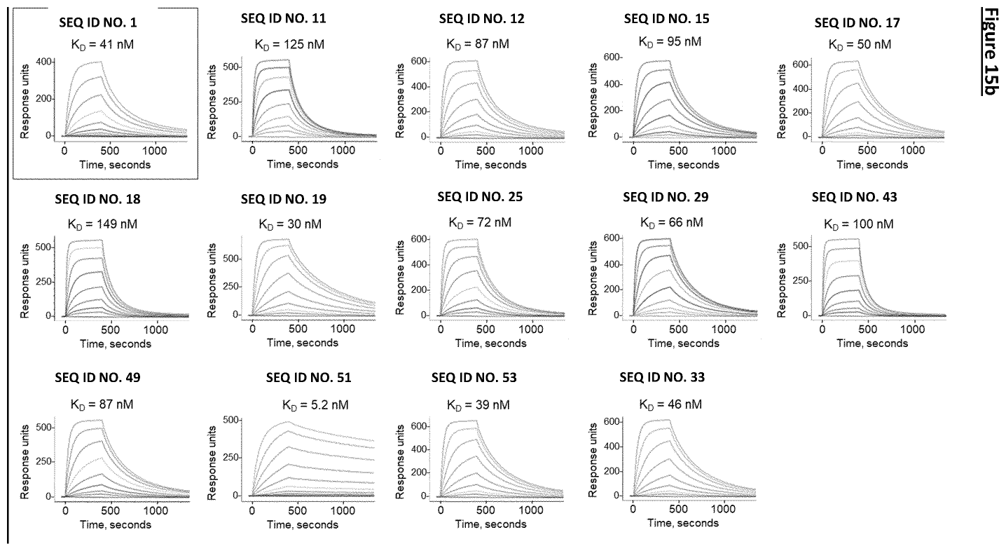

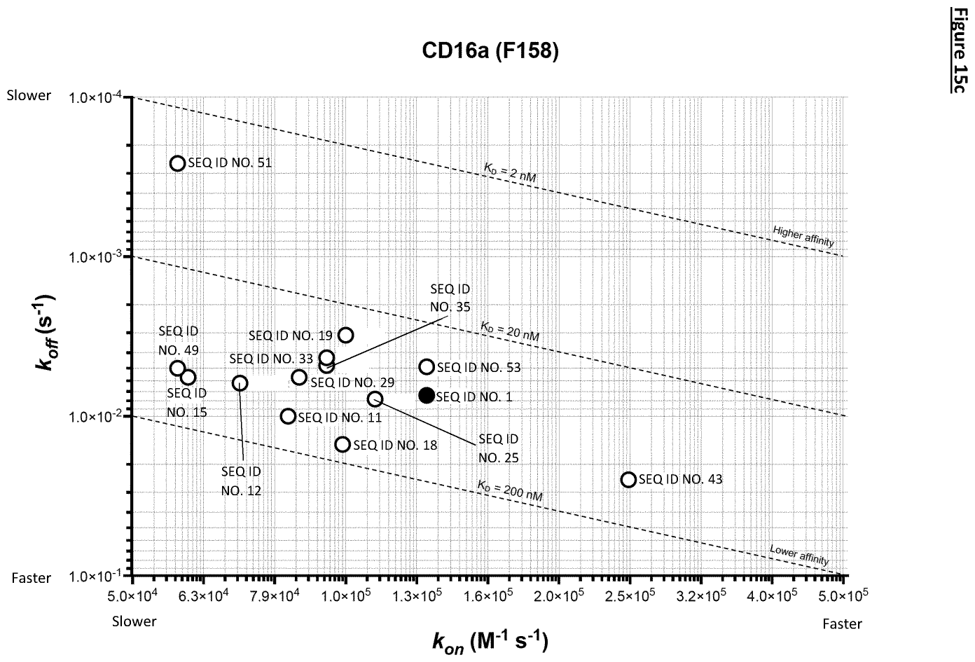

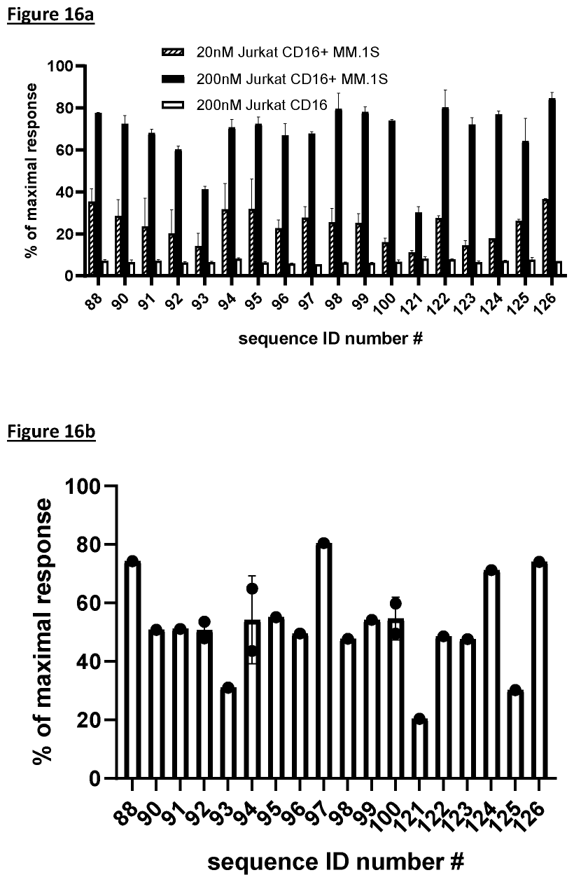

Separate injections of the 14 alanine-substituted variants (polypeptide-YY-His₆ format), at a common concentration of 200 nM), over a sensor chip surface containing CD16a (F158) protein resulted in a series of sensorgrams from which the importance of the original amino acid in the anti-CD16a A10 polypeptide for the binding to CD16a could be deduced. The sensorgrams are shown in FIG. 13. The substitution for alanine at positions 9, 11, 13, 14, 18, 27, 32 [SEQ IDs 2, 3, 4, 5, 6, 8 and 9] did not significantly affect the binding, as resulting sensorgrams resembled that obtained for the original A10 polypeptide with respect to response level and curve form. In contrast, alanine substitution at positions 10, 17, 24, 28 and 31 resulted in variants showing significantly lower binding responses. A few variants, H25A and M35A [SEQ IDs 7 and 10] were moderately affected by the alanine substitution. Analyses by circular dichroism spectrometry showed that all variants had profiles characteristic for proteins with a high content of a helices, with clear minima at 221 and 205 nm (see data in FIG. 14). Thermal melting experiments showed that all but one variant (131A) showed a retained thermal melting point of ca. 50° C. (see data in FIG. 15). Taken together, this indicated that the lower binding responses seen for some variants were not associated with a corresponding loss of overall structure, but rather that key residues for the interaction with CD16a had been addressed.

### Preparative Example 9

### Second-Generation Library Construction, Selection and Phage ELISA

In this Example, a second-generation library of the CD16a binding polypeptide A10 was constructed based on re-randomization of certain variable positions, and to various degrees. The library was used for the identification of second-generation variants of the CD16a binding A10 polypeptide but with a retained ability to bind CD16a with different strengths. The library was devoid of methionine. Individual and unique clones obtained after four phage display selection cycles and subsequent DNA sequencing were assayed for binding to CD16a in a monoclonal phage-ELISA.

### Materials and Methods

### Second-Generation Library of the CD16a Binding Polypeptide A10

An M13 phage display second-generation selection library was prepared, based on the A10 polypeptide. The phagemid vector used were pAffi-1 (Gronwall et al. (2007) J. Biotechnol. 128:162-183). The pAffi-1 phagemid, containing a lac promoter and an OmpA signal peptide, is designed for phage display of encoded polypeptide library members as in-frame fusions to an albumin binding domain (ABD), an amber stop codon and a truncated form (residues 249-406) of the M13 phage coat protein 3.

Two synthetic oligonucleotides based on the A10 sequence were used for gene assembly overlap extension PCR (OE-PCR) and subsequent amplification by PCR: A10-fwd of length 65 bp (5′-AC AAC AAA TTC AAC AAA GAA Z01 Z02 Z03 GCG Z01 Z01 GAG ATC Z04 Z01 CTG CCG AAC CTG AAC [SEQ ID 227]-‘3’), and A10-rev of 69 bp (5′-ACT CTG GCT CGG ATC ATC Z03 CAG Z05 Z01 Z06 GAA TGC Z07 Z01 CTG Z08 Z09 GTT CAG GTT CGG CAG [SEQ ID 228]-3′) encoding amino acid positions 3-23 and 19-41 (reverse complementary strand), respectively, corresponding to the Z domain numbering (Nilsson et al. (1987) Protein Eng. 1:107-113), in which 15 original A10 codons had been re-randomised based on the data obtained in an alanine scan experiment (Example 8), during the synthesis using mixtures of trinucleotide codons building blocks (amino acid codons for Met, Cys, Pro and Gly were excluded); Z01=10% Ala and 90% equally distributed between the remaining 15 allowed amino acids, Z02=10% Gln and 90% equally distributed between the remaining 15 allowed amino acids, Z03=15% Ile and 85% equally distributed between the remaining 15 allowed amino acids, Z04=90% Arg and 10% equally distributed between the remaining 15 allowed amino acids, Z05=80% Ser and 20% Lys, Z06=90% Ile and 10% equally distributed between the remaining 15 allowed amino acids, Z07=90% Phe and 10% equally distributed between the remaining 15 allowed amino acids, Z08=65% His and 35% equally distributed between the remaining 15 allowed amino acids, Z09=90% His and 10% equally distributed between the remaining 15 allowed amino acids). Following OE-PCR, the primers Forward:

[SEQ ID 223]
(5′-GATGAAGCCCTCGAGGTAGACAACAAATTCAACAAAGAA-3′)

and Reverse: (5′-TTAGCTTCTGCTAGCAAGTTAGCGCTTTGGCTTGGGTCATC-3′) [SEQ ID 224] were used for PCR amplification.

Approximately 5.5 g of XhoI and NheI double-cleaved and purified (Qiagen, Germany) PCR product of the assembled gene was ligated to 30 μg of XhoI and NheI double-cleaved and gel-purified pAffi-1 phagemid vector using T4 DNA ligase. The resulting ligation mixture was desalted using column-purification (Qiagen, Germany), divided into 22 portions and used to electroporate (0.1 cm BioRad cuvettes) 25 μl electrocompetent ER2738 E. coli cells (F′, glnV amber suppressor) (Lucigen, USA). 970 μl of Recovery medium (2% tryptone, 0.5% yeast extract, 10 mM NaCl, 2.5 mM KCl, 10 mM MgCl₂, 10 mM MgSO₄, and 20 mM glucose) was added to electroporated cells which were subsequently pooled (eleven electroporations per pool) and incubated at 37° C. for 1 h under shaking, after which pools of cells were titrated via spreading of dilution series on ampicillin plates and transferred to two 5 litre shake flasks, each containing 500 ml of Tryptic Soy Broth+Yeast extract medium (30 g/l tryptic soy broth, 5 g/l yeast extract; TSB+Y) supplemented with 1.5% (w/v) glucose and 100 μg/ml ampicillin. Overnight cultures of cells were pelleted by centrifugation and resuspended in 25 ml of cold 40% glycerol to a final volume of approximately 37 ml, followed by distribution into 20 tubes of approximately 1 ml cell-glycerol solution per tube and 5 tubes of approximately 3 ml cell-glycerol solution per tube. From the post-electroporation titrations and the OD₆₀₀ measurements after the overnight cultivation it was calculated that the library size (diversity) was ca. 5×10⁶ and that each 1 ml aliquot of cells contained a number of cells corresponding to ca. 14 000× the library size. A portion of the cells were used directly for phage stock preparation using M13KO7 helper phage while the remaining tubes with cells were stored at −80° C.

### Phage Stock Preparation.

For the production of an affibody-displaying phage stock, 1.14 ml of the library glycerol stock was distributed and inoculated into two baffled E-flasks, each containing 750 ml TSB+Y, 1% (w/v) glucose, 10 μg/ml Tet (tetracycline) and 100 μg/ml Carb (carbenicillin), and grown at 37° C. with shaking at 150 rpm until the cultures reached an OD₆₀₀=1.30 ml cells per culture flask were infected with a multiplicity of infection (MOI) of 5 of M13KO7 helper phage (New England Biolabs), gently swirled and incubated without shaking for 15 min at 37° C., followed by 70 rpm at 37° C. for 15 min. The cells of each culture were centrifuged and resuspended in two baffled E-flasks, containing 500 ml TSB+Y, 100 μg/ml Carb and 1 mM IPTG (Isopropyl β-D-1-thiogalactopyranoside). 25 μg/ml Kan (kanamycin) was added 2 h after inoculation. Cultures were incubated at 37° C. with shaking at 90 rpm for 16-18 h. The phage library stock was harvested by precipitation with 20% (w/v) PEG6000/2.5 M NaCl twice. The titer of the stock was measured by spot titration and polymerase chain reaction-screening was used to analyse the percentage of clones carrying a phagemid with a correctly sized gene insert.

Selections from Secondary Library of the CD16a Binding Clone A10.

Four cycles of panning were performed using biotinylated hCD16a (biotinylated human hCD16a (F158), Avitag, His tag, Acro Biosystems, cat. no. CDA-H82E8 corresponding to residues 17-208 of Uniprot entry P08637) using 100 nM in four tracks in cycle 1, 50 nM in three tracks in cycle 2 (two tracks from cycle 1 pooled together), 50 nM in five tracks in cycles 3 and 4 (two tracks from cycle 2 divided into two). Additionally in cycle 3, a molar excess of 25 μM of non-biotinylated target [CD16a F158 Y140H]-[Z963]-[AVI-tag]-[His6]: (GMRTEDLPKAVVFLEPQWYRVLEKDSVTLKCQGAYSPEDNSTQWFHNESLI SSQASSYFIDAATVDDSGEYRCQTNLSTLSDPVQLEVHIGWLLLQAPRWVFKE EDPIHLRCHSWKNTALHKVTYLQNGKGRKYFHHNSDFHIPKATLKDSGSYFC RGLFGSKNVSSETVNITITQGLAVSTISSFFPPGYQGGGSGVDNKFNKETQEAS WEIFTLPNLNGRQVAAFISSLLDDPSQSANLLAEAKKLNDAQAPKGGGSGGL NDIFEAQKIEWHEHHHHHH) [SEQ ID NO. 229] was used for competition. SA (streptavidin)-coated paramagnetic beads (Dynabeads M-280 Streptavidin, cat. no. 11205D, Invitrogen, Waltham, Massachusetts, USA) were washed twice with PBS (150 mM NaCl, 8 mM Na₂HPO₄, 2 mM NaH₂PO₄·H₂O, pH 7.4). To avoid enrichment of unspecific binders, all tubes used in the biopanning were pre-treated with 1% (w/v) bovine serum albumin (BSA) in PBS-T (PBS supplemented with 0.05% (v/v) Tween-20, pH 7.4). Furthermore, phage stock in PBS-T was pre-incubated for 30 min at room temperature (RT) under constant end-over-end (eoe) rotation with 0.1% (w/v) BSA and beads, to remove phages carrying binders against SA. Amount of phage stock used was 10¹⁰ colony forming units (cfu) in cycle 1, 10¹¹ cfu in cycles 2 and 3, and 10¹¹⁻¹² cfu in cycle 4, depending on the track.

For the tracks with solid-phase selection, biotinylated target protein was immobilised on 1 mg beads in cycles 1 and 4 or 0.5 mg in cycles 2 and 3, for 1 h at RT and end-over-end (eoe). Target-containing beads were incubated with 1% (w/v) BSA in PBS-T for 30 min at RT and eoe and then washed with PBS-T, before the addition of pre-incubated phage stock and performing the selection for 3 h in cycles 1 and 4, 2 h in cycle 2 and 30 min in cycle 3, at RT and eoe. For the tracks with liquid-phase selection, pre-incubated phage stock was added to biotinylated target protein to perform the selection in solution after which phage antigen complexes were captured by incubation with SA-beads, for 30 min at RT and eoe. Cycle 3 included competition with non-biotinylated target (off-rate selection), which was added after the aforementioned incubation and incubated for an additional 1 h or ON (18 h). SA-bead captured phage antigen complexes were washed with PBS-T eoe at RT for a total of 5 min (cycles 1 and 4), 10 min or 30 min (cycle 2) or 20 min (cycle 3). The final wash volumes were transferred to new 1% (w/v) BSA pre-treated tubes to remove sticky binders attached to the tube walls. Antigen-binding phages were eluted either by incubation with 0.5 M acetic acid, pH 2.8 for 15 min eoe at RT, followed by transfer of the eluate to new pre-treated tubes and neutralization with equal volume 1 M Tris-HCl, pH 8, or by incubating with 0.25 mg/ml trypsin (Gibco Life Technologies) in TBS-T (TRIS buffered saline, 0.1% (v/v) Tween-20) supplemented with 1 mM CaCl₂) for 30 min eoe at RT before transferring the eluate to new pre-treated tubes.

Following cycle 1, new phage stocks for selection round 2 were generated by growing E. coli XL-1 Blue cells (Agilent) in TSB+Y with 10 μg/ml Tet at 37° C. with shaking at 150 rpm until OD₆₀₀=0.5-0.8. 50 ml bacteria were infected with the total phage eluate volume, gently swirled, incubated without shaking for 25 min at 37° C., followed by shaking at 70 rpm at 37° C. for 15 min. The cultures were then centrifuged and resuspended in TSB+Y, before plating on blood agar plates (40 g/l blood agar) with 100 μg/ml Carb and 1% (w/v) glucose, and incubation for 16-18 h at 37° C. Bacterial colonies were collected to TSB+Y by scraping and inoculated to 200 ml TSB+Y with 100 μg/ml Carb and grown at 37° C. with shaking at 150 rpm until OD₆₀₀=0.5. 30 ml were superinfected with M13KO7 helper phage (MOI 5), gently swirled, incubated without shaking for 25 min at 37° C., followed by shaking at 70 rpm at 37° C. for 15 min. The cultures were centrifuged and resuspended in 150 ml TSB+Y with 100 μg/ml Carb and 1 mM IPTG. 25 μg/ml Kan was added 2 h after inoculation. Cultures were incubated at 37° C. with shaking at 150 rpm for 16-18 h. The phage stocks were harvested by precipitation with 20% (w/v) PEG6000/2.5 M NaCl twice. The titre of the stock was measured by spot titration and polymerase chain reaction-screening was used to analyse the percentage of phage particles carrying phagemids with the affibody insert.

For the following cycles 2-3, new phage stocks were generated by growing E. coli XL-1 Blue cells in TSB+Y with 10 μg/ml Tet and 1% (w/v) D-glucose at 37° C. with shaking at 150 rpm until OD₆₀₀=0.5-0.8. 30 ml grown E. coli XL-1 Blue bacteria were infected with half the phage eluate volume and incubated at 37° C. with shaking at 150 rpm for 30 min. An equal volume of TSB+Y with 10 μg/ml Tet and 100 μg/ml Carb was added and the bacteria were then phenotyped for infection by incubating at 37° C. for 1.5 h with shaking at 150 rpm, followed by superinfection with M13KO7 helper phage (MOI 76-382) and additional incubation at 37° C. for 1.5 h with shaking at 150 rpm. The cultures were centrifuged and resuspended in 100 ml TSB+Y with 100 μg/ml Carb, 25 μg/ml Kan and 0.5 mM IPTG. The cultures were incubated at 30° C. with shaking at 150 rpm for 16-18 h. The phage stocks were harvested by precipitation with 20% (w/v) PEG6000/2.5 M NaCl twice. The titre of the stocks were measured by spot titration and polymerase chain reaction-screening was used to analyse the percentage of phage particles carrying phagemids with the affibody insert.

DNA sequencing. Following phage display selection, 25 and 129 individually grown colonies after infecting E. coli XL-1 Blue with phage eluate after cycle 3 and cycle 4, respectively, were sent for DNA sequencing by Sanger sequencing (PlateSeq, Eurofins Genomics, Ebersberg, Germany). 67 unique and new sequences were identified.

Monoclonal phage-ELISA screening. Monoclonal phage supernatant preparation and monoclonal phage-ELISA screening was performed as described in Example 1, using the 67 unique and new sequences identified by Sanger sequencing. In this example, a MaxiSorp ELISA plate (Clear Flat-Bottom Immuno Nonsterile 384-Well Plates, cat. no. 464718, Thermo Fisher Scientific) was coated at 4° C. with slow shaking for 16-18 h with the following antigens: 30 μl 4 μg/ml biotinylated hCD16a, 20 μg/ml human serum albumin (HSA; Sigma product no. SRP6182) (for assessment of proper display of the expression cassette containing a tripartite fusion protein including an affibody, an affibody albumin binding domain and the truncated protein 3), 10 μg/ml SA or 10 g/ml BSA in 100 mM sodium carbonate buffer pH 9.6 per well (¼ of the wells with each coated protein). 53 binding candidates were identified as ELISA-positive.

### Results

Positions in the A10 peptide identified as important for CD16a binding (Q10, R17, H24, F28 and I31) were randomized conservatively. For these positions, trinucleotide codon mixes used for synthesis of the mutagenic oligonucleotides were biased to provide a 90% likelihood for re-insertion of the amino acid found in the A10 peptide, leaving a 10% likelihood for insertion of a different amino acid (any of the 20 natural amino acids, minus the A10 amino acid or methionine, cysteine, proline or glycine). For six positions (9, 13, 14, 18, 27 and 32), at which the original amino acid in the A10 peptide was not found to be critical for CD16a binding, trinucleotide codon mixes used for synthesis of the mutagenic oligonucleotides were biased to only provide a 10% likelihood for re-insertion of the amino acid found in the A10 peptide, leaving a 90% likelihood for insertion of a different amino acid (any of the 20 natural amino acids, minus the A10 amino acid or methionine, cysteine, proline or glycine). For position 25, occupied by a histidine in the A10 peptide, the trinucleotide codon mix used for synthesis of the mutagenic oligonucleotides was biased to provide a 65% likelihood for re-insertion of histidine, leaving a 35% likelihood for insertion of a different amino acid (any of the 20 natural amino acids, minus histidine, methionine, cysteine, proline or glycine). For position 33, occupied by a serine in the A10 peptide, the trinucleotide codon mix used for synthesis of the mutagenic oligonucleotides was biased to provide an 80% likelihood for re-insertion of serine, leaving a 20% likelihood for insertion of lysine. For positions 11 and 35, both occupied by oxidation-prone methionines in the A10 peptide, the trinucleotide codon mix used for synthesis of the mutagenic oligonucleotides was biased to provide a 15% likelihood for insertion of isoleucine, and an 85% likelihood for insertion of a different amino acid (any of the 20 natural amino acids, minus methionine, cysteine, proline or glycine).

Selection for CD16a binding polypeptides from this library would thus have the potential to yield novel A10-related, CD16a binding polypeptides (denoted A10*) of different affinities. It also has the possibility to yield methionine-free CD16a binding polypeptides.

Selections to CD16a (F158) target protein were performed by phage display as described above (Material and methods). Analysis of the selection outputs from the different selection tracks by monoclonal phage-ELISA and DNA sequencing resulted in the identification of 53 unique and ELISA-positive clones. Their sequences are shown in the Table in FIG. 23 as SEQ IDs 11-63. Three further clones of interest (SEQ IDs 64-66) were also identified.

### Biological Example 10A

### Expression, Protein Purification and SPR Analysis of CD16a Binding Polypeptides Identified in Example 9

### Material and Methods

Subcloning of hBCMA×hCD16a-Binding Polypeptide Dual Engager Constructs.

DNA fragments encoding the 53 CD16a-binding polypeptides [SEQ IDs 11-63] identified in Example 9 (A10*) and the hBCMA binding polypeptide referred to herein as 1-E6 were amplified using their respective library pAffi-1 phagemid vectors as templates, using primers introducing overhangs complementary to ends of linearised expression vectors and flexible (GGGGS)₃ encoding linkers between each polypeptide unit. In-Fusion HD Cloning Kit (Takara Bio, Gothenburg, Sweden) was used to assemble the gene fragments into 53 different BCMA×hCD16a dual engager constructs with a C-terminal His6 tag as anti-BCMA-A10*-His₆, where A10*corresponds to the CD16a binding polypeptides identified in Example 9 [SEQ ID 11-63]. The DNA constructs were sequence verified using Sanger DNA sequencing (Eurofins Genomics).

Expression and Protein Purification of hBCMA×hCD16a-Binding Polypeptide Dual Engager Constructs.

E. coli BL21(DE3) cells were transformed with plasmids containing the 53 DNA constructs and cultivated in 500 μl TSB+Y with 25 μg/ml Kan at 37° C. with shaking at 150 rpm for 16-18 h. Cells were also transformed with a DNA construct encoding the BCMA×hCD16a dual engager construct anti-BCMA-A10-His₆ described above in Example 5 [SEQ ID 88]. The cultures were inoculated 1:100 in 2.5 ml TSB+Y with g/ml Kan and grown at 37° C. with shaking at 150 rpm until OD600=0.6-1. IPTG was added to final concentration 1 mM to induce protein expression. The cultivations were incubated at 25° C. with shaking at 150 rpm for 16-18 h and the cells were harvested by centrifugation. The cell pellets were resuspended in denaturing lysis buffer (7 M guanidinium chloride, 47 mM Na₂HPO₄, 2.65 mM NaH₂PO₄, 10 mM TRIS-HCl, 100 mM NaCl, pH 8). After incubation at 37° C. for 2 h with shaking at 150 rpm, the cells were centrifuged and the denatured protein from the supernatant of the cell lysates, supplemented with approximately 15 mM imidazole, was added to tubes containing HisPur Cobalt IMAC Resin (cat. no. 89966, Thermo Scientific). Supernatant was incubated with resin for 30 min at RT with end-over-end rotation. Contaminants were removed by washing three times with wash buffer (PBS with 15 mM imidazole, pH 7.4) and BCMA×hCD16a dual engagers were subsequently eluted with elution buffer (PBS with 400 mM imidazole, pH 7.64). Concentrations were measured by absorbance at 280 nm using extinction coefficients calculated from amino acid sequences. SDS-PAGE analysis was performed for a subset of the purified proteins to confirm the purity and concentrations (Mini-PROTEAN TGX, Tris/Glycine/SDS, Bio-Rad).

Evaluation of CD16a Binding of hBCMA×hCD16a-Binding Polypeptide Dual Engager Constructs.

A Biacore 8K instrument (Cytiva) was used to analyse interactions of hCD16a binding variants with human hCD16a in real-time. The protein ligands hCD16a F158 (Cat. no. CDA-H5220, Acro Biosystems) and hCD16a V158 (Cat. no. CD8-H52H4, Acro Biosystems) were diluted in 10 mM NaOAc, pH 4.5 and immobilised on separate sensor chip surfaces of a Series S CM5 sensor chip (Cytiva). The sensor chip consists of eight flow channels (two used here), each made up of two flow cells arranged in series. For each protein ligand and corresponding flow channel, the first flow cell was activated and deactivated to be used as a reference surface and the second flow cell was used to immobilize the protein ligands to the carboxymethylated dextran surface by amine coupling, using the manufacturer's instructions. The BCMA×hCD16a dual engager His₆ fusion proteins were diluted in the running buffer PBS-T.

### Results

The genes encoding 53 of the polypeptides identified in Example 9 were re-cloned for soluble expression as 1-E6-(GGGSG)₃-A10*-YY-His₆ fusion proteins. The polypeptide herein named 1-E6 is an hBCMA binding polypeptide with the sequence VDNKFNKENQFADEEIAALPNLNFYQKWAFIRKLMDDPSQSANLLAEAKKL NDAQAPK (SEQ ID 226). A10* is used here to denote individual CD16a binding polypeptides identified in Example 9. The A10* sequences are the ones shown for SEQ IDs 11 to 63 in the table in FIG. 23.

Expression was performed at a cultivation scale of 2.5 ml and polypeptide purification was performed by IMAC. A Biacore 8K instrument was used to investigate the binding of the different 1-E6-(GGGSG)₃-A10*—YY-His₆ polypeptides. In the analysis, the polypeptide with SEQ ID 1 was included, also produced as a fusion to the anti-BCMA binding polypeptide 1-E6. IMAC-purified fusion polypeptides were diluted to approximately 500 nM in PBS-T buffer and injected in parallel over sensor chip surfaces containing immobilized CD16a F158 or CD16a V158 fusion protein. The resulting sensorgrams demonstrated binding of all 53 injected analytes to the CD16a allotypes V158 and F158 and that the binding profiles to both allotypes were very similar for each analyte, as also observed for the polypeptide with SEQ ID 1. The measured binding affinities (K_{D}) for each of the polypeptides with CD16a binding sequence of SEQ ID 11-63 are presented in the table in FIG. 23. It is seen that the binding to CD16a F158 or CD16a V158 fusion protein is similar in each case. Binding to CD16b (NA1 and NA2 forms) was also measured.

### Evaluation of Further Variants of A10 [SEQ ID 64-67]

Constructs of Example 9 [SEQ ID 64-66] alongside a further construct of interest with SEQ ID 67 were synthesised and cloned into the NdeI XhoI sites of pET29 for expression as 1-E6-(GGGSG)₃-A10*—YY-His6 fusion proteins. Expression, isolation and characterisation can be found in example 11 and generated constructs SEQ ID 98, 93, 97 and 100, respectively. Binding to CD16a F158 was studied by SPR with the receptor as ligand on a CM-5 Biacore chip (4000 Ru). Engager constructs were diluted to 55 nM in HBS-EP buffer (Cytiva) for binding analysis. The resulting sensorgrams demonstrated binding of all 4 injected analytes.

### Biological Example 10B

### Kinetic Determination of a Subset of 13 Variants Identified in Example 9

### Material and Methods

Determination of Binding Kinetics for a Subset of hBCMA×hCD16a-Binding Polypeptide Dual Engager Constructs.

The binding kinetics of a subset of 13 variants of the hBCMA×hCD16a dual engager His₆ fusion protein variants to hCD16a, using the constructs produced and the same experimental set-up with the Biacore 8K instrument (Cytiva) as described in Example 10A. The subset of 13 variants in the form of anti-BCMA-A10*-His₆ fusion proteins chosen were the ones containing an A10* polypeptide corresponding to SEQ IDs 11, 12, 15, 17, 18, 19, 25, 29, 43, 49, 51, 53, 33. For this subset of 13 variants, as well as for the fusion protein containing the polypeptide with SEQ ID 1, CD16a F158 and V158 binding analyses were performed by injections over hCD16a surfaces using serial dilutions which varied in range for each of the fusion proteins. The total range covered was 0.3 nM-1.4 M. The values for the association rate constants (kₐ), the dissociation rate constants (k_{d}) and dissociation equilibrium constants (K_{D}) were calculated using a 1:1 Langmuir binding model in BIAevaluation software (Cytiva).

### Results

For the subset of 13 variants as well as the fusion protein containing the polypeptide with SEQ ID 1, CD16a F158 and V158 binding analyses were performed using serial dilutions, allowing for a determination of association rate (on-rate) (M⁻¹ s⁻¹) and dissociation rate (off-rate) (s⁻¹) kinetic constants, as well as overall dissociation equilibrium constants, i.e. K_{D} values (M). The results of binding with CD16a F158 are shown in FIG. 15b. Of particular note is the polypeptide denoted [SEQ ID 51] which was determined to have a particularly high affinity (K_{D}).

A plotting of the respective on-rate (M⁻¹ s⁻¹) and off-rate (s⁻¹) kinetic constants (FIG. 15c, and also shown in Table X) showed that the 13 analyzed variants displayed a large distribution in their association rate and dissociation rate constants. Interestingly, some variants with similar overall affinities (K_{D}) (e.g. SEQ ID 43 and SEQ ID 15) were found to have both different on-rate and off-rate kinetics, thus representing alternative binding properties resulting in a similar overall affinity. The variant with SEQ ID 51 showed the highest affinity among the 13 analyzed variants, and it was seen to have relatively slow on-rate kinetics but also markedly slow off-rate kinetics, resulting in an overall high affinity.

| | Clone name | | |
|---|---|---|---|
| SEQ ID | kₐ (M⁻¹ s⁻¹) | k_{d} (s⁻¹) | K_{D} (nM) |
| 1 | 1.3E+05 | 5.3E−03 | 41 |
| 11 | 8.3E+04 | 1.0E−02 | 125 |
| 12 | 7.1E+04 | 6.2E−03 | 87 |
| 15 | 6.0E+04 | 5.7E−03 | 95 |
| 17 | 9.4E+04 | 4.8E−03 | 50 |
| 18 | 9.9E+04 | 1.5E−02 | 149 |
| 19 | 1.0E+05 | 3.1E−03 | 30 |
| 25 | 1.1E+05 | 7.8E−03 | 72 |
| 29 | 8.6E+04 | 5.7E−03 | 66 |
| 43 | 2.5E+05 | 2.5E−02 | 100 |
| 49 | 5.8E+04 | 5.0E−03 | 87 |
| 51 | 6.6E+04 | 3.5E−04 | 5.2 |
| 53 | 1.3E+05 | 4.9E−03 | 39 |
| 33 | 9.4E+04 | 4.3E−03 | 46 |

### Biological Example 11

CD16a Activation and NK Cell Mediated Cell Killing by hBCMA×hCD16a Dual Engager with hCD16a Binding Polypeptide Identified as Described in Example 9 Above

hBCMA×hCD16a dual engagers comprising hCD16a binding polypeptides identified as described in Example 9 above were expressed as soluble gene products in E. coli (DE3) and thereafter characterized for their CD16a activation properties.

The cDNA coding for each hBCMA×hCD16a dual engager harbouring a stop codon was synthesized and ligated into the Ndel Xhol restriction sites of the pET29 vector.

E. coli BL21(DE3) was transformed with vector under Kanamycin selection and constructs were expressed by induction of IPTG at an OD of 0.6 and harvested 16 h later. Soluble cytosolic product was harvested and resuspended in 1×PBS supplemented by 30 mM imidazole. Purification was achieved by IMAC using 1×PBS supplemented by 500 mM imidazole as elution buffer. Further purification was achieved by RP-HPLC and identity/purity was confirmed by SDS-PAGE and analysis by LC-MS/MS.

### CD16 Activation Jurkat-Lucia™ Luciferase Reporter Assay

The propensity of the hBCMA×hCD16a dual engagers to stimulate CD16 activation was assessed in a Lucia Luciferase reporter assay and compared to the antibody dependent cellular cytotoxicity (ADCC) potency of Elotuzumab (clinical grade a-SLAMF7 monoclonal antibody) through evaluation of CD16a activation. Jurkat-Lucia™ NFAT-CD16 cells (InvivoGen) were seeded with MM.1S cells at an effector to target (E:T) ratio of 2:1 in a 96-well flat-bottom plate. 3×10⁵ cells in 200 μl per well were treated with 20 or 200 nM of the reagent of interest for 24 h. The supernatant was harvested, and Lucia luciferase activity, which reflects the induced ADCC response, was assessed using QUANTI Luc™ Gold (InvivoGen). Responses were normalized to the maximal response of Elotuzumab.

### Calcein-Release-Based In Vitro Cytotoxicity Assay

NK cell cytotoxic activity was assessed in calcein-release-based cytotoxicity assay previously described by (Gauthier et al, 2023) PMID 36635380. In brief, MM1.S target cells were pre-labelled with Calcein-AM (Biolegend) and co-cultured with NK cells at a 1:1 ratio in 96-well v-bottom plate. Engagers were added last to the co-culture, after effector and target cells. After 4 h of co-culture, supernatant was collected and transferred to black flat-bottom 96-well plate. Fluorescent signal of calcein-released by dead MM1.S cells was quantified in SpectraMax i3x (Molecular Devices). Percent specific lysis is calculated in Microsoft Excel (Microsoft) using target cells alone for spontaneous release and target cells with detergent for maximum release. NK cell cytotoxic activity in presence of engagers is compared to antibody dependent cellular cytotoxicity activity of Belantamab biosimilar (purchased from IchorBio).

### Results

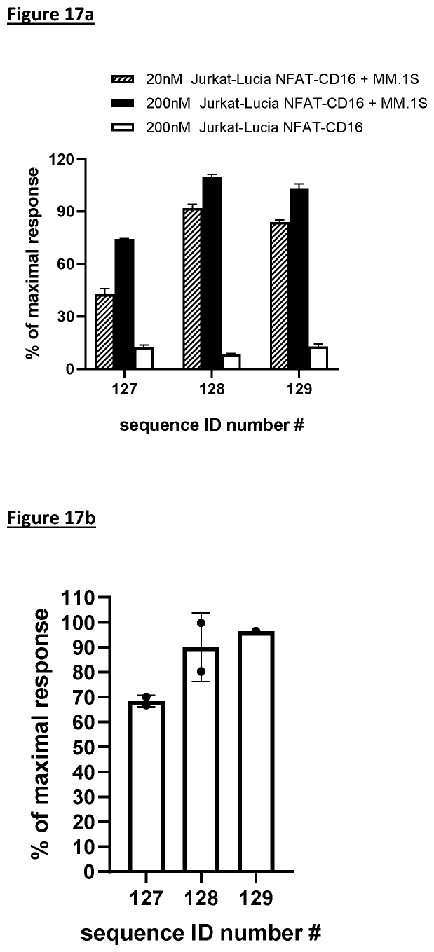

The results are shown in FIG. 16. It is seen that the hBCMA×hCDl16a dual engagers (SEQ IDs 88-100, 121-126) demonstrated CD16 activation in the Lucia Luciferase reporter assay in the presence ofBCMA+MM.1A cells, whereas no detectable activation was seen in the absence of target cells (FIG. 16a). Moreover, the engagers induced NIK cell mediated killing of MM.1S cells (FIG. 16b). Thus, a firm and target specific activation leading to NIK mediated cell killing was seen for all engagers investigated.

| | Descriptive ID | |
|---|---|---|
| SEQ | (with CD16a | |
| ID | binder SEQ ID) | Sequence |
| 88 | H_aBCMA-1- | VDNKFNKENQFADEEIAALPNLNFYQK |
| | E6_15 aa_aCD16- | WAFIRKLMDDPSQSANLLAEAKKLNDA |
| | A10_His6c_OH | QAPKGGGSGGGGSGGGGSGVDNKFNK |
| | (SEQ ID 1) | EVQMAQFEIRKLPNLNHHQSFAFIKSLM |
| | | DDPSQSANLLAEAKKLNDAQAPKYYH |
| | | HHHHHH |
| 90 | H_aBCMA-1- | VDNKFNKENQFADEEIAALPNLNFYQK |
| | E6_15 aa_aCD16- | WAFIRKLMDDPSQSANLLAEAKKLNDA |
| | A10(2a- | QAPKGGGSGGGGSGGGGSGVDNKFNK |
| | F09)_His6c_OH | EVQYAQREIRNLPNLNHHQAFAFIKSLI |
| | (SEQ ID 21) | DDPSQSANLLAEAKKLNDAQAPKYYH |
| | | HHHHH |
| 91 | H_aBCMA-1- | VDNKFNKENQFADEEIAALPNLNFYQK |
| | E6_15 aa_aCD16- | WAFIRKLMDDPSQSANLLAEAKKLNDA |
| | A10(2a- | QAPKGGGSGGGGSGGGGSGVDNKFNK |
| | H10)_His6c_OH | ELQSAQKEIRALPNLNHHQRFAFINSLL |
| | (SEQ ID 25) | DDPSQSANLLAEAKKLNDAQAPKYYH |
| | | HHHHH |
| 92 | H_aBCMA-1- | VDNKFNKENQFADEEIAALPNLNFYQK |
| | E6_15 aa_aCD16- | WAFIRKLMDDPSQSANLLAEAKKLNDA |
| | A10(2b- | QAPKGGGSGGGGSGGGGSGVDNKFNK |
| | G11)_His6c_OH | EVQIAQTEIRSLPNLNHHQVFAFIQSLID |
| | (SEQ ID 38) | DPSQSANLLAEAKKLNDAQAPKYYHH |
| | | HHHH |
| 93 | H_aBCMA-1- | VDNKFNKENQFADEEIAALPNLNFYQK |
| | E6_15 aa_aCD16- | WAFIRKLMDDPSQSANLLAEAKKLNDA |
| | A10(34a-C3- | QAPKGGGSGGGGSGGGGSGVDNKFNK |
| | 1)_His6c_OH | ELQSAQTEIQHLPNLNHAQQFAFILSLID |
| | (SEQ ID 65) | DPSQSANLLAEAKKLNDAQAPKYYHH |
| | | HHHH |
| 94 | H_aBCMA-1- | VDNKFNKENQFADEEIAALPNLNFYQK |
| | E6_15 aa_aCD16- | WAFIRKLMDDPSQSANLLAEAKKLNDA |
| | A10(2a- | QAPKGGGSGGGGSGGGGSGVDNKFNK |
| | E06)_His6c_OH | ELQYAQKEIRRLPNLNHHQVFAFINKLI |
| | (SEQ ID 17) | DDPSQSANLLAEAKKLNDAQAPKYYH |
| | | HHHHH |
| 95 | H_aBCMA-1- | VDNKFNKENQFADEEIAALPNLNFYQK |
| | E6_15 aa_aCD16- | WAFIRKLMDDPSQSANLLAEAKKLNDA |
| | A10(2a- | QAPKGGGSGGGGSGGGGSGVDNKFNK |
| | H04)_His6c_OH | EVQWAQKEIRSLPNLNHHQVFAFIRSLL |
| | (SEQ ID 16) | DDPSQSANLLAEAKKLNDAQAPKYYH |
| | | HHHHH |
| 96 | H_aBCMA-1- | VDNKFNKENQFADEEIAALPNLNFYQK |
| | E6_15 aa_aCD16- | WAFIRKLMDDPSQSANLLAEAKKLNDA |
| | A10(2a- | QAPKGGGSGGGGSGGGGSGVDNKFNK |
| | A06)_His6c_OH | ELQSAQWEIRALTNLNHHQKFAFIESLR |
| | (SEQ ID 11) | DDPSQSANLLAEAKKLNDAQAPKYYH |
| | | HHHHH |
| 97 | H_aBCMA-1- | VDNKFNKENQFADEEIAALPNLNFYQK |
| | E6_15 aa_aCD16- | WAFIRKLMDDPSQSANLLAEAKKLNDA |
| | A10(34b-C3- | QAPKGGGSGGGGSGGGGSGVDNKFNK |
| | 3)_His6c_OH | ELQSAQREIRILPNLNHHQRFAFINSLLD |
| | (SEQ ID 66) | DPSQSANLLAEAKKLNDAQAPKYYHH |
| | | HHHH |
| 98 | H_aBCMA-1- | VDNKFNKENQFADEEIAALPNLNFYQK |
| | E6_15 aa_aCD16- | WAFIRKLMDDPSQSANLLAEAKKLNDA |
| | A10(2b-C3- | QAPKGGGSGGGGSGGGGSGVDNKFNK |
| | 2)_His6c_OH | ETQQAQREIRKLPNLNHHQQFAFIKSLL |
| | (SEQ ID 64) | DDPSQSANLLAEAKKLNDAQAPKYYH |
| | | HHHHH |
| 99 | H_aBCMA-1- | VDNKFNKENQFADEEIAALPNLNFYQK |
| | E6_15 aa_aCD16- | WAFIRKLMDDPSQSANLLAEAKKLNDA |
| | A10(34b-C3- | QAPKGGGSGGGGSGGGGSGVDNKFNK |
| | 2)_His6c_OH | EIQHAQWEIRHLPNLNHHQQFAFIKSLI |
| | (SEQ ID 12) | DDPSQSANLLAEAKKLNDAQAPKYYH |
| | | HHHHH |
| 100 | H_aBCMA-1- | VDNKFNKENQFADEEIAALPNLNFYQK |
| | E6_15 aa_aCD16- | WAFIRKLMDDPSQSANLLAEAKKLNDA |
| | A10(2a-C3-5- | QAPKGGGSGGGGSGGGGSGVDNKFNK |
| | T20P)_His6c_OH | ELQSAQWEIRALPNLNHHQKFAFIESLR |
| | (SEQ ID 67) | DDPSQSANLLAEAKKLNDAQAPKYYH |
| | | HHHHH |
| 121 | H_aBCMA-1- | VDNKFNKENQFADEEIAALPNLNFYQK |
| | E6_15 aa_aCD16- | WAFIRKLMDDPSQSANLLAEAKKLNDA |
| | A10(34a- | QAPKGGGSGGGGSGGGGSGVDNKFNK |
| | A11)_His6c_OH | EQQIAQYEIRKLPNLNHHQTFAFIKSLLD |
| | (SEQ ID 51) | DPSQSANLLAEAKKLNDAQAPKYYHH |
| | | HHHH |
| 122 | H_aBCMA-1- | VDNKFNKENQFADEEIAALPNLNFYQK |
| | E6_15 aa_aCD16- | WAFIRKLMDDPSQSANLLAEAKKLNDA |
| | A10(2b- | QAPKGGGSGGGGSGGGGSGVDNKFNK |
| | E03)_His6c_OH | ELQSAQREIRQLPNLNHHQKFAFINSLL |
| | (SEQ ID 33) | DDPSQSANLLAEAKKLNDAQAPKYYH |
| | | HHHHH |
| 123 | H_aBCMA-1- | VDNKFNKENQFADEEIAALPNLNFYQK |
| | E6_15 aa_aCD16- | WAFIRKLMDDPSQSANLLAEAKKLNDA |
| | A10(34a- | QAPKGGGSGGGGSGGGGSGVDNKFNK |
| | D10)_His6c_OH | EQQEAQLEIRRLPNLNHHQVFAFIKSLL |
| | (SEQ ID 49) | DDPSQSANLLAEAKKLNDAQAPKYYH |
| | | HHHHH |
| 124 | H_aBCMA-1- | VDNKFNKENQFADEEIAALPNLNFYQK |
| | E6_15 aa_aCD16- | WAFIRKLMDDPSQSANLLAEAKKLNDA |
| | A10(2a- | QAPKGGGSGGGGSGGGGSGVDNKFNK |
| | G03)_His6c_OH | ETQVAQKEIRTLPNLNHHQIFAFLNKLI |
| | (SEQ ID 15) | DDPSQSANLLAEAKKLNDAQAPKYYH |
| | | HHHHH |
| 125 | H_aBCMA-1- | VDNKFNKENQFADEEIAALPNLNFYQK |
| | E6_15 aa_aCD16- | WAFIRKLMDDPSQSANLLAEAKKLNDA |
| | A10(34a- | QAPKGGGSGGGGSGGGGSGVDNKFNK |
| | F03)_His6c_OH | EVQIAQLEIRRLPNLNHHQKYAFIKSLA |
| | (SEQ ID 43) | DDPSQSANLLAEAKKLNDAQAPKYYH |
| | | HHHHH |
| 126 | H_aBCMA-1- | VDNKFNKENQFADEEIAALPNLNFYQK |
| | E6_15 aa_aCD16- | WAFIRKLMDDPSQSANLLAEAKKLNDA |
| | A10(2a- | QAPKGGGSGGGGSGGGGSGVDNKFNK |
| | C09)_His6c_OH | EVQIAQREIRSLPNLNHHQVFAFIKKLL |
| | (SEQ ID 19) | DDPSQSANLLAEAKKLNDAQAPKYYH |
| | | HHHHH |

CD16a Activation and NK Cell Mediated Cell Killing by hBCMA×hCD16a Dual Engagers with hCD16a Binding Polypeptide

The hBCMA×hCD16a dual engagers comprising hCD16a binding polypeptides example 127-129 were expressed in E. coli (DE3) and thereafter characterized for their CD16a activation properties.

The cDNA coding for each hBCMA×hCD16a dual engager harbouring a stop codon was synthesized and ligated into the Ndel Xhol restriction sites of the pET29 vector.

E coli BL21(DE3) was transformed with vector under Kanamycin selection and constructs were expressed by induction of IPTG at an OD of 0.6 and harvested 16 h later. Soluble cytosolic product was harvested and resuspended in 1×PBS. Heat denaturation for 7 min at 95° C. was applied and precipitated protein was pelleted by centrifugation at 20,000×g. Further purification was achieved by RP-HPLC and identity/purity was confirmed by SDS-PAGE and LC/MS/MS analysis. The propensity of the hBCMA×hCD16a dual engagers to stimulate CD16a activation and to enhance NK cell mediated killing were assessed according to the protocols described in Biological Example 11.

| | Descriptive ID | |
|---|---|---|
| SEQ | CD16a binder | |
| ID | (SEQ ID) | Sequence |
| 127 | H_Met2_aBCMA- | MMVDNKFNKENQFADEEIAALP |
| | 1-E6_15 aa_aCD16- | NLNFYQKWAFIRKLMDDPSQSA |
| | A10_OH | NLLAEAKKLNDAQAPKGGGSGG |
| | (SEQ ID 1) | GGSGGGGSGVDNKFNKEVQMAQ |
| | | FEIRKLPNLNHHQSFAFIKSLM |
| | | DDPSQSANLLAEAKKLNDAQAP |
| | | K |
| 128 | H_aBCMA-1- | VDNKFNKENQFADEEIAALPNL |
| | E6_10 aa_aBCMA- | NFYQKWAFIRKLMDDPSQSANL |
| | 1-E6_10 aa_aCD16- | LAEAKKLNDAQAPKGGGSGGGG |
| | A10_OH | SGVDNKFNKENQFADEEIAALP |
| | (SEQ ID 1) | NLNFYQKWAFIRKLMDDPSQSA |
| | | NLLAEAKKLNDAQAPKGGGSGG |
| | | GGSGVDNKFNKEVQMAQFEIRK |
| | | LPNLNHHQSFAFIKSLMDDPSQ |
| | | SANLLAEAKKLNDAQAPK |
| 129 | H_aBCMA-1- | VDNKFNKENQFADEEIAALPNL |
| | E6_10 aa_aBCMA- | NFYQKWAFIRKLMDDPSQSANL |
| | 1-E6_10 aa_aCD16- | LAEAKKLNDAQAPKGGGSGGGG |
| | A10(2a-E06)_OH | SGVDNKFNKENQFADEEIAALP |
| | (SEQ ID 17) | NLNFYQKWAFIRKLMDDPSQSA |
| | | NLLAEAKKLNDAQAPKGGGSGG |
| | | GGSGVDNKFNKELQYAQKEIRR |
| | | LPNLNHHQVFAFINKLIDDPSQ |
| | | SANLLAEAKKLNDAQAPK |

### Results

The results are shown in FIG. 17. It is seen that the hBCMA×hCD16a dual engagers (Examples 127-129) demonstrated CD16a activation in the Lucia Luciferase reporter assay in the presence of hBCMA+MM.1S cells, whereas no detectable activation was seen in the absence of target cells (FIG. 17a). Moreover, the engagers induced NK cell mediated killing of MM.1S cells (FIG. 17b). Thus, a firm and target specific activation leading to NK mediated cell killing was seen for the engagers investigated.

### Biological Example 13

hCD16a Activation by a hBCMA×hCD16a Dual Engager Harbouring an IL-15 Cytokine Polypeptide:

### Material and Methods

The cDNA coding for a polypeptide of the invention (SEQ ID 1) harboring a stop codon was synthesized and ligated into the Ndel Xholrestriction sites of the pET29 vector. E. coli BL21(DE3) was transformed with the vector under Kanamycin selection. The Compound, referred to herein as Example 101 was expressed by induction of IPTG at an OD of 0.6 and harvested 16 h later. Example 101 was purified from inclusion bodies by dissolving in 1×PBS supplemented by 30 mM imidazole, 6 M Urea, 10 mM glutathione (reduced). Soluble material was purified by IMAC using 1×PBS supplemented by 500 mM imidazole, 6 M Urea as elution buffer. Example 101 was thereafter refolded by dialysis against 1×PBS.

The propensity of the compound to stimulate CD16a activation was assessed in a Lucia Luciferase reporter assay Jurkat-Lucia™ NFAT-CD16 cells (InvivoGen) and compared to the antibody dependent cellular cytotoxicity (ADCC) CD16a activation of Elotuzumab (Empliciti, Bristol Myers Squibb, clinical grade a-SLAMF7 monoclonal antibody).

### Results

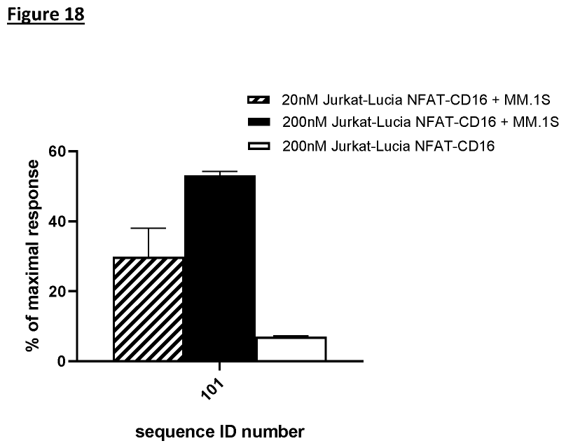

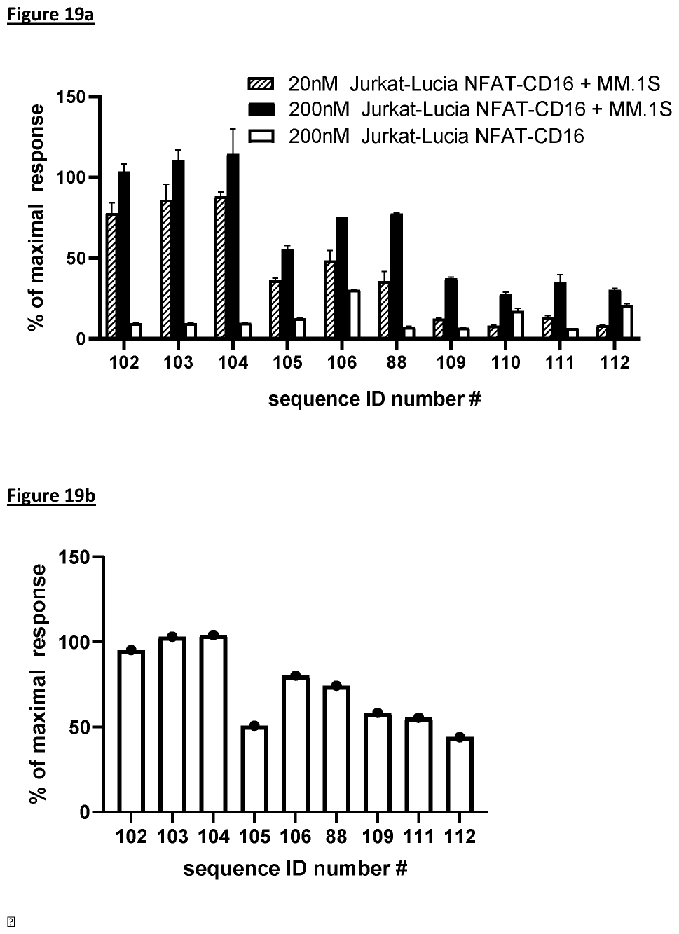

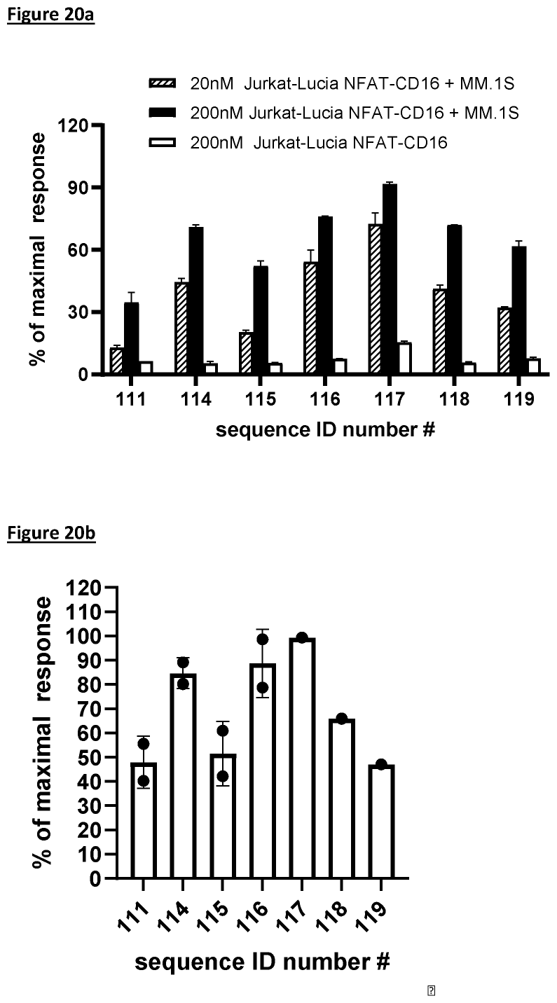

A hBCMA×hCD16a dual engager harbouring an IL-15 cytokine sequence (SEQ ID 101) was constructed and expressed as soluble gene products in E. coli (DE3). The CD16a activation response of the engager construct in the presence and absence of target MM.1s cells is shown in FIG. 18.

It is seen in the Figure that the affibody-based IL-15 containing dual engager can activate CD16a in a target specific manner.

| | Descriptive ID | |
|---|---|---|
| SEQ | CD16a binder | |
| ID | (SEQ ID) | Sequence |
| 101 | H_Met2_aBCMA- | MMVDNKFNKENQFADEEIAALP |
| | 1- | NLNFYQKWAFIRKLMDDPSQSA |
| | E6_15 aa_IL15(49- | NLLAEAKKLNDAQAPKGGGSGG |
| | 162)_15 aa_aCD16- | GGSGGGGSGNWVNVISDLKKIE |
| | A10_His6c_OH | DLIQSMHIDATLYTESDVHPSC |
| | (SEQ ID 1) | KVTAMKCFLLELQVISLESGDA |
| | | SIHDTVENLIILANNSLSSNGN |
| | | VTESGCKECEELEEKNIKEFLQ |
| | | SFVHIVQMFINTSGGGSGGGGS |
| | | GGGGSGVDNKFNKEVQMAQFEI |
| | | RKLPNLNHHQSFAFIKSLMDDP |
| | | SQSANLLAEAKKLNDAQAPKYY |
| | | HHHHHH |

### Biological Example 14

CD16a Activation and NK Cell Mediated Cell Killing of hBCMA×hCD16a Dual Engager Constructs with Different Domain Order and Linkers

Compounds with different distances and ordering of targeting domains in dual engager constructs were prepared. The propensity of the hBCMA×hCD16a dual engagers to stimulate CD16a activation and to enhance NK cell mediated killing were assessed according to the protocols described in Biological Example 11 The hCD16a binding was, in each case, the polypeptide with SEQ ID 1.

| SEQ | | |
|---|---|---|
| ID | Descriptive ID | Sequence |
| 102 | H_Met2_aBCMA- | MMVDNKFNKENQFADEEIAALPNLNFYQK |
| | 1-E6_5 aa_ | WAFIRKLMDDPSQSANLLAEAKKLNDAQA |
| | aBCMA-1-E6_ | PKGGGSGVDNKFNKENQFADEEIAALPNLN |
| | 5 aa_aCD16- | FYQKWAFIRKLMDDPSQSANLLAEAKKLN |
| | A10_His6c_OH | DAQAPKGGGSGVDNKFNKEVQMAQFEIRK |
| | | LPNLNHHQSFAFIKSLMDDPSQSANLLAEA |
| | | KKLNDAQAPKYYHHHHHH |
| 103 | H_Met2_aBCMA- | MMVDNKFNKENQFADEEIAALPNLNFYQK |
| | 1- | WAFIRKLMDDPSQSANLLAEAKKLNDAQA |
| | E6_10 aa_aBCMA- | PKGGGSGGGGSGVDNKFNKENQFADEEIA |
| | 1-E6_10 aa_ | ALPNLNFYQKWAFIRKLMDDPSQSANLLA |
| | aCD16-A10_ | EAKKLNDAQAPKGGGSGGGGSGVDNKFN |
| | His6c_OH | KEVQMAQFEIRKLPNLNHHQSFAFIKSLMD |
| | | DPSQSANLLAEAKKLNDAQAPKYYHHHHH |
| | | H |
| 104 | H_Met2_aBCMA- | MMVDNKFNKENQFADEEIAALPNLNFYQK |
| | 1- | WAFIRKLMDDPSQSANLLAEAKKLNDAQA |
| | E6_15 aa_aBCMA- | PKGGGSGGGGSGGGGSGVDNKFNKENQFA |
| | 1-E6_15 aa_ | DEEIAALPNLNFYQKWAFIRKLMDDPSQSA |
| | aCD16-A10_ | NLLAEAKKLNDAQAPKGGGSGGGGSGGG |
| | His6c_OH | GSGVDNKFNKEVQMAQFEIRKLPNLNHHQ |
| | | SFAFIKSLMDDPSQSANLLAEAKKLNDAQA |
| | | PKYYHHHHHH |
| 105 | H_Met2_aBCMA- | MMVDNKFNKENQFADEEIAALPNLNFYQK |
| | 1-E6_15 aa_ | WAFIRKLMDDPSQSANLLAEAKKLNDAQA |
| | aCD16-A10_ | PKGGGSGGGGSGGGGSGVDNKFNKEVQM |
| | 15 aa_aBCMA- | AQFEIRKLPNLNHHQSFAFIKSLMDDPSQS |
| | 1-E6_His6c_OH | ANLLAEAKKLNDAQAPKGGGSGGGGSGGG |
| | | GSGVDNKFNKENQFADEEIAALPNLNFYQK |
| | | WAFIRKLMDDPSQSANLLAEAKKLNDAQA |
| | | PKYYHHHHHH |
| 106 | H_Met2_aCD16- | MMVDNKFNKEVQMAQFEIRKLPNLNHHQS |
| | A10_15 aa_ | FAFIKSLMDDPSQSANLLAEAKKLNDAQAP |
| | aBCMA-1- | KGGGSGGGGSGGGGSGVDNKFNKENQFA |
| | E6_15 aa_aBCMA- | DEEIAALPNLNFYQKWAFIRKLMDDPSQSA |
| | 1-E6_His6c_OH | NLLAEAKKLNDAQAPKGGGSGGGGSGGG |
| | | GSGVDNKFNKENQFADEEIAALPNLNFYQK |
| | | WAFIRKLMDDPSQSANLLAEAKKLNDAQA |
| | | PKYYHHHHHH |
| 88 | H_aBCMA-1- | VDNKFNKENQFADEEIAALPNLNFYQKWA |
| | E6_15 aa_aCD16- | FIRKLMDDPSQSANLLAEAKKLNDAQAPK |
| | A10_His6c_OH | GGGSGGGGSGGGGSGVDNKFNKEVQMAQ |
| | | FEIRKLPNLNHHQSFAFIKSLMDDPSQSAN |
| | | LLAEAKKLNDAQAPKYYHHHHHH |
| 109 | H_aBCMA-1- | VDNKFNKENQFADEEIAALPNLNFYQKWA |
| | E6_aCD16- | FIRKLMDDPSQSANLLAEAKKLNDAQAPK |
| | A10_His6c_OH | VDNKFNKEVQMAQFEIRKLPNLNHHQSFA |
| | | FIKSLMDDPSQSANLLAEAKKLNDAQAPKY |
| | | YHHHHHH |
| 110 | H_aCD16- | VDNKFNKEVQMAQFEIRKLPNLNHHQSFA |
| | A10_aBCMA-1- | FIKSLMDDPSQSANLLAEAKKLNDAQAPKV |
| | E6_His6c_OH | DNKFNKENQFADEEIAALPNLNFYQKWAFI |
| | | RKLMDDPSQSANLLAEAKKLNDAQAPKYY |
| | | HHHHHH |
| 111 | H_aBCMA-1- | VDNKFNKENQFADEEIAALPNLNFYQKWA |
| | E6_Gly_aCD16- | FIRKLMDDPSQSANLLAEAKKLNDAQAPK |
| | A10_His6c_OH | GVDNKFNKEVQMAQFEIRKLPNLNHHQSF |
| | | AFIKSLMDDPSQSANLLAEAKKLNDAQAPK |
| | | YYHHHHHH |
| 112 | H_aCD16- | VDNKFNKEVQMAQFEIRKLPNLNHHQSFA |
| | A10_Gly_aBCMA- | FIKSLMDDPSQSANLLAEAKKLNDAQAPKG |
| | 1-E6_His6c_OH | VDNKFNKENQFADEEIAALPNLNFYQKWA |
| | | FIRKLMDDPSQSANLLAEAKKLNDAQAPK |
| | | YYHHHHHH |

Design, Construction, Expression, and Protein Purification of hBCMA×hCD16a-Binding Polypeptide Engager Constructs.

hBCMA×hCD16a binding constructs were designed for expression and purification according to Biological Example 11. Both heterodimeric hBCMA XhCD16a dual binding constructs (Example Compounds 88, 109, 110, 111 and 112) and heterotrmeric hBCMA×hCD16a binding constructs containing two BCMA-binding polypeptides were evaluated (Examples 102, 103, 104, 105 and 106). The propensity of the hBCMA×hCDl16a dual engagers to stimulate CD 16a activation and to enhance NIK cell mediated killing were assessed according to the protocols described in Biological Example 11.

### Results

hBCMA×hCD16a dual engager constructs containing one or two hBCMA-binding polypeptides genetically fused to a hCD16a binding arm composed of the polypeptide of SEQ ID 1 and a C-terminal His₆ tag were constructed and expressed as soluble gene products in E. coli (DE3). FIG. 19a shows the hCD16a activation responses of the engager constructs in the presence or absence of target MM.1s cells. Moreover, the engagers induced NK cell mediated killing of MM.1S cells (FIG. 19b).

The results show that the affibody-based hCD16a binding arm, here composed of the polypeptide of SEQ ID 1, can activate hCD16a in a target specific manner when genetically fused either N-terminally or C-terminally of a targeting polypeptide. In addition, the affibody-based hCD16a binding arm could also be fused between two other targeting polypeptide units. hCD16a activation was observed for engagers with linker length varying from 0 and 15 amino acids

### Biological Example 15

### Assessment of Domain Boundaries in BCMA×hCD16a Dual Engager Constructs Using a Jurkat CD16a Activation Reporter Assay and a NK Mediated Cell Killing Assay

hBCMA×hCD16a dual binding constructs with truncations in both the N- and C-terminal of both the BCMA binding polypeptide and the CD16a binding A10 affibody were produced and evaluated in a hCD16a-mediated activation cell-based reporter assay and cell killing. This enabled the domain boundaries of the targeting domains in dual engager constructs to be assessed.

| SEQ | | |
|---|---|---|
| ID | Descriptive ID | Sequence |
| 111 | H_aBCMA-1- | VDNKFNKENQFADEEIAALPNLNFYQKWA |
| | E6_Gly_aCD16- | FIRKLMDDPSQSANLLAEAKKLNDAQAPK |
| | A10_His6c_OH | GVDNKFNKEVQMAQFEIRKLPNLNHHQSF |
| | | AFIKSLMDDPSQSANLLAEAKKLNDAQAPK |
| | | YYHHHHHH |
| 114 | H_Met_aBCMA-1- | MNKENQFADEEIAALPNLNFYQKWAFIRKL |
| | E6-(6- | MDDPSQSANLLAEAKKLNDAQGNKEVQM |
| | 55)_Gly_aCD16- | AQFEIRKLPNLNHHQSFAFIKSLMDDPSQS |
| | A10-(6- | ANLLAEAKKLNDAQAPKYYHHHHHH |
| | 58)_His6c_OH | |
| 115 | H_Met_aBCMA-1- | MNKENQFADEEIAALPNLNFYQKWAFIRKL |
| | E6-(6- | MDDPSQSANLLAEAKKLNDAQGVDNKFN |
| | 55)_Gly_aCD16- | KEVQMAQFEIRKLPNLNHHQSFAFIKSLMD |
| | A10-(1- | DPSQSANLLAEAKKLNDAQYYHHHHHH |
| | 55)_His6c_OH | |
| 116 | H_Met_aBCMA-1- | MNKENQFADEEIAALPNLNFYQKWAFIRKL |
| | E6-(6- | MDDPSQSANLLAEAKKLNDAQGVDNKFN |
| | 55)_Gly_aCD16- | KEVQMAQFEIRKLPNLNHHQSFAFIKSLMD |
| | A10_His6c_OH | DPSQSANLLAEAKKLNDAQAPKYYHHHHH |
| | | H |
| 117 | H_aBCMA-1-E6- | GDNKFNKENQFADEEIAALPNLNFYQKWA |
| | (V1G)_Gly_ | FIRKLMDDPSQSANLLAEAKKLNDAQAPK |
| | aCD16-A10-(6- | GNKEVQMAQFEIRKLPNLNHHQSFAFIKSL |
| | 58)_His6c_OH | MDDPSQSANLLAEAKKLNDAQAPKYYHH |
| | | HHHH |
| 118 | H_aBCMA-1-E6- | GDNKFNKENQFADEEIAALPNLNFYQKWA |
| | (V1G)_Gly_ | FIRKLMDDPSQSANLLAEAKKLNDAQAPK |
| | aCD16-A10-(1- | GVDNKFNKEVQMAQFEIRKLPNLNHHQSF |
| | 55)_His6c_OH | AFIKSLMDDPSQSANLLAEAKKLNDAQYY |
| | | HHHHHH |
| 119 | H_aBCMA-1-E6- | GDNKFNKENQFADEEIAALPNLNFYQKWA |
| | (V1G)_Gly_ | FIRKLMDDPSQSANLLAEAKKLNDAQAPK |
| | aCD16-A10 | GVDNKFNKEVQMAQFEIRKLPNLNHHQSF |
| | His6c_OH | AFIKSLMDDPSQSANLLAEAKKLNDAQAPK |
| | | YYHHHHHH |

Design, Construction, Expression, and Protein Purification of hBCMA×hCD16a CD16a-Binding Polypeptide Dual Engager Constructs.

hBCMA×hCD16a dual binding constructs were designed for expression and purification according to Biological Example 11. The heterodimeric hBCMA hCD16a dual binding constructs that were prepared are listed above (SEQ IDs 113-119).

The propensity of the hBCMA×hCD16a dual engagers to stimulate CD16a activation and to enhance NK cell mediated killing were assessed according to the protocols described in Biological Example 11.

### Results

hBCMA×hCD16a dual engager constructs containing one BCMA-binding polypeptide genetically fused to a hCD16a binding arm composed of the polypeptide of SEQ ID 1 and a C-terminal His₆ tag were constructed and expressed as a soluble gene products in E. coli (DE3). FIG. 20a shows the CD16a activation responses of the engager constructs in the presence or absence of target MM.1s cells. Moreover, the engagers induced NK cell mediated killing of MM.1 S cells (FIG. 20b).

The results show that the hCD16a binding arm, here composed of the polypeptide of SEQ ID 1 can be shortened and still retain hCD16a activation.

### Biological Example 16

### NK Cell Mediated Multiple Myeloma Cell Killing

The ability of a hBCMA×hCD16a dual engager to promote NK cell mediated lysis and cell killing was assessed by a flow cytometry assay and an Incucyte based assay. The compounds investigated were:

| SEQ | Descriptive | |
|---|---|---|
| ID | ID | Sequence |
| 85 | H_aBCMA-1- | VDNKFNKENQFADEEIAALPNLNFYQKWA |
| | E6_15 aa_ | FIRKLMDDPSQSANLLAEAKKLNDAQAPK |
| | aCD16-H09_ | GGGSGGGGSGGGGSGVDNKFNKEFWIAES |
| | 15 aa_ | EIESLPNLNIYQKWAFKYSLADDPSQSANLL |
| | aCD16-A10_ | AEAKKLNDAQAPKGGGSGGGGSGGGGSG |
| | His6c_OH | VDNKFNKEVQMAQFEIRKLPNLNHHQSFA |
| | | FIKSLMDDPSQSANLLAEAKKLNDAQAPKY |
| | | YHHHHHH |
| 86 | H_aBCMA-1- | VDNKFNKENQFADEEIAALPNLNFYQKWA |
| | E6_15 aa_ | FIRKLMDDPSQSANLLAEAKKLNDAQAPK |
| | aCD16-A10_ | GGGSGGGGSGGGGSGVDNKFNKEVQMAQ |
| | 15 aa_aCD16- | FEIRKLPNLNHHQSFAFIKSLMDDPSQSANL |
| | A10_His6c_OH | LAEAKKLNDAQAPKGGGSGGGGSGGGGS |
| | | GVDNKFNKEVQMAQFEIRKLPNLNHHQSF |
| | | AFIKSLMDDPSQSANLLAEAKKLNDAQAPK |
| | | YYHHHHHH |
| 88 | H_aBCMA-1- | VDNKFNKENQFADEEIAALPNLNFYQKWA |
| | E6_15 aa_ | FIRKLMDDPSQSANLLAEAKKLNDAQAPK |
| | aCD16-A10_ | GGGSGGGGSGGGGSGVDNKFNKEVQMAQ |
| | His6c_OH | FEIRKLPNLNHHQSFAFIKSLMDDPSQSANL |
| | | LAEAKKLNDAQAPKYYHHHHHH |

Compounds were produced according to Biological Example 10A.

### Flow Cytometry-Based In Vitro Assays.

NK cell function was assessed in flow cytometry-based assays described previously (Bryceson, Fauriat et al. 2010). In brief, NK cells were co-cultured with target cells for 6 hours at an E:T ratio of 1:1, followed by staining for degranulation marker CD107a and interferon-gamma. BD GolgiStop (BD Biosciences) was added after 1 hour at a concentration of 1/1500. For cytotoxicity assays, target cells were pre-stained with CellTrace Violet (Invitrogen) and co-incubated with NK cells at different E:T ratios for 8-12 hours, based on kinetics from live cell imaging assays. Samples were acquired on either a BD LSR II or a FACSymphony A5 (BD Biosciences). Data were analyzed with FlowJo version 10 (BD Biosciences).

### Incucyte-Based In Vitro Cytotoxicity Assays.

Real-time tumor cell killing was measured on the Incucyte S3 platform (Essen Biosciences). NK cells were co-cultured with MM.1s cells labelled with the red fluorescent protein mCherry, on 96-well plates precoated with Poly-L-Ornithine 0.01% (Sigma-Aldrich) at various E:T ratios depending on the type of assay. For assays involving reagents (e.g. affibody constructs), these were added after the effector and target cells. Scanning was done every 60 minutes for at least 24 hours, using a lOX objective lens, and images were analyzed using Incucyte Controller v2020A (Essen Biosciences). The same masking was used for repeat analyses. In the experiments carried out, the cells were treated with 100 nM of dual engager, null variant (not binding hBCMA), or daratumumab.

### Results

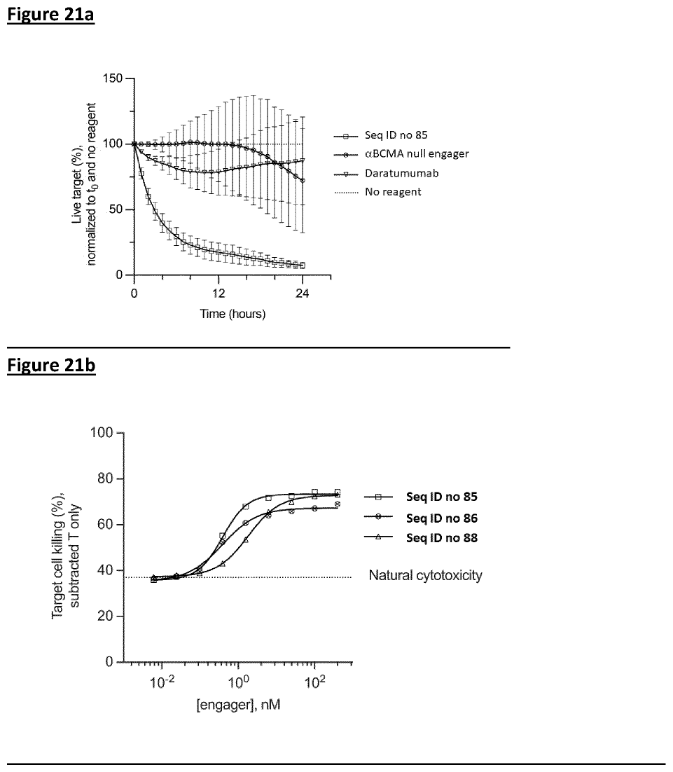

The capacity of the dual engager Example Compound of SEQ ID 85 to induce lysis of MM.1s cells was examined. The results are shown in FIG. 21a. Results are from one representative experiment (n=4). In a 24-hour killing assay, Example compound with SEQ ID 85 elicited rapid and superior lysis of MM.1s cells, compared to both daratumumab and the control null-variant anti-hBCMA-Null-H09-A10-His₆. The bulk cytotoxic effect of the compound with SEQ ID 85 was seen within the first 8 to 12 hours after the start of the co-culture.

Finally, a flow cytometry-based killing assay was implemented to establish functional EC₅₀ and EC₉₀ values of the three constructs with SEQ IDs 85, 86 and 88. The frequency of dead target cells was calculated based on the staining of dead cell marker on target cells pre-incubated with cell trace violet (CTV). The results are shown in FIG. 21b. Results are from two independent experiments (n=8), where spontaneous target cell death is subtracted. The plateau-level cytotoxicity was highest for the compound with SEQ ID 85 and lowest for the compound with SEQ ID 86, in agreement with the degranulation responses, which showed a higher fratricide effect for the latter construct. Compared with the compound with SEQ ID 88, plateau cytotoxicity for the compound with SEQ ID 85 was reached at a lower dose. EC₅₀ values were 0.4 nM for SEQ ID 85 and SEQ ID 86, and 1.8 nM for SEQ ID 88, while EC₉₀ values were 2.0, 3.8, and 14.7 nM, respectively. The data demonstrate that these dual engagers elicit potent responses.

### Biological Example 17

Enhancement of NK Cell Mediated Lysis of Multiple Myeloma Cells in a hBCMA Selective Manner.

The ability of a hBCMA×hCD16a dual engager to enhance NK cell mediated lysis of multiple myeloma cells in a BCMA selective manner was investigated. The compound that had SEQ ID 85.

### Genetic Cell Engineering

A BCMA knock-out variant of cell line MM.1s was made using CRISPR-Cas9. The BCMA overexpression variant of cell line MM.1s and variants expressing the red fluorescent protein mCherry was done through lentiviral transfection, with vectors designed and ordered from VectorBuilder (VectorBuilder Inc.). MM.1s expressing the nuclear red fluorescent protein mCherry: MM.1s cells were transduced with 0.3 MOI lentiviral particles. After transduction, approximately 30% of cells appeared red in the fluorescence microscope. Cells were then sorted by fluorescence-activated cell sorting (FACS) on a Sony SH800 cell sorter (Sony Biotechnology). MM.1s with BCMA gene overexpressed: On day 1, 1 million MM.1s cells were transduced with 10 MOI lentiviral particles (denoted SK—V93, and shown in Figure S6A) and seeded out on a 24-well plate. On day 4, cells were washed in PBS and seeded out at 0.5 million cells per milliliter in fresh media. After sufficient expansion, cells went through FACS. MM.1s with BCMA gene knocked out: The following sgRNA sequences were used: sgRNA #0, gacgagtttaaaaacac (SEQ ID 245); sgRNA #1, gagcttaataatttctt (SEQ ID 246); and sgRNA #2, gtgaccaattcagtgaa (SEQ ID 246). sgRNA and Cas9 were assembled at a ratio of 9:1 on sterile 96-well U-bottom plates. 300.000 MM.1s cells were used per nucleofection. Knockout efficiency was confirmed after five days by PCR and Sanger sequencing, analyzed with Synthego Performance Analysis (ICE) (Synthego). After sufficient expansion, cells went through FACS.

### Flow Cytometry-Based In Vitro Assays.

NK cell function was assessed in flow cytometry-based assays described previously (Bryceson, Fauriat et al. 2010). In brief, NK cells were co-cultured with target cells for 6 hours at an E:T ratio of 1:1, followed by staining for degranulation marker CD107a and interferon-gamma. BD GolgiStop (BD Biosciences) was added after 1 hour at a concentration of 1/1500.

### Results

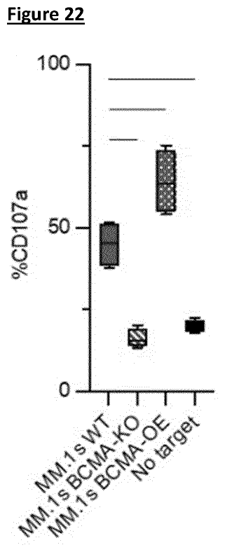

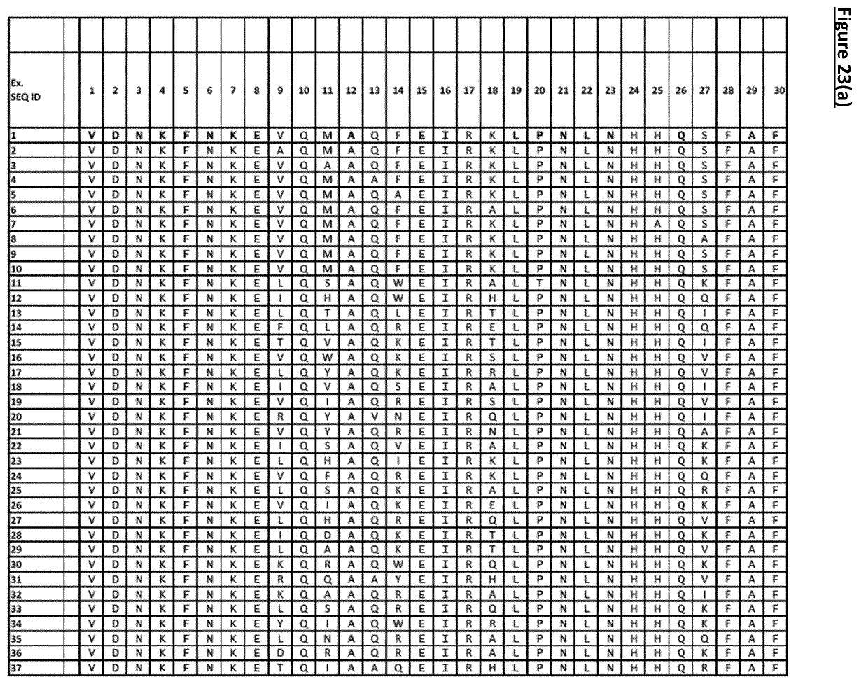

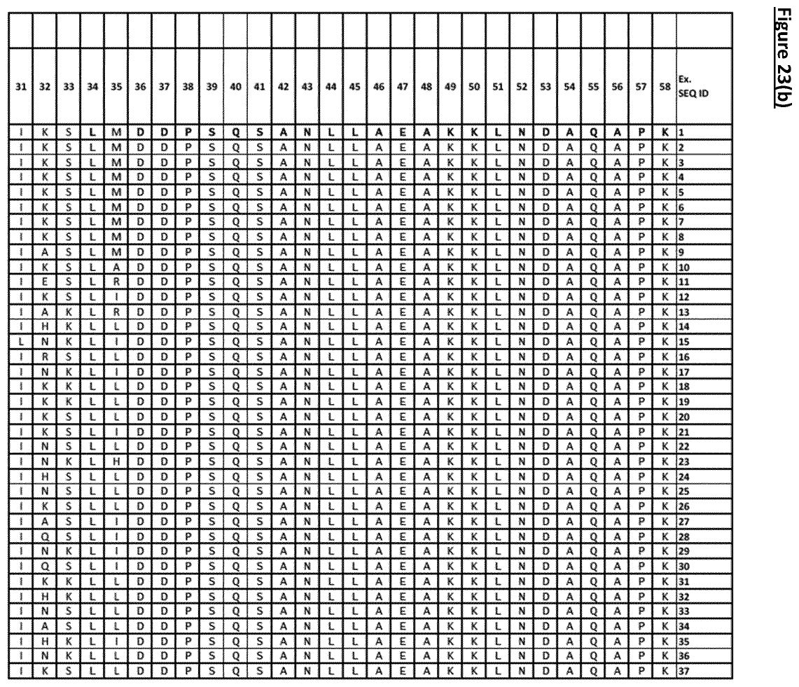

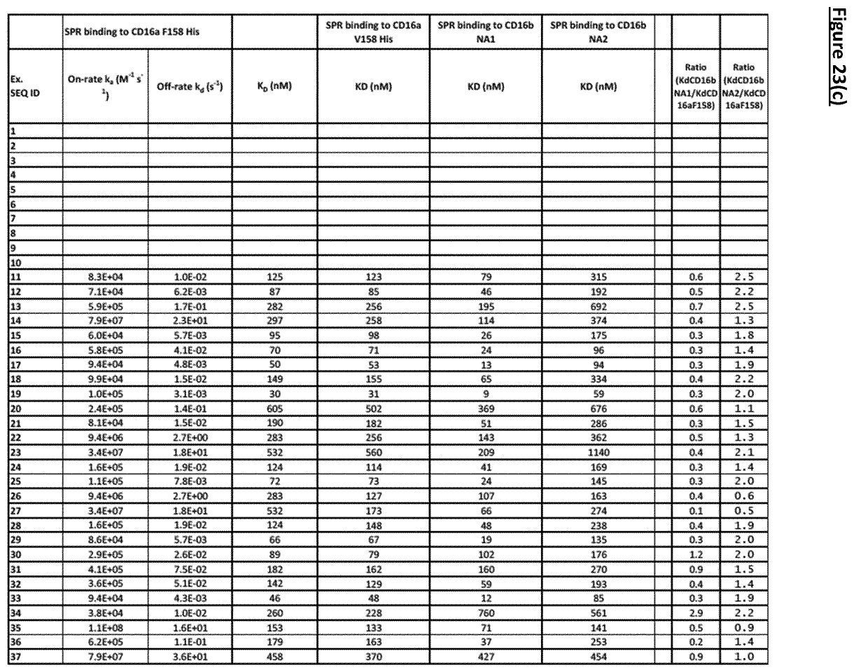

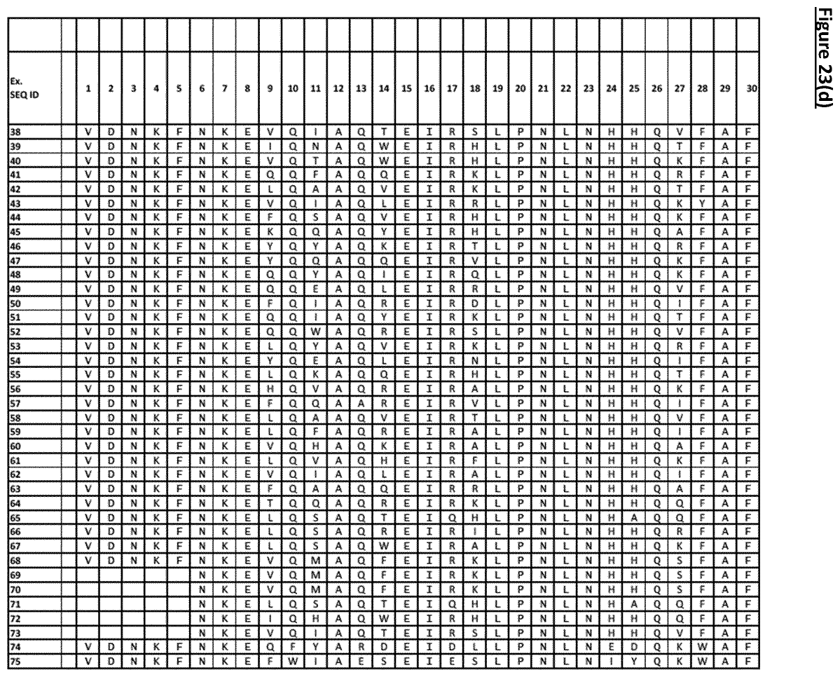

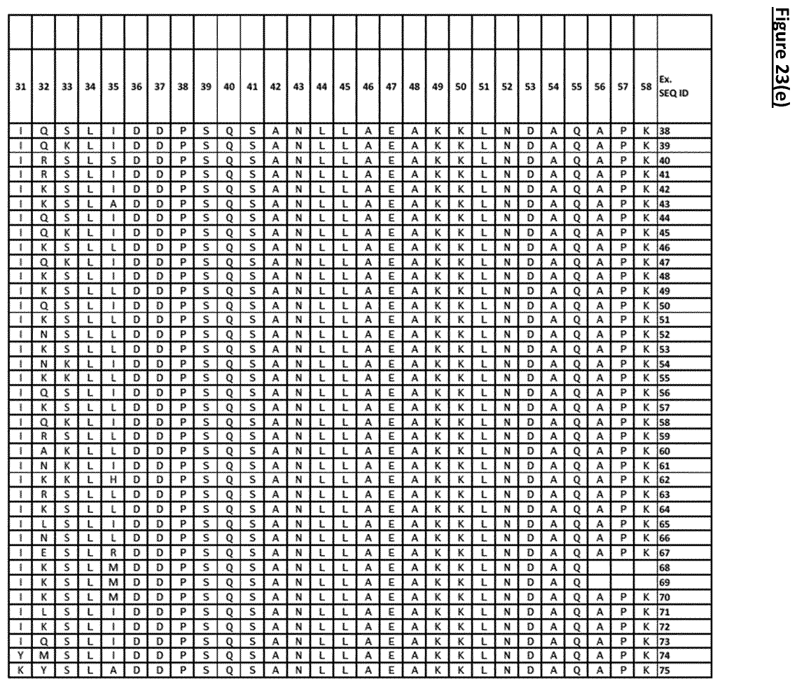

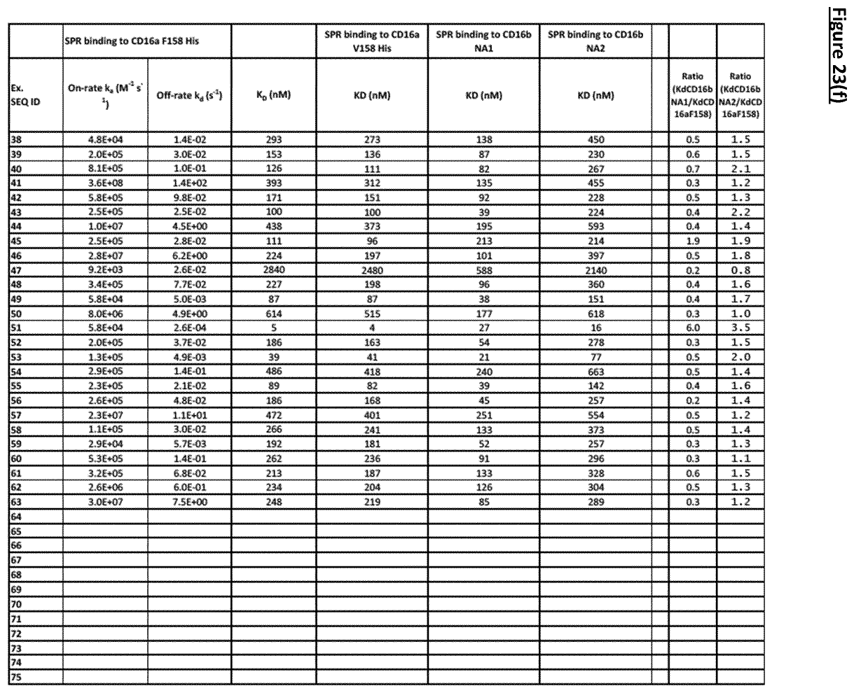

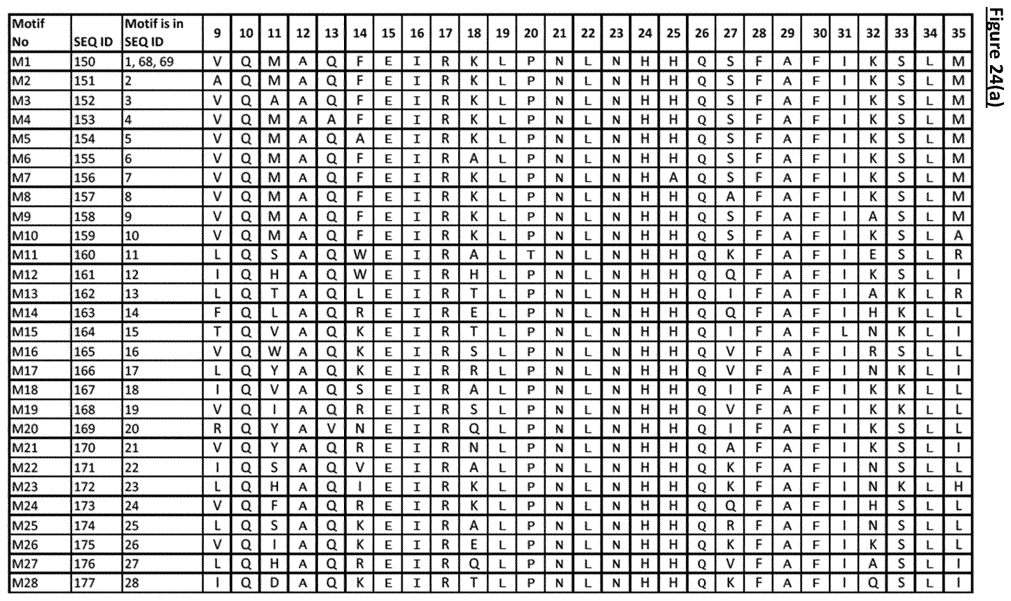

Using engineered MM.1s target cells in which BCMA was either knocked out or overexpressed, a clear correlation between the BCMA expression level and NK cell activation was observed (see FIG. 22). The data demonstrate that the dual engager compound with SEQ ID 85 a concentration of 100 nM elicits responses, with specificity to BCMA. In the figure, the results are shown for MM.1s wild type (WT), BCMA knock-out (KO), and BCMA overexpression (OE). Results are from one representative experiment (n=4).

The following embodiments (Embodiments 1-39) additionally illustrate the invention: Embodiment 1.
A CD16a-binding polypeptide which comprises at least one motif that binds to CD16a, wherein said polypeptide comprises the following structure:
[N-terminal portion]-[Helix 1]-[Separating portion]-[Helix 2]—[C-terminal portion]
the CD16a binding motif being the portion [Helix 1]-[Separating portion]-[Helix 2].

### Embodiment 2

The CD16a-binding polypeptide according to Embodiment 1, wherein:
i) Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂SLX₃₅, wherein,
a*) X₉ is V; X₁₀ is Q; X₁₁ is M; X₁₃ is Q; X₁₄ is F; X₁₇ is R; X₁₈ is K; X₂₄ is H; X₂₅ is H; X₂₇ is S; X₂₈ is F; X₃₁ is I; X₃₂ is K; and X₃₅ is M;
b*) X₉ is Q; X₁₀ is F; X₁₁ is Y; X₁₃ is R; X₁₄ is D; X₁₇ is D; X₁₈ is L; X₂₄ is E; X₂₅ is D; X₂₇ is K; X₂₈ is W; X₃₁ is Y; X₃₂ is M; and X₃₅ is I; or
c*) X₉ is F; X₁₀ is W; X₁₁ is I; X₁₃ is E; X₁₄ is S; X₁₇ is E; X₁₈ is S; X₂₄ is I; X₂₅ is Y; X₂₇ is K; X₂₈ is W; X₃₁ is K; X₃₂ is Y; and X₃₅ is A;
or ii) Helix 1 and Helix 2 are defined as in i), wherein within Helix 1 and Helix 2, at least 1 and no more than 3 of the XI residues are replaced by an alternative residue, and/or at least 1 and no more than 3 of the residues not labelled as X₁ are replaced by an alternative residue.

### Embodiment 3

The CD16a-binding polypeptide according to Embodiment 2, wherein:
i) Helix 1 comprises the sequence NKEVQMAQFEIRKL and Helix 2 comprises the sequence NHHQSFAFIKSLMDD, or ii) Helix 1 and Helix 2 are defined as in i), wherein within Helix 1 and Helix 2, at least 1 and no more than 2 residues are replaced by an alternative residue.

### Embodiment 4

The CD16a-binding polypeptide according to Embodiment 3, wherein the CD16a binding efficacy is at least 10% of SEQ ID NO: 1.

### Embodiment 5

The immune cell-engaging polypeptide according to Embodiment 2, wherein:
i) Helix 1 comprises the sequence NKEQFYARDEIDLL and Helix 2 comprises the sequence NEDQKWAFYMSLIDD,
or ii) Helix 1 and Helix 2 are defined as in i), wherein within Helix 1 and Helix 2, at least 1 and no more than 2 residues are replaced by an alternative residue.

### Embodiment 6

The CD16a-binding polypeptide according to Embodiment 5, wherein the CD16a binding efficacy is at least 10% of SEQ ID NO: 2.

### Embodiment 7

The CD16a-binding polypeptide according to Embodiment 2, wherein:
i) Helix 1 comprises the sequence NKEFWIAESEIESL and Helix 2 comprises the sequence NIYQKWAFKYSLADD,
or ii) Helix 1 and Helix 2 are defined as in i), wherein within Helix 1 and Helix 2, at least 1 and no more than 2 residues are replaced by an alternative residue.

### Embodiment 8

The CD16a-binding polypeptide according to Embodiment 7, wherein the CD16a binding efficacy is at least 10% of SEQ ID NO: 3.

### Embodiment 9

The CD16a-binding polypeptide according to any of Embodiments 1-8,
wherein:
(i) said separating portion has the sequence PNL and said CD16a binding motif is flanked by an N-terminal portion VDNKF and a C-terminal portion PSQSANLLAEAKKLNDAQAPK;
or (ii) the separating portion, N-terminal portion, and C-terminal portion are as defined in (i), wherein within those portions taken together at least 1 and no more than 5 residues are replaced by an alternative residue.

### Embodiment 10

The CD16a-binding polypeptide according to Embodiment 1, wherein the sequence of said at least one CD16a-binding polypeptide is selected from:

[SEQ ID NO: 1]
VDNKFNKEVQMAQFEIRKLPNLNHHQSFAFIKSLMDDPSQSANLLAEA
KKLNDAQAPK;
[SEQ ID NO: 2]
VDNKFNKEQFYARDEIDLLPNLNEDQKWAFYMSLIDDPSQSANLLAEA
KKLNDAQAPK;
or;
[SEQ ID NO: 3]
VDNKFNKEFWIAESEIESLPNLNIYQKWAFKYSLADDPSQSANLLAEA
KKLNDAQAPK.

### Embodiment 11

The CD16a-binding polypeptide according to any of Embodiments 1-10, which comprises at least two CD16a-binding polypeptides.

### Embodiment 12

The CD16a-binding polypeptide according to Embodiment 11, which comprises two CD16a-binding polypeptides, each of which has a sequence as recited in Embodiment 3 or Embodiment 4, for example:

[SEQ ID NO: 1]
VDNKFNKEVQMAQFEIRKLPNLNHHQSFAFIKSLMDDPSQSANLLAEA
KKLNDAQAPK.

### Embodiment 13

The CD16a-binding polypeptide according to Embodiment 11, which comprises two CD16a-binding polypeptides, one having a sequence as recited in Embodiment 5 or Embodiment 6, for example:

[SEQ ID NO: 2]
VDNKFNKEQFYARDEIDLLPNLNEDQKWAFYMSLIDDPSQSANLLAE
AKKLNDAQAPK
and

the other having a sequence as recited in claim 3 or claim 4, for example:

[SEQ ID NO: 1]
VDNKFNKEVQMAQFEIRKLPNLNHHQSFAFIKSLMDDPSQSANLLAEA
KKLNDAQAPK.

### Embodiment 14

The CD16a-binding polypeptide according to Embodiment 11, which comprises two CD16a-binding polypeptides one having a sequence as recited in Embodiment 3 or Embodiment 4, for example:

[SEQ ID NO: 1]
VDNKFNKEVQMAQFEIRKLPNLNHHQSFAFIKSLMDDPSQSANLLAEA
KKLNDAQAPK
and

another having a sequence as recited in Embodiment 7 or Embodiment 8, for example:

[SEQ ID NO: 3]
VDNKFNKEFWIAESEIESLPNLNIYQKWAFKYSLADDPSQSANLLAEA
KKLNDAQAPK

### Embodiment 15

The CD16a-binding polypeptide according to Embodiment 11, which comprises four CD16a-binding polypeptides.

### Embodiment 16

The CD16a-binding polypeptide according to Embodiment 15, wherein two of the CD16a-binding polypeptides have a sequence as recited in Embodiment 3 or Embodiment 4, for example:

[SEQ ID NO: 1]
VDNKFNKEVQMAQFEIRKLPNLNHHQSFAFIKSLMDDPSQSANLLAEA
KKLNDAQAPK

and two of the CD16a-binding polypeptides have a sequence as recited in Embodiment 7 or Embodiment 8, for example:

[SEQ ID NO: 3]
VDNKFNKEFWIAESEIESLPNLNIYQKWAFKYSLADDPSQSANLLAEA
KKLNDAQAPK.

### Embodiment 17

The CD16a-binding polypeptide according to any of Embodiment 11 to 16, wherein the CD16a-binding polypeptides are separated by a linker.

### Embodiment 18

The CD16a-binding polypeptide as claimed in Embodiment 17, wherein the linker comprises the sequence GGGSG.

### Embodiment 19

The CD16a-binding polypeptide according to Embodiment 17 or Embodiment 18, which comprises the sequence

VDNKFNKEVQMAQFEIRKLPNLNHHQSFAFIKSLMDDPSQSANLLAEA
KKLNDAQAPKGGGSGGGGSGGGGSGVDNKFNKEVQMAQFEIRKLPN
LNHHQSFAFIKSLMDDPSQSANLLAEAKKLNDAQAPK.

### Embodiment 20

The CD16a-binding polypeptide according to Embodiment 17 or Embodiment 18, which comprises the sequence

VDNKFNKEQFYARDEIDLLPNLNEDQKWAFYMSLIDDPSQSANLLAE
AKKLNDAQAPKGGGSGGGGSGGGGSGVDNKFNKEVQMAQFEIRKLP
NLNHHQSFAFIKSLMDDPSQSANLLAEAKKLNDAQAPK.

### Embodiment 21

The CD16a-binding polypeptide according to Embodiment 17 or Embodiment 18, which comprises the sequence

VDNKFNKEFWIAESEIESLPNLNIYQKWAFKYSLADDPSQSANLLAEA
KKLNDAQAPKGGGSGGGGSGGGGSGVDNKFNKEVQMAQFEIRKLPN
LNHHQSFAFIKSLMDDPSQSANLLAEAKKLNDAQAPK.

### Embodiment 22

The CD16a-binding polypeptide according to any of Embodiments 1-21, which further comprises one or more additional binding moiety(ies).

### Embodiment 23

The CD16a-binding polypeptide according to Embodiment 22, wherein the one or more additional binding moiety(ies) is specific for a cancer cell surface target, for example a myeloma cell surface antigen, for example BCMA.

### Embodiment 24

The CD16a-binding polypeptide according to Embodiment 22 or Embodiment 23, wherein the additional binding moiety(ies) is separated from the at least one CD16a-binding polypeptide by a linker.

### Embodiment 25

The CD16a-binding polypeptide according to Embodiment 24, wherein the linker comprises the sequence GGGSG.

### Embodiment 26

A nucleic acid molecule encoding the CD16a-binding polypeptide according to any of Embodiments 1 to 25.

### Embodiment 27

An expression vector comprising the nucleic acid molecule according to Embodiment 26.

### Embodiment 28

A host cell comprising the nucleic acid molecule according to Embodiment 26 or the expression vector according to Embodiment 27.

### Embodiment 29

A method of making the CD16a-binding polypeptide according to any of Embodimentsl to 25, the method comprising maintaining the host cell of Embodiment 28 under optimal conditions for expression of the nucleic acid and isolating the CD16a-binding polypeptide.

### Embodiment 30

A pharmaceutical composition comprising the CD16a-binding polypeptide according to any of Embodiments 1 to 25, the nucleic acid molecule according 26 or the expression vector as claimed in Embodiments 27.

### Embodiment 31

The CD16a-binding polypeptide according to any of Embodiments 1 to 25, the nucleic acid molecule according to Embodiment 26, the expression vector according to Embodiment 27, and/or the pharmaceutical composition as according to Embodiment 30, for use in medicine.

### Embodiment 32

The CD16a-binding polypeptide according to any of Embodiments 1 to 25, the nucleic acid molecule according to Embodiment 26, the expression vector according to Embodiment 27, and/or the pharmaceutical composition according to Embodiment 30, for use in the treatment of cancer.

### Embodiment 33

The CD16a-binding polypeptide according to any of Embodiments 1 to 25, the nucleic acid molecule according to Embodiment 26, the expression vector according to Embodiment 27, and/or the pharmaceutical composition according to Embodiment 30, for use according to Embodiment 32, wherein the cancer is multiple myeloma.

### Embodiment 35

Use of the CD16a-binding polypeptide according to any of Embodiments 1 to 25, the nucleic acid molecule according to Embodiment 26, the expression vector according to Embodiment 27, and/or the pharmaceutical composition according to Embodiment 30, for the manufacture of a medicament for the treatment of cancer.

### Embodiment 36

A method of treating cancer, the method comprising administering to a patient in need thereof the CD16a-binding polypeptide according to any of Embodiments 1 to 25, the nucleic acid molecule according to Embodiment 26, the expression vector according to Embodiment 27, and/or the pharmaceutical composition according to Embodiment 30.

### Embodiment 37

A kit comprising the CD16a-binding polypeptide according to any of Embodiments 1 to 25 or the pharmaceutical composition according to Embodiment 30 and, optionally, one or more further therapeutic agent(s).

### Embodiment 38

The kit according to Embodiment 37, wherein the one or more further therapeutic agent(s) is selected from a proteasome inhibitor (for example carlfizomib or bortezomib), an immunomodulatory agent (for example lenalidomide or thalidomide), an alkylator (for example melphalan), a steroid (for example dexamethasone or prednisone), an anti-CD38 agent (for example daratumumab), an immune checkpoint inhibitor (for example a CTLA-4 inhibitor, a PD-1 inhibitor, or a PD-L1 inhibitor), and an ADAM17 inhibitor.

### Embodiment 39

The kit according to Embodiment 37 or Embodiment 38, for use in the treatment of cancer, for example multiple myeloma.

1. A CD16a-binding polypeptide which comprises at least one motif that binds to CD16a, wherein said polypeptide comprises the following structure:
[N-terminal portion]-[Helix 1]-[Separating portion]-[Helix 2]—[C-terminal portion]
the CD16a-binding motif being the portion [Helix 1]-[Separating portion]-[Helix 2].

2. The CD16a-binding polypeptide as claimed in claim 1, wherein:
Helix 1 comprises the sequence X₉X₁₀X₁₁AX₁₃X₁₄EIX₁₇X₁₈ and Helix 2 comprises the sequence X₂₄X₂₅QX₂₇X₂₈AFX₃₁X₃₂X₃₃LX₃₅, [SEQ ID NO 120] wherein,
b) X₉ is A, D, F, H, I, K, L, Q, R, T, V or Y;
X₁₀ is Q;
X₁₁ is A, D, E, F, H, I, K, L, M, N, Q, R, S, T, V, W or Y;
X₁₃ is A, Q or V;
X₁₄ is A, F, H, I, K, L, N, Q, R, S, T, V, W or Y;
X₁₇ is Q or R;
X₁₈ is A, D, E, F, H, I, K, N, Q, R, S, T or V;
X₂₄ is H;
X₂₅ is A or H;
X₂₇ is A, I, K, Q, R, S, Tor V;
X₂₈ is F or Y;
X₃₁ is I or L;
X₃₂ is A, E, H, K, L, N, Q or R;
X₃₃ is K or S;
and X₃₅ is A, H, I, L, M, R or S; or
b) X₉ is V; X₁₀ is Q; X₁₁ is M; X₁₃ is Q; X₁₄ is F; X₁₇ is R; X₁₈ is K; X₂₄ is H; X₂₅ is H; X₂₇ is S; X₂₈ is F; X₃₁ is I; X₃₂ is K; X₃₃ is S and X₃₅ is M, and optionally wherein within Helix 1 and Helix 2, at least 1 and no more than 5 (for example at least 1 and no more than 3) of the X₁ residues are replaced by an alternative residue, and/or at least at least 1 and no more than 5 (for example 1 and no more than 3) of the residues not labelled as Xₙ are replaced by an alternative residue; or
c) X₉ is Q; X₁₀ is F; X₁₁ is Y; X₁₃ is R; X₁₄ is D; X₁₇ is D; X₁₈ is L; X₂₄ is E; X₂₅ is D; X₂₇ is K; X₂₈ is W; X₃₁ is Y; X₃₂ is M; X₃₃ is S and X₃₅ is I, and optionally wherein within Helix 1 and Helix 2, at least 1 and no more than 5 (for example at least 1 and no more than 3) of the X₁ residues are replaced by an alternative residue, and/or at least 1 and no more than 5 (for example at least 1 and no more than 3) of the residues not labelled as X₁ are replaced by an alternative residue; or
d) X₉ is F; X₁₀ is W; X₁₁ is I; X₁₃ is E; X₁₄ is S; X₁₇ is E; X₁₈ is S; X₂₄ is I; X₂₅ is Y; X₂₇ is K; X₂₈ is W; X₃₁ is K; X₃₂ is Y; X₃₃ is S and X₃₅ is A, and optionally wherein within Helix 1 and Helix 2, at least 1 and no more than 5 (for example at least 1 and no more than 3) of the Xₙ residues are replaced by an alternative residue, and/or at least 1 and no more than 5 (for example at least 1 and no more than 3) of the residues not labelled as Xₙ are replaced by an alternative residue.

3. The CD16a-binding polypeptide as claimed in claim 1 or claim 2, wherein:
Helix 1 comprises the sequence X₉X₁₀X₁₁AX₁₃X₁₄EIX₁₇X₁₈ [SEQ ID NO 127] and Helix 2 comprises the sequence X₂₄X₂₅QX₂₇X₂₈AFX₃₁X₃₂X₃₃LX₃₅, [SEQ ID NO 120] wherein,
X₉ is A, D, F, H, I, K, L, Q, R, T, V or Y; X₁₀ is Q; X₁₁ is A, D, E, F, H, I, K, L, M, N, Q, R, S, T, V, W or Y; X₁₃ is A, Q or V; X₁₄ is A, F, H, I, K, L, N, Q, R, S, T, V, W or Y; X₁₇ is Q or R; X₁₈ is A, D, E, F, H, I, K, N, Q, R, S, T or V; X₂₄ is H; X₂₅ is A or H; X₂₇ is A, I, K, Q, R, S, T or V; X₂₈ is F or Y; X₃₁ is I or L; X₃₂ is A, E, H, K, L, N, Q or R; X₃₃ is K or S; and X₃₅ is A, H, I, L, M, R or S.

4. The CD16a-binding polypeptide as claimed in any preceding claim, wherein:
Helix 1 comprises the sequence X₉X₁₀X₁₁AX₁₃X₁₄EIX₁₇X₁₈ [SEQ ID NO 127] and Helix 2 comprises the sequence X₂₄X₂₅QX₂₇X₂₈AFX₃₁X₃₂X₃₃LX₃₅ [SEQ ID NO 120], wherein,
X₉ is D, F, H, I, K, L, Q, R, T, V or Y; X₁₀ is Q; X₁₁ is A, D, E, F, H, I, K, L, M, N, Q, R, S, T, V, W or Y; X₁₃ is A, Q or V; X₁₄ is F, H, I, K, L, N, Q, R, S, T, V, W or Y; X₁₇ is R or Q; X₁₈ is A, D, E, F, H, I, K, N, Q, R, S, T or V; X₂₄ is H; X₂₅ is H or A; X₂₇ is A, I, K, Q, R, T, S or V; X₂₈ is F or Y; X₃₁ is I or L; X₃₂ is A, E, H, K, L, N, Q or R; X₃₃ is K or S; and X₃₅ is A, H, I, L, M, R or S.

5. The CD16a-binding polypeptide as claimed in any preceding claim, wherein:
Helix 1 comprises the sequence X₉X₁₀X₁₁AX₁₃X₁₄EIX₁₇X₁₈ [SEQ ID NO 127] and Helix 2 comprises the sequence X₂₄X₂₅QX₂₇X₂₈AFX₃₁X₃₂X₃₃LX₃₅ [SEQ ID NO 120], wherein,
X₉ is D, F, H, I, K, L, Q, R, T, V or Y; X₁₀ is Q; X₁₁ is A, D, E, F, H, I, K, L, NQ, R, S, T, V, W or Y; X₁₃ is A, Q or V; X₁₄ is H, I, K, L, N, Q, R, S, T, V, W or Y; X₁₇ is R or Q; X₁₈ is A, D, E, F, H, I, K, N, Q, R, S, T or V; X₂₄ is H; X₂₅ is H or A; X₂₇ is A, I, K, Q, R, T or V; X₂₈ is F or Y; X₃₁ is I or L; X₃₂ is A, E, H, K, L, N, Q or R; X₃₃ is K or S; and X₃₅ is A, H, I, L, R or S.

6. The CD16a-binding polypeptide as claimed in claim 5, wherein:
X₉ is D, F, H, I, K, L, Q, R, T, V, or Y; X₁₀ is Q; X₁₁ is A, D, E, F, H, I, K, N, Q, R, S, T, V, W or Y; X₁₃ is A, Q or V; X₁₄ is H, I, K, L, N, Q, R, S, V, W, or Y; X₁₇ is R; X₁₈ is A, D, E, F, H, K, N, Q, R, S or T; X₂₄ is H; X₂₅ is H; X₂₇ is A, I, K, Q, R, T or V; X₂₈ is F; X₃₁ is I or L; X₃₂ is A, E, H, K, N, Q or R; X₃₃ is K or S; and X₃₅ is H, I, L, R or S.

7. The CD16a-binding polypeptide as claimed in claim 5, wherein:
X₉ is D, F, H, I, K, L, Q, T, V, or Y; X₁₀ is Q; X₁₁ is A, D, E, F, H, I, K, L, N, Q, R, S, T, V, W or Y; X₁₃ is A or Q; X₁₄ is H, I, K, L, Q, R, S, T, V, W or Y; X₁₇ is R; X₁₈ is A, D, E, F, H, K, N, Q, R, S, T or V; X₂₄ is H; X₂₅ is H; X₂₇ is A, I, K, Q, R, T or V; X₂₈ is F or Y; X₃₁ is I or L; X₃₂ is A, E, H, K, N, Q or R; X₃₃ is K or S; and X₃₅ is A, H, I, L or R.

8. The CD16a-binding polypeptide as claimed in claim 5, wherein:
X₉ is D, F, H, I, K, L, Q, R, T, V or Y; X₁₀ is Q; X₁₁ is A, D, E, F, H, I, K, N, Q, R, S, T, V, W or Y; X₁₃ is A or Q; X₁₄ is H, I, K, L, Q, R, S, V, W or Y; X₁₇ is R; X₁₈ is A, F, H, K, N, Q, R, S or T; X₂₄ is H; X₂₅ is H; X₂₇ is A, I, K, Q, R, T or V; X₂₈ is F or Y; X₃₁ is I or L; X₃₂ is E, H, K, N, Q or R; X₃₃ is K or S; and X₃₅ is A, H, I, L, R or S.

9. The CD16a-binding polypeptide as claimed in claim 5, wherein:
X₉ is F, L, Q, T or Y; X₁₀ is Q; X₁₁ is A, F, H, I, L, N, Q, S or Y; X₁₃ is A or Q; X₁₄ is I, K, Q, R or V; X₁₇ is R; X₁₈ is A, E, H, K, Q, R, T or V; X₂₄ is H; X₂₅ is H; X₂₇ is A, I, K, Q, R or V; X₂₈ is F; X₃₁ is I; X₃₂ is A, H, K, N, Q or R; X₃₃ is K or S; and X₃₅ is H, I or L.

10. The CD16a-binding polypeptide as claimed in claim 5, wherein:
X₉ is I, L, Q, T or V; X₁₀ is Q; X₁₁ is A, E, F, H, I, S, V or Y; X₁₃ is Q; X₁₄ is K, L, R, V, W or Y; X₁₇ is R; X₁₈ is A, H, K, Q, R, S or T; X₂₄ is H; X₂₅ is H; X₂₇ is I, K, Q, R, T or V; X₂₈ is F; X₃₁ is I or L; X₃₂ is K, N or R; X₃₃ is K or S; and X₃₅ is I or L.

11. The CD16a-binding polypeptide as claimed in claim 5, wherein:
X₉ is I, L, Q or V; X₁₀ is Q; X₁₁ is A, E, H, I, S, W or Y; X₁₃ is Q; X₁₄ is K, L, R, V, W or Y; X₁₇ is R; X₁₈ is A, H, K, Q, R, S or T; X₂₄ is H; X₂₅ is H; X₂₇ is K, Q, R, T or V; X₂₈ is F; X₃₁ is I; X₃₂ is K, N or R; X₃₃ is K or S; and X₃₅ is I or L;
for example wherein:
X₉ is I, L, Q or V; X₁₀ is Q; X₁₁ is A, E, H, I, S or Y; X₁₃ is Q; X₁₄ is K, L, R, V, W or Y; X₁₇ is R; X₁₈ is H, K, Q, R, S or T; X₂₄ is H; X₂₅ is H; X₂₇ is K, Q, R, T or V; X₂₈ is F; X₃₁ is I; X₃₂ is N or K; X₃₃ is K or S; and X₃₅ is I or L.

12. The CD16a-binding polypeptide as claimed in claim 5, wherein:
X₉ is L or V; X₁₀ is Q; X₁₁ is A, I, S or Y; X₁₃ is Q; X₁₄ is K, R or V; X₁₇ is R; X₁₈ is K, Q, R, S or T; X₂₄ is H; X₂₅ is H; X₂₇ is K, R or V; X₂₈ is F; X₃₁ is I; X₃₂ is N or K; X₃₃ is K or S; and X₃₅ is I or L.

13. The CD16a-binding polypeptide as claimed in claim 5, wherein:
X₉ is D, H, I, K, L, Q, T or V; X₁₀ is Q; X₁₁ is A, D, E, F, H, I, K, N, R, S, V, W or Y; X₁₃ is Q; X₁₄ is K, L, Q, R, S, V, W or Y; X₁₇ is R; X₁₈ is A, H, K, N, Q, R, S or T; X₂₄ is H; X₂₅ is H; X₂₇ is A, I, K, Q, R, T or V; X₂₈ is F or Y; X₃₁ is I or L; X₃₂ is A, E, H, K, N, Q or R; X₃₃ is K or S; and X₃₅ is A, I, L, or R; or
X₉ is K, Q or Y; X₁₀ is Q; X₁₁ is I or Q; X₁₃ is Q; X₁₄ is W or Y; X₁₇ is R; X₁₈ is H, K or R; X₂₄ is H; X₂₅ is H; X₂₇ is A, K or T; X₂₈ is F; X₃₁ is I; X₃₂ is A, K or Q; X₃₃ is K or S; and X₃₅ is I or L; or
X₉ is L, V or Y; X₁₀ is Q; X₁₁ is I, N or Q; X₁₃ is Q; X₁₄ is K, R or Q; X₁₇ is R; X₁₈ is E, A or V; X₂₄ is H; X₂₅ is H; X₂₇ is K or Q; X₂₈ is F; X₃₁ is I; X₃₂ is H, K or Q; X₃₃ is K or S; and X₃₅ is I or L.

14. The CD16a-binding polypeptide as claimed in claim 5, wherein:
X₉ is L, Q, T or V; X₁₀ is Q; X₁₁ is I, V or Y; X₁₃ is Q; X₁₄ is K, R or Y; X₁₇ is R; X₁₈ is K, R, S or T; X₂₄ is H; X₂₅ is H; X₂₇ is I, T or V; X₂₈ is F; X₃₁ is I or L; X₃₂ is K or N; X₃₃ is K or S; and X₃₅ is I or L; for example wherein X₉ is L, T or V; X₁₀ is Q; X₁₁ is I, V, or Y; X₁₃ is Q; X₁₄ is K or R; X₁₇ is R; X₁ is R, S or T; X₂₄ is H; X₂₅ is H; X₂₇ is I or V; X₂₈ is F; X₃₁ is I or L; X₃₂ is K or N; X₃₃ is K; and X₃₅ is I or L.

15. The CD16a-binding polypeptide as claimed in claims 2-14, wherein the CD16a binding efficacy is at least 10% of SEQ ID NO: 1; and/or wherein the CD16a-binding polypeptide competes with SEQ ID NO: 1.

16. The CD16a-binding polypeptide as claimed in claim 2 to 14, wherein:
Helix 1 comprises the sequence X₆X₇X₈X₉X₁₀X₁₁AX₁₃X₁₄EIX₁₇X₁₈X₁₉ and/or Helix 2 comprises the sequence X₂₃X₂₄X₂₅QX₂₇X₂₈AFX₃₁X₃₂X₃₃LX₃₅X₃₆X₃₇ [SEQ ID NO 128], wherein,
X₆ is any naturally occurring amino acid (preferably D, E, N or Q; more preferably N) or is absent;
X₇ is any naturally occurring amino acid (preferably H, K or R; more preferably K) or is absent;
X₈ is any naturally occurring amino acid (preferably D, E, N or Q; more preferably E) or is absent;
X₁₉ is any naturally occurring amino acid (preferably G, A, V, L or I; more preferably L) or is absent;
X₂₃ is any naturally occurring amino acid (preferably D, E, N or Q; more preferably N) or is absent;
X₃₆ is any naturally occurring amino acid (preferably D, E, N or Q; more preferably D) or is absent; and
X₃₇ is any naturally occurring amino acid (preferably D, E, N or Q; more preferably D) or is absent;
and preferably wherein X₆ is N; X₇ is K; and X₈ is E; and/or wherein X₃₆ is D; and X₃₇ is D;
for example wherein X₆ is N; X₇ is K; X₈ is E; X₁₉ is L; X₂₃ is N; X₃₆ is D; and X₃₇ is D.

17. The CD16a-binding polypeptide as claimed in any of claims 2 to 4, wherein:
Helix 1 comprises the sequence NKEVQMAQFEIRKL [SEQ ID NO 129] and Helix 2 comprises the sequence NHHQSFAFIKSLMDD [SEQ ID NO 130];
and optionally wherein, at least 1 and no more than 5 (for example 1, 2, 3, 4 or 5) (for example, at least 1 and no more than 3 (for example 1, 2, or 3)) residues in the sequence of Helix 1 and/or Helix 2 are replaced by an alternative residue (for example replaced by an alternative residue that is a conservative replacement).

18. The CD16a-binding polypeptide as claimed in claim 17, wherein the CD16a binding efficacy is at least 10% of SEQ ID NO: 1; and/or wherein the CD16a-binding polypeptide competes with SEQ ID NO: 1.

19. The CD16a-binding polypeptide as claimed in claim 2, wherein:
Helix 1 comprises the sequence NKEQFYARDEIDLL [SEQ ID NO 131] and Helix 2 comprises the sequence NEDQKWAFYMSLIDD [SEQ ID NO 132];
and optionally wherein, at least 1 and no more than 5 (for example 1, 2, 3, 4 or 5) (for example, at least 1 and no more than 3 (for example 1, 2, or 3)) residues in the sequence of Helix 1 and/or Helix 2 are replaced by an alternative residue (for example replaced by an alternative residue that is a conservative replacement).

20. The CD16a-binding polypeptide as claimed in claim 19, wherein the CD16a binding efficacy is at least 10% of SEQ ID NO: 74; and/or wherein the CD16a-binding polypeptide competes with SEQ ID NO: 74.

21. The CD16a-binding polypeptide as claimed in claim 2, wherein:
Helix 1 comprises the sequence NKEFWIAESEIESL [SEQ ID NO 133] and Helix 2 comprises the sequence NIYQKWAFKYSLADD [SEQ ID NO 134];
and optionally wherein, at least 1 and no more than 5 (for example 1, 2, 3, 4 or 5) (for example, at least 1 and no more than 3 (for example 1, 2, or 3) residues in the sequence of Helix 1 and/or Helix 2 are replaced by an alternative residue (for example replaced by an alternative residue that is a conservative replacement).

22. The CD16a-binding polypeptide as claimed in claim 21, wherein the CD16a binding efficacy is at least 10% of SEQ ID NO: 75; and/or wherein the CD16a-binding polypeptide competes with SEQ ID NO: 75.

23. The CD16a-binding polypeptide as claimed in any preceding claim wherein the separating portion is a sequence of 1 to 5 (preferably 2 to 5, for example 2, 3, 4 or 5; or for example 3 to 5, for example 3, 4 or 5, and more preferably 3) naturally occurring amino acids.

24. The CD16a-binding polypeptide as claimed in any preceding claim wherein the separating portion has the sequence X₂₀X₂₁X₂₂, wherein
X₂₀ is any naturally occurring amino acid (preferably S, T, M, P, F, Y or W; more preferably P or T);
X₂₁ is any naturally occurring amino acid (preferably D, E, N or Q; more preferably N);
X₂₂ is any naturally occurring amino acid (preferably G, A, V, L or I; more preferably L); and wherein optionally one or two (for example optionally 1) of X₂₀, X₂₁, X₂₂ are absent;
and preferably wherein X₂₀ is P or T; X₂₁ is N; and X₂₂ is L (for example, the separating portion has the sequence PNL or TNL).

25. The CD16a-binding polypeptide as claimed in any preceding claim, wherein the N-terminal portion is
absent or
is a sequence of 1 to 15 (preferably 1 to 10 or 1 to 8, more preferably 1 to 5, for example 1, 2, 3 4 or 5) naturally occurring amino acids.

26. The CD16a-binding polypeptide as claimed in any preceding claim wherein the N-terminal portion has the sequence X₁X₂X₃X₄X₅, wherein
X₁ is any naturally occurring amino acid (preferably G, A, V, L or I; more preferably V or G; and most preferably V) or is absent;
X₂ is any naturally occurring amino acid (preferably D, E, N or Q; more preferably D) or is absent;
X₃ is any naturally occurring amino acid (preferably D, E, N or Q; more preferably N) or is absent;
X₄ is any naturally occurring amino acid (preferably H, K or R; more preferably K) or is absent; and
X₈ is any naturally occurring amino acid (preferably F, Y or W; more preferably F) or is absent;
and preferably wherein X₁ is V, G or absent (preferably V or absent), X₂ is D or absent, X₃ is N or absent, X₄ is K or absent, and X₈ is F or absent.

27. The CD16a-binding polypeptide as claimed in any preceding claim, wherein the N-terminal portion is absent, or the N-terminal portion has the sequence X₁X₂X₃X₄X₅ wherein
X₁ is V or G (preferably V), X₂ is D, X₃ is N, X₄ is K, and X₈ is F; or
X₁ is absent, X₂ is D, X₃ is N, X₄ is K, and X₈ is F; or
X₁ is absent, X₂ is absent, X₃ is N, X₄ is K, and X₈ is F; or
X₁ is absent, X₂ is absent, X₃ is absent, X₄ is K, and X₈ is F; or
X₁ is absent, X₂ is absent, X₃ is absent, X₄ is absent, and X₈ is F.

28. The CD16a-binding polypeptide as claimed in any preceding claim, wherein the C-terminal portion is
absent or
is a sequence of 1 to 50 (for example 1 to 35, 1 to 30, 15 to 25 or 18 to 22) naturally occurring amino acids.

29. CD16a-binding polypeptide as claimed in claim 28 wherein the C-terminal portion has the sequence X₃₈X₃₉QSANLLAEAKKLNDAQX₅₆X₅₇X₅₈ [SEQ ID NO 135], wherein,
X₃₈ is a sequence of 1 to 14 (preferably 1 to 9 or 1 to 7, more preferably 1 to 4, for example 1, 2, 3 or 4) naturally occurring amino acids (and preferably X₃₈ is any naturally occurring amino acid (most preferably X₃₈ is P));
X₃₉ is any naturally occurring amino acid (preferably S, T, M, P, F, Y or W; more preferably S);
X₅₆ is any naturally occurring amino acid (preferably G, A, V, L or I; more preferably A) or is absent;
X₅₇ is any naturally occurring amino acid (preferably P) or is absent; and
X₅₈ is any naturally occurring amino acid (preferably H, K or R; more preferably K) or is absent;
(for example X₃₈ is P; X₃₉ is S; X₅₆ is A or absent; X₅₇ is P or absent; X₅₈ is K or absent)
and optionally wherein, at least 1 and no more than 5 (for example 1, 2, 3 4 or 5) (preferably at least 1 and no more than 3 (for example 1, 2, or 3)) of the residues in the sequence QSANLLAEAKKLNDAQ [SEQ ID NO 136] are replaced by an alternative residue;
and preferably optionally wherein, at least 1 and no more than 5 (for example 1, 2, 3 4 or 5) (preferably at least 1 and no more than 3 (for example 1, 2, or 3)) of the residues in the sequence QSANLLAEAKKLNDAQ [SEQ ID NO 136] are replaced by an alternative residue that is a conservative replacement.

30. CD16a-binding polypeptide as claimed in claim 29 wherein the C-terminal portion has the sequence X₃₈X₃₉QSANLLAEAKKLNDAQXs₆X₅₇X₅₈ [SEQ ID NO 135], wherein
X₅₆ is A, X₅₇ is P, and X₅₈ is K;
X₅₆ is A; or X₅₇ is P, and X₅₈ is absent;
X₅₆ is A, X₅₇ is absent; and X₅₈ is absent; or
X₅₆ is absent, X₅₇ is absent, and X₅₈ is absent;
and optionally wherein, at least 1 and no more than 3 (for example 1, 2, or 3) of the residues in the sequence QSANLLAEAKKLNDAQ [SEQ ID NO 136] are replaced by an alternative residue;
and preferably optionally wherein, at least 1 and no more than 3 (for example 1, 2, or 3) of the residues in the sequence QSANLLAEAKKLNDAQ [SEQ ID NO 136] are replaced by an alternative residue that is a conservative replacement; and
preferably wherein X₃₈ is P and X₃₉ is S.

31. The CD16a-binding polypeptide as claimed in any preceding claim, wherein:
(i) the separating portion has the sequence X₂₀X₂₁X₂₂; and/or
the N-terminal portion has the sequence X₁X₂X₃X₄X₅; and/or the C-terminal portion has the sequence PSQSANLLAEAKKLNDAQX₅₆X₅₇X₅₈ [SEQ ID NO 137];
wherein, in said separating portion,
X₂₀ is P or T; X₂₁ is N; X₂₂ is L;
wherein in said N-terminal portion,
X₁ is V or G (preferably V), or absent; X₂ is D or absent; X₃ is N or absent; X₄ is K or absent; X₈ is F or absent;
and wherein in said C-terminal portion,
X₅₆ is A or absent; X₅₇ is P or absent; X₅₈ is K or absent;
or (ii) the separating portion, N-terminal portion, and C-terminal portion are as defined in (i), wherein optionally
(a) within each portion 1, 2 or 3 residues are replaced by an alternative residue; or
(b) within those portions taken together at least 1 and no more than 10 (for example, not more than 5, for example 1, 2, 3, 4, or 5) residues are replaced by an alternative residue.

32. The CD16a-binding polypeptide as claimed in any preceding claim, wherein said separating portion has the sequence PNL or TNL; and/or wherein said N-terminal portion has the sequence VDNKF [SEQ ID NO 138].

33. The CD16a-binding polypeptide as claimed in any preceding claim. which consists of a CD16a-binding polypeptide as defined in any preceding claim; and optionally comprising an additional binding moiety.

34. A CD16a-binding polypeptide as claimed in any preceding claim, wherein the CD16a-binding motif sequence is selected from SEQ ID NOs. 150 to 221;
preferably from SEQ ID NOs. 166, 168, 178, 182, or 202; or
preferably from SEQ ID NOs. 164, 166, 168, or 200 (for example 164, 166 or 168);
and wherein optionally from 1 to 5 (preferably 1, 2 or 3) residues in the sequence are replaced by an alternative residue, and preferably a residue that is a conservative replacement.

35. The CD16a-binding polypeptide as claimed in claim 1 or 2, which comprises a sequence selected from SEQ ID NOs. 1-75, and wherein optionally from 1 to 5 (preferably 1, 2 or 3) residues in the sequence are replaced by an alternative residue, and preferably optionally from 1 to 5 (preferably 1, 2 or 3) residues in the sequence are replaced by an alternative residue that is a conservative replacement; or
the CD16a-binding polypeptide as claimed in any one of claims 1 to 3, which comprises a sequence selected from SEQ ID NOs. 1-73, and wherein optionally from 1 to 5 (preferably 1, 2 or 3) residues in the sequence are replaced by an alternative residue, and preferably optionally from 1 to 5 (preferably 1, 2 or 3) residues in the sequence are replaced by an alternative residue that is a conservative replacement; or
the CD16a-binding polypeptide as claimed in any one of claims 1 to 4, which comprises a sequence selected from SEQ ID NOs. 1 or 11 to 67, and wherein optionally from 1 to 5 (preferably 1, 2 or 3) residues in the sequence are replaced by an alternative residue, and preferably optionally from 1 to 5 (preferably 1, 2 or 3) residues in the sequence are replaced by an alternative residue that is a conservative replacement; or
the CD16a-binding polypeptide as claimed in any one of claims 1 to 5, which comprises a sequence selected from SEQ ID NOs. 11 to 67, and wherein optionally from 1 to 5 (preferably 1, 2 or 3) residues in the sequence are replaced by an alternative residue, and preferably optionally from 1 to 5 (preferably 1, 2 or 3) residues in the sequence are replaced by an alternative residue that is a conservative replacement.

36. The CD16a-binding polypeptide as claimed in claims 1 to 4, wherein the sequence of the CD16a-binding polypeptide is selected from:
[SEQ ID NO: 1]
VDNKFNKEVQMAQFEIRKLPNLNHHQSFAFIKSLMDDPSQSANLLAEA
KKLNDAQAPK
or
[SEQ ID NO: 51]
VDNKFNKEQQIAQYEIRKLPNLNHHQTFAFIKSLLDDPSQSANLLAEA
KKLNDAQAPK

37. The CD16a-binding polypeptide as claimed in claim 1 or claim 2, wherein the sequence of the CD16a-binding polypeptide is selected from:
[SEQ ID NO: 74]
VDNKFNKEQFYARDEIDLLPNLNEDQKWAFYMSLIDDPSQSANLLAE
AKKLNDAQAPK;
and;
[SEQ ID NO: 75]
VDNKFNKEFWIAESEIESLPNLNIYQKWAFKYSLADDPSQSANLLAEA
KKLNDAQAPK.

38. The CD16a-binding polypeptide as claimed in claim 1 or claim 2, wherein the sequence of the CD16a-binding polypeptide is selected from SEQ ID NOs. 12, 17, 19, 29, 33, 49, 51, and 53; for example SEQ ID NOs. 17, 19, 29, 33, and 53;
or wherein the sequence of the CD16a-binding polypeptide is selected from SEQ ID NOs. 19, 33, 17, 29, 53, 16, 25, 15, 51, 36, 49, 55, 43, 24, 56, 12, 28, 21, 59, 52, 32, 18, 27, 35 or11; or34, 45 or51; or26, 35 or47

39. The CD16a-binding polypeptide as claimed in claim 1 or claim 2, wherein the sequence of the CD16a-binding polypeptide is selected from SEQ ID NOs.15, 17, 19, and 51; for example 15, 19 or 17.

40. The CD16a-binding polypeptide according to any preceding claim, wherein the CD16a-binding polypeptide does not comprise methionine.

41. The CD16a-binding polypeptide with the sequence according to any preceding claim, wherein, at a position at which a methionine residue is recited, the polypeptide has the sequence with the methionine residue independently substituted for a different naturally occurring amino acid or an unnatural amino acid (for example a different naturally occurring amino acid or norleucine); and preferably each methionine residue is independently substituted for an amino acid selected from isoleucine (I), leucine (L), glutamine (Q) and norleucine; and more preferably each methionine residue is independently substituted for a norleucine or isoleucine.

42. The CD16a-binding polypeptide according to any preceding claim, wherein one or more residues (for example 1 to 5 residues, for example 1, 2, 3, 4 or 5) of the CD16a-binding polypeptide is/are substituted for an unnatural amino acid, for example norleucine;
and/or wherein one or more methionine residues, when present (for example 1 or 2 methionine residue(s) when present), is/are substituted for an unnatural amino acid, for example norleucine;
and/or wherein one or more leucine residues, when present (for example 1 to 5 leucine residues, when present), is/are substituted for norleucine;
and/or wherein one or more methionine residues, when present (for example 1 or 2 methionine residue(s) when present), is/are oxidised (for example is/are Met(O)).

43. A CD16a-binding oligomer, which comprises at least two CD16a-binding polypeptides as defined in any one of claims 1 to 42, for example 2, 3, 4, or 5 CD16a-binding polypeptides as defined in any one of claims 1 to 42.

44. The CD16a-binding oligomer as claimed in claim 43, which comprises at least two CD16a-binding polypeptides, wherein a first CD16a-binding polypeptide is as defined in any of claims 2-42, and a second CD16a-binding polypeptide is as defined in any of claims 2-42.

45. The CD16a-binding oligomer as claimed in claim 44, which comprises at least two CD16a-binding polypeptides, wherein the first and second CD16a-binding polypeptide have the same sequence.

46. The CD16a-binding oligomer as claimed in claim 44, which comprises at least two CD16a-binding polypeptides, wherein the first and second CD16a-binding polypeptide have a different sequence.

47. The CD16a-binding oligomer according to claim 43, which comprises at least two CD16a-binding polypeptides, wherein:
a first CD16a-binding polypeptide comprises a first CD16a binding motif selected from SEQ ID Nos. 150-221 or comprises a sequence selected from SEQ ID NOs. 1-75;
and a second CD16a-binding polypeptide comprises a second CD16a binding motif selected from SEQ ID NOs. 150-221 or comprises a sequence selected from SEQ ID NOs. 1-75.

48. The CD16a-binding oligomer according to claim 47, which comprises at least two CD16a-binding polypeptides, wherein the first and second CD16a-binding polypeptides have the same sequence, or the first and second CD16a binding motifs selected have the same sequence.

49. The CD16a-binding oligomer according to claim 47, which comprises at least two CD16a-binding polypeptides, wherein the first and second binding motif selected or first and second CD16a-binding polypeptide have a different sequence.

50. The CD16a-binding oligomer as claimed in any of claims 43-49, wherein the CD16a-binding polypeptides are each separated by a linker.

51. The CD16a-binding oligomer as claimed claim 50, wherein the linker is a sequence of 1 to 50 (for example 1 to 25) naturally occurring amino acids;
preferably 1 to 25 (for example 1 to 20) naturally occurring amino acids selected from the group consisting of G, S and T (preferably G and S).

52. The CD16a-binding oligomer as claimed in claims 50 or 51, wherein the linker is G or comprises the sequence GGGSG [SEQ ID NO 139], GGGGS [SEQ ID NO 140], GGSGG [SEQ ID NO 141], GSGGG [SEQ ID NO 142] and/or SGGGG [SEQ ID NO 143]; for example wherein the linker is G or comprises or has the sequence GGGSG, GGGSGGGGSG [SEQ ID NO 144], GGGSGGGGSGGGGSG [SEQ ID NO 145], GGGSGGGGSGGGGSGGGGSG [SEQ ID NO 146], GGSGG, GGSGGGGSGG [SEQ ID NO 147], GGSGGGGSGGGGSGG [SEQ ID NO 148] or GGSGGGGSGGGGSGGGGSGG [SEQ ID NO 149].

53. The CD16a-binding oligomer as claimed in any of claims 50-52, wherein the CD16a-binding oligomer comprises at least 2 (for example 2) CD16a-binding polypeptides, and the CD16a-binding oligomer comprises the following structure:
[N-terminal portion]-[Helix 1]-[Separating portion]-[Helix 2]—[C-terminal portion]-[Linker]-[N-terminal portion]-[Helix 1]-[Separating portion]-[Helix 2]—[C-terminal portion];
wherein the linker is a sequence of 1 to 50 naturally occurring amino acids, preferably 1 to 25 (for example 1-20) naturally occurring amino acids selected from the group consisting of G, S and T (preferably G and S), for example, wherein the linker is G or comprises the sequence GGGSG, GGGGS, GGSGG, GSGGG and/or SGGGG; for example wherein the linker is G or comprises or has the sequence GGGSG, GGGSGGGGSG, GGGSGGGGSGGGGSG, GGGSGGGGSGGGGSGGGGSG, GGSGG, GGSGGGGSGG, GGSGGGGSGGGGSGG or GGSGGGGSGGGGSGGGGSGG;
wherein each N-terminal portion in the oligomer may have the same sequence or have different sequences;
each C-terminal portion in the oligomer may have the same sequence or have different sequences;
each separating portion in the oligomer may have the same sequence or have different sequences;
each Helix 1 portion in the oligomer may have the same sequence or have different sequences; and
each Helix 2 portion in the oligomer may have the same sequence or have different sequences.

54. The CD16a-binding oligomer as claimed in any one of claims 50-53, wherein the CD16a-binding oligomer comprises at least 3 (for example 3) CD16a-binding polypeptides, and the CD16a-binding oligomer comprises the following structure
[N-terminal portion]-[Helix 1]-[Separating portion]-[Helix 2]—[C-terminal portion]-[Linker]-[N-terminal portion]-[Helix 1]-[Separating portion]-[Helix 2]—[C-terminal portion]-[Linker]-[N-terminal portion]-[Helix 1]-[Separating portion]-[Helix 2]—[C-terminal portion];
wherein each linker is a sequence of 1 to 50 naturally occurring amino acids, preferably 1 to 25 (for example 1-20) naturally occurring amino acids selected from the group consisting of G, S and T (preferably G and S) (for example, wherein the linker is G or comprises the sequence GGGSG, GGGGS, GGSGG, GSGGG and/or SGGGG; for example wherein the linker is G or comprises or has the sequence GGGSG, GGGSGGGGSG, GGGSGGGGSGGGGSG, GGGSGGGGSGGGGSGGGGSG, GGSGG, GGSGGGGSGG, GGSGGGGSGGGGSGG or GGSGGGGSGGGGSGGGGSGG;
and wherein each linker portion in the oligomer may have the same sequence or have different sequences;
each N-terminal portion in the oligomer may have the same sequence, have different sequences, or two may have the same sequence and one may have a different sequence;
each C-terminal portion may have the same sequence, have different sequences, or two may have the same sequence and one may have a different sequence;
each separating portion in the oligomer may have the same sequence, have different sequences, or two may have the same sequence and one may have a different sequence;
each Helix 1 portion in the oligomer may have the same sequence, have different sequences, or two may have the same sequence and one may have a different sequence; and
each Helix 2 portion in the oligomer may have the same sequence, have different sequences, or two may have the same sequence and one may have a different sequence.

55. The CD16a-binding oligomer as claimed in claim 43, 44 or 53, which comprises the sequence
[SEQ ID NO. 242]
VDNKFNKEVQMAQFEIRKLPNLNHHQSFAFIKSLMDDPSQSANLLAEA
KKLNDAQAPKGGGSGGGGSGGGGSGVDNKFNKEVQMAQFEIRKLPN
LNHHQSFAFIKSLMDDPSQSANLLAEAKKLNDAQAPK.

56. The CD16a-binding oligomer as claimed in claim 43, 44 or 53, which comprises the sequence
SEQ ID NO. 243]
VDNKFNKEQFYARDEIDLLPNLNEDQKWAFYMSLIDDPSQSANLLAE
AKKLNDAQAPKGGGSGGGGSGGGGSGVDNKFNKEVQMAQFEIRKLP
NLNHHQSFAFIKSLMDDPSQSANLLAEAKKLNDAQAPK.

57. The CD16a-binding oligomer as claimed in claim 43, 44 or 53, which comprises the sequence
SEQ ID NO. 244]
VDNKFNKEFWIAESEIESLPNLNIYQKWAFKYSLADDPSQSANLLAEA
KKLNDAQAPKGGGSGGGGSGGGGSGVDNKFNKEVQMAQFEIRKLPN
LNHHQSFAFIKSLMDDPSQSANLLAEAKKLNDAQAPK.

58. The CD16a-binding polypeptide as claimed in one of claims 2 to 42, or CD16a-binding oligomer as claimed in any one of claims 43 to 57, which further comprises an additional functional portion (for example at least one, at least two, or at least three; for example 1, 2, 3, 4 or 5 additional functional portions).

59. The CD16a-binding polypeptide or CD16a-binding oligomer as claimed in claim 58, wherein the additional functional portion comprises an immune signalling molecule, for example a cytokine, for example IL-15 or derivatives thereof.

60. The CD16a-binding polypeptide or CD16a-binding oligomer as claimed in claim 58, wherein the additional functional portion comprises an additional binding moiety.

61. The CD16a-binding polypeptide or CD16a-binding oligomer as claimed in claim 60, wherein the additional binding moiety is specific for a cancer cell surface target, for example a myeloma cell surface antigen, for example BCMA, or is specific for a NK cell target.

62. The CD16a-binding polypeptide or CD16a-binding oligomer as claimed in claim 58, wherein the CD16a-binding polypeptide or CD16a-binding oligomer comprises at least two (for example 2, 3, 4 or 5) additional functional portions, wherein each additional functional portion may be the same or may be different.

63. The CD16a-binding polypeptide or CD16a-binding oligomer as claimed in claim 58, wherein the CD16a-binding polypeptide or CD16a-binding oligomer comprises at least two (for example 2, 3, 4 or 5) additional functional portions, wherein a first additional functional portion comprises an additional binding moiety (for example an additional binding moiety specific for a cancer cell surface target, for example a myeloma cell surface antigen, for example BCMA, or is specific for a NK cell target); and wherein the second additional functional portion comprises an immune signalling molecule, for example a cytokine, for example IL-15 or derivatives thereof; or
wherein a first additional functional portion comprises an additional binding moiety (for example an additional binding moiety specific for a cancer cell surface target, for example a myeloma cell surface antigen, for example BCMA, or is specific for a NK cell target); and wherein the second additional functional portion comprises an additional binding moiety (for example an additional binding moiety specific for a cancer cell surface target, for example a myeloma cell surface antigen, for example BCMA, or is specific for a NK cell target).

64. The CD16a-binding polypeptide or CD16a-binding oligomer as claimed in claim 63 comprising an additional functional portion, wherein the CD16a-binding polypeptide or CD16a-binding oligomer comprises at least 3 (for example 3, 4 or 5) additional functional portions, wherein a third additional functional portion comprises an additional binding moiety (for example an additional binding moiety specific for a cancer cell surface target, for example a myeloma cell surface antigen, for example BCMA, or is specific for a NK cell target), or comprises an immune signalling molecule, for example a cytokine, for example IL-15 or derivatives thereof; and preferably wherein a third additional functional portion comprises an additional binding moiety (for example an additional binding moiety specific for a cancer cell surface target, for example a myeloma cell surface antigen, for example BCMA, or is specific for a NK cell target).

65. The CD16a-binding polypeptide or CD16a-binding oligomer as claimed in any of claims 58-64, wherein the additional functional portion(s) is/are separated from the CD16a-binding polypeptide or the or CD16a-binding oligomer by a linker,
for example wherein the linker is a sequence of 1 to 50 naturally occurring amino acids, preferably 1 to 25 (for example 1 to 20), naturally occurring amino acids selected from the group consisting of G, S and T (preferably G and S).

66. The CD16a-binding polypeptide or CD16a-binding oligomer as claimed in claim 65, wherein the linker is G or comprises the sequence GGGSG, GGGGS, GGSGG, GSGGG and/or SGGGG; for example wherein the linker is G or comprises or has the sequence GGGSG, GGGSGGGGSG, GGGSGGGGSGGGGSG, GGGSGGGGSGGGGSGGGGSG, GGSGG, GGSGGGGSGG, GGSGGGGSGGGGSGG or GGSGGGGSGGGGSGGGGSGG.

67. The CD16a-binding polypeptide or CD16a-binding oligomer as claimed in claims 65 or 66 comprising an additional functional portion, wherein the CD16a-binding polypeptide comprises the following structure:
[N-terminal portion]-[Helix 1]-[Separating portion]-[Helix 2]—[C-terminal portion]-[linker]-[additional functional portion]; or
[additional functional portion]-[linker]-[N-terminal portion]-[Helix 1]-[Separating portion]-[Helix 2]—[C-terminal portion]; or
[N-terminal portion]-[Helix 1]-[Separating portion]-[Helix 2]—[C-terminal portion]-[linker]-[additional functional portion]-[additional functional portion];
[additional functional portion]-[additional functional portion]-[linker]-[N-terminal portion]-[Helix 1]-[Separating portion]-[Helix 2]—[C-terminal portion]; or
[additional functional portion]-[linker]-[N-terminal portion]-[Helix 1]-[Separating portion]-[Helix 2]—[C-terminal portion]-[additional functional portion];
(and preferably
[additional functional portion]-[linker]-[N-terminal portion]-[Helix 1]-[Separating portion]-[Helix 2]—[C-terminal portion];
[additional functional portion]-[additional functional portion]-[linker]-[N-terminal portion]-[Helix 1]-[Separating portion]-[Helix 2]—[C-terminal portion])
wherein each linker is a sequence of 1 to 50 (for example 1-25) naturally occurring amino acids, preferably 1 to 25 (for example 1-20) naturally occurring amino acids selected from the group consisting of G, S and T (preferably G and S) (for example, wherein the linker is G or comprises the sequence GGGSG, GGGGS, GGSGG, GSGGG and/or SGGGG; for example wherein the linker is G or comprises or has the sequence GGGSG, GGGSGGGGSG, GGGSGGGGSGGGGSG, GGGSGGGGSGGGGSGGGGSG, GGSGG, GGSGGGGSGG, GGSGGGGSGGGGSGG or GGSGGGGSGGGGSGGGGSGG).
and wherein, when more than one additional functional portion is present, each additional functional may be the same, or be different.

68. A nucleic acid molecule encoding the CD16a-binding polypeptide as claimed in any of claims 1 to 42 or 58 to 67; or encoding the CD16a-binding oligomer as claimed in any of claims 43 to 67.

69. An expression vector comprising the nucleic acid molecule as claimed in claim 68.

70. A host cell comprising the nucleic acid molecule as claimed in claim 68 or the expression vector as claimed in claim 69.

71. A method of making the CD16a-binding polypeptide as claimed in any of claims 1 to 42 or 58 to 67, or the CD16a-binding oligomer as claimed in any of claims 43 to 67, the method comprising maintaining the host cell of claim 70 under optimal conditions for expression of the nucleic acid and isolating the CD16a-binding polypeptide.

72. A CD16a binder-drug conjugate comprising the CD16a-binding polypeptide as claimed in any of claims 1 to 42 or 58 to 67, or the CD16a-binding oligomer as claimed in any of claims 43 to 67, and an additional therapeutic agent.

73. The CD16a binder-drug conjugate as claimed in claim 72, wherein the additional therapeutic agent is a cytotoxic drug, for example MMAF, MMAE, doxorubicin, pyrrolobenzodiazepine, amanitin, maytansinoids, duostatins, mitomycin C, desmethyltopotecan or SN-38.

74. The CD16a binder-drug conjugate as claimed in claim 72 or claim 73, wherein the hBCMA-binding polypeptide is connected to the additional therapeutic agent via a linker.

75. A pharmaceutical composition comprising the CD16a-binding polypeptide as defined in any of claims 1 to 42 or 58 to 67, the CD16a-binding oligomer as claimed in any of claims 43 to 67, the nucleic acid molecule as claimed in claim 68 or the expression vector as claimed in claim 67.

76. The CD16a-binding polypeptide as claimed in any of claims 1 to 42 or 58 to 67, the CD16a-binding oligomer as claimed in any of claims 43 to 67, the nucleic acid molecule as claimed in claim 68, the expression vector as claimed in claim 69, the CD16a binder-drug conjugate as claimed in any of claims 72 to 74, and/or the pharmaceutical composition as claimed in claim 75, for use in medicine.

77. The CD16a-binding polypeptide as claimed in any of claims 1 to 42 or 58 to 67, the CD16a-binding oligomer as claimed in any of claims 43 to 67, the nucleic acid molecule as claimed in claim 68, the expression vector as claimed in claim 69, the CD16a binder-drug conjugate as claimed in any of claims 72 to 74, and/or the pharmaceutical composition as claimed in claim 75, for use in the treatment of cancer.

78. The CD16a-binding polypeptide as claimed in any of claims 1 to 42 or 58 to 67, the CD16a-binding oligomer as claimed in any of claims 43 to 67, the nucleic acid molecule as claimed in claim 68, the expression vector as claimed in claim 69, the CD16a binder-drug conjugate as claimed in any of claims 72 to 74, and/or the pharmaceutical composition as claimed in claim 75, for use as claimed in claim 77, wherein the cancer is multiple myeloma.

79. Use of the CD16a-binding polypeptide as claimed in any of claims 1 to 42 or 58 to 67, the CD16a-binding oligomer as claimed in any of claims 43 to 67, the nucleic acid molecule as claimed in claim 68, the expression vector as claimed in claim 69, the CD16a binder-drug conjugate as claimed in any of claims 72 to 74, and/or the pharmaceutical composition as claimed in claim 75, for the manufacture of a medicament for the treatment of cancer.

80. A method of treating cancer, the method comprising administering to a patient in need thereof the CD16a-binding polypeptide as claimed in any of claims 1 to 42 or 58 to 67, the CD16a-binding oligomer as claimed in any of claims 43 to 67, the nucleic acid molecule as claimed in claim 68, the expression vector as claimed in claim 69, the CD16a binder-drug conjugate as claimed in any of claims 72 to 74, and/or the pharmaceutical composition as claimed in claim 75.

81. A kit comprising the CD16a-binding polypeptide as claimed in any of claims 1 to 42 or 58 to 67, the CD16a-binding oligomer as claimed in any of claims 43 to 67, the CD16a binder-drug conjugate as claimed in any of claims 72 to 74, or the pharmaceutical composition as claimed in claim 75 and, optionally, one or more further therapeutic agent(s).

82. The kit as claimed in claim 81, wherein the one or more further therapeutic agent(s) is selected from a proteasome inhibitor (for example carlfizomib or bortezomib), an immunomodulatory agent (for example lenalidomide or thalidomide), an alkylator (for example melphalan or melflufen), a steroid (for example dexamethasone or prednisone), an anti-CD38 agent (for example daratumumab), an immune checkpoint inhibitor (for example a CTLA-4 inhibitor, a PD-1 inhibitor, or a PD-L1 inhibitor), and an ADAM17 inhibitor.

83. The kit as claimed in claim 81 or claim 82, for use in the treatment of cancer, for example multiple myeloma.
